(19) United States
(12) Patent Application Publication
(10) Pub. No.: US 2023/0027929 A1
(43) Pub. Date: Jan. 26, 2023

(54) MrgprX2 Antagonists and Uses Thereof

(71) Applicant: Dermira, Inc., Menlo Park, CA (US)

(72) Inventors: Ferda Cevikbas, Menlo Park, CA (US); Christopher Pearson, Menlo Park, CA (US); Donnya Etheridge, Menlo Park, CA (US); Laura Gleave, Menlo Park, CA (US); Mark Duggan, Menlo Park, CA (US); Nina Connelly Ursinyova, Menlo Park, CA (US); Vasileios Roumpelakis, Menlo Park, CA (US)

(21) Appl. No.: 17/773,404

(22) PCT Filed: Nov. 5, 2020
(86) PCT No.: PCT/US2020/059226

§ 371 Date: Apr. 29, 2022

Related U.S. Application Data
(60) Provisional application No. 62/931,174, filed on Nov. 5, 2019.
(60) Provisional application No. 62/931,627, filed on Nov. 6, 2019.
(60) Provisional application No. 63/046,476, filed on Jun. 30, 2020.

(51) Int. Cl. C07D 213/75; A61K 31/4412; A61K 31/4433; C07D 405/12; A61K 31/443; A61K 31/444; A61K 31/4439; C07D 401/12; A61K 31/4427; C07D 413/04; C07D 241/20; A61K 31/4965; A61K 31/436; C07D 498/04; C07D 239/47; A61K 31/513; A61K 31/506; C07D 403/12; C07D 403/04; C07D 401/06; A61K 9/00

(52) U.S. Cl. CPC C07D 213/75; A61K 31/4412; A61K 31/4433; C07D 405/12; A61K 31/443; A61K 31/444; A61K 31/4439; C07D 401/12; A61K 31/4427; C07D 413/04; C07D 241/20; A61K 31/4965; A61K 31/436; C07D 498/04; C07D 239/47; A61K 31/513; A61K 31/506; C07D 403/12; C07D 403/04; C07D 401/06; A61K 9/0014; A61K 9/0053

(57) ABSTRACT

The present disclosure is directed to use of MrgprX2 antagonists in the treatment of inflammatory disorders, e.g., inflammatory disorders of the skin. This invention is also directed to pharmaceutical compositions comprising a MrgprX2 antagonist and a pharmaceutically or orally acceptable carrier for administration.

### CROSS-REFERENCE TO RELATED APPLICATIONS

This application claims priority to and the benefit of U.S. Provisional Application Ser. Nos. 62/931,174, filed on Nov. 5, 2019, 62/931,627, filed on Nov. 6, 2019, and 63/046,476, filed on Jun. 30, 2020, the contents of each of which are hereby incorporated by reference in their entireties.

### BACKGROUND

Atopic dermatitis (AD) is the most common inflammatory skin disease with an overall prevalence of 6% in adults in the US, and 1-3% of adults and 15-20% of children worldwide. 17.8 million Americans suffer from AD. The disease onset is typically in childhood, and skin manifestations are visible by the age of 1 year in 60% of the patients. Clinical manifestations are erythematous papules and plaques, oozing, crust, hypopigmentation and lichenification. The hallmark symptom of AD, however, is intense chronic itch that persists more than 6 weeks. Despite high prevalence of chronic itch in AD patients, there is no effective first-line treatment available with a good safety profile. Itch has a significant impact on the quality of life of these patients, including sleep impairment, ultimately leading to poor performance at work or school. Health-related quality of life in children is inversely correlated with the severity of the disease. Sleep is affected by persisting nocturnal pruritus.

Oral anti-histamines provide modest symptomatic relief due to their sedative effects without directly altering pruritus. Topical calcineurin inhibitors (TCI) as well as topical corticosteroids (TCS) might be helpful in reducing the pruritus. However, their adverse effects (skin atrophy, hypopigmentation, and telangiectasia in case of TCS, and the black box warning on TCI regarding skin cancer malignancies) makes them a less preferable treatment option for chronic use, particularly for young children. Hence there is a medical need to find new treatment options for itch is very high among patients and their families. In addition, relief of the chronic itch will disturb the itch-scratch cycle, which has secondary beneficial effects such as improving the skin barrier and may lead to improvement in skin lesions and erythema.

Finding both a cure and effective treatments for chronic itch in AD has been a significant challenge. Histamine is not a major pruritogen in AD, and thus histamine-blocking agents only work in AD patients through their sedative effects, particularly for nocturnal itch. Proteases that are released from immune and skin cells of AD patients and act on GPCRs have been investigated as major pruritogenic contributors in AD. Cathepsin S has been described in the literature as a highly pro-inflammatory and itch-triggering protease. Overexpression of Cathepsin S results in an AD phenotype in mice with severe chronic itch. Recently, one group reported that Cathepsin S evokes itch via MrgprX2. Nevertheless, knowledge is limited regarding the key itch mediators in AD, although several have been identified and postulated to play a role.

Another pruritogenic neuropeptide is Substance P, released by neuronal and non-neuronal dermal cells, is a pro-inflammatory and vasoactive neuropeptide that also acts as a pruritogen. Hence, targeting its cognate receptor NK1 was considered as an ideal therapeutic approach and has been pursued with aprepitant. However, despite pre-clinical data in mice, the NK1R antagonist aprepitant failed to significantly block itch in humans.

MrgprX2 is a promising target due to its promiscuous ligand binding properties to various pruritic mediators. Multiple pruritic mediators known or speculated to be relevant players in the pathogenesis of AD appear to bind MrgprX receptor rather than the cognate receptors.

There is an unmet need for effective treatments for AD, and its symptoms. This invention is directed to this, as well as to other important ends.

### SUMMARY

Described herein are compositions comprising MrgprX2 antagonists and methods for using the MrgprX2 antagonists for the treatment of inflammatory conditions such as AD.

Therefore, in a first aspect, the present disclosure provides for compounds that are MrgprX2 antagonists.

In a second aspect, the present disclosure provides for a composition comprising a MrgprX2 antagonist, and a pharmaceutically acceptable excipient.

In a third aspect, the present disclosure provides for a method for treating an inflammatory disorder, the method comprising administering to a subject in need thereof a topical or oral composition having a therapeutically effective amount of a MrgprX2 antagonist (e.g. a MrgprX2 antagonist according to the present disclosure); and a dermatologically or orally acceptable excipient.

In a fourth aspect, the present disclosure provides a method for reducing inflammation in mammalian skin, the method comprising administering to the mammalian skin an effective amount of a topical or oral composition including an MrgprX2 antagonist (e.g. a MrgprX2 antagonist according to the present disclosure) and a dermatologically or orally acceptable excipient to a subject in need thereof.

In a fifth aspect, the present disclosure provides a method for reducing the incidence of or severity of itch in a subject in need thereof, the method comprising administering to the mammalian skin a therapeutically effective amount of a topical or oral composition including a MrgprX2 antagonist (e.g. a MrgprX2 antagonist according to the present disclosure) to a subject in need thereof.

### DETAILED DESCRIPTION

Provided herein are topical or oral compositions for treating inflammatory conditions, e.g., skin disorders characterized by inflammation. In particular, the pharmaceutical compositions include compounds that are antagonists of the Mas-related G protein-coupled receptor MrgprX2.

### MrgprX2 Antagonists for use in the Compositions and Methods of the Present Disclosure

In several embodiments, the present disclosure provides for a Compound [Compound 1] that is a MrgprX2 antagonist having the Formula I:

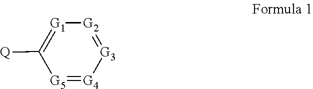

wherein:

Q is

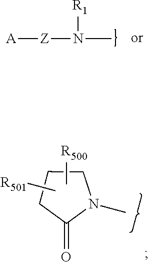

Z is —C(═O)—(CR₂₀R₂₁)ₙ or —S(═O)₂—;

R₁ is H or C₁₋₃ alkyl;

n is 0 or 1;

each R₂₀ and R₂₁ is independently H or C₁₋₃ alkyl;

G₁, G₂, G₃, G₄ and G₅ are each independently N or —C—L₁—Mi, provided that at least one of G₁, G₂, G₃, G₄ and G₅ is N;

each L₁ is independently a bond, O, —C(═O), —C(═O)—NH—, —CH₂—, —O—(CH₂)_{w}— where w is 1, 2 or 3, or —N(R₉₀)—; or any two L₁—M groups on adjacent carbon atoms can together form a group of formula —O—(CH₂)ᵥ—O— where v is 1 or 2;

each R₉₀ is independently H or C₁₋₃ alkyl;

each M₁is independently H, —OH, halogen, cyano, C₆₋₁₀ aryl; 5-10 member heteroaryl having 1-3 ring heteroatoms independently selected from N, O and S; C₁₋₆ alkyl; C₃₋₆ cycloalkyl; ——NR₅₀R₅₁; 4-10 member heterocycloalkyl having 1-3 ring heteroatoms independently selected from N, O and S; wherein each of the C₆₋₁₀ aryl; 5-10 member heteroaryl; C₁₋₆ alkyl; C₁₋₆ cycloalkyl and 4-10 member heterocycloalkyl is each optionally substituted with 1, 2 or 3 substituents independently selected from the group consisting of halogen, cyano, —OH, C₁₋₃ alkyl, C₁₋₃ haloalkyl, C₁₋₃ alkoxy, and —C(═O)—N(N₉₁)(R₉₂);

each R₉₁ and R₉₂ is independently selected from the group consisting of H and C₁₋₃ alkyl;

each R₅₀ and R₅₁ is independently selected from the group consisting of H, C₁₋₃ alkyl, and C₆₋₁₀ aryl;

A is —L₂—M₂;

L₂ is selected from a bond and —(CR₆₀R₆₁)ₖ—;

R₆₀ and R₆₁are each independently H or C₁₋₃ alkyl optionally substituted with 1, 2 or 3 substituents independently selected from —OH and halogen;

k is 1, 2 or 3;

M₂ is C₁₋₆ alkyl; C₃₋₆ cycloalkyl; C₆₋₁₀ spiroalkyl; 4-10 member heterocycloalkyl having 1-3 ring heteroatoms independently selected from N, O and S; —N(R₈₁)(R₈₂); and C₆₋₁₀ aryl; wherein each C₁₋₆ alkyl, C₃₋₆ cycloalkyl, C₆₋₁₀ spiroalkyl, 5-10 member heteroaryl, 4-10 member heterocycloalkyl, and C₆₋₁₀ aryl is each optionally substituted with 1, 2, 3 or 4 independently selected R₂₀₀ groups;

each R₂₀₀ is independently selected from C₁₋₆ alkyl; C₁₋₆ hydroxyalkyl; C₃₋₆ cycloalkyl; 5-10 member heterocycloalkyl having 1-3 ring heteroatoms independently selected from N, O and S; C₁₋₆ mono-, di- or trihaloalkyl; halogen; cyano; —OH; C₁₋₆ alkoxy; —S(═O)₂NR₅₀₂R₅₀₃ and C₆₋₁₀ aryl;

R₇₀ and R₇₁ are each independently H or C₁₋₃ alkyl;

each R₈₁ and R₈₂ is independently selected from H; C₁₋₆ alkyl and C₃₋₆ cycloalkyl; wherein the C₁₋₆ alkyl and C₃₋₆ cycloalkyl are optionally substituted with 1, 2, 3, or 4 substituents independently selected from —OH and halogen;

R₅₀₀ and R₅₀₁ are independently absent or C₁₋₆ alkyl;

R₅₀₂ and R₅₀₃ are independently H or C₁₋₆ alkyl;

or a stereoisomer, solvates, tautomers, or pharmaceutically acceptable salts thereof.

The present disclosure further provides compounds as follows:
1.1 Compound 1, wherein G₁ is N;
1.2 Compound 1 or 1.1, wherein G₂ is N;
1.3 Compound 1 or 1.1, wherein G₁ and G₄ are N;
1.4 Compound 1 or 1.1, wherein G₁ and G₂ are N;
1.5 Compound 1 or 1.1, wherein G₁ and G₅ are N;
1.6 Any of the preceding compounds, wherein G₃ is —C—L₁—M₁;
1.7 Any of the preceding compounds, wherein G₄ is —C—L₁—M₁;
1.8 Any of the preceding compounds, wherein G₂ is —C—L₁—M₁;
1.9 Any of the preceding compounds, wherein L₁ is O;
1.10 Any of the preceding compounds, wherein L₁ is —CH₂—;
1.11 Any of the preceding compounds, wherein L₁ is a bond;
1.12 Any of the preceding compounds, wherein L₁ is —C(═O);
1.13 Any of the preceding compounds, wherein L₁ is —C(═O)—NH—;
1.14 Any of the preceding compounds, wherein L₁ is —N(R₉₀)—;
1.15 Any of the preceding compounds, wherein R₁ is H;
1.16 Any of the preceding compounds, wherein n is 0;
1.17 Any of the preceding compounds, wherein n is 1;
1.18 Any of the preceding compounds, wherein M₁is selected from C₆ aryl; C₆ heteroaryl having 1 or 2 ring heteroatoms independently selected from N and O, C₅ or C₆ heterocycloalkyl having 1 or 2 ring heteroatoms independently selected from N and O; and a 5-10 member heterocycloalkyl having 1-3 ring heteroatoms independently selected from N, O and S; each of the foregoing of which is optionally substituted;
1.19 Any of the preceding compounds, wherein M₁is phenyl, pyridyl, pyrrolidine, pyridazine, tetrahydrofuran, tetrahydropyran or dihydroindiole, each of which is optionally substituted;
1.20 Compound 1.19, wherein the substituents for M₁are independently selected from halogen; CN; —OH; —C(═O)—NH₂; CF₃; and —OCH₃;
1.21 Any of the preceding compounds, wherein M₁is optionally substituted phenyl;
1.22 Compound 1.21, wherein the phenyl is substituted in the 4-position;
1.23 Compound 1.21, wherein the phenyl is substituted in the 3-position and the 4-position;
1.24 Compound 1.21, wherein the phenyl is substituted in the 3-position and the 5-position;
1.25 Any of the preceding compounds, wherein M₁is optionally substituted pyridyl;
1.26 Any of the preceding compounds, wherein M₁is optionally substituted pyridyl-4-yl;
1.27 Any of the preceding compounds, wherein M₁is optionally substituted pyridyl-3-yl;
1.28 Compound 1.26, wherein the pyridyl is substituted at a carbon ortho (i.e., adjacent) to the pyridyl nitrogen;
1.29 Compound 1.27 wherein the pyridyl is substituted at a carbon meta to the pyridyl nitrogen;
1.30 Compound 1.27, wherein the pyridyl is substituted at a carbon ortho (i.e., adjacent) to the pyridyl nitrogen;
1.31 Compound 1.26, wherein the pyridyl is substituted at a carbon meta to the pyridyl nitrogen;
1.32 Any of the preceding compounds, wherein M₁is optionally substituted heterocycloalkyl;
1.33 Any of the preceding compounds, wherein M₁is optionally substituted pyrrolidinyl;
1.34 Any of the preceding compounds, wherein M₁is optionally substituted pyrrolidine-1-yl;
1.35 Any of the preceding compounds, wherein M₁is optionally substituted tetrahydropyranyl;
1.36 Any of the preceding compounds, wherein M₁is optionally substituted tetrahydropyran-4-yl;
1.37 Any of the preceding compounds, wherein M₁is optionally substituted cycloalkyl;
1.38 Any of the preceding compounds, wherein M₁is optionally substituted C₁₋₆ alkyl;
1.39 Any of the preceding compounds, wherein M₁is —NR₅₀R₅₁;
1.40 Any of the preceding compounds, wherein L₂ is a bond;
1.41 Any of the preceding compounds, wherein L₂ is —(CR₆₀R₆₁)ₖ—;
1.42 Any of the preceding compounds, wherein M₂ is optionally substituted C₁₋₆ alkyl;
1.43 Any of the preceding compounds, wherein M₂ is optionally substituted isopropyl;
1.44 Any of the preceding compounds, wherein M₂ is optionally substituted C₃₋₆ cycloalkyl;
1.45 Any of the preceding compounds, wherein M₂ is cyclopropyl or cyclobutyl, each of which is optionally substituted with from 1 to 4 methyl groups;
1.46 Any of the preceding compounds, wherein M₂ is cyclopropyl or cyclobutyl, each of which is optionally substituted with 1 or 2 substituents independently selected from methyl, halogen, mono-, di- or trihalomethyl, cyano, hydroxymethyl and hydroxy;
1.47 Any of the preceding compounds, wherein M₂ is cyclopropyl optionally substituted with 1 to 4 methyl groups, halogen or trihalomethyl;
1.48 Any of the preceding compounds, wherein M₂ is heterocycloalkyl optionally substituted with 1 or 2 groups independently selected from methyl and hydroxy;
1.49 Any of the preceding compounds, wherein M₂ is tetrahydrofuran, pyrrolidine, tetrahydropyran or morpholine, each of which is optionally substituted with 1 or 2 groups independently selected from methyl and hydroxy;
1.50 Any of the preceding compounds, wherein M₂ is —N(R₈₁)(R₈₂);
1.51 Any of the preceding compounds, wherein R₈₁ and R₈₂ are independently selected from C₁₋₃ alkyl and C3-4 cycloalkyl; each of which is optionally substituted with 1 or 2 substituents independently selected from —OH and halogen;
1.52 Any of the preceding compounds, wherein the compound selected from the Compounds in Table 1 herein, or a stereoisomer, solvates, tautomers, or pharmaceutically acceptable salts thereof.

Also provided in accordance with the present disclosure is a topical or oral composition [Composition 1] comprising a MrgprX2 antagonist and a dermatologically or orally acceptable excipient. In some embodiments, the MrgprX2 antagonist is Compound I, having Formula I described above.

The present disclosure further provides compositions as follows:

1.1 Composition 1, wherein the MrgprX2 antagonist is Compound I, having Formula I described above;
1.2 Composition 1.1, wherein G₁ is N;
1.3 Composition 1.1, wherein G₂ is N;
1.4 Composition 1.1, wherein G₁ and G₄ are N;
1.5 Composition 1.1, wherein G₁ and G₂ are N;
1.6 Composition 1.1, wherein G₁ and G₅ are N;
1.7 Composition 1.1, wherein G₃ is —C—L₁—M₁;
1.8 Composition 1.1, wherein G₄ is —C—L₁—M₁;
1.9 Composition 1.1, wherein G₂ is —C—L₁—M₁;
1.10 Any of the preceding compositions, wherein L₁ is O;
1.11 Any of the preceding compositions, wherein L₁ is —CH—;
1.12 Any of the preceding compositions, wherein L₁ is a bond;
1.13 Any of the preceding compositions, wherein L₁ is —C(═O);
1.14 Any of the preceding compositions, wherein L₁ is —C(═O)—NH—;
1.15 Any of the preceding compositions, wherein L₁ is —N(R₉₀)—;
1.16 Any of the preceding compositions, wherein R₁ is H;
1.17 Any of the preceding compositions, wherein n is 0;
1.18 Any of the preceding compositions, wherein n is 1;
1.19 Any of the preceding compositions, wherein M₁is selected from C₆ aryl; C₆ heteroaryl having 1 or 2 ring heteroatoms independently selected from N and O, C₅ or C₆ heterocycloalkyl having 1 or 2 ring heteroatoms independently selected from N and O; and a 5-10 member heterocycloalkyl having 1-3 ring heteroatoms independently selected from N, O and S; each of the foregoing of which is optionally substituted;
1.20 Any of the preceding compositions, wherein M₁is phenyl, pyridyl, pyrrolidine, pyridazine, tetrahydrofuran, tetrahydropyran or dihydroindiole, each of which is optionally substituted;
1.21 Composition 1.20, wherein the substituents for M₁are independently selected from halogen; CN; —OH; —C(═O)—NH₂; CF₃; and —OCH₃;
1.22 Any of the preceding compositions, wherein M₁is optionally substituted phenyl;
1.23 Composition 1.22, wherein the phenyl is substituted in the 4-position;
1.24 Composition 1.22, wherein the phenyl is substituted in the 3-position and the 4-position;
1.25 Composition 1.22, wherein the phenyl is substituted in the 3-position and the 5-position;
1.26 Any of the preceding compositions, wherein M₁is optionally substituted pyridyl;
1.27 Any of the preceding compositions, wherein M₁is optionally substituted pyridyl-4-yl;
1.28 Any of the preceding compositions, wherein M₁is optionally substituted pyridyl-3-yl;
1.29 Composition 1.27, wherein the pyridyl is substituted at a carbon ortho (i.e., adjacent) to the pyridyl nitrogen;
1.30 Composition 1.27, wherein the pyridyl is substituted at a carbon meta to the pyridyl nitrogen;
1.31 Composition 1.28, wherein the pyridyl is substituted at a carbon ortho (i.e., adjacent) to the pyridyl nitrogen;
1.32 Composition 1.28, wherein the pyridyl is substituted at a carbon meta to the pyridyl nitrogen;
1.33 Any of the preceding compositions, wherein M₁is optionally substituted heterocycloalkyl;
1.34 Any of the preceding compositions, wherein M₁is optionally substituted pyrrolidinyl;
1.35 Any of the preceding compositions, wherein M₁is optionally substituted pyrrolidine-1-yl;
1.36 Any of the preceding compositions, wherein M₁is optionally substituted tetrahydropyranyl;
1.37 Any of the preceding compositions, wherein M₁is optionally substituted tetrahydropyran-4-yl;
1.38 Any of the preceding compositions, wherein M₁is optionally substituted cycloalkyl;
1.39 Any of the preceding compositions, wherein M₁is optionally substituted C₁₋₆ alkyl;
1.40 Any of the preceding compositions, wherein M₁is —NR₅₀R₅₁;
1.41 Any of the preceding compositions, wherein L₂ is a bond;
1.42 Any of the preceding compositions, wherein L₂ is —(CR₆₀R₆₁)ₖ—;
1.43 Any of the preceding compositions, wherein M₂ is optionally substituted C₁₋₆ alkyl;
1.44 Any of the preceding compositions, wherein M₂ is optionally substituted isopropyl;
1.45 Any of the preceding compositions, wherein M₂ is optionally substituted C₃₋₆ cycloalkyl;
1.46 Any of the preceding compositions, wherein M₂ is cyclopropyl or cyclobutyl, each of which is optionally substituted with from 1 to 4 methyl groups;
1.47 Any of the preceding compositions, wherein M₂ is cyclopropyl or cyclobutyl, each of which is optionally substituted with 1 or 2 substituents independently selected from methyl, halogen, mono-, di- or trihalomethyl, cyano, hydroxymethyl and hydroxy;
1.48 Any of the preceding compositions, wherein M₂ is cyclopropyl optionally substituted with 1 to 4 methyl groups, halogen or trihalomethyl;
1.49 Any of the preceding compositions, wherein M₂ is heterocycloalkyl optionally substituted with 1 or 2 groups independently selected from methyl and hydroxy;
1.50 Any of the preceding compositions, wherein M₂ is tetrahydrofuran, pyrrolidine, tetrahydropyran or morpholine, each of which is optionally substituted with 1 or 2 groups independently selected from methyl and hydroxy;
1.51 Any of the preceding compositions, wherein M₂ is —N(R₈₁)(R₈₂);
1.52 Any of the preceding compositions, wherein R₈₁ and R₈₂ are independently selected from C₁₋₃ alkyl and C₃₋₄ cycloalkyl; each of which is optionally substituted with 1 or 2 substituents independently selected from —OH and halogen;
1.53 Any of the preceding compositions, wherein the MrgprX2 antagonist is a compound selected from the Compounds in Table 1 herein, or a stereoisomer, solvates, tautomers, or pharmaceutically acceptable salts thereof;
1.54 Any of the preceding compositions, wherein the composition is in the form of a cream, a gel, a spray or an ointment.
1.55 Any of the preceding compositions, wherein the MrgprX2 antagonist is present at a concentration of about 0.001 wt. % to about 10 wt. %, based on the total weight of the composition.
1.56 Any of the preceding compositions, wherein the MrgprX2 antagonist is present at a concentration of about 0.1 wt. % to about 5 wt. %, based on the total weight of the composition.
1.57 Any of the preceding compositions, further comprising a skin absorption enhancer.
1.58 Any of the preceding compositions, further comprising a skin absorption enhancer comprising one or more of mannitol, sulphoxides (e.g., dimethylsulphoxide, DMSO), Azones (e.g. laurocapram), pyrrolidones (e.g., 2-pyrrolidone, 2P), alcohols and alkanols (e.g., ethanol, or decanol), glycols (e.g., propylene glycol, hexylene glycol, polyoxyethylene glycol, diethylene glycol), surfactants (also common in dosage forms) and terpenes.
1.59 Any of the preceding compositions, wherein the composition is applied to a patient's skin once daily.
1.60 Any of the preceding compositions, wherein the composition is applied to a patient's skin twice daily.
1.61 Any of the preceding compositions, wherein the composition is applied to a patient's skin three times daily.
1.62 Any of the preceding compositions, wherein the composition is administered to a patient suffering from an inflammatory disorder.
1.63 The preceding composition, wherein the inflammatory disorder is a disorder of the skin.
1.64 The preceding composition, wherein the skin is human skin.
1.65 Any of compositions 1.64-1.66, wherein the inflammatory disorder activates or is consequent to activation, of MrgprX2.
1.66 The preceding composition, wherein the inflammatory disorder is atopic dermatitis (e.g., Asian atopic dermatitis, European atopic dermatitis), chronic urticaria, pseudo-allergic reactions triggered by small molecules for example anaphylactoid drug reactions, anaphylactic shock, rosacea, asthma, systemic itch such as cholestatic or uremic itch, chronic itch triggered by systemic diseases, drug-adverse reactions.
1.67 Any of compositions 1.63-1.67, wherein the inflammatory disorder is atopic dermatitis (e.g., Asian atopic dermatitis, European atopic dermatitis).
1.68 Any of the preceding compositions, wherein the subject is a human.
1.69 Any of the preceding compositions, wherein the mammalian skin is human skin.
1.70 Any one of the preceding compositions, wherein the composition is for oral administration.

As used herein, “topical composition” refers to a formulation of a compound of the invention and a medium generally accepted in the art for the delivery of the biologically active compound to mammalian skin, e.g., human skin. Such a medium includes all dermatologically acceptable carriers, diluents or excipients therefor.

“Stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

“Solvate” refers to a form of a compound complexed by solvent molecules.

“Tautomers” refers to two molecules that are structural isomers that readily interconvert.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

The compounds of the invention, or their pharmaceutically acceptable salts may contain one or more asymmetric centres and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)- isomers may be prepared using chiral synthons or chiral reagents, or resolved using conventional techniques, for example, chromatography and fractional crystallisation. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

“Dermatologically acceptable excipient” includes without limitation any adjuvant, carrier, vehicle, excipient, glidant, sweetening agent, diluent, preservative, dye/colorant, flavor enhancer, surfactant, wetting agent, dispersing agent, suspending agent, stabilizer, isotonic agent, solvent, or emulsifier, including those approved by the United States Food and Drug Administration as being acceptable for dermatological use on humans or domestic animals, or which are known, or are suitable for use in dermatological compositions.

“Optional” or “optionally” means that the subsequently described event of circumstances may or may not occur, and that the description includes instances where said event or circumstance occurs and instances in which it does not. When a functional group is described as “optionally substituted,” and in turn, substituents on the functional group are also “optionally substituted” and so on, for the purposes of this invention, such iterations are limited to three.

The term “alkyl” is intended to mean a straight or branched carbon radical containing the indicated number of carbon atoms. Some embodiments contain 1 to 5 carbons. Some embodiments contain 1 to 4 carbons. Some embodiments contain 1 to 3 carbons. Some embodiments contain 1 or 2 carbons. Examples of alkyl groups include, but are not limited to, methyl, ethyl, n-propyl, isopropyl, n-butyl, s-butyl, isobutyl, t-butyl, pentyl, isopentyl, t-pentyl, neopentyl, 1-methylbutyl [i.e., —CH(CH₃)CH₂CH₂CH₃], 2-methylbutyl [i.e., —CH₂CH(CH₃)CH₂CH₃], n-hexyl, and the like.

The term “cycloalkyl” is intended to mean a saturated ring radical containing the indicated number of carbon atoms. Some embodiments contain 3 to 6 carbons. Some embodiments contain 3 to 5 carbons. Some embodiments contain 5 to 7 carbons. Some embodiments contain 3 to 4 carbons. Examples include cyclopropyl, cyclobutyl, cyclopentyl, cyclohexyl, cycloheptyl, and the like.

The term “haloalkyl” is intended to mean a radical comprising an alkyl group having the indicated number of carbon atoms, substituted with one or more halogens. For example, C₁-C₆ haloalkyl may be fully substituted in which case it can be represented by the formula CₙL₂ₙ₊₁, wherein L is a halogen and “n” is 1, 2, 3, 4, 5 or 6. When more than one halogen is present then they may be the same or different and selected from: fluorine, chlorine, bromine, and iodine. In some embodiments, haloalkyl contains 1 to 5 carbons. In some embodiments, haloalkyl contains 1 to 4 carbons. In some embodiments, haloalkyl contains 1 to 3 carbons. In some embodiments, haloalkyl contains 1 or 2 carbons. Examples of haloalkyl groups include, but are not limited to, fluoromethyl, difluoromethyl, trifluoromethyl, chlorodifluoromethyl, 2,2,2-trifluoroethyl, pentafluoroethyl, and the like. When used without a prefix indicating the number of halo substituents, “haloalkyl” groups contain 1, 2 or 3 halogen atoms.

The term “hydroxyalkyl” is intended to mean a radical comprising an alkyl group having the indicated number of carbon atoms, substituted with one or more hydroxy (i.e., —OH) groups. When used without a prefix indicating the number of hydroxy substituents, “hydroxyalkyl” groups contain 1, 2 or 3 hydroxy groups.

The term “halogen” is intended to mean to a fluoro, chloro, bromo or iodo group.

The term “aryl” is intended to mean a ring system containing 6 to 10 carbon atoms, that may contain a single ring or two fused rings, and wherein at least one ring is aromatic. Examples include phenyl, indanyl, and naphthyl.

The term “heteroaryl” is intended to mean a ring system containing 5 to 14 ring atoms, that may contain a single ring, two fused rings or three fused rings, and wherein at least one ring is aromatic and at least one ring atom is a heteroatom selected from, for example: O, S and N. Some embodiments contain 5 to 6 ring atoms for example furanyl, thienyl, pyrrolyl, imidazolyl, oxazolyl, thiazolyl, isoxazolyl, pyrazolyl, isothiazolyl, oxadiazolyl, triazolyl, tetrazolyl, thiadiazolyl, pyridinyl, pyrazinyl, pyrimidinyl, pyridazinyl, triazinyl, and the like. Some embodiments contain 8 to 14 ring atoms for example quinolizinyl, quinolinyl, isoquinolinyl, cinnolinyl, phthalazinyl, quinazolinyl, quinoxalinyl, triazinyl, indolyl, isoindolyl, indazolyl, indolizinyl, purinyl, naphthyridinyl, pteridinyl, carbazolyl, acridinyl, phenazinyl, phenothiazinyl, phenoxazinyl, benzoxazolyl, benzothiazolyl, 1H-benzimidazolyl, imidazopyridinyl, benzothienyl, benzofuranyl, isobenzofuran, 2,3-dihydrobenzofuranyl, 4H-benzo[1,3]dioxinyl, 3,4-dihydro-1H-isoquinolinyl, 1,4,6,7-tetrahydro-imidazo[4,5-c]pyridinyl, 7,8-dihydro-5H-[1,6]naphthyridinyl, 5,6-dihydro-8H-[1,2,4]triazolo[4,3-a]pyrazinyl, benzo[1,3]dioxolyl, pyrazolo[1,5-a]pyrimidinyl, 1,2,3,4-tetrahydroquinolinyl, and the like.

The term “cyano” means a —CN group.

The term “alkoxy” means a group of formula -0-alkyl, having the indicated number of carbon atoms.

As used herein the term “heterocycloalkyl” is intended to mean a saturated or partially unsaturated non-aromatic 3-6-membered heterocyclic ring optionally fused to a 3-6 member saturated, partially unsaturated, or aromatic aryl or heteroaryl ring. Examples non-aromatic 3-6-membered heterocyclic rings include oxirane, azinidine, oxetane, tetrahydrofuran, dihydrofuran, pyrrolidine, piperidine, tetrahydropyran, morpholine, piperazine, hexahydropyrimidine, hexahydropyridazine, and the like. Heterocycloalkyl groups can contain one or more oxo (i.e. —C═O—) groups within the ring, and sulfur ring heteroatoms can be present as sulfur diones. Examples of such heterocycloalkyl rings include sulfolane, tetrahydro-2H-thiopyran-1,1,-dione, thiomorpholine 1,1-dioxide, 2-pyrrolidione, piperidin-2-one, piperazine-2-one, morpholine -3-one, and the like. Examples of heterocycloalkyls having a fused ring include dihydroindoles such as 1,3 dihydroindole.

The term “spiroalkyl” is intended to mean a structure of two or more rings in which two of the rings share one common atom, and wherein at least one of the rings is a cycloalkyl ring, containing the indicated number of carbon atoms. Examples include spirocyclopropane and spirocyclobutane.

### Methods of Using the Compounds of the Invention

The Compounds of the Invention are useful in the treatment of inflammatory disorders, e.g., atopic dermatitis (e.g., Asian atopic dermatitis, European atopic dermatitis), chronic urticaria, pseudo-allergic reactions triggered by small molecules for example anaphylactoid drug reactions, anaphylactic shock, rosacea, asthma, systemic itch such as cholestatic or uremic itch, chronic itch triggered by systemic diseases, drug-adverse reactions. Therefore, administration or use of a preferred MrgprX2 antagonist as described herein, e.g., a MrgprX2 antagonist as hereinbefore described, e.g., a Compound of Formula I, provides a means to ameliorate symptoms of, and/or provide treatment for, various inflammatory diseases and disorders.

For example, in one embodiment the present disclosure provides for a method [Method 1] for treating an inflammatory disorder, the method comprising administering to a subject in need thereof a topical or oral composition comprising a therapeutically effective amount of a MrgprX2 antagonist (e.g. a MrgprX2 antagonist according to the present disclosure); and a dermatologically or orally acceptable excipient.

The present disclosure further provides further embodiments of Method 1 as follows:
1.1 Method 1, wherein the MrgprX2 antagonist is a compound according to Formula I described above;
1.2 Method 1.1, wherein the MrgprX2 antagonist is a compound according to any of

Compounds 1.1-1.55 described above;
1.3 Any of the preceding methods, wherein the MrgprX2 antagonist is a compound selected from the Compounds in Table 1 herein, or a stereoisomer, solvates, tautomers, or pharmaceutically acceptable salts thereof;
1.4 Any of the preceding methods, wherein the composition is in the form of a cream, a gel, a spray or an ointment.
1.5 Any of the preceding methods, wherein the MrgprX2 antagonist is present at a concentration of about 0.001 wt. % to about 10 wt. %, based on the total weight of the composition.
1.6 Any of the preceding methods, wherein the MrgprX2 antagonist is present at a concentration of about 0.1 wt. % to about 5 wt. %, based on the total weight of the composition.
1.7 Any of the preceding methods, further comprising a skin absorption enhancer.
1.8 Any of the preceding methods, further comprising a skin absorption enhancer comprising one or more of mannitol, sulphoxides (e.g., dimethylsulphoxide, DMSO), Azones (e.g. laurocapram), pyrrolidones (e.g., 2-pyrrolidone, 2P), alcohols and alkanols (e.g., ethanol, or decanol), glycols (e.g., propylene glycol, hexylene glycol, polyoxyethylene glycol, diethylene glycol), surfactants (also common in dosage forms) and terpenes.
1.9 Any of the preceding methods, wherein the composition is applied to a patient's skin once daily.
1.10 Any of the preceding methods, wherein the composition is applied to a patient's skin twice daily.
1.11 Any of the preceding methods, wherein the composition is applied to a patient's skin three times daily.
1.12 Any of the preceding methods, wherein the composition is administered to a patient suffering from an inflammatory disorder.
1.13 The preceding methods, wherein the inflammatory disorder is a disorder of the skin.
1.14 The preceding methods, wherein the skin is human skin.
1.15 Any of methods 1.12-1.14, wherein the inflammatory disorder activates or is consequent to activation, of MrgprX2.
1.16 The preceding methods, wherein the inflammatory disorder is atopic dermatitis (e.g., Asian atopic dermatitis, European atopic dermatitis), chronic urticaria, pseudo-allergic reactions triggered by small molecules for example anaphylactoid drug reactions, anaphylactic shock, rosacea, asthma, systemic itch such as cholestatic or uremic itch, chronic itch triggered by systemic diseases, or drug-adverse reactions.
1.17 Any of methods 1.12-1.16, wherein the inflammatory disorder is atopic dermatitis (e.g., Asian atopic dermatitis, European atopic dermatitis).
1.18 Any of the preceding methods, wherein the subject is a human.
1.19 Any of the preceding methods, wherein the mammalian skin is human skin.
1.20 Any of the preceding methods, wherein the composition is for oral administration.

In another embodiment, the present disclosure provides a method [Method 2] for reducing inflammation in mammalian skin, the method comprising administering to the mammalian skin an effective amount of a topical or oral composition including a MrgprX2 antagonist according to the present disclosure and a dermatologically or orally acceptable excipient to a subject in need thereof.

The present disclosure further provides further embodiments of Method 2 as follows:
2.1 Method 2, wherein the MrgprX2 antagonist is a compound according to Formula I described above;
2.2 Method 2 or 2.1, wherein the MrgprX2 antagonist is a compound according to any of Compounds 1.1-1.55 described above;
2.3 Any of the preceding methods, wherein the MrgprX2 antagonist is a compound selected from the Compounds in Table 1 herein, or a stereoisomer, solvates, tautomers, or pharmaceutically acceptable salts thereof;
2.4 Any of the preceding methods, wherein the inflammation is consequent to activation of MrgprX2;
2.5 Any of the preceding methods, wherein the composition is in the form of a cream, a gel, a spray or an ointment.
2.6 Any of the preceding methods, wherein the MrgprX2 antagonist is present at a concentration of about 0.001 wt. % to about 10 wt. %, based on the total weight of the composition.
2.7 Any of the preceding methods, wherein the MrgprX2 antagonist is present at a concentration of about 0.1 wt. % to about 5 wt. %, based on the total weight of the composition.
2.8 Any of the preceding methods, further comprising a skin absorption enhancer.
2.9 Any of the preceding methods, further comprising a skin absorption enhancer comprising one or more of mannitol, sulphoxides (e.g., dimethylsulphoxide, DMSO), Azones (e.g. laurocapram), pyrrolidones (e.g., 2-pyrrolidone, 2P), alcohols and alkanols (e.g., ethanol, or decanol), glycols (e.g., propylene glycol, hexylene glycol, polyoxyethylene glycol, diethylene glycol), surfactants (also common in dosage forms) and terpenes.
2.10 Any of the preceding methods, wherein the composition is applied to a patient's skin once daily.
2.11 Any of the preceding methods, wherein the composition is applied to a patient's skin twice daily.
2.12 Any of the preceding methods, wherein the composition is applied to a patient's skin three times daily.
2.13 Any of the preceding methods, wherein the composition is administered to a patient suffering from an inflammatory disorder.
2.14 The preceding methods, wherein the inflammatory disorder is a disorder of the skin.
2.15 The preceding methods, wherein the skin is human skin.
2.16 Any of methods 1.12-1.14, wherein the inflammatory disorder activates or is consequent to activation, of MrgprX2.
2.17 The preceding methods, wherein the inflammatory disorder is atopic dermatitis (e.g., Asian atopic dermatitis, European atopic dermatitis), chronic urticaria, pseudo-allergic reactions triggered by small molecules for example anaphylactoid drug reactions, anaphylactic shock, rosacea, asthma, systemic itch such as cholestatic or uremic itch, chronic itch triggered by systemic diseases, or drug-adverse reactions.
2.18 Any of methods 1.12-1.16, wherein the inflammatory disorder is atopic dermatitis (e.g., Asian atopic dermatitis, European atopic dermatitis).
2.19 Any of the preceding methods, wherein the subject is a human.
2.20 Any of the preceding methods, wherein the mammalian skin is human skin.
2.21 Any of the preceding methods, wherein the composition is for oral administration.

A further embodiment provides a method [Method 3] for reducing the incidence of or severity of itch, the method comprising administering to the mammalian skin a therapeutically effective amount of a topical or oral composition according to any of Compositions 1 and 1.1-1.73.

The present disclosure further provides further embodiments of Method 3 as follows:
3.1 Method 3, wherein the severity of itch is reduced within 5 minutes of administration.
3.2 Method 3 or 3.1, wherein the severity of itch is reduced for a period of 6 hours from administration.
3.3 Method 3 or 3.1, wherein the severity of itch is reduced for a period of 12 hours from administration.
3.4 Method 3 or 3.1, wherein the severity of itch is reduced for a period of 18 hours from administration.
3.5 Method 3 or 3.1, wherein the severity of itch is reduced for a period of 24 hours from administration.
3.6 Any of the preceding methods, wherein the MgrprX2 antagonist is a compound selected from the Compounds in Table 1 herein, or a stereoisomer, solvates, tautomers, or pharmaceutically acceptable salts thereof.
3.7 Any of the preceding methods, wherein the composition is in the form of a cream, a gel, a spray or an ointment.
3.8 Any of the preceding methods, wherein the MgrprX2 antagonist is present at a concentration of about 0.001 wt. % to about 10 wt. %, based on the total weight of the composition.
3.9 Any of the preceding methods, wherein the MgrprX2 antagonist is present at a concentration of about 0.1 wt. % to about 5 wt. %, based on the total weight of the composition.
3.10 Any of the preceding methods, further comprising a skin absorption enhancer.
3.11 The preceding method, wherein the skin absorption enhancer comprises one or more of mannitol, sulphoxides (e.g., dimethylsulphoxide, DMSO), Azones (e.g. laurocapram), pyrrolidones (e.g., 2-pyrrolidone, 2P), alcohols and alkanols (e.g., ethanol, or decanol), glycols (e.g., propylene glycol, hexylene glycol, polyoxyethylene glycol, diethylene glycol), surfactants (also common in dosage forms) and terpenes.
3.12 Any of the preceding methods, wherein the composition is applied to a patient's skin once daily.
3.13 Any of the preceding methods, wherein the composition is applied to a patient's skin twice daily.
3.14 Any of the preceding methods, wherein the composition is applied to a patient's skin three times daily.
3.15 Any of the preceding methods, wherein the composition is administered to a patient suffering from an inflammatory disorder.
3.16 Any of the preceding methods, wherein the inflammatory disorder is a disorder of the skin.
3.17 Any of the preceding methods, wherein the skin is human skin.
3.18 Any of methods 1.12-1.14, wherein the inflammatory disorder activates or is consequent to activation, of MrgprX2.
3.19 The preceding methods, wherein the inflammatory disorder is atopic dermatitis (e.g., Asian atopic dermatitis, European atopic dermatitis), chronic urticaria, pseudo-allergic reactions triggered by small molecules for example anaphylactoid drug reactions, anaphylactic shock, rosacea, asthma, systemic itch such as cholestatic or uremic itch, chronic itch triggered by systemic diseases, or drug-adverse reactions.
3.20 Any of methods 1.12-1.16, wherein the inflammatory disorder is atopic dermatitis (e.g., Asian atopic dermatitis, European atopic dermatitis).
3.21 Any of the preceding methods, wherein the subject is a human.
3.22 Any of the preceding methods, wherein the mammalian skin is human skin.
3.23 Any of the preceding methods, wherein the composition is for oral administration.

“Atopic dermatitis” refers to a skin condition involving chronic inflammation, and symptoms of atopic dermatitis include a red, itchy rash. Atopic dermatitis may be present on the skin of any part of the body, but is common on the hands, feet, upper chest, and in the bends of elbows or knees. Additional symptoms of atopic dermatitis may include small raised bumps or thickened, scaly skin.

“Psoriasis” is a chronic skin condition related to an overactive immune response. Psoriasis may be present on may be present on the skin of any part of the body. Symptoms of psoriasis include local inflammation, skin flaking, and thick white or red patches of skin.

“Alopecia” is an autoimmune skin disease, causing hair loss on the scalp, face and sometimes on other areas of the body. In alopecia areata, for example, T cell lymphocytes cluster around affected follicles, causing inflammation and subsequent hair loss.

“Chronic Urticaria” (Hives) is a common skin rash triggered by many things including certain foods, medications, and stress. Symptoms can include itchy, raised, red, or skin-colored welts on the skin's surface. Given the role of mast cells in chronic idiopathic urticaria, MrgprX2 partakes a key function in the mast cell activation. Antimicrobial host defense peptides, neuropeptides, major basic protein, eosinophil peroxidase, and some FDA-approved peptidergic drugs activate human MrgprX2. Unique features of MrgprX2 that distinguish it from other GPCRs include their presence both on the plasma membrane and intracellular sites and their selective expression in MCs. Furthermore, small-molecule inhibitors of MrgprX2 could benefit the treatment of MC-dependent allergic and inflammatory disorders such as chronic urticaria which is currently treated by targeting the IgE axis of mast cell activity. However, a variety of MC-activity relies on ligand binding to MrgprX2 (Subramanian H et al., 2016, The Journal of Allergy and Clinical Immunology, 138(3), 700-710; https://doi.org/10.1016/j.jaci.2016.04.051) suggesting that targeting MRGPRX2 might indeed be a treatment option for IgE-independent and resistant chronic urticaria.

“Anaphylactic Shock” is an extreme, often life-threatening allergic reaction to an antigen to which the body has become hypersensitive. Mast cell activation via MrgprB2 has gained attention for its IgE independent mast cell activation and nonhistaminergic itch (Meixiong J. et al., 2019, Immunity, 50(5), 1163-1171.e5. https://doi.org/10.1016/j.immuni.2019.03.013). Activation of MrgprB2 by proadrenomedullin N-terminal peptide 9-20 (PAMP9-20) induced the release of multiple bioactive mediators from mast cells which in turn activated itch-sensing neurons suggesting the mast-cell specific MrgprB2 is key in mast-cell degranulation and related non-histaminergic itch. Mast cell MrgprB2 and MrgrpX2 are activated by SP, compound 48/80 and pseudoallergy inducing drugs such as icatibant (McNeil, B.D. et al., 2015, Nature, 519(7542), 237-241; https://doi.org/10.1038/nature14022) placing MrgprX2 at the center stage of non-histaminergic mast cell activation and various allergic and nonallergic diseases as well as pseudoallergic reactions.

“Rosacea” is condition that causes redness and often small, red, pus-filled bumps on the face. MrgrpX2has also been identified as the receptor for endogenous host defense peptide, including cathelicidin (LL-37) and □-defensin (Subramanian, H. et al., 2011, The Journal of Biological Chemistry, 286(52), 44739-44749; https://doi.org/10.1074/jbc.M111.277152 and Subramanian, H. et al., 2013, Journal of Immunology (Baltimore, Md.: 1950), 191(1), 345-352; https://doi.org/10.4049/jimmunol.1300023) raising the possibility that mast-cell MrgprX2 could partake in antimicrobial host defense. Pituitary adenylate cyclase activating peptide (PACAP), an effective mast cell degranulator (Baun, M. et al., 2012, Cephalalgia: An International Journal of Headache, 32(4), 337-345; https://doi.org/10.1177/0333102412439354 and Seebeck, J. et al., 1998, Annals of the New York Academy of Sciences, 865, 141-146. https://doi.org/10.1111/j.1749-6632.1998.tb11172.x), has been shown to activate MrgprX2 (Tatemoto K. et al., 2006, Biochemical and Biophysical Research Communications, 349(4), 1322-1328; https://doi.org/10.1016/j.bbrc.2006.08.177; and McNeil, B. D. et al., 2015, Nature, 519(7542), 237-241; https://doi.org/10.1038/nuture14022). These findings suggest that MrgprX2 may also function in innate immunity by regulating host defense responses. Given that MrgprX2 is activated by peptides such as LL-37 and the neuropeptide PACAP, both of which are crucially involved in rosacea and function as trigger peptides to affect mast cell activity and vasodilation. Together these findings suggest MrgprX2 as an emerging receptor in the pathophysiology of rosacea.

“Asthma” is a condition in which a person's airways become inflamed, narrow and swell, and produce extra mucus, which makes it difficult to breathe. Mast cells (MC), which also subside in close vicinity with smooth muscle, T cells and leukocytes, are important effector cells in airway hyperresponsiveness and inflammation, a phenomenon characteristic of asthma. Even though in healthy states only low amounts of transcripts are present, the levels of MrgprX2 transcripts increase in severe asthma which is characterize by a phenotypic switch of MCTC from MCT. In contrast to MCT, the mast cell MCTC population in severe asthma is expressing MrgprX2 (Fajt M. L. et al, 2013; The Journal of Allergy and Clinical Immunology, 131(6), 1504-1512; https://doi.org/10.1016/j.jaci.2013.01.035 nd Balzar, S. et al., 2011, American Journal of Respiratory and Critical Care Medicine, 183(3), 299-309; https://doi.org/10.1164rrcm.201002-0295OC). Given that the SP levels are increased in the lung of severe asthma patients which activates MrgprX2, the treatment with small molecule antagonists will benefit severe asthma patients (van Diest, SA. et al., 2012, Biochimica et Biophysica Acta, 1822(1), 74-84; https://doi.org/10.1016/j.bbadis2011.03.019).

“Mammal” or “mammalian” includes humans and both domestic animals such as laboratory animals and household pets, (e.g., cats, dogs, swine, cattle, sheep, goats, horses, rabbits), and non-domestic animals such as wildlife and the like.

“Therapeutically effective amount” refers to that amount of a compound of the invention which, when administered to a mammal, preferably a human, is sufficient to effect treatment of the disease or condition of interest in a mammal, preferably a human, having the disease or condition. The amount of a compound of the invention which constitutes a “therapeutically effective amount” will vary depending on the compound, the disease or condition and its severity, the manner of administration, and the age of the mammal to be treated, but can be determined routinely by one of ordinary skill in the art having regard to his own knowledge and to this disclosure. Preferably, for purposes of this invention, a “therapeutically effective amount” is that amount of a compound of invention which is sufficient to inhibit inflammation of the skin.

“Treating” or “treatment”, as used herein, covers the treatment of the disease or condition of interest in a mammal, preferably a human, and includes:
(i) preventing the disease or condition from occurring in the mammal;
(ii) inhibiting the disease or condition in the mammal, i.e., arresting its development;
(iii) relieving the disease or condition in the mammal, i.e., causing regression of the disease or condition; or
(iv) relieving the symptoms of the disease or condition in the mammal, i.e., relieving the symptoms without addressing the underlying disease or condition.

As used herein, the terms “disease,” “disorder,” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

In the present description, the term “about” means ±20% of the indicated range, value, or structure, unless otherwise indicated.

In some embodiments, the MrgprX2 antagonist (e.g. a MrgprX2 antagonist according to the present disclosure) is present in the topical or oral composition at a concentration of about 0.05% to about 5% by weight.

In certain embodiments, the pharmaceutical compositions described herein further include a dermatologically acceptable excipient. The dermatologically acceptable excipients may be one or more solvents that solubilize and/or stabilize the active ingredient (e.g., MrgprX2 antagonist) contained therein. The dermatologically acceptable excipients may also include skin penetration enhancers, preservatives, viscosity enhancers, pH adjusters, film forming agents and the like. Non-limiting examples of the suitable excipients include water, PEG 200, PEG 400, ethanol, glycerol, Transcutol P (diethylene glycol monoethyl ether), propylene glycol, 1,3-dimethyl-2-imidazolidinone (DMI), sodium metabisulfite, butylated hydroxytoluene (BHT), benzyl alcohol, sodium benzoate, isopropyl myristate, diisopropyl adipate, crodamol OHS (ethylhexyl hydroxystearate), mineral oil, Betadex, TWEEN 20, Brij S20 (polyoxyethylene (20) stearyl ether).

More detailed description of certain suitable excipients is described below. As will be appreciated, components of the pharmaceutical formulations described herein can possess multiple functions. For example, a given substance may act as both a viscosity increasing agent and as an emulsifying agent.

The skin (especially stratum corneum) provides a physical barrier to the harmful effects of the external environment. In doing so, it also interferes with the absorption or transdermal delivery of topical therapeutic drugs. Thus, a suitable dermatologically acceptable excipient may include one or more penetration enhancers (or permeation enhancers), which are substances that promote the diffusion of the therapeutic drugs (e.g., the MrgprX2 antagonists described herein) through the skin barrier. They typically act to reduce the impedance or resistance of the skin to allow improved permeation of the therapeutic drugs. In particular, substances which would perturb the normal structure of the stratum corneum are capable of disrupting the intercellular lipid organization, thus reducing its effectiveness as a barrier. These substances could include any lipid material which would partition into the stratum corneum lipids causing a direct effect or any material which would affect the proteins and cause an indirect perturbation of the lipid structure. Furthermore, solvents, such as ethanol, can remove lipids from the stratum corneum, thus destroying its lipid organization and disrupting its barrier function.

Examples of penetration enhancers or barrier function disrupters include, but are not limited to, alcohol-based enhancers, such as alkanols with one to sixteen carbons, benzyl alcohol, butylene glycol, diethylene glycol, glycofurol, glycerides, glycerin, glycerol, phenethyl alcohol, polypropylene glycol, polyvinyl alcohol, and phenol; amide-based enhancers, such as N-butyl-N-dodecylacetamide, crotamiton, N,N-dimethylformamide, N,N-dimethylacetamide, N-methyl formamide, and urea; amino acids, such as L-a-amino acids and water soluble proteins; azone and azone-like compounds, such as azacycloalkanes; essential oils, such as almond oil, amyl butyrate, apricot kernel oil, avocado oil, camphor, castor oil, 1-carvone, coconut oil, corn oil, cotton seed oil, eugenol, menthol, oil of anise, oil of clove, orange oil, peanut oil, peppermint oil, rose oil, safflower oil, sesame oil, shark liver oil (squalene), soybean oil, sunflower oil, and walnut oil; vitamins and herbs, such as aloe, allantoin, black walnut extract, chamomile extract, panthenol, papain, tocopherol, and vitamin A palmitate; waxes, such as candelilla wax, carnuba wax, ceresin wax, beeswax, lanolin wax, jojoba oil, petrolatum; mixes, such as primary esters of fractionated vegetable oil fatty acids with glycerine or propylene glycol, and interesterified medium chain triglyceride oils; fatty acids and fatty acid esters, such as amyl caproate, butyl acetate, caprylic acid, cetyl ester, diethyl sebacate, dioctyl malate, elaidic acid ethyl caprylate, ethyl glycol palmitostearate, glyceryl beheate, glucose glutamate, isobutyl acetate, laureth-4, lauric acid, malic acid, methyl caprate, mineral oil, myristic acid, oleic acid, palmitic acid, PEG fatty esters, polyoxylene sorbitan monooleate, polypropylene glycols, propylene glycols, saccharose disterate, salicylic acid, sodium citrate, stearic acid, soaps, and caproic-, caprylic-, capric-, and lauric-triglycerides; macrocylics, such as butylated hydroxyanisole, cyclopentadecanolide, cyclodextrins; phospholipid and phosphate enhancers, such as dialkylphosphates, ditetradecyl phosphate, lecithin, 2-pyrrolidone derivatives, such as alkyl pyrrolidone-5-carboxylate esters, pyroglutamic acid esters, N-methyl pyrrolidone, biodegradable soft penetration enhancers, such as dioxane derivatives and dioxolane derivatives; sulphoxide enhancers, such as dimethyl sulphoxide and decylmethyl sulphoxide; acid enhancers, such as alginic acid, sorbic acid, and succinic acid; cyclic amines; imidazolinones; imidazoles; ketones, such as acetone, dimethicone, methyl ethyl ketone, and pentanedione; lanolin derivatives, such as lanolin alcohol, PEG 16 lanolin, and acetylated lanolin; oxazolines; oxazolindinones; proline esters; pyrroles, urethanes; and surfactants, such as nonoxynols, polysorbates, polyoxylene alcohols, polyoxylene fatty acid esters, sodium lauryl sulfate, and sorbitan monostearate.

The topical compositions described herein typically contain one or more carriers, which preferably have a vapor pressure greater than or equal to 23.8 mm Hg at 25° C. Preferred concentration range of a single carrier or the total of a combination of carriers can be from about 0.1 wt. % to about 10 wt. %, more preferably from about 10 wt. % to about 50 wt. %, more specifically from about 50 wt. % to about 95 wt. % of the dermatological composition. Non-limiting examples of the solvent include water (e.g., deionized water) and lower alcohols, including ethanol, 2-propanol and n-propanol.

A dermatological composition of the invention can contain one or more hydrophilic co-solvents, which are miscible with water and/or lower chain alcohols and preferably have a vapor pressure less than water at 25° C. (˜23.8 mm Hg). The carrier typically has a vapor pressure greater than or equal to the hydrophilic co-solvent as to concentrate the active ingredient (e.g., a MrgprX2 antagonist of the present disclosure) on the skin. A hydrophilic co-solvent may be a glycol, specifically propylene glycol. In particular, the propylene glycol can be from the class of polyethylene glycols, specifically polyethylene glycols ranging in molecular weight from 200 to 20000. Preferably, the solvent would be part of a class of glycol ethers. More specifically, a hydrophilic co-solvent of the invention would be diethylene glycol monoethyl ether (transcutol). As used herein, “diethylene glycol monoethyl ether” (“DGME”) or “transcutol” refers to 2-(2-ethoxyethoxy)ethanol {CAS NO 001893} or ethyoxydiglycol. Another preferred co-solvent is 1,3-dimethyl-2-imidazolidinone (DMI).

The topical compositions described herein may also contain one or more “humectant(s)” used to provide a moistening effect. Preferably the humectant remains stable in the composition. Any suitable concentration of a single humectant or a combination of humectants can be employed, provided that the resulting concentration provides the desired moistening effect. Typically, the suitable amount of humectant will depend upon the specific humectant or humectants employed. Preferred concentration range of a single humectant or the total of a combination of humectants can be from about 0.1 wt. % to about 70 wt. %, more preferably from about 5.0 wt. % to about 30 wt. %, more specifically from about 10 wt. % to about 25 wt. % of the dermatological composition. Non-limiting examples for use herein include glycerin, polyhydric alcohols and silicone oils. More preferably, the humectant is glycerin, propylene glycol and/or cyclomethicone. Specifically, the filler would be glycerine and/or cyclomethicone.

In certain embodiments, the pharmaceutical compositions include a viscosity enhancing agent or an emulsifier. Gelling agents are used to increase the viscosity of the final composition. Emulsifiers are substances that stabilize an emulsion. The viscosity increasing agent can also act as an emulsifying agent. Typically, the concentration and combination of viscosity increasing agents will depend on the physical stability of the finished product. Preferred concentration range of a viscosity increasing agent can be from about 0.01 wt. % to about 20 wt. %, more preferably from about 0.1 wt. % to about 10 wt. %, more specifically from about 0.5 wt. % to about 5 wt. % of the dermatological composition. Non-limiting examples of viscosity increasing agents for use herein include classes of celluloses, acrylate polymers and acrylate crosspolymers, such as, hydroxypropyl cellulose, hydroxymethyl cellulose, Pluronic PF127 polymer, carbomer 980, carbomer 1342 and carbomer 940, more preferably hydroxypropyl cellulose, Pluronic PF127 carbomer 980 and carbomer 1342, more specifically hydroxypropyl cellulose (Klucel® EF, GF and/or HF), Pluronic PF127, carbomer 980 and/or carbomer 1342 (Pemulen® TR-1, TR-2 and/or Carbopol® ETD 2020). Examples of emulsifiers for use herein include polysorbates, laureth-4, and potassium cetyl sulfate.

The topical or oral compositions described herein may contain one or more anti-oxidants, radical scavengers, and/or stabilizing agents, preferred concentration range from about 0.001 wt. % to about 0.1 wt. %, more preferably from about 0.1 wt. % to about 5 wt. % of the dermatological composition. Non-limiting examples for use herein include butylatedhydroxytoluene, butylatedhydroxyanisole, ascorbyl palmitate, citric acid, vitamin E, vitamin E acetate, vitamin E-TPGS, ascorbic acid, tocophersolan and propyl gallate. More specifically the anti-oxidant can be ascorbyl palmitate, vitamin E acetate, vitamin E-TPGS, vitamin E or butylatedhydroxy toluene.

The topical or oral compositions described herein may also contain preservatives that exhibit anti-bacterial and/or anti-fungal properties. Preservatives can be present in a gelled dermatological composition of the invention to minimize bacterial and/or fungal over its shelf-life. Preferred concentration range of preservatives in a dermatological composition of the invention can be from about 0.001 wt. % to about 0.01 wt. %, more preferably from about 0.01 wt. % to about 0.5 wt. % of the dermatological composition. Non-limiting examples for use herein include diazolidinyl urea, methylparaben, propylparaben, tetrasodium EDTA, and ethylparaben. More specifically the preservative would be a combination of methylparaben and propylparaben.

The topical compositions described herein may optionally include one or more chelating agents. As used herein, the term “chelating agent” or “chelator” refers to those skin benefit agents capable of removing a metal ion from a system by forming a complex so that the metal ion cannot readily participate in or catalyze chemical reactions. The chelating agents for use herein are preferably formulated at concentrations ranging from about 0.001 wt. % to about 10 wt. %, more preferably from about 0.05 wt. % to about 5.0 wt. % of the dermatological composition. Non-limiting examples for use herein include EDTA, disodium edeate, dipotassium edeate, cyclodextrin, trisodium edetate, tetrasodium edetate, citric acid, sodium citrate, gluconic acid and potassium gluconate. Specifically, the chelating agent can be EDTA, disodium edeate, dipotassium edate, trisodium edetate or potassium gluconate.

The topical or oral compositions described herein may include one or more compatible cosmetically acceptable adjuvants commonly used, such as colorants, fragrances, emollients, and the like, as well as botanicals, such as aloe, chamomile, witch hazel and the like.

Alternatively, other pharmaceutical delivery systems may be employed for the pharmaceutical compositions of the invention. Liposomes and emulsions are well-known examples of delivery vehicles that may be used to deliver active compound(s) or prodrug(s). Certain organic solvents such as dimethylsulfoxide (DMSO) may also be employed.

The topical compositions described herein may be provided in any cosmetically suitable form, preferably as a lotion, a cream, or a ointment, as well as a sprayable liquid form (e.g., a spray that includes the MrgprX2 antagonist in a base, vehicle or carrier that dries in a cosmetically acceptable way without the greasy appearance that a lotion or ointment would have when applied to the skin).

Any suitable amount of a MrgprX2 antagonist (e.g., a compound according to the present disclosure) can be employed in such dermatological compositions, provided the amount effectively reduces local inflammation and/or vascular dysfunction, and remains stable in the composition over a prolonged period of time. Preferably, the stability is over a prolonged period of time, e.g., up to about 3 years, up to 1 year, or up to about 6 months, which is typical in the manufacturing, packaging, shipping and/or storage of dermatologically acceptable compositions. A compound of the present disclosure can be in solution, partially in solution with an undissolved portion or completely undissolved suspension. A compound of the present disclosure can be present in a dermatological composition of the invention in a concentration range from about 0.001 wt. % to about 80 wt. %, from about 0.001 wt. % to about 50 wt. %, from about 0.001 wt. % to about 25 wt. %, or from about 0.001 wt. % to about 6 wt. % of the dermatological composition. In one embodiment, a compound of the present disclosure can be present in a concentration range of from about 0.001 wt. % to about 10 wt. %, from about 0.1 wt. % to about 10 wt. % or from about 1.0 wt. % to about 5.0 wt. % of the dermatological composition.

In treating the inflammatory disorders, e.g., atopic dermatitis (e.g., Asian atopic dermatitis, European atopic dermatitis), chronic urticaria, pseudo-allergic reactions triggered by small molecules for example anaphylactoid drug reactions, anaphylactic shock, rosacea, asthma, systemic itch such as cholestatic or uremic itch, chronic itch triggered by systemic diseases, or drug-adverse reactions, the topical composition comprising a compound of the present disclosure is preferably administered directly to the affected area of the skin (e.g., the skin that itches) of the human in need thereof. When such compositions are in use (e.g., when a dermatological composition comprising a compound of the present disclosure) and a dermatologically acceptable excipient is placed upon the skin of the human in need thereof, the MrgprX2 antagonist of is in continuous contact with the skin of the patient, thereby effecting penetration and treatment.

In topically administering the pharmaceutical compositions of the invention, the skin of the human to be treated can be optionally pre-treated (such as washing the skin with soap and water or cleansing the skin with an alcohol-based cleanser) prior to administration of the dermatological composition of the invention.

The pharmaceutical compositions of the invention may, if desired, be presented in a pack or dispenser device which may contain one or more unit dosage forms containing the active compound(s). The topical composition described herein may also be provided in a patch with the topical composition on the side of the patch that directly contacts the skin. Dermatologically acceptable adhesives may be used to affix the patch to the skin for an extended period of time.

Oral Administration

In some embodiments, the pharmaceutical compositions herein are provided for oral administration. Thus, provided in accordance with the present disclosure are solid, semisolid, or liquid dosage forms for oral administration comprising a compound as described herein. Suitable oral dosage forms include, but are not limited to, tablets, capsules, pills, troches, pellets, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, enteric coatings, film costing agents, modified release agents, coloring agents, dye-migration inhibitors, sweetening agents, and flavoring agents.

Binders or granulators impart cohesiveness to a tablet to ensure that the tablet remains intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, ethylcellulose, carboxymethylcellulose, methylcellulose, methyl paraben, polyalkyleneoxides, povidone, polyvinylpyrrolidone (PVP), crospovidones, Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxy ethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, PA); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, trehalose, lysine, leucine, lecithin, starch, kaolin, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W. R. Grace Co., Baltimore, Md.) and CAB-O-SIL® (Cabot Co. of Boston, Ma.); and mixtures thereof. The pharmaceutical compositions provided herein may contain about 0.1 to about 5% by weight of a lubricant.

Suitable glidants include colloidal silicon dioxide, CAB-O-SIL® (Cabot Co. of Boston, Ma.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation.

The pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric- coated tablets are compressed tablets coated with substances that resist the action of stomach acid but dissolve or disintegrate in the intestine, thus protecting the active ingredients from the acidic environment of the stomach. Enteric coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in- water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration may be also provided in the forms of liposomes, micelles, microspheres, or nanosystems. Micellar dosage forms can be prepared as described in U.S. Pat. No. 6,350,458.

The pharmaceutical compositions provided herein may be provided as non- effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein may be formulated as immediate or modified release dosage forms, including delayed-, sustained, pulsed-, controlled, targeted-, and programmed-release forms. Thus, in some preferred embodiments, the active ingredient(s) (i.e., the calcium channel blocker, or L-arginine, or a combination of a calcium channel blocker and L-arginine, or pharmaceutically acceptable salts, hydrates, solvates and prodrugs thereof), is administered in a pharmaceutical composition which is an immediate release oral dosage form, preferably but not necessarily including an enteric coating. In some preferred embodiments, the active ingredients(s) are administered in a pharmaceutical composition which is an extended release oral dosage form, preferably but not necessarily including an enteric coating. In further preferred embodiments, the active ingredients are administered in a pharmaceutical composition which contains both an immediate release dose and an extended release dose or pulsed release dose of the calcium channel blocker, preferably but not necessarily also including an enteric coating. Such dual release dosage forms achieve release of an initial dose of active ingredient, followed late in time by another pulsed release, or by a sustained release dose. Methodologies for preparing such dual release dosage forms are well known in the art.

In some embodiments, the active ingredients are formulated into a controlled release matrix tablet, which contains one or more polymeric matrix materials that promote the sustained, delayed or pulsed release profile. Non-limiting examples of such polymeric matrix materials include cellulosic materials as described above, and carbomers, for example those sold by Lubrizol Corporation under the name Carbopol®, for example Carbopol® 71G NF, Carbopol® 971P NF and Carbopol® 974P NF polymers.

Some preferred examples of extended release compositions suitable for use in the methods and compositions of the invention include, for example and not limitation, extended release compositions found in nifedipine formulations such as Adalat CC®, Procardia® XL, Afeditab® CR and Nifedical® XL; and in diltiazem formulations such as Cardizem® CD, Cardizem® LA, Cardizem® SR, Cartia® XT and Dilacor® XR.

In some embodiments, the present disclosure provides pharmaceutical compositions for oral administration, for use in treating the conditions and disorders described herein.

Dosages

The compositions provided herein contain therapeutically effective amounts of one or more of the compounds provided herein that are useful in the prevention, treatment, or amelioration of one or more of the symptoms of diseases or disorders described herein and a vehicle. Vehicles suitable for administration of the compounds provided herein include any such carriers known to those skilled in the art to be suitable for the particular mode of administration, preferably topically, orally or via injection. In addition, the compounds may be formulated as the sole active ingredient in the composition or may be combined with other active ingredients.

The active compound is included in the vehicle in an amount sufficient to exert a therapeutically useful effect in the absence of undesirable side effects on the patient treated. The therapeutically effective concentration may be predicted empirically by testing the compounds in in vitro and in vivo systems well known to those of skill in the art and then extrapolated there from for dosages for humans. Human doses are then typically fine-tuned in clinical trials and titrated to response.

The concentration of active compound in the composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders as described herein.

In some embodiments, a therapeutically effective dosage should be from about 0.0001 mg to about 1000 mg per day. In some embodiments, 0.001-50 mg of active ingredient (MgrprX2 antagonist as described herein) per kilogram of body weight per day, delivered topically, orally or by injection as descried herein. In some embodiments, the MgrprX2 antagonist is administered at a dosage of up to 1500 mg/day, for example 1200 mg/day, 900 mg/day, 850 mg/day, 800 mg/day, 750 mg/day, 700 mg/day, 650 mg/day, 600 mg/day, 550 mg/day, 500 mg/day, 450 mg/day, 400 mg/day, 350 mg/day, 300 mg/day, 250 mg/day, 200 mg/day, 150 mg/day, 1000 mg/day, 50 mg/day, 25 mg/day, 10mg/day, or 9, 8, 7, 6, 5, 3, 2, 1, 0.75, 0.5, 0.25, 0.10, 0.05 or 0.01 mg/day.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data or subsequent clinical testing. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions and that the concentration ranges set forth herein are exemplary only and are not intended to limit the scope or practice of the claimed compositions.

Dosage forms or compositions containing active ingredient in the range of 0.005% to 100% with the balance made up from vehicle or carrier may be prepared. Methods for preparation of these compositions are known, or will be apparent, to those skilled in this art; for example, see Remington's Pharmaceutical Sciences, Mack Publishing Company, Easton, Pa., 15th Edition, 1975 or later editions thereof.

Oral Dosage

The oral dosage forms of the invention that contain the MrgprX2 antagonists of the present disclosure will typically be administered at dosages described above.

In some preferred embodiments, the daily dose is administered once per day. In some embodiments, the dosage form is an extended release composition.

In some embodiments, the daily dose is administered in a single dose. In other embodiments, the daily dose is administered in smaller increments given multiple times per day, for example twice or three times per day, in amounts that combined equal the daily values above

In some preferred embodiments, the daily dose is administered in a single dose that provides efficacy for up to 12, up to 18, or up to 24 hours.

Topical Dosages

In some embodiments, topical formulations including the compounds of the present disclosure will contain the MgrprX2 antagonist at a concentration of from 0.001% to 20% by weight of the composition, for example 0.001%-10%, for example 0.001%-8%, for example 0.001%-5%, for example 0.001%-4%, for example 0.001%-3%, for example 0.001%-2%, for example 0.001%-1%, by weight of the of the composition.

The compounds or derivatives may be packaged as articles of manufacture containing packaging material, a compound or derivative thereof provided herein, which is effective for treatment, prevention or amelioration of one or more symptoms of the diseases or disorders, supra, within the packaging material, and a label that indicates that the compound or composition or derivative thereof, is used for the treatment, prevention or amelioration of one or more symptoms of the diseases or disorders, supra.

The articles of manufacture provided herein contain packaging materials. Packaging materials for use in packaging products are well known to those of skill in the art. See, e.g., U.S. Pat. Nos. 5,323,907, 5,052,558 and 5,033,252. Examples of packaging materials include, but are not limited to, blister packs, bottles, tubes, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder described herein.

The following Examples may be used by one skilled in the art to determine the effectiveness of the compounds of the invention in treating a human having a dermatological condition characterized by inflammation.

### EXAMPLES

### Example 1

### Preparation of Compounds According to the Present Disclosure

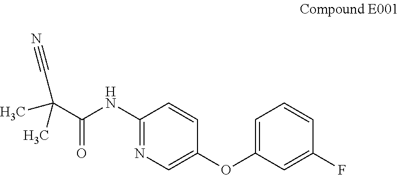 [SMILES: CC(C)(C#N)C(=O)Nc1ccc(Oc2cccc(F)c2)cn1]

### 2-cyano-N-[5-(3-fluorophenoxy)-2-pyridyl]-2-methyl-propanamide

Step 1

5-bromo-2-nitro-pyridine (750 mg, 3.69 mmol), cesium carbonate (2.4 g, 7.39 mmol) and 3-fluorophenol (335 uL, 3.69 mmol) were mixed in DMSO (7.5 mL), purged with nitrogen and stirred in a sealed vial at 50° C. for 4 hours. The reaction mixture was diluted with water and extracted twice with EtOAc. The combined organic extracts were dried over MgSO4, filtered, concentrated under reduced pressure and purified by column chromatography [Biotage SNAP cartridge KP-Sil 50 g; 0-50% EtOAc in heptane]. The mixed fractions were purified further by prep HPLC (Method F) to afford 5-(3-fluorophenoxy)-2-nitro-pyridine as an off-white solid (361 mg, 42% yield). 1H NMR (500 MHz, DMSO-d6) δ 8.46 (d,J=2.8 Hz, 1H), 8.35 (d,J=8.9 Hz, 1H), 7.73 (dd,J=9.0, 2.9 Hz, 1H), 7.54 (td,J=8.3, 6.8 Hz, 1H), 7.22 (dt,J=10.0, 2.4 Hz, 1H), 7.17 (tdd,J=8.6, 2.5, 0.7 Hz, 1H), 7.10 (dd,J=8.2, 2.2 Hz, 1H)

Step 2: 5-(3-fluorophenoxy)pyridin-2-amine

To a solution of 5-(3-fluorophenoxy)-2-nitro-pyridine (361 mg, 1.54 mmol) in Ethanol (5 mL) was added 10% Pd/C (164 mg, 0.154 mmol) and the reaction mixture was put under a balloon of hydrogen and was stirred at RT for 4 hrs. It was then filtered through Celite, washed with EtOAc and concentrated under reduced pressure to afford 5-(3-fluorophenoxy)pyridin-2-amine as an off while solid (300 mg, 95% yield). 1H NMR (500 MHz, DMSO-d6) δ 7.78 (d,J=2.9 Hz, 1H), 7.41-7.30 (m, 1H), 7.24 (dd,J=8.9, 3.0 Hz, 1H), 6.87 (tdd,J=8.6, 2.2, 0.9 Hz, 1H), 6.77-6.68 (m, 2H), 6.51 (d,J=8.9 Hz, 1H), 5.93 (s, 2H).

Step 3

To a solution of 5-(3-fluorophenoxy)pyridin-2-amine (50 mg, 0.245 mmol) and 2-cyano-2-methylpropanoic acid (28 mg, 0.245 mmol) in Ethyl acetate (2 mL) was added N-ethyl-N-isopropyl-propan-2-amine (0.13 mL, 0.735 mmol) and T3P (50% in EtOAc) (0.22 mL, 0.367 mmol) and the reaction was stirred at 70° C. for 6 h. It was then cooled to RT, washed with sat. aq. NaHCO3, dried over MgSO4, filtered, concentrated under reduced pressure and purified by prep HPLC (Method F) to afford the title compound as a pale yellow oil (39 mg). 1H NMR (500 MHz, DMSO-d6) δ 10.75 (s, 1H), 8.25 (d, J=2.9 Hz, 1H), 8.04 (d, J=9.0 Hz, 1H), 7.63 (dd, J=9.0, 3.0 Hz, 1H), 7.43 (ddd, J=8.3, 8.3, 7.0 Hz, 1H), 7.04-6.95 (m, 1H), 6.96-6.92 (m, 1H), 6.89-6.81 (m, 1H), 1.68 (s, 6H).

LCMS: m/z 300.2 [M+H]+, (ESI+), RT=3.35 (Method A)

TABLE 1
The following compounds were Synthesized using a similar method to that used in
Compound E001

| Compound | | | LCMS |
|---|---|---|---|
| number | Structure | Analytical data | Method |
| E002 | 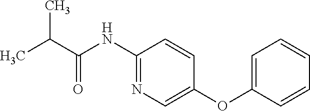 [SMILES: CC(C)C(=O)Nc1ccc(Oc2ccccc2)cn1] | 1H NMR (500 MHz, DMSO-d6) δ 10.45 (s, 1H), 8.18-8.04 (m, 2H), 7.50 (dd, J = 9.0, 3.0 Hz, 1H), 7.43-7.34 (m, 2H), 7.17-7.09 (m, 1H), 7.05- 6.98 (m, 2H), 2.74 (hept, J = 6.8 Hz, 1H), 1.09 (d, J = 6.8 Hz, 6H). LCMS: m/z 257.1 [M + H]+, (ESI+), | A |
| | | RT = 3.19 | |
| E003 | 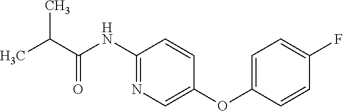 [SMILES: CC(C)C(=O)Nc1ccc(Oc2ccc(F)cc2)cn1] | 1H NMR (500 MHz, DMSO-d6) δ 10.45 (s, 1H), 8.21-8.02 (m, 2H), 7.49 (dd, J = 9.0, 3.0 Hz, 1H), 7.31-7.17 (m, 2H), 7.13-7.00 (m, 2H), 2.74 (hept, J = 6.9 Hz, 1H), 1.09 (d, J = 6.8 Hz, 6H). LCMS: m/z 275.3 [M + H]+, (ESI+), | A |
| | | RT = 3.24 | |
| E004 | 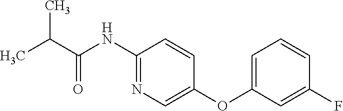 [SMILES: CC(C)C(=O)Nc1ccc(Oc2cccc(F)c2)cn1] | 1H NMR (500 MHz, DMSO-d6) δ 10.50 (s, 1H), 8.21-8.10 (m, 2H), 7.58 (dd, J = 9.0, 3.0 Hz, 1H), 7.41 (td, J = 8.3, 6.9 Hz, 1H), 7.01-6.94 (m, 1H), 6.90 (dt, J = 10.5, 2.4 Hz, 1H), 6.84 (dd, J = 8.3, 2.1 Hz, 1H), 2.76 (hept, J = 6.8 Hz, 1H), 1.10 (d, J = 6.8 Hz, 6H). | A |
| | | LCMS: m/z 275.2 [M + H]+, (ESI+), | |
| | | RT = 3.32 | |
| E005 | 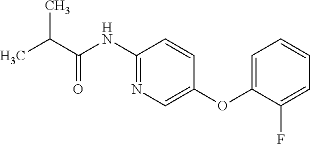 [SMILES: CC(C)C(=O)Nc1ccc(Oc2ccccc2F)cn1] | 1H NMR (500 MHz, DMSO-d6) δ 10.45 (s, 1H), 8.18-8.05 (m, 2H), 7.48 (dd, J = 9.0, 3.1 Hz, 1H), 7.44-7.36 (m, 1H), 7.27-7.13 (m, 3H), 2.74 (sept, J = 6.8 Hz, 1H), 1.09 (d, J = 6.8 Hz, 6H). LCMS: m/z 275.1 [M + H]+, (ESI+), RT = 3.19 | A |
| E006 | 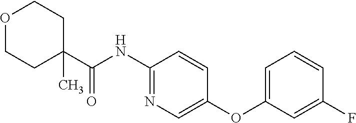 [SMILES: CC1(C(=O)Nc2ccc(Oc3cccc(F)c3)cn2)CCOCC1] | 1H NMR (500 MHz, DMSO-d6) δ 10.06 (s, 1H), 8.22-8.17 (m, 1H), 8.15- 8.08 (m, 1H), 7.59 (dd, J = 9.0, 3.0 Hz, 1H), 7.41 (ddd, J = 8.3, 8.3, 6.9 Hz, 1H), 6.98 (dddd, J = 8.6, 8.6, 2.5, 0.8 Hz, 1H), 6.92 (ddd, J = 10.5, 2.4, 2.4 Hz, 1H), 6.88-6.81 (m, 1H), 3.67 (ddd, J = 11.5, 3.9, 3.9 Hz, 2H), 3.45 | A |
| | | (ddd, J = 11.7, 9.1, 2.7 Hz, 2H), 2.21- | |
| | | 2.02 (m, 2H), 1.50 (ddd, J = 13.3, 9.1, | |
| | | 3.8 Hz, 2H), 1.28 (s, 3H). | |
| | | LCMS: m/z 331.1 [M + H]+, (ESI+), | |
| | | RT = 3.22 | |
| E007 | 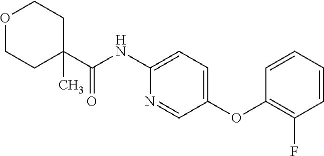 [SMILES: CC1(C(=O)Nc2ccc(Oc3ccccc3F)cn2)CCOCC1] | 1H NMR (500 MHz, DMSO-d6) δ 10.02 (s, 1H), 8.15 (d, J = 3.0 Hz, 1H), 8.10-8.01 (m, 1H), 7.49 (dd, J = 9.1, 3.0 Hz, 1H), 7.45-7.34 (m, 1H), 7.28- 7.14 (m, 3H), 3.66 (ddd, J = 11.5, 4.0, 4.0 Hz, 2H), 3.44 (ddd, J = 11.7, 9.2, 2.7 Hz, 2H), 2.18-2.03 (m, 2H), 1.49 (ddd, J = 13.3, 9.1, 3.7 Hz, 2H), 1.27 (s, 3H). LCMS: m/z 331.1 [M + H]+, (ESI+), | A |
| | | RT = 3.11 | |
| E008 | 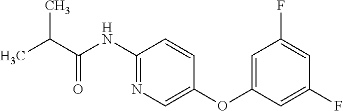 [SMILES: CC(C)C(=O)Nc1ccc(Oc2cc(F)cc(F)c2)cn1] | 1H NMR (500 MHz, DMSO-d6) δ 10.52 (s, 1H), 8.20 (d, J = 3.0 Hz, 1H), 8.18 (d, J = 9.1 Hz, 1H), 7.63 (dd, J = 9.0, 3.0 Hz, 1H), 6.99 (tt, J = 9.3, 2.2 Hz, 1H), 6.84-6.70 (m, 2H), 2.75 (hept, J = 6.8 Hz, 1H), 1.09 (d, J = 6.8 Hz, 6H). | A |
| | | LCMS: m/z 293.1 [M + H]+, (ESI+), | |
| | | RT = 3.47 | |
| E009 | 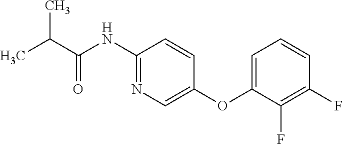 [SMILES: CC(C)C(=O)Nc1ccc(Oc2cccc(F)c2F)cn1] | 1H NMR (500 MHz, DMSO-d6) δ 10.49 (s, 1H), 8.20 (d, J = 2.9 Hz, 1H), 8.14 (d, J = 9.1 Hz, 1H), 7.58 (dd, J = 9.1, 3.0 Hz, 1H), 7.31-7.16 (m, 2H), 6.97-6.88 (m, 1H), 2.74 (hept, J = 6.8 Hz, 1H), 1.09 (d, J = 6.8 Hz, 6H). LCMS: m/z 293.1 [M + H]+, (ESI+), RT = 3.32 | A |
| E010 | 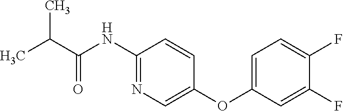 [SMILES: CC(C)C(=O)Nc1ccc(Oc2ccc(F)c(F)c2)cn1] | 1H NMR (500 MHz, DMSO-d6) δ 10.48 (s, 1H), 8.17-8.12 (m, 2H), 7.55 (dd, J = 9.0, 3.1 Hz, 1H), 7.50-7.39 (m, 1H), 7.22 (ddd, J = 11.8, 6.8, 3.0 Hz, 1H), 6.89-6.83 (m, 1H), 2.74 (hept, J = 6.8 Hz, 1H), 1.08 (d, J = 6.8 Hz, 6H). | A |
| | | LCMS: m/z 293.1 [M + H]+, (ESI+), | |
| | | RT = 3.37 | |
| E011 | 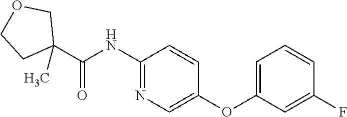 [SMILES: CC1(C(=O)Nc2ccc(Oc3cccc(F)c3)cn2)CCOC1] | 1H NMR (500 MHz, DMSO-d6) δ 10.21 (s, 1H), 8.23-8.18 (m, 1H), 8.17- 8.06 (m, 1H), 7.60 (dd, J = 9.0, 3.0 Hz, 1H), 7.41 (ddd, J = 8.3, 8.3, 7.0 Hz, 1H), 7.01-6.93 (m, 1H), 6.91 (ddd, J = 10.5, 2.4, 2.4 Hz, 1H), 6.86-6.81 (m, 1H), 4.09 (d, J = 8.7 Hz, 1H), 3.84- 3.70 (m, 2H), 3.51 (d, J = 8.7 Hz, 1H), | A |
| | | 2.48-2.40 (m, 1H), 1.84 (ddd, J = | |
| | | 12.6, 7.6, 6.2 Hz, 1H), 1.39 (s, 3H). | |
| | | LCMS: m/z 317.1 [M + H]+, (ESI+), | |
| | | RT = 3.19 | |
| E012 | 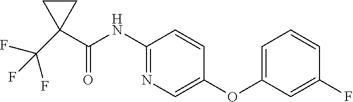 [SMILES: O=C(Nc1ccc(Oc2cccc(F)c2)cn1)C1(C(F)(F)F)CC1] | 1H NMR (500 MHz, DMSO-d6) δ 10.35 (s, 1H), 8.22 (dd, J = 3.0, 0.6 Hz, 1H), 8.01 (d, J = 9.0 Hz, 1H), 7.61 (dd, J = 9.0, 3.0 Hz, 1H), 7.42 (td, J = 8.3, 6.9 Hz, 1H), 7.01-6.97 (m, 1H), 6.93 (dt, J = 10.5, 2.4 Hz, 1H), 6.85 (dd, J = 8.3, 2.1 Hz, 1H), 1.55-1.50 (m, 2H), | A |
| | | 1.34-1.29 (m, 2H). | |
| | | LCMS: m/z 341.2 [M + H]+, (ESI+), | |
| | | RT = 3.83 | |
| E013 | 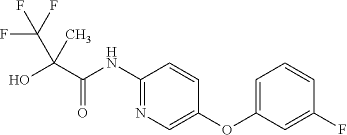 [SMILES: CC(O)(C(=O)Nc1ccc(Oc2cccc(F)c2)cn1)C(F)(F)F] | 1H NMR (500 MHz, Chloroform-d) δ 9.12 (s, 1H), 8.28 (d, J = 9.1 Hz, 1H), 8.11 (d, J = 2.8 Hz, 1H), 7.46 (dd, J = 9.0, 2.9 Hz, 1H), 7.31 (m, J = 8.3, 6.6 Hz, 1H), 6.85 (m, J = 8.4, 2.4 Hz, 1H), 6.77 (dd, J = 8.3, 2.3 Hz, 1H), 6.71 (m, J = 10.0, 2.4 Hz, 1H), 1.76 (s, 3H), 1.25 (s, 1H). LCMS: m/z 345.2 [M + H]+, (ESI+), | A |
| | | RT = 3.41 | |
| E014 | 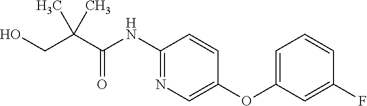 [SMILES: CC(C)(CO)C(=O)Nc1ccc(Oc2cccc(F)c2)cn1] | 1H NMR (500 MHz, Chloroform-d) δ 8.96 (s, 1H), 8.25 (d, J = 9.0 Hz, 1H), 8.09 (d, J = 2.7 Hz, 1H), 7.41 (dd, J = 9.0, 2.9 Hz, 1H), 7.31-7.26 (m, 1H), 6.83-6.79 (m, 1H), 6.74 (dd, J = 8.3, 2.3 Hz, 1H), 6.70-6.66 (m, 1H), 3.72 (s, 2H), 2.96 (s, 1H), 1.31 (s, 6H). | A |
| | | LCMS: m/z 305.2 [M + H]+, (ESI+), | |
| | | RT = 3.02 | |
| E015 | 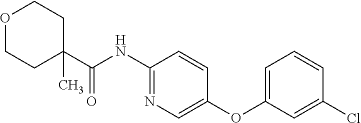 [SMILES: CC1(C(=O)Nc2ccc(Oc3cccc(Cl)c3)cn2)CCOCC1] | 1H NMR (500 MHz, Chloroform-d) δ 8.29 (d, J = 9.0 Hz, 1H), 8.08 (d, J = 2.8 Hz, 1H), 7.94 (s, 1H), 7.41 (dd, J = 9.0, 2.9 Hz, 1H), 7.28-7.24 (m, 1H), 7.28- 7.24 (m, 1H), 7.09 (dd, J = 7.7, 1.6 Hz, 1H), 6.98-6.95 (m, 1H), 6.87 (dd, J = 8.3, 2.4 Hz, 1H), 3.81 (ddd, J = 11.7, 5.9, 3.8 Hz, 2H), 3.69 (ddd, J = 11.7, | A |
| | | 8.4, 3.0 Hz, 2H), 2.13 (m, J = 18.1, 3.6 | |
| | | Hz, 2H), 1.66 (ddd, J = 13.1, 8.6, 3.8 | |
| | | Hz, 2H), 1.37 (s, 3H). | |
| | | LCMS: m/z 347.2 [M + H]+, (ESI+), | |
| | | RT = 3.52 | |
| E016 | 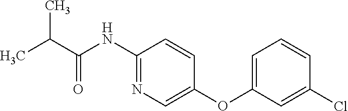 [SMILES: CC(C)C(=O)Nc1ccc(Oc2cccc(Cl)c2)cn1] | 1H NMR (500 MHz, Chloroform-d) δ 8.27 (d, J = 9.0 Hz, 1H), 8.08-8.06 (m, 1H), 7.40 (dd, J = 9.0, 2.9 Hz, 1H), 7.28- 7.23 (m, 1H), 7.09 (ddd, J = 8.0, 1.9, 0.9 Hz, 1H), 6.98-6.94 (m, 1H), 6.86 (ddd, J = 8.3, 2.4, 0.9 Hz, 1H), 2.56 (hept, J = 6.9 Hz, 1H), 1.28 (d, J = 6.9 | A |
| | | Hz, 6H). | |
| | | LCMS: m/z 291.2 [M + H]+, (ESI+), | |
| | | RT = 3.59 | |
| E017 | 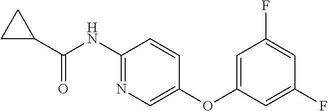 [SMILES: O=C(Nc1ccc(Oc2cc(F)cc(F)c2)cn1)C1CC1] | 1H NMR (500 MHz, DMSO-d6) δ 10.88 (s, 1H), 8.23-8.18 (m, 1H), 8.14 (d, J = 9.1 Hz, 1H), 7.61 (dd, J = 9.0, 3.0 Hz, 1H), 7.06-6.94 (m, 1H), 6.81- 6.72 (m, 2H), 2.00 (tt, J = 7.3, 5.2 Hz, 1H), 0.90-0.72 (m, 4H). LCMS: m/z 291.4 [M + H]+, (ESI+), | A |
| | | RT = 3.33 | |
| E018 | 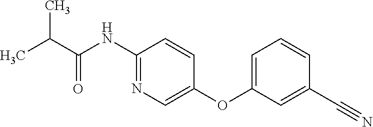 [SMILES: CC(C)C(=O)Nc1ccc(Oc2cccc(C#N)c2)cn1] | 1H NMR (500 MHz, DMSO-d6) δ 10.51 (s, 1H), 8.21-8.14 (m, 2H), 7.63- 7.54 (m, 3H), 7.54-7.50 (m, 1H), 7.39-7.33 (m, 1H), 2.75 (hept,J = 6.9 Hz, 1H), 1.09 (d,J = 6.8 Hz, 6H). LCMS: m/z 282.1 [M + H]+, (ESI)+, RT = 2.99 | A |
| E019 | 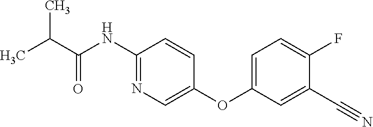 [SMILES: CC(C)C(=O)Nc1ccc(Oc2ccc(F)c(C#N)c2)cn1] | 1H NMR (500 MHz, DMSO-d6) δ 10.50 (s, 1H), 8.19-8.11 (m, 2H), 7.70- 7.65 (m, 1H), 7.60-7.51 (m, 2H), 7.49-7.44 (m, 1H), 2.75 (hept,J = 6.8 Hz, 1H), 1.09 (d,J = 6.8 Hz, 6H). LCMS: m/z 300.1 [M + H]+, (ESI+), RT = 3.09 | A |
| E020 | 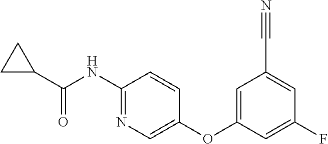 [SMILES: N#Cc1cc(F)cc(Oc2ccc(NC(=O)C3CC3)nc2)c1] | 1H NMR (400 MHz, DMSO-d6) δ 10.90 (s, 1H), 8.27-8.20 (m, 1H), 8.20- 8.13 (m, 1H), 7.68-7.58 (m, 2H), 7.49-7.41 (m, 1H), 7.40-7.27 (m, 1H), 2.06-1.98 (m, 1H), 0.87-0.77 (m, 4H). LCMS: m/z 298.1 [M + H]+, (ESI+), RT = 3.04 | C |
| E021 | 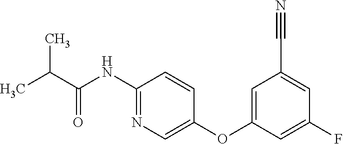 [SMILES: CC(C)C(=O)Nc1ccc(Oc2cc(F)cc(C#N)c2)cn1] | 1H NMR (400 MHz, DMSO-d6) δ 10.54 (s, 1H), 8.29-8.12 (m, 2H), 7.74- 7.55 (m, 2H), 7.43-7.40 (m, 1H), 7.38-7.31 (m, 1H), 2.75 (hept, J = 6.8 Hz, 1H), 1.09 (d, J = 6.8 Hz, 6H). LCMS: m/z 300.1 [M + H]+, (ESI+), RT = 3.17 | C |
| E022 | 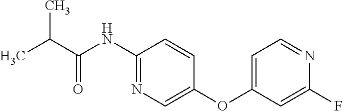 [SMILES: CC(C)C(=O)Nc1ccc(Oc2ccnc(F)c2)cn1] | 1HNMR(500 MHz, DMSO-d6) δ 10.60 (s, 1H), 8.28 (d,J = 2.9 Hz, 1H), 8.22 (d,J = 9.0 Hz, 1H), 8.14 (d,J = 5.8 Hz, 1H), 7.73 (dd,J = 9.0, 2.9 Hz, 1H), 6.96- 6.92 (m, 1H), 6.76-6.72 (m, 1H), 2.76 (hept,J = 6.8 Hz, 1H), 1.10 (d,J = 6.8 Hz, 6H). | A |
| | | LCMS: m/z 276.2 [M + H]+, (ESI+), | |
| | | RT = 3.67 | |
| E023 | 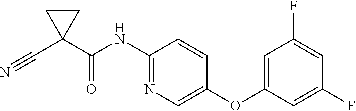 [SMILES: N#CC1(C(=O)Nc2ccc(Oc3cc(F)cc(F)c3)cn2)CC1] | 1H NMR (400 MHz, DMSO-d6) δ 10.44 (s, 1H), 8.30-8.23 (m, 1H), 8.08- 7.92 (m, 1H), 7.77-7.61 (m, 1H), 7.08-6.96 (m, 1H), 6.90-6.75 (m, 2H), 1.76-1.71 (m, 2H), 1.71-1.66 (m, 2H). LCMS: m/z 316.1 [M + H]+, (ESI+), | A |
| | | RT = 3.46 | |
| E024 | 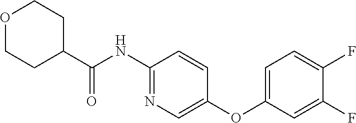 [SMILES: O=C(Nc1ccc(Oc2ccc(F)c(F)c2)cn1)C1CCOCC1] | 1H NMR (500 MHz, DMSO-d6) δ 10.53 (s, 1H), 8.17-8.11 (m, 2H), 7.58- 7.53 (m, 1H), 7.49-7.40 (m, 1H), 7.27-7.20 (m, 1H), 6.89-6.84 (m, 1H), 3.93-3.85 (m, 2H), 3.35-3.33 (m, 1H), 3.31-3.29 (m, 1H), 2.79- 2.70 (m, 1H), 1.72-1.58 (m, 4H). LCMS: m/z 334.9 [M + H]+, (ESI+), | A |
| | | RT = 3.02 | |
| E025 | 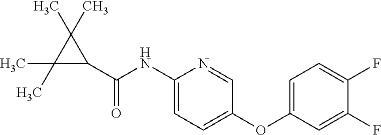 [SMILES: CC1(C)C(C(=O)Nc2ccc(Oc3ccc(F)c(F)c3)cn2)C1(C)C] | 1H NMR (400 MHz, Chloroform-d) δ 8.29 (s, 1H), 8.22 (d, J = 9.1 Hz, 1H), 8.03-7.98 (m, 1H), 7.35 (dd, J = 9.1, 2.9 Hz, 1H), 7.17-7.06 (m, 1H), 6.85- 6.76 (m, 1H), 6.72-6.63 (m, 1H), 1.32 (s, 6H), 1.23 (s, 6H), 1.06 (s, 1H). LCMS: m/z 347.2 [M + H]+, (ESI+), RT = 4.36 | A |
| E026 | 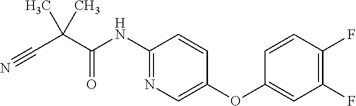 [SMILES: CC(C)(C#N)C(=O)Nc1ccc(Oc2ccc(F)c(F)c2)cn1] | 1H NMR (400 MHz, DMSO-d6) δ 10.74 (s, 1H), 8.25-8.21 (m, 1H), 8.05- 7.97 (m, 1H), 7.63-7.57 (m, 1H), 7.51-7.41 (m, 1H), 7.32-7.23 (m, 1H), 6.96-6.87 (m, 1H), 1.68 (s, 6H). LCMS: m/z 318.2 [M + H]+, (ESI+), RT = 3.41 | A |
| E027 | 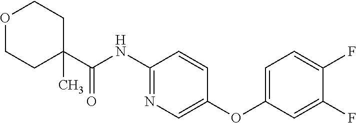 [SMILES: CC1(C(=O)Nc2ccc(Oc3ccc(F)c(F)c3)cn2)CCOCC1] | 1H NMR (400 MHz, DMSO-d6) δ 10.07 (s, 1H), 8.20-8.17 (m, 1H), 8.13- 8.09 (m, 1H), 7.60-7.55 (m, 1H), 7.50-7.42 (m, 1H), 7.30-7.22 (m, 1H), 6.92-6.86 (m, 1H), 3.72-3.63 (m, 2H), 3.51-3.41 (m, 2H), 2.17- 2.08 (m, 2H), 1.56-1.45 (m, 2H), 1.30- 1.28 (m, 3H). | A |
| | | LCMS: m/z 349.2 [M + H]+, (ESI+), | |
| | | RT = 3.30 | |
| E028 | 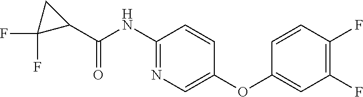 [SMILES: O=C(Nc1ccc(Oc2ccc(F)c(F)c2)cn1)C1CC1(F)F] | 1H NMR (400 MHz, DMSO-d6) δ 11.06 (s, 1H), 8.20-8.17 (m, 1H), 8.11- 8.06 (m, 1H), 7.61-7.54 (m, 1H), 7.51-7.41 (m, 1H), 7.30-7.23 (m, 1H), 6.93-6.86 (m, 1H), 3.02-2.93 (m, 1H), 2.09-1.94 (m, 2H). | A |
| | | LCMS: m/z 327.1 [M + H]+, (ESI+), | |
| | | RT = 3.44 | |
| E029 | 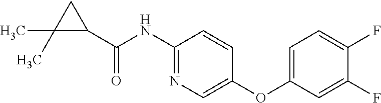 [SMILES: CC1(C)CC1C(=O)Nc1ccc(Oc2ccc(F)c(F)c2)cn1] | 1HNMR(500 MHz, DMSO-d6) δ 10.68 (s, 1H), 8.14 (d,J = 2.7 Hz, 1H), 8.12 (d,J = 9.1 Hz, 1H), 7.53 (dd,J = 9.1, 3.0 Hz, 1H), 7.49-7.40 (m, 1H), 7.23 (ddd,J = 11.8, 6.8, 3.0 Hz, 1H), 6.91- 6.83 (m, 1H), 1.87 (dd,J = 7.9, 5.5 Hz, 1H), 1.14 (d,J = 3.7 Hz, 6H), 0.99 (dd, | A |
| | | J = 5.3, 4.0 Hz, 1H), 0.80 (dd,J = 7.9, 3.8 | |
| | | Hz, 1H). | |
| | | LCMS: m/z 319.1 [M + H]+, (ESI+), | |
| | | RT = 3.92 | |
| E030 | 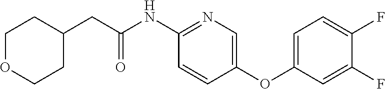 [SMILES: O=C(CC1CCOCC1)Nc1ccc(Oc2ccc(F)c(F)c2)cn1] | 1H NMR (400 MHz, DMSO-d6) δ 10.53 (s, 1H), 8.20-8.09 (m, 2H), 7.59- 7.52 (m, 1H), 7.49-7.39 (m, 1H), 7.28-7.16 (m, 1H), 6.91-6.83 (m, 1H), 3.86-3.76 (m, 2H), 3.30-3.24 (m, 2H), 2.36-2.30 (m, 2H), 2.05- | A |
| | | 1.92 (m, 1H), 1.62-1.53 (m, 2H), 1.30- | |
| | | 1.17 (m, 2H). | |
| | | LCMS: m/z 349.2 [M + H]+, (ESI+), | |
| | | RT = 3.13 | |
| E031 | 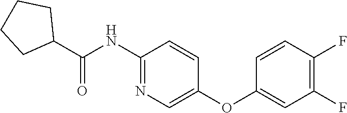 [SMILES: O=C(Nc1ccc(Oc2ccc(F)c(F)c2)cn1)C1CCCC1] | 1H NMR (500 MHz, Chloroform-d) δ 8.25 (d, J = 9.0 Hz, 1H), 8.04 (d, J = 2.9 Hz, 1H), 7.86 (s, 1H), 7.36 (dd, J = 9.0, 2.9 Hz, 1H), 7.15-7.08 (m, 1H), 6.81 (ddd, J = 11.1, 6.5, 3.0 Hz, 1H), 6.69 (dtt, J = 8.3, 3.2, 1.8 Hz, 1H), 2.73 (p, J = 8.1 Hz, 1H), 2.02-1.86 (m, 4H), | B |
| | | 1.84-1.75 (m, 2H), 1.69-1.59 (m, | |
| | | 2H). | |
| | | LCMS: m/z 319.2 [M + H]+, (ESI+), | |
| | | RT = 3.90 | |
| E032 | 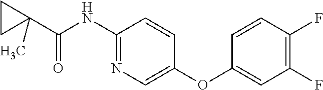 [SMILES: CC1(C(=O)Nc2ccc(Oc3ccc(F)c(F)c3)cn2)CC1] | 1H NMR (500 MHz, Chloroform-d) δ 8.23 (d, J = 9.1 Hz, 1H), 8.14 (s, 1H), 8.06 (d, J = 2.9 Hz, 1H), 7.36 (dd, J = 9.0, 2.9 Hz, 1H), 7.16-7.08 (m, 1H), 6.81 (ddd, J = 11.1, 6.6, 3.0 Hz, 1H), 6.69 (dtt, J = 8.3, 3.2, 1.8 Hz, 1H), 1.49 | B |
| | | (s, 3H), 1.37-1.28 (m, 2H), 0.78- | |
| | | 0.69 (m, 2H). | |
| | | LCMS: m/z 305.2 [M + H]+, (ESI+), | |
| | | RT = 3.71 | |
| E033 | 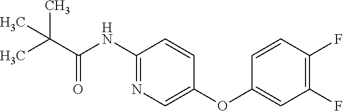 [SMILES: CC(C)(C)C(=O)Nc1ccc(Oc2ccc(F)c(F)c2)cn1] | 1H NMR (500 MHz, Chloroform-d) δ 8.27 (d, J = 9.0 Hz, 1H), 8.06 (d, J = 2.9 Hz, 1H), 8.01 (s, 1H), 7.37 (dd, J = 9.0, 2.9 Hz, 1H), 7.17-7.08 (m, 1H), 6.81 (ddd, J = 11.1, 6.5, 3.0 Hz, 1H), 6.69 (dtd, J = 9.0, 3.2, 1.8 Hz, 1H), 1.33 (s, 9H). | B |
| | | LCMS: m/z 307.2 [M + H]+, (ESI+), | |
| | | RT = 3.84 | |
| E034 | 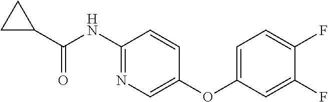 [SMILES: O=C(Nc1ccc(Oc2ccc(F)c(F)c2)cn1)C1CC1] | 1H NMR (500 MHz, Chloroform-d) δ 8.20 (d, J = 9.0 Hz, 1H), 8.11 (s, 1H), 8.05 (d, J = 2.9 Hz, 1H), 7.35 (dd, J = 9.0, 2.9 Hz, 1H), 7.16-7.08 (m, 1H), 6.82 (ddd, J = 11.1, 6.6, 3.0 Hz, 1H), 6.70 (dtd, J = 9.0, 3.2, 1.8 Hz, 1H), 1.56-1.50 (m, 1H), 1.14-1.09 (m, 2H), | B |
| | | 0.94-0.88 (m, 2H). | |
| | | LCMS: m/z 291.1 [M + H]+, (ESI+), | |
| | | RT = 3.40 | |
| E035 | 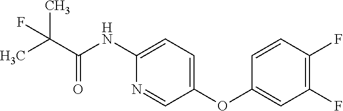 [SMILES: CC(C)(F)C(=O)Nc1ccc(Oc2ccc(F)c(F)c2)cn1] | 1H NMR (500 MHz, DMSO-d6) δ 10.05-9.99 (m, 1H), 8.24-8.20 (m, 1H), 8.07-8.02 (m, 1H), 7.63-7.58 (m, 1H), 7.51-7.42 (m, 1H), 7.31- 7.23 (m, 1H), 6.93-6.87 (m, 1H), 1.61 (s, 3H), 1.57 (s, 3H). LCMS: m/z 311.1 [M + H]+, (ESI+), | A |
| | | RT = 3.64 | |
| E036 | 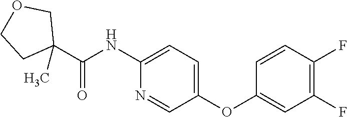 [SMILES: CC1(C(=O)Nc2ccc(Oc3ccc(F)c(F)c3)cn2)CCOC1] | 1H NMR (500 MHz, DMSO-d6) δ 10.22 (s, 1H), 8.20-8.16 (m, 1H), 8.12- 8.06 (m, 1H), 7.59-7.55 (m, 1H), 7.49-7.42 (m, 1H), 7.27-7.22 (m, 1H), 6.91-6.86 (m, 1H), 4.11-4.05 (m, 1H), 3.82-3.73 (m, 2H), 3.53- 3.48 (m, 1H), 2.47-2.40 (m, 1H), 1.87- 1.79 (m, 1H), 1.39 (s, 3H). | A |
| | | LCMS: m/z 335.1 [M + H]+, (ESI+), | |
| | | RT = 3.27 | |
| E037 | 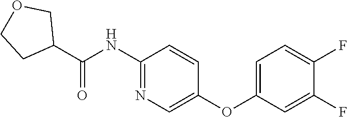 [SMILES: O=C(Nc1ccc(Oc2ccc(F)c(F)c2)cn1)C1CCOC1] | 1H NMR (500 MHz, DMSO-d6) δ 10.68 (s, 1H), 8.18-8.10 (m, 2H), 7.59- 7.54 (m, 1H), 7.49-7.41 (m, 1H), 7.27-7.21 (m, 1H), 6.90-6.85 (m, 1H), 3.95-3.89 (m, 1H), 3.81-3.75 (m, 1H), 3.74-3.67 (m, 2H), 3.30- 3.25 (m, 1H), 2.10-2.04 (m, 2H). LCMS: m/z 321.2 [M + H]+, (ESI+), | A |
| | | RT = 2.95 | |
| E038 | 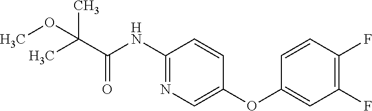 [SMILES: COC(C)(C)C(=O)Nc1ccc(Oc2ccc(F)c(F)c2)cn1] | 1H NMR (500 MHz, DMSO-d6) δ 9.46 (s, 1H), 8.19-8.16 (m, 1H), 8.11- 8.08 (m, 1H), 7.62-7.58 (m, 1H), 7.50- 7.42 (m, 1H), 7.29-7.23 (m, 1H), 6.91-6.86 (m, 1H), 3.27 (s, 3H), 1.38 (s, 6H). LCMS: m/z 323.2 [M + H]+, (ESI+), | A |
| | | RT = 3.68 | |
| E039 | 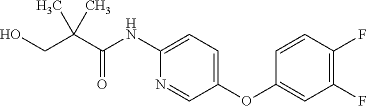 [SMILES: CC(C)(CO)C(=O)Nc1ccc(Oc2ccc(F)c(F)c2)cn1] | 1HNMR(400 MHz, Chloroform-d) δ 9.14 (s, 1H), 8.26 (d,J = 9.1 Hz, 1H), 8.05 (d,J = 2.9 Hz, 1H), 7.38 (dd,J = 9.1, 2.9 Hz, 1H), 7.12 (q,J = 9.1 Hz, 1H), 6.85-6.77 (m, 1H), 6.73-6.65 (m, 1H), 3.79-3.62 (m, 2H), 3.46- 3.35 (m, 1H), 1.30 (s, 6H).LCMS: m/z | A |
| | | 323.1 [M + H]+, (ESI+), RT = 3.08 | |
| E040 | 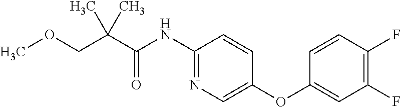 [SMILES: COCC(C)(C)C(=O)Nc1ccc(Oc2ccc(F)c(F)c2)cn1] | 1H NMR (500 MHz, DMSO-d6) δ 9.77 (s, 1H), 8.18-8.16 (m, 1H), 8.11- 8.08 (m, 1H), 7.58-7.55 (m, 1H), 7.49- 7.41 (m, 1H), 7.27-7.21 (m, 1H), 6.90-6.85 (m, 1H), 3.48 (s, 2H), 1.18 (s, 6H). LCMS: m/z 337.2 [M + H]+, (ESI+), | A |
| | | RT = 3.81 | |
| E041 | 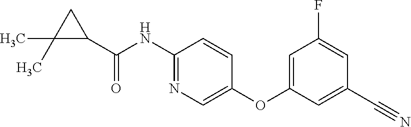 [SMILES: CC1(C)CC1C(=O)Nc1ccc(Oc2cc(F)cc(C#N)c2)cn1] | 1H NMR (500 MHz, DMSO-d6) δ 10.75 (s, 1H), 8.21 (dd, J = 3.0, 0.5 Hz, 1H), 8.17 (d, J = 9.1 Hz, 1H), 7.69- 7.55 (m, 2H), 7.44-7.39 (m, 1H), 7.35 (ddd, J = 10.2, 2.3, 2.3 Hz, 1H), 1.89 (dd, J = 7.9, 5.5 Hz, 1H), 1.15 (s, 3H), 1.14 (s, 3H), 1.00 (dd, J = 5.3, 4.0 Hz, 1H), 0.81 (dd, J = 7.9, 3.8 Hz, 1H). | A |
| | | LCMS: m/z 326.1 [M + H]+, (ESI+), | |
| | | RT = 3.73 | |
| E042 | 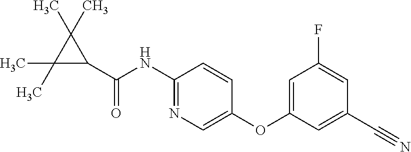 [SMILES: CC1(C)C(C(=O)Nc2ccc(Oc3cc(F)cc(C#N)c3)cn2)C1(C)C] | 1H NMR (500 MHz, DMSO-d6) δ 10.52 (s, 1H), 8.19 (d, J = 2.9 Hz, 1H), 8.14 (d, J = 9.1 Hz, 1H), 7.65-7.56 (m, 2H), 7.43-7.38 (m, 1H), 7.33 (ddd, J = 10.2, 2.3, 2.3 Hz, 1H), 1.56 (s, 1H), 1.24 (s, 6H), 1.17 (s, 6H). LCMS: m/z 354.3 [M + H]+, (ESI+), RT = 4.19 | A |
| E043 | 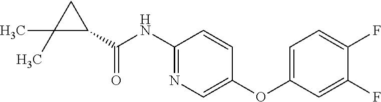 [SMILES: CC1(C)C[C@@H]1C(=O)Nc1ccc(Oc2ccc(F)c(F)c2)cn1] | 1H NMR (400 MHz, DMSO-d6) δ 10.68 (s, 1H), 8.17-8.07 (m, 2H), 7.53 (dd, J = 9.1, 3.0 Hz, 1H), 7.48-7.39 (m, 1H), 7.23 (ddd, J = 11.8, 6.8, 3.0 Hz, 1H), 6.89-6.84 (m, 1H), 1.90- 1.84 (m, 1H), 1.16-1.12 (m, 6H), 1.01- 0.97 (m, 1H), 0.83-0.77 (m, 1H). | A |
| | | LCMS: m/z 319.1 [M + H]+, (ESI+), | |
| | | RT = 3.81 | |
| E044 | 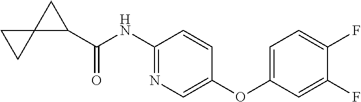 [SMILES: O=C(Nc1ccc(Oc2ccc(F)c(F)c2)cn1)C1CC12CC2] | 1H NMR (400 MHz, DMSO-d6) δ 10.64 (s, 1H), 8.16-8.09 (m, 2H), 7.53 (dd, J = 9.2, 2.9 Hz, 1H), 7.49-7.40 (m, 1H), 7.23 (ddd, J = 11.9, 6.8, 3.0 Hz, 1H), 6.89-6.84 (m, 1H), 2.36- 2.33 (m, 1H), 1.41-1.36 (m, 1H), 1.34- 1.30 (m, 1H), 0.93-0.81 (m, 3H), | A |
| | | 0.78-0.71 (m, 1H). | |
| | | LCMS: m/z 317.1 [M + H]+, (ESI+), | |
| | | RT = 3.51 | |
| E045 | 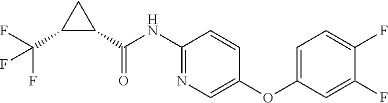 [SMILES: O=C(Nc1ccc(Oc2ccc(F)c(F)c2)cn1)[C@H]1C[C@H]1C(F)(F)F] | 1H NMR (400 MHz, DMSO-d6) δ 11.00 (s, 1H), 8.17 (dd, J = 3.0, 0.6 Hz, 1H), 8.07 (d, J = 9.1 Hz, 1H), 7.55 (dd, J = 9.0, 3.0 Hz, 1H), 7.49-7.40 (m, 1H), 7.26 (ddd, J = 11.8, 6.8, 3.0 Hz, 1H), 6.92-6.86 (m, 1H), 2.42-2.34 (m, 1H), 2.30-2.16 (m, 1H), 1.56- | A |
| | Relative cis stereochemistry | 1.49 (m, 1H), 1.36-1.27 (m, 1H). | |
| | | LCMS: m/z 359.1 [M + H]+, (ESI+), | |
| | | RT = 3.50 | |
| E046 | 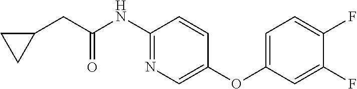 [SMILES: O=C(CC1CC1)Nc1ccc(Oc2ccc(F)c(F)c2)cn1] | 1H NMR (400 MHz, DMSO-d6) δ 10.43 (s, 1H), 8.17-8.12 (m, 2H), 7.56 (dd, J = 9.1, 2.9 Hz, 1H), 7.49-7.40 (m, 1H), 7.23 (ddd, J = 11.8, 6.8, 3.0 Hz, 1H), 6.91-6.84 (m, 1H), 2.27 (d, J = 7.1 Hz, 2H), 1.12-0.98 (m, 1H), | A |
| | | 0.50-0.43 (m, 2H), 0.22-0.15 (m, | |
| | | 2H). | |
| | | LCMS: m/z 305.1 [M + H]+, (ESI+), | |
| | | RT = 3.39 | |
| E047 | 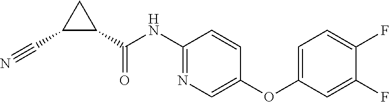 [SMILES: N#C[C@@H]1C[C@@H]1C(=O)Nc1ccc(Oc2ccc(F)c(F)c2)cn1] | 1H NMR (400 MHz, DMSO-d6) δ 11.15 (s, 1H), 8.21-8.16 (m, 1H), 8.13 (d, J = 9.1 Hz, 1H), 7.58 (dd, J = 9.0, 3.0 Hz, 1H), 7.50-7.41 (m, 1H), 7.26 (ddd, J = 11.8, 6.8, 3.0 Hz, 1H), 6.95- 6.84 (m, 1H), 2.56-2.51 (m, 1H), 2.29- 2.21 (m, 1H), 1.50-1.39 (m, 2H). | A |
| | Relative cis stereochemistry | LCMS: m/z 316.1 [M + H]+, (ESI+), | |
| | | RT = 2.96 | |
| E048 | 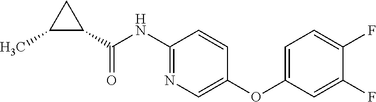 [SMILES: C[C@@H]1C[C@@H]1C(=O)Nc1ccc(Oc2ccc(F)c(F)c2)cn1] | 1H NMR (500 MHz, DMSO-d6) δ 10.75 (s, 1H), 8.16-8.11 (m, 2H), 7.53 (dd, J = 9.0, 3.0 Hz, 1H), 7.49-7.40 (m, 1H), 7.24 (ddd, J = 11.8, 6.8, 3.0 Hz, 1H), 6.91-6.83 (m, 1H), 2.01 (td, J = 8.1, 5.4 Hz, 1H), 1.33-1.20 (m, 1H), 1.11 (d, J = 6.2 Hz, 3H), 0.96 (td, J = | A |
| | Relative cis stereochemistry | 8.1, 3.8 Hz, 1H), 0.83-0.74 (m, 1H). | |
| | | LCMS: m/z 305.1 [M + H]+, (ESI+), | |
| | | RT = 3.54 | |
| E049 | 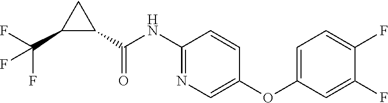 [SMILES: O=C(Nc1ccc(Oc2ccc(F)c(F)c2)cn1)[C@H]1C[C@@H]1C(F)(F)F] | 1HNMR(500 MHz, DMSO-d6) δ 11.10 (s, 1H), 8.18 (d,J = 3.0 Hz, 1H), 8.08 (d,J = 9.0 Hz, 1H), 7.57 (dd,J = 9.0, 3.0 Hz, 1H), 7.51-7.40 (m, 1H), 7.26 (ddd,J = 11.8, 6.8, 3.0 Hz, 1H), 6.95- 6.83 (m, 1H), 2.34-2.21 (m, 1H), 1.35- 1.26 (m, 2H). | A |
| | Relative trans stereochemistry | LCMS: m/z 359.2 [M + H]+, (ESI+), | |
| | | RT = 3.82 | |
| E050 | 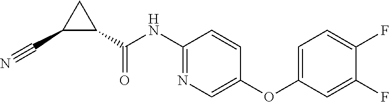 [SMILES: N#C[C@H]1C[C@@H]1C(=O)Nc1ccc(Oc2ccc(F)c(F)c2)cn1] | 1HNMR(500 MHz, DMSO-d6) δ 11.17 (s, 1H), 8.19 (d,J = 2.8 Hz, 1H), 8.06 (d,J = 9.0 Hz, 1H), 7.57 (dd,J = 9.0, 3.0 Hz, 1H), 7.51-7.40 (m, 1H), 7.26 (ddd,J = 11.8, 6.8, 3.0 Hz, 1H), 6.93- 6.84 (m, 1H), 2.72-2.66 (m, 1H), 2.12 (ddd,J = 9.4, 6.1, 4.3 Hz, 1H), 1.58 | A |
| | Relative trans stereochemistry | (ddd,J = 8.7, 6.1, 4.4 Hz, 1H), 1.40 | |
| | | (ddd,J = 10.2, 5.9, 4.5 Hz, 1H). | |
| | | LCMS: m/z 316.2 [M + H]+, (ESI+), | |
| | | RT = 3.24 | |
| E051 | 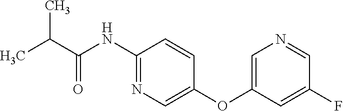 [SMILES: CC(C)C(=O)Nc1ccc(Oc2cncc(F)c2)cn1] | 1H NMR (400 MHz, Chloroform-d) δ 8.32 (d, J = 9.0 Hz, 1H), 8.26 (d, J = 2.4 Hz, 1H), 8.23 (d, J = 2.2 Hz, 1H), 8.11 (d, J = 2.8 Hz, 1H), 7.92 (s, 1H), 7.44 (dd, J = 9.0, 2.9 Hz, 1H), 6.99 (dt, J = 9.5, 2.4 Hz, 1H), 2.57 (hept, J = 6.9 Hz, 1H), 1.28 (d, J = 6.9 Hz, 6H). | A |
| | | LCMS: m/z 276.1 [M + H]+, (ESI+), | |
| | | RT = 2.51 | |
| E052 | 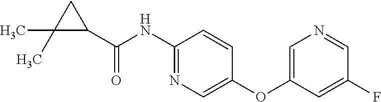 [SMILES: CC1(C)CC1C(=O)Nc1ccc(Oc2cncc(F)c2)cn1] | 1H NMR (400 MHz, Chloroform-d) δ 8.32-8.21 (m, 3H), 8.14-8.04 (m, 2H), 7.43 (dd, J = 9.0, 2.9 Hz, 1H), 6.99 (dt, J = 9.5, 2.4 Hz, 1H), 1.45 (dd, J = 7.9, 5.3 Hz, 1H), 1.28-1.23 (m, 4H), 1.22 (s, 3H), 0.91 (dd, J = 7.9, 4.4 Hz, 1H). | A |
| | | LCMS: m/z 302.2 [M + H]+, (ESI+), | |
| | | RT = 3.07 | |
| E053 | 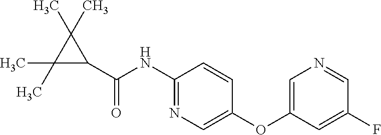 [SMILES: CC1(C)C(C(=O)Nc2ccc(Oc3cncc(F)c3)cn2)C1(C)C] | 1H NMR (400 MHz, Chloroform-d) δ 8.29-8.21 (m, 3H), 8.09 (d, J = 2.6 Hz, 1H), 8.02 (s, 1H), 7.41 (dd, J = 9.0, 2.9 Hz, 1H), 6.98 (dt, J = 9.5, 2.4 Hz, 1H), 1.33 (s, 6H), 1.23 (s, 6H), 1.04 (s, 1H). LCMS: m/z 330.2 [M + H]+, (ESI+), RT = 3.71 | A |
| E054 | 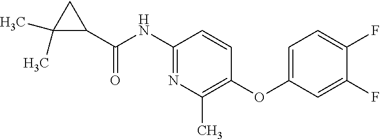 [SMILES: Cc1nc(NC(=O)C2CC2(C)C)ccc1Oc1ccc(F)c(F)c1] | 1HNMR(400 MHz, Chloroform-d) δ 8.07 (d,J = 8.8 Hz, 1H), 8.01 (s, 1H), 7.28 (d,J = 8.8 Hz, 1H), 7.16-7.07 (m, 1H), 6.74 (ddd,J = 11.3, 6.5, 3.0 Hz, 1H), 6.61 (dtd,J = 9.0, 3.2, 1.8 Hz, 1H), 2.37 (s, 3H), 1.46 (dd,J = 7.9, 5.3 Hz, 1H), 1.27 (d,J = 4.8 Hz, 1H), 1.25 (s, 3H), 1.22 (s, 3H), 0.90 (dd,J = 7.9, 4.4 Hz, 1H). | A |
| | | LCMS: m/z 333.5 [M + H]+, (ESI+), | |
| | | RT = 4.05 | |
| E055 | 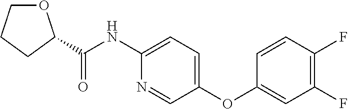 [SMILES: O=C(Nc1ccc(Oc2ccc(F)c(F)c2)cn1)[C@@H]1CCCO1] | 1H NMR (500 MHz, DMSO-d6) δ 9.88 (s, 1H), 8.18 (d, J = 2.7 Hz, 1H), 8.11 (d, J = 9.0 Hz, 1H), 7.59 (dd, J = 9.0, 3.0 Hz, 1H), 7.50-7.40 (m, 1H), 7.29- 7.21 (m, 1H), 6.91-6.85 (m, 1H), 4.52- 4.44 (m, 1H), 4.01-3.93 (m, 1H), 3.86-3.78 (m, 1H), 2.26-2.15 (m, | B |
| | | 1H), 2.01-1.81 (m, 3H). | |
| | | LCMS: m/z 321.1 [M + H]+, (ESI+), | |
| | | RT = 3.47 | |
| E056 | 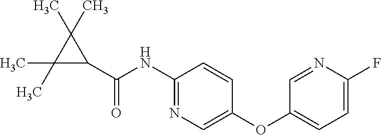 [SMILES: CC1(C)C(C(=O)Nc2ccc(Oc3ccc(F)nc3)cn2)C1(C)C] | 1H NMR (500 MHz, Chloroform-d) δ 8.21 (d, J = 9.1 Hz, 1H), 8.05 (d, J = 2.8 Hz, 1H), 7.99-7.91 (m, 2H), 7.40 (ddd, J = 9.2, 6.4, 3.1 Hz, 1H), 7.34 (dd, J = 9.1, 2.9 Hz, 1H), 6.92 (dd, J = 8.8, 3.5 Hz, 1H), 1.32 (s, 6H), 1.23 (s, 6H), 1.03 (s, 1H). LCMS: m/z 330.3 [M + H]+, (ESI+), RT = 3.79 | |
| E057 | 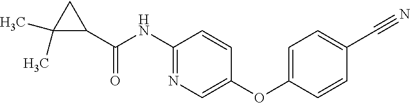 [SMILES: CC1(C)CC1C(=O)Nc1ccc(Oc2ccc(C#N)cc2)cn1] | 1H NMR (400 MHz, DMSO-d6) δ 10.74 (s, 1H), 8.24-8.14 (m, 2H), 7.89- 7.80 (m, 2H), 7.63 (dd, J = 9.0, 3.0 Hz, 1H), 7.18-7.08 (m, 2H), 1.93- 1.85 (m, 1H), 1.15 (d, J = 3.1 Hz, 6H), 1.04-0.97 (m, 1H), 0.85-0.77 (m, 1H). | A |
| | | LCMS: m/z 308.3 [M + H]+, (ESI+), | |
| | | RT = 3.46 | |
| E058 | 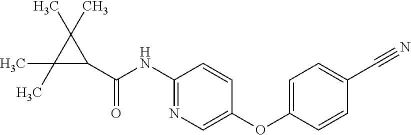 [SMILES: CC1(C)C(C(=O)Nc2ccc(Oc3ccc(C#N)cc3)cn2)C1(C)C] | 1H NMR (400 MHz, DMSO-d6) δ 10.51 (s, 1H), 8.19 (d, J = 2.9 Hz, 1H), 8.14 (d, J = 9.1 Hz, 1H), 7.88-7.80 (m, 2H), 7.60 (dd, J = 9.1, 3.0 Hz, 1H), 7.17- 7.08 (m, 2H), 1.55 (s, 1H), 1.24 (s, 6H), 1.17 (s, 6H). LCMS: m/z 336.3 [M + H]+, (ESI+), RT = 4.16 | A |
| E059 | 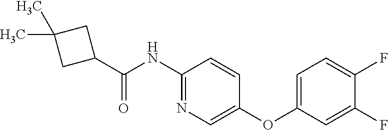 [SMILES: CC1(C)CC(C(=O)Nc2ccc(Oc3ccc(F)c(F)c3)cn2)C1] | 1H NMR (400 MHz, Chloroform-d) δ 8.20 (d, J = 9.0 Hz, 1H), 7.97 (d, J = 2.8 Hz, 1H), 7.67 (s, 1H), 7.30 (dd, J = 9.0, 2.9 Hz, 1H), 7.10-7.00 (m, 1H), 6.75 (ddd, J = 11.1, 6.6, 3.0 Hz, 1H), 6.66- 6.59 (m, 1H), 3.04 (p, J = 8.8 Hz, 1H), 2.13-2.06 (m, 2H), 2.00-1.93 (m, 2H), 1.14 (s, 3H), 1.06 (s, 3H). | B |
| | | LCMS: m/z 333.3 [M + H]+, (ESI+), | |
| | | RT = 4.05 | |
| E060 | 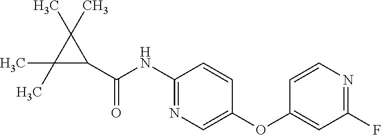 [SMILES: CC1(C)C(C(=O)Nc2ccc(Oc3ccnc(F)c3)cn2)C1(C)C] | 1H NMR (500 MHz, Chloroform-d) δ 8.34-8.29 (m, 1H), 8.14-8.09 (m, 2H), 8.03 (s, 1H), 7.47 (dd, J = 9.0, 2.8 Hz, 1H), 6.76 (ddd, J = 5.8, 2.1, 0.9 Hz, 1H), 6.41-6.38 (m, 1H), 1.36 (s, 6H), 1.26 (s, 6H), 1.07 (s, 1H).LCMS: m/z 330.1 [M + H]+, (ESI+), RT = 3.82 | A |
| E061 | 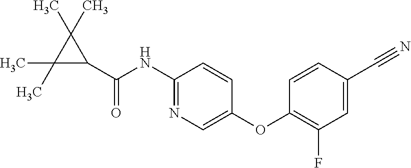 [SMILES: CC1(C)C(C(=O)Nc2ccc(Oc3ccc(C#N)cc3F)cn2)C1(C)C] | 1H NMR (500 MHz, Chloroform-d) δ 8.26 (d, J = 9.0 Hz, 1H), 8.09 (d, J = 2.7 Hz, 1H), 7.98 (s, 1H), 7.49 (dd, J = 10.1, 1.9 Hz, 1H), 7.43-7.35 (m, 2H), 6.94 (t, J = 8.3 Hz, 1H), 1.33 (s, 6H), 1.23 (s, 6H), 1.04 (s, 1H). LCMS: m/z 354.3 [M + H]+, (ESI+), RT = 4.09 | A |
| E062 | 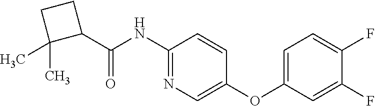 [SMILES: CC1(C)CCC1C(=O)Nc1ccc(Oc2ccc(F)c(F)c2)cn1] | 1H NMR (400 MHz, DMSO-d6) δ 10.27 (s, 1H), 8.19-8.11 (m, 2H), 7.54 (dd, J = 9.0, 3.0 Hz, 1H), 7.48-7.39 (m, 1H), 7.29-7.19 (m, 1H), 6.92- 6.82 (m, 1H), 3.06 (t, J = 7.9 Hz, 1H), 2.29-2.18 (m, 1H), 1.89-1.79 (m, 1H), 1.74-1.62 (m, 2H), 1.23 (s, 3H), | A |
| | | 0.97 (s, 3H). | |
| | | LCMS: m/z 333.3 [M + H]+, (ESI+), | |
| | | RT = 4.12 | |
| E063 | 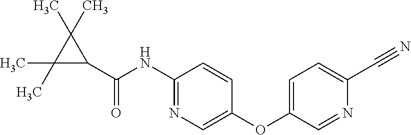 [SMILES: CC1(C)C(C(=O)Nc2ccc(Oc3ccc(C#N)nc3)cn2)C1(C)C] | 1H NMR (400 MHz, DMSO-d6) δ 10.54 (s, 1H), 8.57 (dd, J = 2.9, 0.5 Hz, 1H), 8.26 (dd, J = 3.0, 0.5 Hz, 1H), 8.19- 8.12 (m, 1H), 8.03 (dd, J = 8.7, 0.5 Hz, 1H), 7.67 (dd, J = 9.1, 3.0 Hz, 1H), 7.54 (dd, J = 8.7, 2.9 Hz, 1H), 1.56 (s, 1H), 1.24 (s, 6H), 1.17 (s, 6H). LCMS: m/z 337.3 [M + H]+, (ESI+), RT = 3.87 | B |
| E064 | 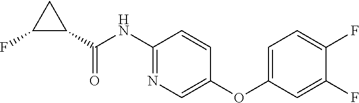 [SMILES: O=C(Nc1ccc(Oc2ccc(F)c(F)c2)cn1)[C@H]1C[C@H]1F] | 1H NMR (500 MHz, Chloroform-d) δ 8.37 (s, 1H), 8.14 (d, J = 9.0 Hz, 1H), 8.09-8.03 (m, 1H), 7.36 (dd, J = 9.0, 2.9 Hz, 1H), 7.17-7.07 (m, 1H), 6.82 (ddd, J = 11.1, 6.5, 2.9 Hz, 1H), 6.70 (dtd, J = 9.0, 3.3, 1.8 Hz, 1H), 4.88 (dddd, J = 64.0, 6.0, 3.5, 1.5 Hz, 1H), | B |
| | Relative trans stereochemistry | 2.10-1.96 (m, 1H), 1.58-1.42 (m, | |
| | | 2H). | |
| | | LCMS: m/z 309.2 [M + H]+, (ESI+), | |
| | | RT = 3.47 | |
| E065 | 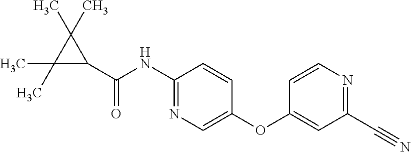 [SMILES: CC1(C)C(C(=O)Nc2ccc(Oc3ccnc(C#N)c3)cn2)C1(C)C] | (500 MHz, Chloroform-d) δ 8.55 (d, J = 5.7 Hz, 1H), 8.33 (d, J = 9.1 Hz, 1H), 8.11-8.08 (m, 1H), 8.01 (s, 1H), 7.44 (dd, J = 9.0, 2.9 Hz, 1H), 7.22 (d, J = 2.5 Hz, 1H), 7.00 (dd, J = 5.7, 2.5 Hz, 1H), 1.33 (s, 6H), 1.24 (s, 6H), 1.05 (s, 1H). LCMS: m/z 337.3 [M + H]+, (ESI+), RT = 3.71 | B |
| E066 | 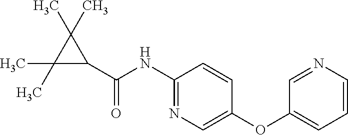 [SMILES: CC1(C)C(C(=O)Nc2ccc(Oc3cccnc3)cn2)C1(C)C] | (500 MHz, Chloroform-d) δ 8.40 (d, J = 1.7 Hz, 1H), 8.38 (dd, J = 4.1, 1.8 Hz, 1H), 8.21 (d, J = 9.0 Hz, 1H), 8.07 (d, J = 2.9 Hz, 1H), 7.98 (s, 1H), 7.37 (dd, J = 9.0, 2.9 Hz, 1H), 7.29-7.26 (m, 1H), 7.26-7.23 (m, 1H), 1.32 (s, 6H), 1.23 (s, 6H), 1.03 (s, 1H). LCMS: m/z 312.3 [M + H]+, (ESI+), RT = 3.10 | A |
| E067 | 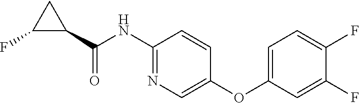 [SMILES: O=C(Nc1ccc(Oc2ccc(F)c(F)c2)cn1)[C@@H]1C[C@H]1F] | 1H NMR (500 MHz, Chloroform-d) δ 8.24 (d, J = 9.0 Hz, 1H), 8.11 (s, 1H), 8.08-8.03 (m, 1H), 7.37 (dd, J = 9.0, 2.9 Hz, 1H), 7.17-7.09 (m, 1H), 6.83 (ddd, J = 11.1, 6.6, 3.0 Hz, 1H), 6.70 (dtd, J = 9.0, 3.2, 1.8 Hz, 1H), 4.82 (dtd, J = 65.1, 6.2, 3.8 Hz, 1H), 1.92 | A |
| | Relative cis stereochemistry | (dtd, J = 22.8, 7.3, 3.8 Hz, 1H), 1.86- | |
| | | 1.78 (m, 1H), 1.25 (dddd, J = 12.3, 9.4, | |
| | | 7.3, 6.1 Hz, 1H). | |
| | | LCMS: m/z 309.2 [M + H]+, (ESI+), | |
| | | RT = 3.12 | |
| E068 | 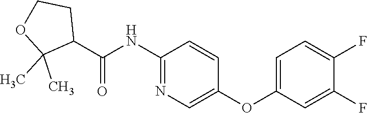 [SMILES: CC1(C)OCCC1C(=O)Nc1ccc(Oc2ccc(F)c(F)c2)cn1] | 1H NMR (400 MHz, DMSO-d6) δ 10.59 (s, 1H), 8.19-8.10 (m, 2H), 7.55 (dd, J = 9.0, 3.0 Hz, 1H), 7.49-7.40 (m, 1H), 7.28-7.21 (m, 1H), 6.91- 6.85 (m, 1H), 3.91-3.83 (m, 1H), 3.76- 3.68 (m, 1H), 3.05-2.97 (m, 1H), 2.31-2.21 (m, 1H), 2.13-2.01 (m, | A |
| | | 1H), 1.28 (s, 3H), 1.09 (s, 3H). | |
| | | LCMS: m/z 349.3 [M + H]+, (ESI+), | |
| | | RT = 3.39 | |
| E069 | 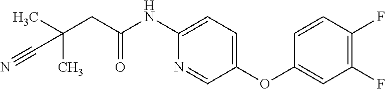 [SMILES: CC(C)(C#N)CC(=O)Nc1ccc(Oc2ccc(F)c(F)c2)cn1] | (400 MHz, Chloroform-d) δ 8.23 (d, J = 8.9 Hz, 1H), 8.12 (s, 1H), 8.06 (d, J = 2.8 Hz, 1H), 7.37 (dd, J = 9.0, 2.9 Hz, 1H), 7.13 (q, J = 9.0 Hz, 1H), 6.83 (ddd, J = 11.0, 6.5, 2.9 Hz, 1H), 6.75- 6.67 (m, 1H), 2.64 (s, 2H), 1.54 (s, 6H). | B |
| | | LCMS: m/z 332.2 [M + H]+, (ESI+), | |
| | | RT = 3.23 | |
| E070 | 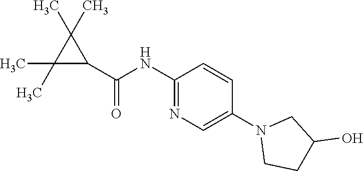 [SMILES: CC1(C)C(C(=O)Nc2ccc(N3CCC(O)C3)cn2)C1(C)C] | (500 MHz, Chloroform-d) δ 8.00 (d, J = 8.9 Hz, 1H), 7.76 (s, 1H), 7.59 (d, J = 2.9 Hz, 1H), 6.88 (dd, J = 9.0, 3.0 Hz, 1H), 4.62 (tt, J = 4.6, 2.2 Hz, 1H), 3.53- 3.45 (m, 2H), 3.32 (td, J = 8.8, 3.4 Hz, 1H), 3.26 (d, J = 10.2 Hz, 1H), 2.23-2.14 (m, 1H), 2.11-2.04 (m, 1H), 1.91 (s, 1H), 1.31 (s, 6H), 1.20 (s, 6H), 1.00 (s, 1H). LCMS: m/z 304.2 [M + H]+, (ESI+), RT = 1.78 | A |
| E071 | 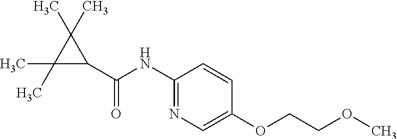 | 1H NMR (500 MHz, DMSO-d6) δ 10.23 (s, 1H), 8.05-7.97 (m, 1H), 7.94 (d, J = 9.1 Hz, 1H), 7.38 (dd, J = 9.1, 3.1 Hz, 1H), 4.20-4.07 (m, 2H), 3.74- 3.59 (m, 2H), 3.30 (s, 3H), 1.48 (s, 1H), 1.23 (s, 6H), 1.15 (s, 6H). LCMS: m/z 293.3 [M + H]+, (ESI+), RT = 2.87 | A |
| E072 | 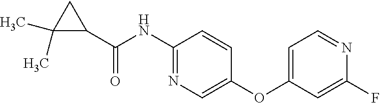 [SMILES: CC1(C)CC1C(=O)Nc1ccc(Oc2ccnc(F)c2)cn1] | 1H NMR (500 MHz, DMSO-d6) δ 10.79 (s, 1H), 8.30-8.24 (m, 1H), 8.20 (d, J = 9.1 Hz, 1H), 8.14 (d, J = 5.8 Hz, 1H), 7.71 (dd, J = 9.0, 3.0 Hz, 1H), 6.94 (ddd, J = 5.8, 2.1, 0.9 Hz, 1H), 6.74 (d, J = 2.1 Hz, 1H), 1.90 (dd, J = 7.9, 5.5 Hz, 1H), 1.15 (s, 3H), 1.15 (s, 3H), | B |
| | | 1.03-0.98 (m, 1H), 0.86-0.77 (m, 1H). | |
| | | LCMS: m/z 302.2 [M + H]+, (ESI+), | |
| | | RT = 3.27 | |
| E073 | 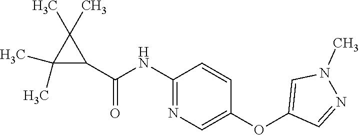 [SMILES: Cn1cc(Oc2ccc(NC(=O)C3C(C)(C)C3(C)C)nc2)cn1] | 1H NMR (400 MHz, Chloroform-d) δ 8.11 (d, J = 9.1 Hz, 1H), 8.04 (d, J = 3.0 Hz, 1H), 7.86 (s, 1H), 7.33 (dd, J = 9.1, 3.0 Hz, 1H), 7.31 (s, 1H), 7.23 (s, 1H), 3.88 (s, 3H), 1.31 (s, 6H), 1.22 (s, 6H), 1.01 (s, 1H). LCMS: m/z 315.3 [M + H]+, (ESI+), RT = 3.19 | A |
| E074 | 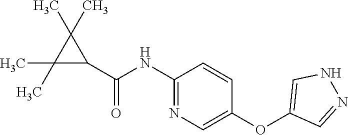 [SMILES: CC1(C)C(C(=O)Nc2ccc(Oc3cn[nH]c3)cn2)C1(C)C] | 1H NMR(400 MHz, Chloroform-d) δ 8.57 (s, 1H), 8.20 (d,J = 9.1 Hz, 1H), 8.04 (d,J = 2.9 Hz, 1H), 7.48 (s, 2H), 7.40 (dd,J = 9.1, 3.0 Hz, 1H), 1.34 (s, 6H), 1.25 (s, 6H), 1.10 (s, 1H). LCMS: m/z 301.2 [M + H]+, (ESI+), RT = 2.92 | A |
| E075* | 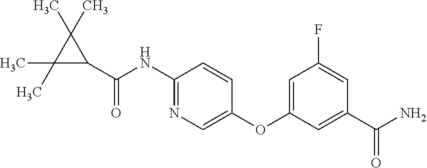 [SMILES: CC1(C)C(C(=O)Nc2ccc(Oc3cc(F)cc(C(N)=O)c3)cn2)C1(C)C] | 1H NMR (500 MHz, DMSO-d6) δ 10.50 (s, 1H), 8.20-8.16 (m, 1H), 8.13 (d, J = 9.0 Hz, 1H), 8.08 (s, 1H), 7.64- 7.52 (m, 2H), 7.45 (ddd, J = 9.4, 2.3, 1.4 Hz, 1H), 7.33-7.25 (m, 1H), 7.10 (ddd, J = 9.8, 2.3, 2.3 Hz, 1H), 1.55 (s, 1H), 1.24 (s, 6H), 1.17 (s, 6H). LCMS: m/z 372.3 [M + H]+, (ESI+), RT = 3.39 | A |
| E076** | 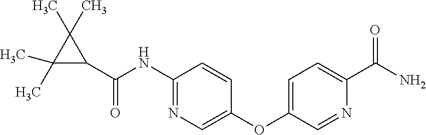 [SMILES: CC1(C)C(C(=O)Nc2ccc(Oc3ccc(C(N)=O)nc3)cn2)C1(C)C] | 1H NMR (400 MHz, DMSO-d6) δ 10.51 (s, 1H), 8.41 (d, J = 2.4 Hz, 1H), 8.23 (d, J = 2.5 Hz, 1H), 8.14 (d, J = 9.0 Hz, 1H), 8.05-7.98 (m, 2H), 7.63 (dd, J = 9.1, 3.0 Hz, 1H), 7.56 (s, 1H), 7.50 (dd, J = 8.7, 2.9 Hz, 1H), 1.55 (s, 1H), 1.24 (s, 6H), 1.17 (s, 6H).LCMS: m/z 355.4 [M + H]+, (ESI+), RT = 3.35 | B |
| E077 | 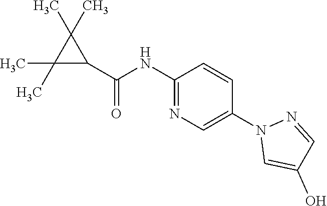 [SMILES: CC1(C)C(C(=O)Nc2ccc(-n3cc(O)cn3)cn2)C1(C)C] | 1HNMR(400 MHz, DMSO-d6) δ 10.48 (s, 1H), 8.98 (s, 1H), 8.67 (dd,J = 2.7, 0.7 Hz, 1H), 8.16-8.09 (m, 1H), 8.07 (dd,J = 9.0, 2.8 Hz, 1H), 7.94 (d,J = 0.7 Hz, 1H), 7.37 (d,J = 0.7 Hz, 1H), 1.55 (s, 1H), 1.25 (s, 6H), 1.18 (s, 6H). LCMS: m/z 301.2 [M + H]+, (ESI+), RT = 2.95 | A |

*E075 was formed as a byproduct in the synthesis of E042
**E076 was formed as a byproduct in the synthesis of E063

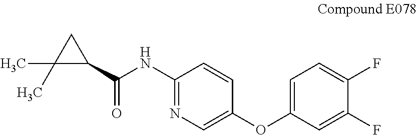 [SMILES: CC1(C)C[C@H]1C(=O)Nc1ccc(Oc2ccc(F)c(F)c2)cn1]

### (1R)-N-[5-(3,4-difluorophenoxy)-2-pyridyl]-2,2-dimethyl-cyclopropanecarboxamide

Synthesized from Compound E029 using chiral separation with the following conditions: Chiralpak AD-H column (20×250 mm, 5 μm), Methanol mobile phase (9 mL/min) to afford a colourless oil as the second-eluting component. Comparison with Compound E043 confirmed absolute stereochemistry. The product was further purified by column chromatography (10 g Biotage SNAP cartridge, 0-40% gradient of ethyl acetate in heptane) to afford the title compound as a colourless gum. 1H NMR (400 MHz, DMSO-d6) δ 10.67 (s, 1H), 8.14 (dd, J=3.0, 0.5 Hz, 1H), 8.12 (d, J=9.1 Hz, 1H), 7.53 (dd, J=9.1, 3.0 Hz, 1H), 7.44 (dt, J=10.5, 9.2 Hz, 1H), 7.23 (ddd, J=11.8, 6.8, 3.0 Hz, 1H), 6.87 (dtt, J=8.4, 3.2, 1.8 Hz, 1H), 1.87 (dd, J=7.9, 5.5 Hz, 1H), 1.14 (d, J=3.0 Hz, 6H), 0.99 (dd, J=5.3, 3.9 Hz, 1H), 0.80 (dd, J=7.9, 3.8 Hz, 1H). LCMS: m/z 319.2 [M+H]+, (ESI+), RT=3.82 (Method A)

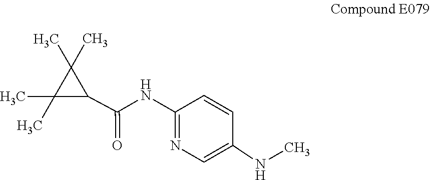

### 2,2,3,3-tetramethyl-N-[5-(methylamino)-2-pyridyl]cyclopropanecarboxamide

Step 1

5-bromo-2-nitro-pyridine (250 mg, 1.23 mmol), cesium carbonate (803 mg, 2.46 mmol) and N-methyl-l-phenyl-methanamine (0.16 mL, 1.23 mmol) were mixed in DMSO (6.25 mL), purged with nitrogen and stirred in a RBF at 50° C. for 3 hours. The reaction temperature was raised to 80° C. and the reaction continued to stir for 3h. The reaction mixture was diluted with water (50 mL). The mixture was extracted with EtOAc (3×25 ml). The combined organics were dried (hydrophobic frit) and concentrated under reduced pressure. The crude product was purified by flash column chromatography (50 g SiO2 column, 0-80% EtOAc in heptane) to afford N-benzyl-N-methyl-6-nitro-pyridin-3-amine (60 mg, 0.230 mmol, 19% Yield) as a yellow oil. 1HNMR(400 MHz, DMSO-d6) δ 8.14 (d,J=9.2 Hz, 1H), 8.06 (d,J=3.1 Hz, 1H), 7.36 (ddd,J=7.5, 6.3, 1.2 Hz, 2H), 7.31-7.20 (m, 4H), 4.82 (s, 2H), 3.25 (s, 3H).

Step 2

To a solution of N-benzyl-N-methyl-6-nitro-pyridin-3-amine (60 mg, 0.230 mmol) in 3:1 Et0H/H20 (4 mL) was added iron (128 mg, 2.30 mmol) and ammonium chloride (123 mg, 2.30 mmol). The reaction mixture was heated to 70° C. for 2 hours. The reaction mixture was then cooled, filtered through a pad of Celite, washing with EtOAc (2×25 ml). The filtrate was diluted with water (25 ml) and the layers were separated and the aqueous layer was extracted twice with EtOAc (2×25 ml).The combined organic extracts were washed with brine, dried (hydrophobic frit) and concentrated under reduced pressure to afford N5-benzyl-N5-methyl-pyridine-2,5-diamine (90.0%) (52 mg, 0.219 mmol, 95% Yield) as a black oil. 1H NMR (400 MHz, DMSO-d6) δ 7.51 (d,J=2.7 Hz, 1H), 7.31 (ddd,J=9.7, 4.4, 2.4 Hz, 2H), 7.22 (td,J=5.2, 4.7, 2.2 Hz, 3H), 7.10 (dd,J=8.9, 3.1 Hz, 1H), 6.39 (dd,J=8.8, 0.6 Hz, 1H), 5.21 (s, 2H), 4.30 (s, 2H), 2.76 (s, 3H).

Step 3

To a solution of N5-benzyl-N5-methyl-pyridine-2,5-diamine (90%, 52 mg, 0.219 mmol)and N-ethyl-N-isopropyl-propan-2-amine (0.077 mL, 0.439 mmol) in THF-Anhydrous (3 mL) was added 2,2,3,3-tetramethylcyclopropanecarbonyl chloride (42 mg, 0.263 mmol) with stirring at RT for 2 hours after which time, MeOH (1 mL) and 1M NaOH (1 mL) were added and the reaction mixture stirred at rt for 2 h. The solvent was removed under reduced pressure. The residue was re-suspended in brine (15 mL) and EtOAc (15 mL). The organic layer was separated and the aqueous extracted with further EtOAc (2×15 ml). The combined organic extracts were dried (hydrophobic frit) and concentrated in vacuo to afford N-[5-[benzyl(methyl)amino]-2-pyridyl]-2,2,3,3-tetramethyl-cyclopropanecarboxamide (92.0%) (75 mg, 0.203 mmol, 93% Yield) as a red brown oil. 1H NMR (400 MHz, Chloroform-d) δ 7.91 (d, J=9.1 Hz, 1H), 7.76 (s, 1H), 7.70 (d, J=3.1 Hz, 1H), 7.24 (t, J=7.3 Hz, 2H), 7.16 (d, J=7.4 Hz, 1H), 7.13 (d, J=7.1 Hz, 2H), 7.01 (dd, J=9.1, 3.1 Hz, 1H), 4.41 (s, 2H), 2.92 (s, 3H), 1.23 (s, 6H), 1.12 (s, 6H), 0.93 (s, 1H).

Step 4

A stirred solution of N-[5-[benzyl(methyl)amino]-2-pyridyl]-2,2,3,3-tetramethyl-cyclopropanecarboxamide (92%, 75 mg, 0.203 mmol) in Ethyl acetate (10 mL) was put under a balloon of hydrogen and was stirred at RT for 16 hours. A further portion of 10% Palladium on Carbon (4.3 mg, 0.0406 mmol) was added and the mixture was put under a balloon of hydrogen and was stirred at RT for 5 hours. The reaction mixture was filtered through Celite which was washed with further Dioxane (50 ml). The filtrate was concentrated under reduced pressure. The crude residue was dissolved in methanol (5m1). The solution was passed through the H-Cube flow hydrogenator fitted with a 10% Pd/C cartridge at a flow rate of 1 ml/min with a reaction temperature of 80° C. The generated hydrogen was supplied to the flow at 80 bar. The crude mixture was then passed through the H-Cube two more times under the same conditions but with the addition of Acetic Acid (5% (v/v) to the reaction mixture. The mixture was concentrated under reduced pressure and purified by prep HPLC (Method E) followed by SCX-cartridge (1 g) eluting with Methanol (3CV) then 2M Ammonia in Methanol (3CV). The ammonia containing fractions were then combined and concentrated to afford the title compound (5.4 mg, 0.0214 mmol, 11% Yield) as a pale brown solid. 1HNMR(500 MHz, DMSO-d6) δ 9.90 (s, 1H), 7.77 (d,J=8.8 Hz, 1H), 7.62 (d,J=2.8 Hz, 1H), 6.91 (dd,J=8.9, 3.0 Hz, 1H), 5.56 (d,J=5.2 Hz, 1H), 2.68 (d,J=5.1 Hz, 3H), 1.44 (s, 1H), 1.23 (s, 6H), 1.15 (s, 6H). LCMS: m/z 248.2 [M+H]+, (ESI+), RT=1.83 (Method A)

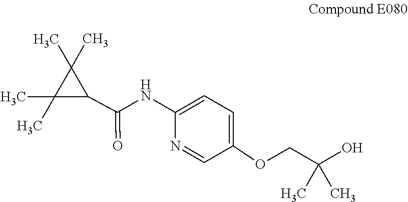 [SMILES: CC(C)(O)COc1ccc(NC(=O)C2C(C)(C)C2(C)C)nc1]

### N-[5-(2-hydroxy-2-methyl-propoxy)-2-pyridyl]-2,2,3,3-tetramethyl-cyclopropanecarboxamide

Synthesized using a similar method to that used in Compound E079, starting from 2-methyl-2-(phenylmethoxy)-1-propanol. 1H NMR (500 MHz, Chloroform-d) δ 8.11 (d, J=9.0 Hz, 1H), 7.95 (d, J=2.9 Hz, 1H), 7.89 (s, 1H), 7.26-7.22 (m, 1H), 3.80 (s, 2H), 2.18 (s, 1H), 1.35 (s, 6H), 1.31 (s, 6H), 1.21 (s, 6H), 1.00 (s, 1H). LCMS: m/z 307.5 [M+H]+, (ESI+), RT=2.73 (Method A)

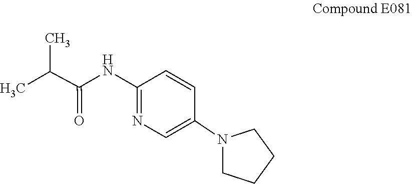 [SMILES: CC(C)C(=O)Nc1ccc(N2CCCC2)cn1]

### 2-methyl-N-(5-pyrrolidin-1-yl-2-pyridyl)propanamide

Step 1

A suspension of 5-bromo-2-nitro-pyridine (1 g, 4.93 mmol) in pyrrolidine (2.1 mL, 24.6 mmol) was heated to 120° C. for 1 hour in a microwave reactor with stirring. The reaction mixture was diluted with water (40 mL) and extracted with EtOAc (2×50 mL). The combined organic layers was dried over sodium sulfate, filtered and evaporated to dryness. Purification by flash chromatography (50 g Biotage KP-Sil cartridge, 10-60% EtOAc in heptane), gave 2-nitro-5-pyrrolidin-1-yl-pyridine as a yellow solid (486 mg, 2.52 mmol, 51% Yield). 1H NMR (250 MHz, Chloroform-d) δ 8.15 (d, J=2.4 Hz, 1H), 7.47 (dd, J=9.0, 2.5 Hz, 1H), 6.25 (d, J=9.0 Hz, 1H), 3.56-3.26 (m, 4H), 2.10-1.85 (m, 4H).

Step 2

To a degassed suspension of 2-nitro-5-pyrrolidin-l-yl-pyridine (486 mg, 2.52 mmol) in Ethanol (10 mL) at RT was added 10% palladium on charcoal (50 mg, 0.470 mmol) and stirred under an atmosphere of hydrogen for 4 h. The reaction mixture was filtered through cellite (5 g) and evaporated to dryness to give a brown solid, 5-pyrrolidin-l-ylpyridin-2-amine (466 mg, 2.31 mmol, 92% Yield). 1H NMR (500 MHz, DMSO-d6) δ 7.35 (d, J=2.9 Hz, 1H), 6.84 (dd, J=8.8, 3.0 Hz, 1H), 6.40 (d, J=8.8 Hz, 1H), 5.01 (s, 2H), 3.14-3.04 (m, 4H), 1.96-1.84 (m, 4H).

Step 3

To a stirred solution of 5-pyrrolidin-l-ylpyridin-2-amine (58 mg, 0.288 mmol) and N-ethyl-N-isopropyl-propan-2-amine (0.10 mL, 0.573 mmol) in DCM (5 mL) at rt was added a solution of 2-methylpropanoyl chloride (0.060 mL, 0.573 mmol) in DCM (1 mL) and stirred for 2 h. The reaction mixture was washed with sat. NaHCO3 (2 mL), dried over sodium sulfate, filtered and evaporated to dryness. The residue was dissolved in MeOH (3 mL) and 1 N NaOH solution (2 mL) an stirred at rt for 1 h before evaporating in vacuo. Purification by prep HPLC (Method H) followed by lyophilisation gave the title product as a white solid (31 mg, 0.132 mmol, 46% Yield). 1H NMR (250 MHz, DMSO-d6) δ 9.95 (s, 1H), 7.88 (d, J=8.9 Hz, 1H), 7.63 (d, J=2.8 Hz, 1H), 6.96 (dd, J=9.0, 3.1 Hz, 1H), 3.28-3.15 (m, 4H), 2.77-2.58 (m, 1H), 2.01-1.88 (m, 4H), 1.06 (d, J=6.8 Hz, 6H). LCMS: m/z 234.1 [M+H]+, (ESI+), RT=1.49 (Method A)

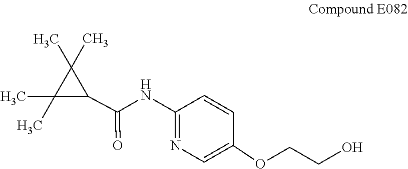 [SMILES: CC1(C)C(C(=O)Nc2ccc(OCCO)cn2)C1(C)C]

### N[5-(2-hydroxyethoxy)pyridin-2-yl]-2,2,3,3- tetramethylcyclopropane-l-carboxamide

Synthesized using a similar method to that used in Compound E078, starting from benzyloxyethanol, with the exception that sodium hydride was used as the base and DMF was used as the solvent in Step 1. 1HNMR(500 MHz, Chloroform-d) δ 8.13 (d,J=9.1 Hz, 1H), 7.98 (d,J=2.8 Hz, 1H), 7.87 (s, 1H), 7.28-7.25 (m, 1H), 4.15-4.09 (m, 2H), 4.02-3.96 (m, 2H), 2.04 (s, 1H), 1.34 (s, 6H), 1.23 (s, 6H), 1.03 (s, 1H). LCMS: m/z 279.0 [M+H]+, (ESI+), RT=2.26 (Method A)

TABLE 2
The following compounds were Synthesized using a similar method to that used in
Compound E001, with the exception that sodium hydride was used as the
base and DMF was used as the solvent in Step 1.

| Compound | | | LCMS |
|---|---|---|---|
| number | Structure | Analytical data | Method |
| E083 | 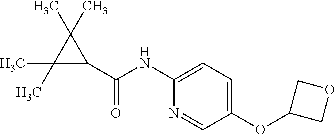 [SMILES: CC1(C)C(C(=O)Nc2ccc(OC3COC3)cn2)C1(C)C] | 1H NMR (500 MHz, DMSO-d6) δ 10.23 (s, 1H), 7.96 (d, J = 9.1 Hz, 1H), 7.86 (dd, J = 3.1, 0.5 Hz, 1H), 7.23 (dd, J = 9.1, 3.1 Hz, 1H), 5.36-5.24 (m, 1H), 4.96-4.87 (m, 2H), 4.59-4.50 (m, 2H), 1.49 (s, 1H), 1.22 (s, 6H), 1.15 (s, 6H). LCMS: m/z 291.2 [M + H]+, (ESI+), RT = 2.96 | A |
| E084 | 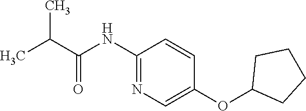 [SMILES: CC(C)C(=O)Nc1ccc(OC2CCCC2)cn1] | 1H NMR (250 MHz, DMSO-d6) δ 10.19 (s, 1H), 8.05-7.90 (m, 2H), 7.36 (dd, J = 9.0, 3.1 Hz, 1H), 4.89-4.76 (m, 1H), 2.76-2.61 (m, 1H), 1.98- 1.81 (m, 2H), 1.76-1.50 (m, 6H), 1.07 (d, J = 6.8 Hz, 6H). LCMS: m/z 249.1 [M + H]+, (ESI+), | A |
| | | RT = 2.93 | |
| E085 | 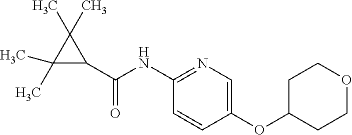 [SMILES: CC1(C)C(C(=O)Nc2ccc(OC3CCOCC3)cn2)C1(C)C] | 1H NMR (400 MHz, Chloroform-d) δ 8.70 (s, 1H), 8.20-8.11 (m, 1H), 7.88 (d, J = 2.9 Hz, 1H), 7.29 (dd, J = 9.1, 3.0 Hz, 1H), 4.46-4.38 (m, 1H), 4.02- 3.94 (m, 2H), 3.60-3.52 (m, 2H), 2.04-1.97 (m, 2H), 1.82-1.73 (m, 2H), 1.31 (s, 6H), 1.22 (s, 6H), 1.08 (s, 1H). LCMS: m/z 319.3 [M + H]+, (ESI+), RT = 3.13 | A |
| E086 | 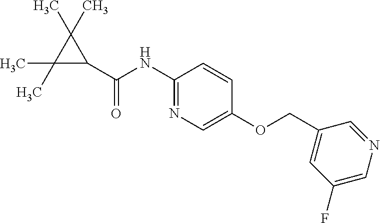 [SMILES: CC1(C)C(C(=O)Nc2ccc(OCc3cncc(F)c3)cn2)C1(C)C] | 1HNMR(400 MHz, Chloroform-d) δ 8.51 (s, 1H), 8.49 (d,J = 2.7 Hz, 1H), 8.16 (d,J = 9.1 Hz, 1H), 8.02 (d,J = 2.9 Hz, 1H), 7.87 (s, 1H), 7.54 (d,J = 8.9 Hz, 1H), 7.32 (dd,J = 9.1, 3.0 Hz, 1H), 5.14 (s, 2H), 1.34 (s, 6H), 1.24 (s, 6H), 1.03 (s, 1H). LCMS: m/z 344.3 [M + H]+, (ESI+), RT = 3.32 | A |
| E087 | 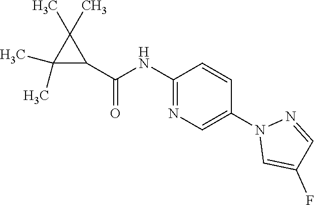 [SMILES: CC1(C)C(C(=O)Nc2ccc(-n3cc(F)cn3)cn2)C1(C)C] | 1H NMR (500 MHz, Chloroform-d) δ 8.54 (d, J = 2.6 Hz, 1H), 8.30 (d, J = 9.0 Hz, 1H), 8.01 (s, 1H), 7.90 (dd, J = 9.0, 2.7 Hz, 1H), 7.76 (d, J = 4.8 Hz, 1H), 7.59 (d, J = 4.1 Hz, 1H), 1.33 (s, 6H), 1.23 (s, 6H), 1.04 (s, 1H). LCMS: m/z 303.2 [M + H]+, (ESI+), RT = 3.72 | A |
| E088 | 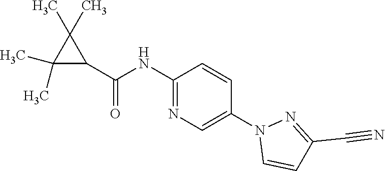 [SMILES: CC1(C)C(C(=O)Nc2ccc(-n3ccc(C#N)n3)cn2)C1(C)C] | 1H NMR (500 MHz, Chloroform-d) δ 8.62 (dd, J = 2.7, 0.6 Hz, 1H), 8.38- 8.33 (m, 1H), 8.06 (s, 1H), 7.97 (dd, J = 9.0, 2.7 Hz, 1H), 7.93 (d, J = 2.6 Hz, 1H), 6.89 (d, J = 2.6 Hz, 1H), 1.34 (s, 6H), 1.24 (s, 6H), 1.05 (s, 1H). LCMS: m/z 310.2 [M + H]+, (ESI+), RT = 3.76 | A |
| E089* | 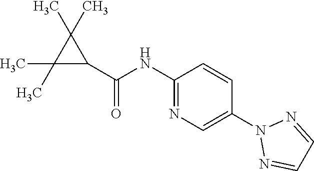 [SMILES: CC1(C)C(C(=O)Nc2ccc(-n3nccn3)cn2)C1(C)C] | 1H NMR (500 MHz, Chloroform-d) δ 8.98 (dd, J = 2.2, 1.1 Hz, 1H), 8.35- 8.32 (m, 2H), 8.09 (s, 1H), 7.83 (s, 2H), 1.34 (s, 6H), 1.24 (s, 6H), 1.05 (s, 1H). LCMS: m/z 286.2 [M + H]+, (ESI+), RT = 3.70 | A |
| E090* | 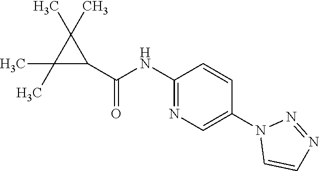 [SMILES: CC1(C)C(C(=O)Nc2ccc(-n3ccnn3)cn2)C1(C)C] | 1H NMR (500 MHz, Chloroform-d) δ 8.67-8.63 (m, 1H), 8.42-8.36 (m, 1H), 8.08 (s, 1H), 8.01 (dd, J = 9.0, 2.7 Hz, 1H), 7.97 (d, J = 1.1 Hz, 1H), 7.88 (d, J = 1.1 Hz, 1H), 1.34 (s, 6H), 1.25 (s, 6H), 1.06 (s, 1H). LCMS: m/z 286.2 [M + H]+, (ESI+), RT = 3.07 | A |
| E091 | 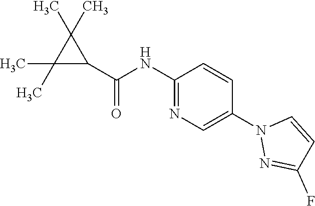 [SMILES: CC1(C)C(C(=O)Nc2ccc(-n3ccc(F)n3)cn2)C1(C)C] | 1H NMR (400 MHz, DMSO-d6) δ 10.57 (s, 1H), 8.69 (d, J = 2.8 Hz, 1H), 8.44 (t, J = 2.6 Hz, 1H), 8.17 (d, J = 9.1 Hz, 1H), 8.08 (dd, J = 9.1, 2.8 Hz, 1H), 6.34 (dd, J = 5.7, 2.7 Hz, 1H), 1.55 (s, 1H), 1.24 (s, 6H), 1.17 (s, 6H). LCMS: m/z 303.2 [M + H]+, (ESI+), RT = 3.91 | B |
| E092 | 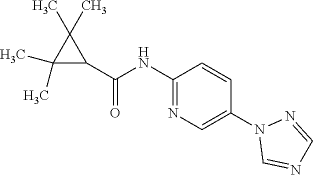 [SMILES: CC1(C)C(C(=O)Nc2ccc(-n3cncn3)cn2)C1(C)C] | 1H NMR (500 MHz, Chloroform-d) δ 8.60 (dd, J = 2.7, 0.6 Hz, 1H), 8.51 (s, 1H), 8.37 (dd, J = 9.0, 0.5 Hz, 1H), 8.13 (s, 1H), 8.05 (s, 1H), 7.95 (dd, J = 9.0, 2.7 Hz, 1H), 1.34 (s, 6H), 1.25 (s, 6H), 1.05 (s, 1H). LCMS: m/z 286.2 [M + H]+, (ESI+), RT = 3.13 | B |

*Compounds E089 and E090 were isolated as products from the same reaction

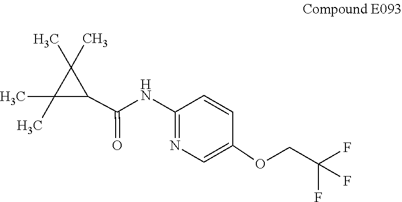 [SMILES: CC1(C)C(C(=O)Nc2ccc(OCC(F)(F)F)cn2)C1(C)C]

### 2,2,3,3-tetramethyl-N-[5-(2,2,2-trifluoroethoxy)-2-pyridyl]cyclopropanecarboxamide

Step 1

To a stirred mixture of 6-aminopyridin-3-ol (150 mg, 1.36 mmol) and cesium carbonate (0.67 g, 2.04 mmol) in DMF-Anhydrous (3.6 mL) was added drop-wise 2,2,2-trifluoroethyl trifluoromethanesulfonate (0.22 mL, 1.50 mmol). After stirring for 6 hours at room temperature the mixture was concentrated to dryness under reduced pressure. To the residue was added in EtOAc (20 ml) and water (20 ml). The organics were separated and the aqueous extracted with further EtOAc (2×20 ml). The organics were combined, dried (MgSO4) and concentrated under reduced pressure. The crude product was purified by flash column chromatography (SiO2, 25 g, eluting with 20-100% EtOAc in heptane) to afford 5-(2,2,2-trifluoroethoxy)pyridin-2-amine (60 mg, 22% Yield) as a brown solid. 1H NMR(500 MHz, DMSO-d6) δ 7.74 (d,J=3.0 Hz, 1H), 7.22 (dd,J=8.9, 3.1 Hz, 1H), 6.43 (d,J=8.9 Hz, 1H), 5.65 (s, 2H), 4.62 (q,J=9.0 Hz, 2H)

Step 2

To a solution of 5-(2,2,2-trifluoroethoxy)pyridin-2-amine (60 mg, 0.309 mmol) and N-ethyl-N-isopropyl-propan-2-amine (108 uL, 0.618 mmol) in THF-Anhydrous (2.5 mL) was added 2,2,3,3-tetramethylcyclopropanecarbonyl chloride (60 mg, 0.371 mmol) with stirring at RT for 16 hour after which time, MeOH (1 mL), 1M NaOH (1 mL) were added and the reaction mixture stirred at RT for 0.5 h. The reaction mixture was diluted with brine (10 mL) and extracted with EtOAc (3×10 mL) and the combined organic extracts concentrated in vacuo. The crude product was purified by flash column chromatography (SiO2, 50 g, 0-30% EtOAc in Heptane) followed prep HPLC (Method G) to afford the title compound (8.2 mg, 0.0259 mmol, 8.4% Yield) as a colourless solid. 1H NMR (500 MHz, Chloroform-d) δ 8.18 (d,J=9.1 Hz, 1H), 8.02 (d,J=2.9 Hz, 1H), 7.87 (s, 1H), 7.31 (dd,J=9.1, 3.0 Hz, 1H), 4.38 (q,J=8.0 Hz, 2H), 1.34 (s, 6H), 1.24 (s, 6H), 1.03 (s, 1H). LCMS: m/z 317.2 [M+H]+, (ESI+), RT=3.78 (Method A)

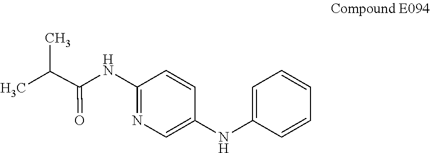 [SMILES: CC(C)C(=O)Nc1ccc(Nc2ccccc2)cn1]

### N-(5-anilino-2-pyridyl)-2-methyl-propanamide

Step 1

To a stirred solution of isobutyric anhydride (359 uL, 2.17 mmol) and 5-bromopyridin-2-amine (250 mg, 1.45 mmol) in THF (15.453 mL) was added N-ethyl-N-isopropyl-propan-2-amine (505 uL, 2.89 mmol) followed by N,N-dimethylpyridin-4-amine (18 mg, 0.145 mmol) and stirred at 80° C. in a sealable pressure tube for 2 h. The reaction mixture was then evaporated to dryness. Purification by flash chromatography (50 g KP-Sil Biotage SNAP cartridge, 10-100% EtOAc in heptane) gave N-(5-bromo-2-pyridyl)-2-methyl-propanamide (330 mg, 1.34 mmol, 93% Yield) as a white solid. 1H NMR (250 MHz, Chloroform-d) δ 8.30 (d, J=2.4 Hz, 1H), 8.18 (d, J=8.9 Hz, 1H), 7.90 (s, 1H), 7.79 (dd, J=8.9, 2.4 Hz, 1H), 2.55 (hept, J=6.9 Hz, 1H), 1.26 (d, J=6.9 Hz, 6H).

Step 2

To a mixture of N-(5-bromo-2-pyridyl)-2-methyl-propanamide (70 mg, 0.285 mMol) and sodium tert-butoxide (41 mg, 0.428 mMol) was added toluene (3 mL). Aniline (29 mg, 0.314 mMol) was then added and the mixture degassed under nitrogen for 10 min at RT. Pd2(dba)3 (26 mg, 0.0283 mMol) and dicyclohexyl-[2-(2,4,6-triisopropylphenyl)phenyl]phosphane (40.8 mg, 0.0855 mMol) were then added to the mixture and the reaction vessel was sealed and heated to 100° C. for 4 hours with stirring before allowing to cool to RT. The reaction mixture was filtered through cellite with EtOAc (30 mL). This was then washed with sat NaHCO3 (30 mL), followed by brine (30 mL), dried over sodium sulfate, filtered and evaporated to dryness. Purification by prep HPLC (Method F) afforded the title compound as a white solid (3.6 mg, 5%). 1H NMR (500 MHz, DMSO-d6) δ 10.18 (s, 1H), 8.13 (s, 1H), 8.08 (d, J=2.4 Hz, 1H), 7.98 (d, J=8.9 Hz, 1H), 7.51 (dd, J=8.9, 2.9 Hz, 1H), 7.25-7.19 (m, 2H), 6.99 (dd, J=8.6, 1.0 Hz, 2H), 6.81 (tt, J =7.4, 1.1 Hz, 1H), 2.71 (hept, J=6.8 Hz, 1H), 1.08 (d, J=6.8 Hz, 6H). LCMS: m/z 256.2 [M+H]+, (ESI+), RT=2.30 (Method A)

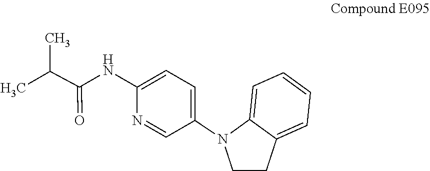 [SMILES: CC(C)C(=O)Nc1ccc(N2CCc3ccccc32)cn1]

### N-(5-indolin-1-yl-2-pyridyl)-2-methyl-propanamide

Synthesized using a similar method to that used in Compound E094. 1H NMR (250 MHz, DMSO-d6) δ 10.28 (s, 1H), 8.24 (d, J=2.4 Hz, 1H), 8.08 (d, J=9.4 Hz, 1H), 7.70 (dd, J=9.2, 3.0 Hz, 1H), 7.18 (d, J=7.4 Hz, 1H), 7.10-6.94 (m, 2H), 6.77-6.68 (m, 1H), 3.93 (t, J=8.6 Hz, 2H), 3.10 (t, J=8.6 Hz, 2H), 2.77 (m, J=1.9 Hz, 1H), 1.09 (d, J=6.8 Hz, 6H). LCMS: m/z 282.2 [M+H]+, (ESI+), RT=3.02 (Method A)

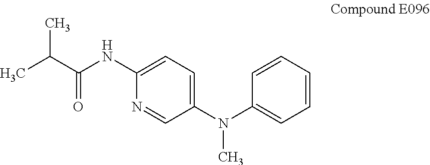 [SMILES: CC(C)C(=O)Nc1ccc(N(C)c2ccccc2)cn1]

### 2-methyl-N-[5-(N-methylanilino)-2-pyridyl]propanamide

Step 1

To a solution of 5-bromo-2-nitro-pyridine (200 mg, 0.985 mmol) and Pd2(dba)3 (45 mg, 0.0493 mmol) in DME (5 mL) was added cesium carbonate (482 mg, 1.48 mmol) and degassed under nitrogen for 10 min at RT. To this was added N-methylaniline (0.13 mL, 1.18 mmol) and (R)-BINAP (61 mg, 0.0985 mmol) and the reaction vessel was sealed and heated to 100° C. for 4 hours with stirring before allowing to cool to RT. The reaction mixture was diluted with water and extracted with EtOAc, the aqueous layer was then extracted with EtOAc, the organic extracts combined, dried over sodium sulfate, filtered and evaporated to dryness to afford N-methyl-6-nitro-N-phenyl-pyridin-3-amine as a dark yellow solid (187.7 mg, 83.1%). 1H NMR (500 MHz, DMSO-d6) δ 8.15 (d, J=9.2 Hz, 1H), 7.97 (d, J=3.0 Hz, 1H), 7.55-7.50 (m, 2H), 7.38-7.34 (m, 3H), 7.21 (dd, J=9.2, 3.0 Hz, 1H), 3.43 (s, 3H).

Step 2

To a solution of N-methyl-6-nitro-N-phenyl-pyridin-3-amine (188 mg, 0.819 mmol) in Ethanol (5 mL) was added 10% Pd/C (87 mg, 0.0819 mmol) and the reaction mixture was put under a balloon of hydrogen and stirred at RT for 4 hrs. It was then filtered through Celite, washed with EtOAc and concentrated under reduced pressure to afford N5-methyl-N5-phenyl-pyridine-2,5-diamine as a colourless oil (138 mg, 85%). 1H NMR (500 MHz, DMSO-d6) δ 7.76 (dd, J=2.7, 0.6 Hz, 1H), 7.22 (dd, J=8.7, 2.7 Hz, 1H), 7.15-7.10 (m, 2H), 6.68-6.60 (m, 3H), 6.49 (dd, J=8.7, 0.7 Hz, 1H), 5.88 (s, 2H), 3.13 (s, 3H).

Step 3

To a stirred solution of N-ethyl-N-isopropyl-propan-2-amine (140 uL, 0.803 mmol) and N,N-dimethylpyridin-4-amine (4.9 mg, 0.0402 mmol) in THF-Anhydrous (2.5 mL) was added of isobutyric anhydride (100 uL, 0.602 mmol) followed by N5-methyl-N5-phenyl-pyridine-2,5-diamine (80 mg, 0.402 mmol) and stirred at 80° C. in a sealable pressure tube for 48 hr. The reaction mixture was then evaporated to dryness and purified by column chromatography (Biotage SNAP cartridge KP-Sil 10 g; 0-100% EtOAc in heptane) to afford the title compound (57.3 mg, 53%) as a light brown solid. 1H NMR (500 MHz, DMSO-d6) δ 10.31 (s, 1H), 8.09-8.01 (m, 2H), 7.49 (dd, J=8.9, 2.9 Hz, 1H), 7.30-7.20 (m, 2H), 6.97-6.84 (m, 3H), 3.25 (s, 3H), 2.73 (hept, J=6.8 Hz, 1H), 1.08 (d, J=6.8 Hz, 6H). LCMS: m/z 270.2 [M+H]+, (ESI+), RT=2.59 (Method A)

TABLE 3
The following compounds were Synthesized using a similar method to that used in
Compound E096.

| Compound | | | LCMS |
|---|---|---|---|
| number | Structure | Analytical data | Method |
| E097 | 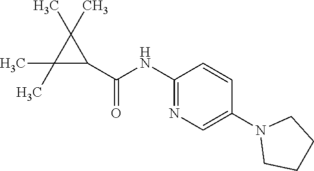 [SMILES: CC1(C)C(C(=O)Nc2ccc(N3CCCC3)cn2)C1(C)C] | 1H NMR (500 MHz, Chloroform-d) δ 7.99 (d, J = 8.9 Hz, 1H), 7.92 (s, 1H), 7.60 (d, J = 2.9 Hz, 1H), 6.87 (dd, J = 9.0, 3.0 Hz, 1H), 3.30-3.22 (m, 4H), 2.06-1.96 (m, 4H), 1.31 (s, 6H), 1.19 (s, 6H), 1.00 (s, 1H). LCMS: m/z 288.3 [M + H]+, (ESI+), RT = 2.38 | A |
| E098 | 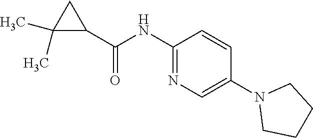 [SMILES: CC1(C)CC1C(=O)Nc1ccc(N2CCCC2)cn1] | (500 MHz, Chloroform-d) δ 8.02 (d, J = 8.9 Hz, 1H), 7.97 (s, 1H), 7.59 (d, J = 2.6 Hz, 1H), 6.90 (dd, J = 9.0, 2.9 Hz, 1H), 3.27 (t, J = 6.5 Hz, 4H), 2.07- 1.97 (m, 4H), 1.42 (dd, J = 7.7, 5.5 Hz, 1H), 1.22 (s, 3H), 1.20 (d, J = 4.7 Hz, 1H), 1.18 (s, 3H), 0.82 (dd, J = 7.9, 4.4 Hz, 1H). LCMS: m/z 260.2 [M + H]+, (ESI+), | A |
| | | RT = 1.98 | |
| E099 | 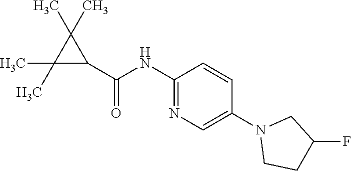 [SMILES: CC1(C)C(C(=O)Nc2ccc(N3CCC(F)C3)cn2)C1(C)C] | 1H NMR (400 MHz, DMSO-d6) δ 9.96 (s, 1H), 7.86 (d, J = 8.9 Hz, 1H), 7.66 (d, J = 2.8 Hz, 1H), 6.98 (dd, J = 9.0, 3.0 Hz, 1H), 5.53-5.34 (m, 1H), 3.60- 3.40 (m, 4H), 2.28-2.09 (m, 2H), 1.45 (s, 1H), 1.23 (s, 6H), 1.15 (s, 6H). LCMS: m/z 306.3 [M + H]+, (ESI+), RT = 3.66 | B |
| E100 | 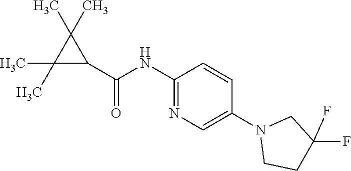 [SMILES: CC1(C)C(C(=O)Nc2ccc(N3CCC(F)(F)C3)cn2)C1(C)C] | 1H NMR (400 MHz, DMSO-d6) δ 10.03 (s, 2H), 7.88 (d, J = 9.0 Hz, 1H), 7.70 (d, J = 2.8 Hz, 1H), 7.06 (dd, J = 9.0, 3.1 Hz, 1H), 3.68 (t, J = 13.4 Hz, 2H), 3.47 (t, J = 7.2 Hz, 2H), 2.57- 2.52 (m, 1H), 1.46 (s, 1H), 1.23 (s, 6H), 1.15 (s, 6H). LCMS: m/z 324.3 [M + H]+, (ESI+), RT = 3.86 | B |
| E101 | 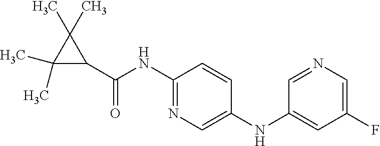 [SMILES: CC1(C)C(C(=O)Nc2ccc(Nc3cncc(F)c3)cn2)C1(C)C] | 1H NMR (500 MHz, Chloroform-d) δ 8.19 (d, J = 8.9 Hz, 1H), 8.12 (d, J = 2.7 Hz, 1H), 8.08 (dd, J = 2.1, 1.3 Hz, 1H), 7.98 (d, J = 2.4 Hz, 1H), 7.90 (s, 1H), 7.50 (dd, J = 8.9, 2.8 Hz, 1H), 6.91 (dt, J = 10.6, 2.4 Hz, 1H), 5.68 (s, 1H), 1.33 (s, 6H), 1.23 (s, 6H), 1.03 (s, 1H). LCMS: m/z 329.3 [M + H]+, (ESI+), RT = 2.96 | A |

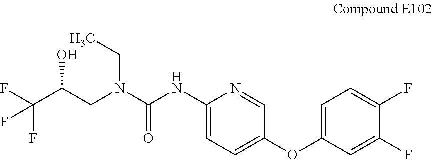 [SMILES: CCN(C[C@@H](O)C(F)(F)F)C(=O)Nc1ccc(Oc2ccc(F)c(F)c2)cn1]

### 3-[5-(3,4-difluorophenoxy)-2-pyridyl]-1-ethyl-1-[(2R)-3,3,3-trifluoro-2-hydroxy-propyl]urea

Step 1

5-bromo-2-nitro-pyridine (2.00 g, 9.85 mmol), cesium carbonate (6.42 g, 19.7 mmol) and 3,4-difluorophenol (1.28 g, 9.85 mmol) were mixed in DMSO (25 mL), purged with nitrogen and stirred in a RBF at 50° C. for 2 hours. The reaction mixture was allowed to cool, diluted with water (75 mL) resulting in formation of a beige/grey precipitate. This was filtered, washed with water, and purified by FCC (Biotage SNAP KP-Sil 25 g, 0-50% EtOAc in heptane) to afford 5-(3,4-difluorophenoxy)-2-nitro-pyridine (2.09 g, 81% Yield) as an off white solid. 1H NMR (400 MHz, Chloroform-d) δ 8.33 (d, J=2.8 Hz, 1H), 8.27 (d, J=8.9 Hz, 1H), 7.44 (dd, J=8.9, 2.8 Hz, 1H), 7.32-7.21 (m, 1H), 7.00 (ddd, J=9.9, 6.5, 2.9 Hz, 1H), 6.88 (dq, J=8.6, 3.1 Hz, 1H).

Step 2

To a solution of 5-(3,4-difluorophenoxy)-2-nitro-pyridine (2 g, 8 mmol) in EtOH (50 mL) and H2O(10 mL) was added ammonium chloride (4.43 g, 82.9 mmol). The reaction mixture was heated to 50° C. and iron (4.63 g, 82.9 mmol) was added. The reaction was then stirred at 70° C. for 25 minutes. The reaction mixture was then cooled, filtered through a pad of Celite, washing with EtOH (50 mL) and EtOAc (150 mL). The filtrate was diluted with water (100 mL) and the layers were separated and the aqueous layer was extracted with EtOAc (2×50 mL). The combined organic extracts were washed with brine (75 mL), dried over MgSO4 and concentrated under reduced pressure to yield 5-(3,4-difluorophenoxy)pyridin-2-amine (Intermediate I01, 1.87 g, quantitative yield) as a brown oil. 1H NMR (500 MHz, Chloroform-d) δ 7.89 (d, J=2.8 Hz, 1H), 7.18 (dd, J=8.8, 2.9 Hz, 1H), 7.11-7.03 (m, 1H), 6.75 (ddd, J=11.5, 6.6, 3.0 Hz, 1H), 6.64 (dtt, J=8.3, 3.2, 1.8 Hz, 1H), 6.55-6.50 (m, 1H), 4.43 (s, 2H).

Step 3

To 2 M ethanamine in THF (4.0 mL, 8.03 mmol) was added (2R)-2-(trifluoromethyl)oxirane (0.23 mL, 2.68 mmol) and the reaction was stirred at RT overnight. Solvent was then removed under reduced pressure to afford the of (2R)-3-(ethylamino)-1,1,1-trifluoro-propan-2-ol as a dark yellow solid (Intermediate I02, 530 mg, 94%, 75% purity). This was used as such in the next step without purification. 1H NMR (500 MHz, Chloroform-d) δ 3.98-3.91 (m, 1H), 2.99-2.93 (m, 1H), 2.87-2.81 (m, 1H), 2.77-2.64 (m, 2H), 1.13 (t, J=7.1 Hz, 3H). (OH and NH not observed).

Step 4

A solution of pyridine (40 uL, 0.495 mmol) and 5-(3,4-difluorophenoxy)pyridin-2-amine (100 mg, 0.450 mmol) in THF-anhydrous (3 mL) was added to a stirred solution of (4-nitrophenyl) carbonochloridate (100 mg, 0.495 mmol) in THF-anhydrous (3 mL). The reaction mixture was stirred for 4.5 hours at RT. A solution of (2R)-3-(ethylamino)-1,1,1-trifluoro-propan-2-ol (92 mg, 0.585 mmol) and N-ethyl-N-isopropyl-propan-2-amine (118 uL, 0.675 mmol) in THF-anhydrous (3 mL) was added to the reaction mixture and stirred overnight at RT. The product was purified by silica flash column chromatography (0-40% EtOAc in heptane) followed by prep HPLC (Method G) to give the title compound (77 mg, 0.189 mmol, 42% Yield) as a clear glass. 1H NMR (400 MHz, DMSO-d6) δ 9.02 (s, 1H), 8.09 (d, J=2.9 Hz, 1H), 7.83 (d, J=9.1 Hz, 1H), 7.55-7.37 (m, 2H), 7.24-7.14 (m, 1H), 7.01 (s, 1H), 6.89-6.79 (m, 1H), 4.34-4.22 (m, 1H), 3.59 (dd, J=14.9, 2.6 Hz, 1H), 3.50-3.38 (m, 3H), 1.11 (t, J=7.0 Hz, 3H). LCMS: m/z 406.2 [M+H]+, (ESI+), RT=3.61 (Method B)

TABLE 4
The following compounds were Synthesized using a similar method to that used in
Compound E102 using either commercial amines or amino-alcohols Synthesized as per
Compound E102, Step3

| Compound | | | LCMS |
|---|---|---|---|
| number | Structure | Analytical data | Method |
| E103 | 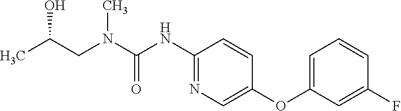 [SMILES: C[C@H](O)CN(C)C(=O)Nc1ccc(Oc2cccc(F)c2)cn1] | 1H NMR (500 MHz, DMSO- d6) δ 9.29 (s, 1H), 8.07 (d, J = 2.9 Hz, 1H), 7.84 (d, J = 9.1 Hz, 1H), 7.52 (dd, J = 9.1, 3.0 Hz, 1H), 7.44-7.35 (m, 1H), 7.00-6.90 (m, 1H), 6.87- 6.83 (m, 1H), 6.81-6.78 (m, | A |
| | | 1H), 5.50 (s, 1H), 4.05-3.85 | |
| | | (m, 1H), 3.30-3.21 (m, 2H), | |
| | | 2.95 (s, 3H), 1.10 (d, J = 6.3 | |
| | | Hz, 3H). | |
| | | LCMS: m/z 320.2 [M + H]+, | |
| | | (ESI+), RT = 2.52 | |
| E104 | 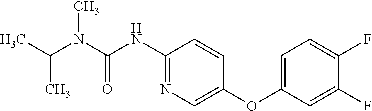 [SMILES: CC(C)N(C)C(=O)Nc1ccc(Oc2ccc(F)c(F)c2)cn1] | 1H NMR (500 MHz, DMSO- d6) δ 8.79 (s, 1H), 8.08 (dd, J = 3.0, 0.5 Hz, 1H), 7.87-7.80 (m, 1H), 7.48 (dd, J = 9.1, 3.0 Hz, 1H), 7.44 (ddd, J = 10.4, 9.2, 9.2 Hz, 1H), 7.19 (ddd, J = 11.9, 6.8, 3.0 Hz, 1H), 6.87- | A |
| | | 6.78 (m, 1H), 4.47 (hept, J = | |
| | | 6.7 Hz, 1H), 2.80 (s, 3H), 1.07 | |
| | | (d, J = 6.7 Hz, 6H). | |
| | | LCMS: m/z 322.3 [M + H]+, | |
| | | (ESI+), RT = 3.15 | |
| E105 | 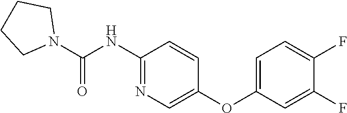 [SMILES: O=C(Nc1ccc(Oc2ccc(F)c(F)c2)cn1)N1CCCC1] | 1H NMR (500 MHz, DMSO- d6) δ 8.72 (s, 1H), 8.08 (dd, J = 3.0, 0.5 Hz, 1H), 7.96-7.87 (m, 1H), 7.49 (dd, J = 9.1, 3.0 Hz, 1H), 7.44 (ddd, J = 10.4, 9.2, 9.2 Hz, 1H), 7.19 (ddd, J = 11.9, 6.8, 3.0 Hz, 1H), 6.86- | A |
| | | 6.80 (m, 1H), 3.44-3.36 (m, | |
| | | 4H), 1.90-1.74 (m, 4H). | |
| | | LCMS: m/z 320.2 [M + H]+, | |
| | | (ESI+), RT = 2.87 | |
| E106 | 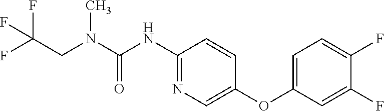 [SMILES: CN(CC(F)(F)F)C(=O)Nc1ccc(Oc2ccc(F)c(F)c2)cn1] | 1H NMR (500 MHz, DMSO- d6) δ 9.25 (s, 1H), 8.13 (d, J = 2.9 Hz, 1H), 7.84 (d, J = 9.1 Hz, 1H), 7.52 (dd, J = 9.1, 3.0 Hz, 1H), 7.48-7.40 (m, 1H), 7.26-7.18 (m, 1H), 6.88- 6.83 (m, 1H), 4.25 (q, J = 9.5 | B |
| | | Hz, 2H), 3.11 (s, 3H). | |
| | | LCMS: m/z 362.1 [M + H]+, | |
| | | (ESI+), RT = 3.64 | |
| E107 | 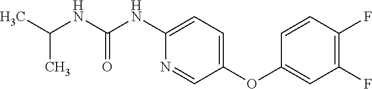 [SMILES: CC(C)NC(=O)Nc1ccc(Oc2ccc(F)c(F)c2)cn1] | 1H NMR (400 MHz, Chloroform-d) δ 8.79 (s, 1H), 7.97 (d, J = 2.8 Hz, 1H), 7.56 (s, 1H), 7.31 (dd, J = 8.9, 2.9 Hz, 1H), 7.17-7.06 (m, 1H), 6.85-6.78 (m, 1H), 6.77 (d, | A |
| | | J = 8.8 Hz, 1H), 6.72-6.63 (m, | |
| | | 1H), 4.08 (dq, J = 13.2, 6.6 Hz, | |
| | | 1H), 1.26 (d, J = 6.6 Hz, 6H). | |
| | | LCMS: m/z 308.2 [M + H]+, | |
| | | (ESI+), RT = 3.36 | |
| E108 | 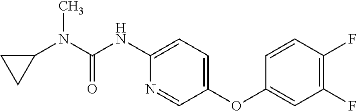 [SMILES: CN(C(=O)Nc1ccc(Oc2ccc(F)c(F)c2)cn1)C1CC1] | 1H NMR (400 MHz, Chloroform-d) δ 8.15 (dd, J = 9.1, 0.5 Hz, 1H), 8.05-8.00 (m, 1H), 7.99 (s, 1H), 7.34 (dd, J = 9.1, 2.9 Hz, 1H), 7.15- 7.05 (m, 1H), 6.80 (ddd, J = 11.2, 6.6, 3.0 Hz, 1H), 6.68 | A |
| | | (dtd, J = 9.0, 3.2, 1.8 Hz, 1H), | |
| | | 3.00 (s, 3H), 2.67 (ddd, J = | |
| | | 10.4, 6.7, 3.8 Hz, 1H), 1.02 (m, | |
| | | 2H), 0.91-0.84 (m, 2H). | |
| | | LCMS: m/z 320.1 [M + H]+, | |
| | | (ESI+), RT = 3.36 | |
| E109 | 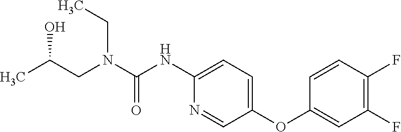 [SMILES: CCN(C[C@H](C)O)C(=O)Nc1ccc(Oc2ccc(F)c(F)c2)cn1] | 1H NMR (500 MHz, Chloroform-d) δ 8.52 (s, 1H), 8.04-7.96 (m, 2H), 7.33 (dd, J = 9.1, 2.9 Hz, 1H), 7.13-7.05 (m, 1H), 6.78 (ddd, J = 11.3, 6.6, 3.0 Hz, 1H), 6.69-6.63 (m, 1H), 4.17-4.07 (m, 1H), 3.51-3.28 (m, 4H), 3.23 (dd, J = 15.4, 2.1 Hz, 1H), 1.29-1.21 | A |
| | | (m, 6H). | |
| | | LCMS: m/z 352.3 [M + H]+, | |
| | | (ESI+), RT = 2.93 | |
| E110 | 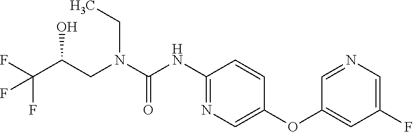 [SMILES: CCN(C[C@@H](O)C(F)(F)F)C(=O)Nc1ccc(Oc2cncc(F)c2)cn1] | (500 MHz, Chloroform-d) δ 8.25 (d, J = 2.4 Hz, 1H), 8.23 (d, J = 2.2 Hz, 1H), 8.07 (dd, J = 6.0, 2.8 Hz, 2H), 7.56 (s, 1H), 7.43 (dd, J = 9.2, 2.8 Hz, 1H), 6.99 (dt, J = 9.5, 2.4 Hz, 1H), 5.23 (s, 1H), 4.25-4.13 (m, 1H), 3.88-3.76 (m, 1H), 3.57- 3.45 (m, 2H), 3.45-3.33 (m, | A |
| | | 1H), 1.33 (t, J = 7.2 Hz, 3H). | |
| | | LCMS: m/z 389.2 [M + H]+, | |
| | | (ESI+), RT = 2.73 | |
| E111 | 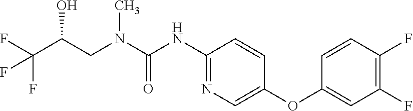 [SMILES: CN(C[C@@H](O)C(F)(F)F)C(=O)Nc1ccc(Oc2ccc(F)c(F)c2)cn1] | (500 MHz, Chloroform-d) δ 8.04-7.98 (m, 2H), 7.44 (s, 1H), 7.36 (dd, J = 9.0, 3.0 Hz, 1H), 7.12 (q, J = 9.0 Hz, 1H), 6.85-6.78 (m, 1H), 6.72- 6.66 (m, 1H), 5.15 (s, 1H), 4.24- 4.14 (m, 1H), 3.80 (dd, J = | A |
| | | 15.2, 7.9 Hz, 1H), 3.64 (dd, J = | |
| | | 15.2, 2.2 Hz, 1H), 3.16 (s, | |
| | | 3H)LCMS: m/z 392.2 [M + H]+, | |
| | | (ESI+), RT = 3.16 | |
| E112 | 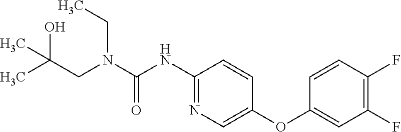 [SMILES: CCN(CC(C)(C)O)C(=O)Nc1ccc(Oc2ccc(F)c(F)c2)cn1] | (500 MHz, Chloroform-d) δ 8.04 (d, J = 9.1 Hz, 1H), 8.01 (d, J = 2.8 Hz, 1H), 7.32 (dd, J = 9.1, 2.9 Hz, 1H), 7.13-7.04 (m, 1H), 6.78 (ddd, J = 11.3, 6.6, 3.0 Hz, 1H), 6.69-6.62 (m, 1H), 3.43 (q, J = 7.0 Hz, 2H), 3.35 (s, 2H), 1.33 (s, 6H), 1.24 (t, J = 7.1 Hz, 3H). | A |
| | | LCMS: m/z 366.2 [M + H]+, | |
| | | (ESI+), RT = 3.16 | |
| E113 | 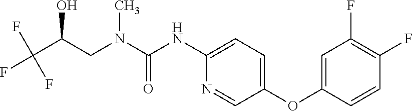 [SMILES: CN(C[C@H](O)C(F)(F)F)C(=O)Nc1ccc(Oc2ccc(F)c(F)c2)cn1] | 1H NMR (500 MHz, Chloroform-d) δ 8.15-7.81 (m, 2H), 7.47 (s, 1H), 7.36 (dd, J = 9.0, 3.0 Hz, 1H), 7.19- 7.04 (m, 1H), 6.81 (ddd, J = 11.1, 6.5, 3.0 Hz, 1H), 6.69 (dddd, J = 9.0, 3.2, 3.2, 1.8 Hz, | A |
| | | 1H), 5.18 (s, 1H), 4.27-4.12 | |
| | | (m, 1H), 3.80 (dd, J = 15.2, 7.9 | |
| | | Hz, 1H), 3.64 (dd, J = 15.2, 2.2 | |
| | | Hz, 1H), 3.15 (s, 3H). | |
| | | LCMS: m/z 392.3 [M + H]+, | |
| | | (ESI+), RT = 3.16 | |
| E114 | 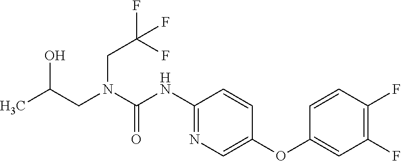 [SMILES: CC(O)CN(CC(F)(F)F)C(=O)Nc1ccc(Oc2ccc(F)c(F)c2)cn1] | (400 MHz, Chloroform-d) δ 9.39 (s, 1H), 8.02 (d, J = 2.8 Hz, 1H), 7.97 (d, J = 9.1 Hz, 1H), 7.34 (dd, J = 9.1, 2.9 Hz, 1H), 7.10 (q, J = 9.1 Hz, 1H), 6.79 (ddd, J = 11.2, 6.5, 2.9 Hz, 1H), 6.72-6.63 (m, 1H), 4.52 (dq, J = 15.7, 9.3 Hz, 1H), 4.30- 4.20 (m, 1H), 3.78-3.61 (m, 2H), 3.27 (d, J = 16.0 Hz, 1H), | A |
| | | 2.86 (d, J = 2.4 Hz, 1H), 1.30 | |
| | | (d, J = 6.3 Hz, 3H). | |
| | | LCMS: m/z 406.2 [M + H]+, | |
| | | (ESI+), RT = 3.44 | |
| E115 | 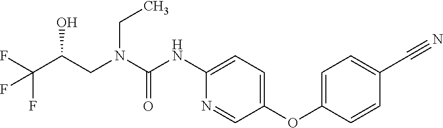 [SMILES: CCN(C[C@@H](O)C(F)(F)F)C(=O)Nc1ccc(Oc2ccc(C#N)cc2)cn1] | 1H NMR (500 MHz, DMSO- d6) δ 9.10 (s, 1H), 8.15 (dd, J = 2.9, 0.5 Hz, 1H), 7.91-7.85 (m, 1H), 7.87-7.80 (m, 2H), 7.60 (dd, J = 9.1, 3.0 Hz, 1H), 7.14-7.08 (m, 2H), 7.03 (s, 1H), 4.32-4.23 (m, 1H), 3.59 (dd, J = 14.9, 2.5 Hz, 1H), 3.49- | B |
| | | 3.42 (m, 3H), 1.11 (t, J = 7.0 | |
| | | Hz, 3H). | |
| | | LCMS: m/z 395.3 [M + H]+, | |
| | | (ESI+), RT = 3.26 | |
| E116 | 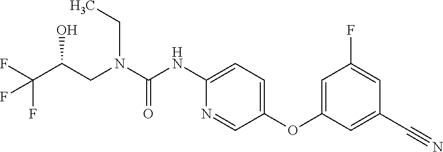 [SMILES: CCN(C[C@@H](O)C(F)(F)F)C(=O)Nc1ccc(Oc2cc(F)cc(C#N)c2)cn1] | 1H NMR (400 MHz, DMSO- d6) δ 9.08 (s, 1H), 8.15 (d, J = 2.8 Hz, 1H), 7.88 (d, J = 9.1 Hz, 1H), 7.63-7.56 (m, 2H), 7.37 (s, 1H), 7.30 (dt, J = 10.3, 2.3 Hz, 1H), 7.04 (s, 1H), 4.33- 4.23 (m, 1H), 3.62-3.56 (m, 1H), 3.48-3.40 (m, 3H), 1.11 (t, J = 7.0 Hz, 3H). LCMS: m/z 413.3 [M + H]+, | B |
| | | (ESI+), RT = 3.43 | |
| E117 | 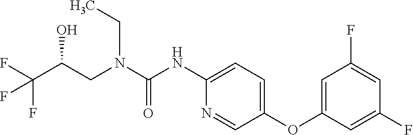 [SMILES: CCN(C[C@@H](O)C(F)(F)F)C(=O)Nc1ccc(Oc2cc(F)cc(F)c2)cn1] | 1H NMR (500 MHz, DMSO- d6) δ 9.08 (s, 1H), 8.14 (dd, J = 3.0, 0.6 Hz, 1H), 7.91-7.82 (m, 1H), 7.58 (dd, J = 9.1, 3.0 Hz, 1H), 7.03 (s, 1H), 6.98 (ddd, J = 9.3, 7.1, 2.3 Hz, 1H), 6.79-6.66 (m, 2H), 4.40- 4.16 (m, 1H), 3.59 (dd, J = 14.9, 2.4 Hz, 1H), 3.52-3.38 | A |
| | | (m, 3H), 1.11 (t, J = 7.0 Hz, | |
| | | 3H). | |
| | | LCMS: m/z 406.0 [M + H]+, | |
| | | (ESI+), RT = 3.53 | |
| E118 | 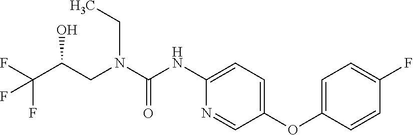 [SMILES: CCN(C[C@@H](O)C(F)(F)F)C(=O)Nc1ccc(Oc2ccc(F)cc2)cn1] | 1H NMR (500 MHz, DMSO- d6) δ 8.99 (s, 1H), 8.05 (dd, J = 3.0, 0.5 Hz, 1H), 7.88-7.72 (m, 1H), 7.44 (dd, J = 9.1, 3.0 Hz, 1H), 7.26-7.17 (m, 2H), 7.11-6.86 (m, 3H), 4.38- 4.15 (m, 1H), 3.58 (dd, J = 14.9, 2.6 Hz, 1H), 3.49-3.37 (m, 3H), 1.10 (t, J = 7.0 Hz, | A |
| | | 3H). | |
| | | LCMS: m/z 388.0 [M + H]+, | |
| | | (ESI+), RT = 3.29 | |
| E119 | 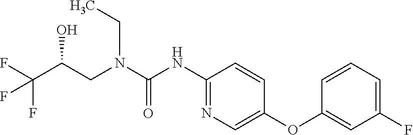 [SMILES: CCN(C[C@@H](O)C(F)(F)F)C(=O)Nc1ccc(Oc2cccc(F)c2)cn1] | (400 MHz, Chloroform-d) δ 8.02 (dd, J = 5.9, 2.8 Hz, 2H), 7.62 (s, 1H), 7.40 (dd, J = 9.2, 2.8 Hz, 1H), 7.29 (dd, J = 8.3, 6.7 Hz, 1H), 6.84-6.77 (m, 1H), 6.74 (dd, J = 8.3, 2.2 Hz, 1H), 6.68 (dt, J = 10.1, 2.4 Hz, 1H), 4.22-4.13 (m, 1H), 3.83 (dd, J = 15.2, 8.6 Hz, 1H), 3.55- | A |
| | | 3.46 (m, 2H), 3.46-3.35 (m, | |
| | | 1H), 1.32 (t, J = 7.2 Hz, 3H). | |
| | | LCMS: m/z 388.2 [M + H]+, | |
| | | (ESI+), RT = 3.36 | |
| E120 | 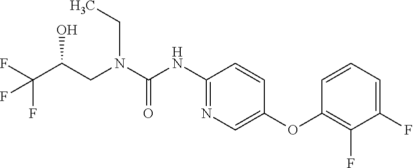 [SMILES: CCN(C[C@@H](O)C(F)(F)F)C(=O)Nc1ccc(Oc2cccc(F)c2F)cn1] | 1HNMR(400 MHz, Chloroform-d) δ 8.06 (d,J = 2.8 Hz, 1H), 8.02 (d,J = 9.1 Hz, 1H), 7.50 (s, 1H), 7.38 (dd,J = 9.1, 2.9 Hz, 1H), 7.08-6.94 (m, 2H), 6.80-6.73 (m, 1H), 5.32 (s, 1H), 4.25-4.13 (m, 1H), 3.85 (dd,J = 15.2, 8.6 Hz, 1H), 3.57-3.36 (m, 3H), 1.34 (t,J = 7.2 Hz, 3H). LCMS: m/z 406.2 [M + H]+, | A |
| | | (ESI+), RT = 3.41 | |
| E121 | 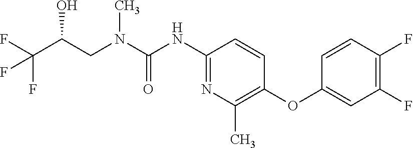 [SMILES: Cc1nc(NC(=O)N(C)C[C@@H](O)C(F)(F)F)ccc1Oc1ccc(F)c(F)c1] | 1HNMR(400 MHz, Chloroform-d) δ 7.85 (d,J =8.9 Hz, 1H), 7.38 (s, 1H), 7.28 (d, J = 8.8 Hz, 1H), 7.11 (q,J = 9.1 Hz, 1H), 6.77-6.70 (m, 1H), 6.64-6.57 (m, 1H), 5.27 (s, 1H), 4.27-4.16 (m, 1H), 3.82 (dd,J = 15.2, 7.8 Hz, 1H), 3.67 (dd,J = 15.2, 2.4 Hz, 1H), 3.19 | A |
| | | (s, 3H), 2.36 (s, 3H). | |
| | | LCMS: m/z 406.2 [M + H]+, | |
| | | (ESI+), RT = 3.34 | |
| E122 | 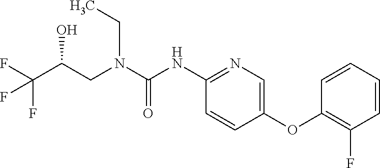 [SMILES: CCN(C[C@@H](O)C(F)(F)F)C(=O)Nc1ccc(Oc2ccccc2F)cn1] | 1H NMR (400 MHz, Chloroform-d) δ 8.01 (d, J = 2.8 Hz, 1H), 7.96 (d, J = 9.1 Hz, 1H), 7.51 (s, 1H), 7.32 (dd, J = 9.1, 2.9 Hz, 1H), 7.23- 7.07 (m, 3H), 7.06-6.95 (m, 1H), 5.48 (s, 1H), 4.33-4.05 (m, 1H), 3.82 (dd, J = 15.2, 8.6 Hz, 1H), 3.57-3.30 (m, 3H), 1.31 (t, J = 7.2 Hz, 3H). LCMS: m/z 388.3 [M + H]+, | A |
| | | (ESI+), RT = 3.26 | |
| E123 | 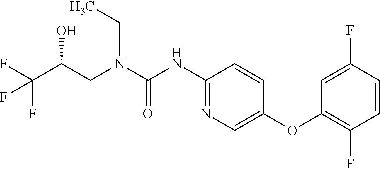 [SMILES: CCN(C[C@@H](O)C(F)(F)F)C(=O)Nc1ccc(Oc2cc(F)ccc2F)cn1] | (500 MHz, Chloroform-d) δ 8.07-7.98 (m, 2H), 7.51 (s, 1H), 7.37 (dd, J = 9.1, 2.9 Hz, 1H), 7.17-7.10 (m, 1H), 6.80 (ddt, J = 9.1, 7.5, 3.2 Hz, 1H), 6.70 (ddd, J = 9.2, 6.5, 3.0 Hz, 1H), 5.34 (s, 1H), 4.17 (q, J = 6.6 Hz, 1H), 3.82 (dd, J = 15.2, 8.6 Hz, 1H), 3.57-3.44 (m, 2H), 3.44-3.33 (m, 1H), 1.32 (t, J = 7.2 Hz, 3H). | A |
| | | LCMS: m/z 406.2 [M + H]+, | |
| | | (ESI+), RT = 3.35 | |
| E124 | 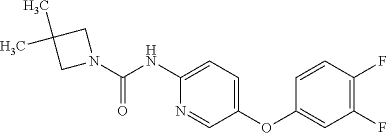 [SMILES: CC1(C)CN(C(=O)Nc2ccc(Oc3ccc(F)c(F)c3)cn2)C1] | 1H NMR (400 MHz, DMSO- d6) δ 9.09 (s, 1H), 8.07 (d, J = 3.0 Hz, 1H), 7.95 (d, J = 9.1 Hz, 1H), 7.49 (dd, J = 9.1, 3.0 Hz, 1H), 7.49-7.37 (m, 1H), 7.24-7.13 (m, 1H), 6.88- 6.78 (m, 1H), 3.66 (s, 4H), 1.22 (s, 6H). LCMS: m/z 332.4 [M − H]−, | B |
| | | (ESI−), RT = 3.72 | |
| E125 | 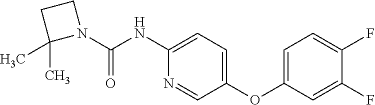 [SMILES: CC1(C)CCN1C(=O)Nc1ccc(Oc2ccc(F)c(F)c2)cn1] | 1H NMR (400 MHz, DMSO- d6) δ 8.55 (s, 1H), 8.06 (d, J = 3.0 Hz, 1H), 7.94 (d, J = 9.1 Hz, 1H), 7.50-7.39 (m, 2H), 7.22-7.14 (m, 1H), 6.86- 6.79 (m, 1H), 3.96 (t, J = 7.5 Hz, 2H), 2.02-1.93 (m, 2H), | B |
| | | 1.45 (s, 6H). | |
| | | LCMS: m/z 334.2 [M + H]+, | |
| | | (ESI+), RT = 3.59 | |
| E126 | 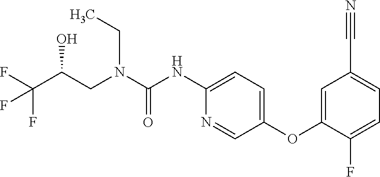 [SMILES: CCN(C[C@@H](O)C(F)(F)F)C(=O)Nc1ccc(Oc2cc(C#N)ccc2F)cn1] | 1H NMR (500 MHz, DMSO- d6) δ 9.06 (s, 1H), 8.14 (d, J = 3.0 Hz, 1H), 7.84 (d, J = 9.1 Hz, 1H), 7.73 (ddd, J = 8.5, 4.3, 2.0 Hz, 1H), 7.67 (dd, J = 7.8, 2.0 Hz, 1H), 7.64 (dd, J = 10.8, 8.5 Hz, 1H),7.55 (dd, J = 9.1, 3.0 Hz, 1H), 7.01 (s, 1H), 4.38- 4.18 (m, 1H), 3.59 (dd, J = 14.9, 2.5 Hz, 1H), 3.51-3.36 (m, 3H), 1.11 (t, J = 7.0 Hz, | A |
| | | 3H). | |
| | | LCMS: m/z 413.2 [M + H]+, | |
| | | (ESI+), RT = 3.14 | |
| E127 | 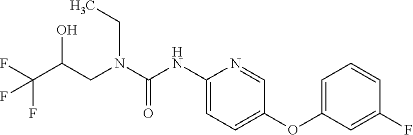 [SMILES: CCN(CC(O)C(F)(F)F)C(=O)Nc1ccc(Oc2cccc(F)c2)cn1] | 1HNMR(400 MHz, Chloroform-d) δ 8.07-8.01 (m, 2H), 7.65 (s, 1H), 7.42 (dd,J = 9.0, 2.9 Hz, 1H), 7.31 (dd,J = 8.4, 6.8 Hz, 1H), 6.83 (td,J = 8.3, 2.3 Hz, 1H), 6.76 (dd,J = 8.3, 2.2 Hz, 1H), 6.70 (dt,J = 10.1, 2.4 Hz, 1H), 5.61 (s, 1H), 4.26-4.13 (m, 1H), | A |
| | | 3.84 (dd,J = 15.3, 8.5 Hz, 1H), | |
| | | 3.57-3.38 (m, 3H), 1.34 (t,J = | |
| | | 7.2 Hz, 3H). | |
| | | LCMS: m/z 388.5 [M + H]+, | |
| | | (ESI+), RT = 3.40 | |
| E128 | 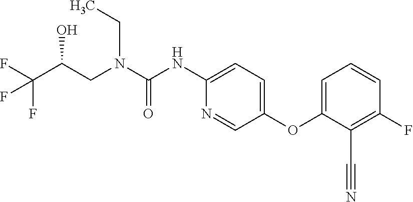 [SMILES: CCN(C[C@@H](O)C(F)(F)F)C(=O)Nc1ccc(Oc2cccc(F)c2C#N)cn1] | 1H NMR (500 MHz, DMSO- d6) δ 9.15 (s, 1H), 8.24 (d, J = 2.8 Hz, 1H), 7.91 (d, J = 9.1 Hz, 1H), 7.74-7.64 (m, 2H), 7.24 (dd, J = 8.7, 8.7 Hz, 1H), 7.03 (s, 1H), 6.75 (d, J = 8.6 Hz, 1H), 4.38-4.21 (m, 1H), 3.66-3.55 (m, 1H), 3.52- 3.38 (m, 3H), 1.12 (t, J = 7.0 Hz, 3H). LCMS: m/z 413.2 [M + H]+, (ESI+), RT = 3.31 | A |
| E129 | 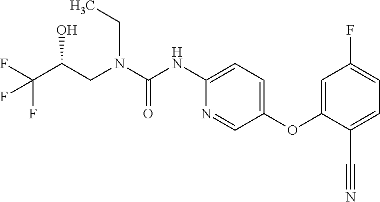 [SMILES: CCN(C[C@@H](O)C(F)(F)F)C(=O)Nc1ccc(Oc2cc(F)ccc2C#N)cn1] | 1H NMR (500 MHz, DMSO- d6) δ 9.14 (s, 1H), 8.23 (d, J = 2.7 Hz, 1H), 8.01 (dd, J = 8.7, 6.2 Hz, 1H), 7.91 (d, J = 9.1 Hz, 1H), 7.67 (dd, J = 9.1, 3.0 Hz, 1H), 7.16 (ddd, J = 8.4, 8.4, 2.4 Hz, 1H), 7.04 (s, 1H), 6.84 (dd, J = 10.3, 2.4 Hz, 1H), 4.37- 4.15 (m, 1H), 3.60 (dd, J = 15.0, 2.3 Hz, 1H), 3.55-3.39 (m, 3H), 1.12 (t, J = 7.0 Hz, 3H). LCMS: m/z 413.1 [M + H]+, | A |
| | | (ESI+), RT = 3.27 | |
| E130 | 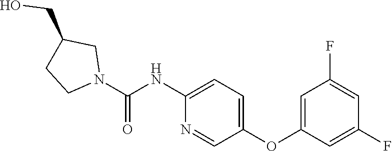 [SMILES: O=C(Nc1ccc(Oc2cc(F)cc(F)c2)cn1)N1CC[C@@H](CO)C1] | (500 MHz, Chloroform-d) δ 8.16 (d, J = 9.0 Hz, 1H), 8.02 (d, J = 2.8 Hz, 1H), 7.40 (dd, J = 9.1, 2.9 Hz, 1H), 7.07 (s, 1H), 6.52 (tt, J = 8.9, 2.3 Hz, 1H), 6.49-6.42 (m, 2H), 3.76- 3.58 (m, 4H), 3.51 (dt, J = 9.8, 7.6 Hz, 1H), 3.34 (dd, J = 10.0, 6.8 Hz, 1H), 2.56 (dt, J = 14.0, 7.0 Hz, 1H), 2.13 (dt, J = 12.2, | A |
| | | 6.2 Hz, 1H), 1.84 (dd, J = 12.4, | |
| | | 7.8 Hz, 1H), 1.71 (s, 1H). | |
| | | LCMS: m/z 350.2 [M + H]+, | |
| | | (ESI+), RT = 2.47 | |
| E131 | 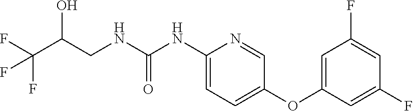 [SMILES: O=C(NCC(O)C(F)(F)F)Nc1ccc(Oc2cc(F)cc(F)c2)cn1] | (500 MHz, Chloroform-d) δ 9.73 (s, 1H), 8.22 (s, 1H), 8.05 (d, J = 2.8 Hz, 1H), 7.42 (dd, J = 8.9, 2.9 Hz, 1H), 6.83 (d, J = 8.9 Hz, 1H), 6.57 (tt, J = 8.8, 2.3 Hz, 1H), 6.51-6.43 (m, 2H), 5.10 (d, J = 3.4 Hz, 1H), | A |
| | | 4.18 (s, 1H), 3.81-3.66 (m, | |
| | | 2H). | |
| | | LCMS: m/z 378.1 [M + H]+, | |
| | | (ESI+), RT = 3.16 | |
| E132 | 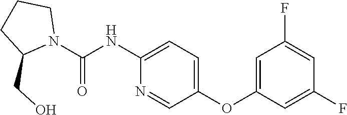 [SMILES: O=C(Nc1ccc(Oc2cc(F)cc(F)c2)cn1)N1CCC[C@@H]1CO] | 1HNMR(400 MHz, Chloroform-d) δ 8.13 (d,J = 9.3 Hz, 1H), 8.05 (d,J = 2.7 Hz, 1H), 7.42 (dd,J = 9.1, 2.9 Hz, 1H), 6.55 (tt,J = 8.8, 2.3 Hz, 1H), 6.51-6.42 (m, 2H), 4.31 (s, 1H), 4.21 (s, 1H), 3.78 (d,J = | A |
| | | 10.1 Hz, 1H), 3.74-3.59 (m, | |
| | | 2H), 3.57-3.47 (m, 1H), 2.19- | |
| | | 2.05 (m, 1H), 2.05-1.94 (m, | |
| | | 2H), 1.77-1.62 (m, 1H). | |
| | | LCMS: m/z 350.2 [M + H]+, | |
| | | (ESI+), RT = 2.81 | |
| E133 | 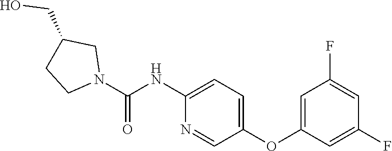 [SMILES: O=C(Nc1ccc(Oc2cc(F)cc(F)c2)cn1)N1CC[C@H](CO)C1] | 1HNMR(400 MHz, Chloroform-d) δ 8.18 (dd,J = 9.1, 5.9 Hz, 1H), 8.04 (d,J = 2.7 Hz, 1H), 7.42 (dd,J = 9.1,2.9 Hz, 1H), 7.12 (s, 1H), 6.55 (tt, J = 8.9, 2.3 Hz, 1H), 6.51-6.42 (m, 2H), 3.79-3.60 (m, 4H), 3.53 (dt,J = 9.9, 7.6 Hz, 1H), 3.37 (dd,J = 10.0, 6.8 Hz, 1H), 2.58 (dt,J = 14.1, 7.1 Hz, 1H), | A |
| | | 2.14 (dq,J = 12.2, 7.1 Hz, 1H), | |
| | | 1.86 (dq,J = 15.6, 7.8 Hz, 1H). | |
| | | LCMS: m/z 350.1 [M + H]+, | |
| | | (ESI+), RT = 2.65 | |
| E134 | 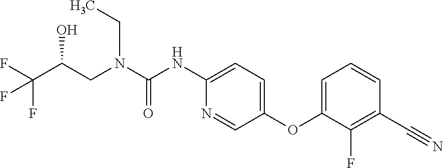 [SMILES: CCN(C[C@@H](O)C(F)(F)F)C(=O)Nc1ccc(Oc2cccc(C#N)c2F)cn1] | 1H NMR (400 MHz, DMSO- d6) δ 9.06 (s, 1H), 8.16 (d, J = 2.7 Hz, 1H), 7.84 (d, J = 9.1 Hz, 1H), 7.72-7.65 (m, 1H), 7.56 (dd, J = 9.1, 3.0 Hz, 1H), 7.45 (td, J = 8.3, 1.7 Hz, 1H), 7.40-7.33 (m, 1H), 7.01 (s, 1H), 4.33-4.22 (m, 1H), 3.59 (dd, J = 15.0, 2.8 Hz, 1H), 3.49- 3.39 (m, 3H), 1.11 (t, J = 7.0 Hz, 3H). | B |
| | | LCMS: m/z 413.3 [M + H]+, | |
| | | (ESI+), RT = 3.30 | |
| E135 | 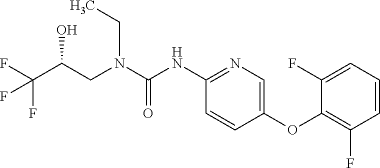 [SMILES: CCN(C[C@@H](O)C(F)(F)F)C(=O)Nc1ccc(Oc2c(F)cccc2F)cn1] | 1H NMR (500 MHz, DMSO- d6) δ 8.97 (s, 1H), 8.04 (d, J = 3.1 Hz, 1H), 7.76 (d, J = 9.1 Hz, 1H), 7.41-7.27 (m, 4H), 6.96 (s, 1H), 4.32-4.17 (m, 1H), 3.58 (dd, J = 14.9, 2.6 Hz, 1H), 3.47-3.36 (m, 3H), 1.09 (t, J = 7.0 Hz, 3H). LCMS: m/z 406.3 [M + H]+, (ESI+), RT = 3.45 | B |
| E136 | 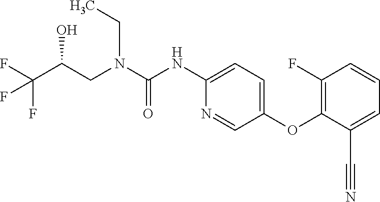 [SMILES: CCN(C[C@@H](O)C(F)(F)F)C(=O)Nc1ccc(Oc2c(F)cccc2C#N)cn1] | 1H NMR (400 MHz, DMSO- d6) δ 9.01 (s, 1H), 8.11 (d, J = 3.1 Hz, 1H), 7.85-7.77 (m, 3H), 7.54-7.43 (m, 2H), 6.97 (s, 1H), 4.26 (s, 1H), 3.58 (dd, J = 15.0, 2.6 Hz, 1H), 3.49-3.38 (m, 3H), 1.10 (t, J = 7.0 Hz, 3H). LCMS: m/z 413.3 [M + H]+, (ESI+), RT = 3.21 | B |
| E137 | 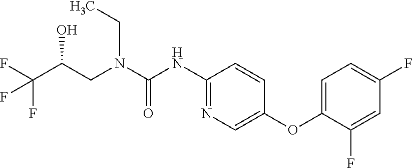 [SMILES: CCN(C[C@@H](O)C(F)(F)F)C(=O)Nc1ccc(Oc2ccc(F)cc2F)cn1] | 1H NMR (400 MHz, DMSO- d6) δ 8.97 (s, 1H), 8.05 (d, J = 2.8 Hz, 1H), 7.79 (d, J = 9.1 Hz, 1H), 7.51-7.44 (m, 1H), 7.42 (dd, J = 9.1, 3.0 Hz, 1H), 7.24 (td, J = 9.3, 5.6 Hz, 1H), 7.10 (tdd, J = 9.2, 3.0, 1.7 Hz, 1H), 6.99 (s, 1H), 4.26 (s, 1H), 3.58 (dd, J = 14.9, 2.7 Hz, 1H), 3.46-3.38 (m, 3H), 1.10 (t, J = 7.0 Hz, 3H). | A |
| | | LCMS: m/z 406.2 [M + H]+, | |
| | | (ESI+), RT = 3.37 | |
| E138 | 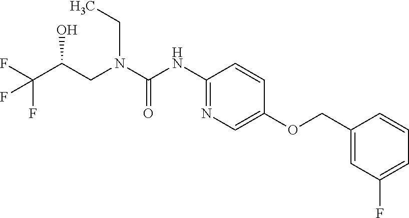 [SMILES: CCN(C[C@@H](O)C(F)(F)F)C(=O)Nc1ccc(OCc2cccc(F)c2)cn1] | (400 MHz, Chloroform-d) δ 7.96-7.88 (m, 2H), 7.52 (s, 1H), 7.38-7.33 (m, 1H), 7.31 (dd, J = 9.1, 3.0 Hz, 1H), 7.20- 7.11 (m, 2H), 7.03 (td, J = 8.3, 2.2 Hz, 1H), 5.06 (s, 2H), 4.19- 4.10 (m, 1H), 3.81 (dd, J = 15.2, 8.6 Hz, 1H), 3.52-3.32 (m, 3H), 1.29 (t, J = 7.2 Hz, 3H). LCMS: m/z 402.2 [M + H]+, (ESI+), RT = 3.09 | A |

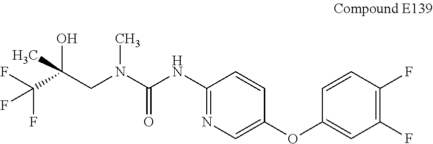 [SMILES: CN(C[C@@](C)(O)C(F)(F)F)C(=O)Nc1ccc(Oc2ccc(F)c(F)c2)cn1]

### 3-[5-(3,4-difluorophenoxy)-2-pyridyl]-1-methyl-1-[(2R)-3,3,3-trifluoro-2-hydroxy-2-methyl-propyl]urea

Step 1

To a three-necked RBF under N2 was added (2R)-2-(trifluoromethyl)oxirane (200 mg, 1.78 mmol) followed by THF-Anhydrous (8 mL) and the stirred solution was cooled to −100° C. with a Et20/dry ice bath. 1.6 M butyllithium (1.2 mL, 1.96 mmol) was then added dropwise followed by stirring at this temperature for 10 minutes. Iodomethane (0.17 mL, 2.68 mmol) was then added and the reaction was stirred for 2 h at this temperature, warmed up to ˜0° C. with an ice bath and to this was added 2 M methanamine (3.6 mL, 7.14 mmol) and the reaction was allowed to warm up to RT and was stirred overnight and left standing over the weekend. Solvent was then removed under reduced pressure to afford (2R)-1,1,1-trifluoro-2-methyl-3-(methylamino)propan-2-ol as an orange viscous gum (1.10 g). 1H NMR (500 MHz, Methanol-d4) δ 2.82 (d, J=12.3 Hz, 1H), 2.70-2.64 (m, 1H), 2.44 (s, 3H), 1.38-1.35 (m, 3H).

Step 2

To a solution of 4-nitrophenyl carbonochloridate (47 mg, 0.233 mmol) in anhydrous THF (1.5 mL) was added a solution of 5-(3,4-difluorophenoxy)pyridin-2-amine (Intermediate I01, 50 mg, 0.212 mmol) and pyridine (19 uL, 0.233 mmol) in anhydrous THF (1 mL) and the reaction was stirred at RT for 3 hours. Next, (2R)-1,1,1-trifluoro-2-methyl-3-(methylamino)propan-2-ol (25% pure, 133 mg, 0.212 mmol) and N-ethyl-N-isopropyl-propan-2-amine (55 uL, 0.317 mmol) in anhydrous THF (1.5 mL) were added and the reaction was stirred at RT for 45 min. It was then concentrated under reduced pressure and purified by prep HPLC (Method G) to afford the title compound (25 mg, 29% Yield) as a pale yellow solid. 1H NMR, (500 MHz, Chloroform-d) δ 8.05-7.99 (m, 2H), 7.47 (s, 1H), 7.36 (dd, J=9.0, 3.0 Hz, 1H), 7.12 (q, J=9.0 Hz, 1H), 6.81 (ddd, J=11.1, 6.5, 3.0 Hz, 1H), 6.73-6.66 (m, 1H), 5.66 (s, 1H), 3.77-3.60 (m, 2H), 3.18 (s, 3H), 1.39 (s, 3H). LCMS: m/z 406.2 [M+H]+, (ESI+), RT=3.37 (Method A)

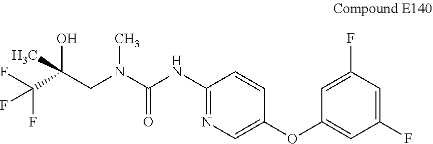 [SMILES: CN(C[C@@](C)(O)C(F)(F)F)C(=O)Nc1ccc(Oc2cc(F)cc(F)c2)cn1]

### 3-[5-(3,5-difluorophenoxy)-2-pyridyl]-1-methyl-1-[(2R)-3,3,3-trifluoro-2-hydroxy-2-methyl-propyl]urea

Synthesized using a similar method to that used in Compound E139. 1H NMR (500 MHz, DMSO-d6) δ 9.28 (s, 1H), 8.14 (d, J=2.8 Hz, 1H), 7.86 (d, J=9.0 Hz, 1H), 7.59 (dd, J=9.1, 3.0 Hz, 1H), 6.98 (tt, J=9.3, 2.3 Hz, 1H), 6.78-6.68 (m, 2H), 3.72 (d, J=13.9 Hz, 1H), 3.46 (d, J=14.4 Hz, 1H), 3.06 (s, 3H), 1.30 (s, 3H). LCMS: m/z 406.3 [M+H]+, (ESI+), RT=3.64 (Method B)

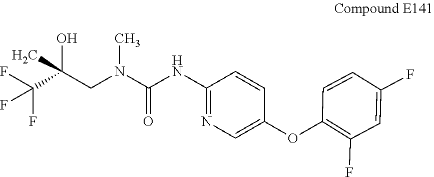 [SMILES: CN(C[C@@](C)(O)C(F)(F)F)C(=O)Nc1ccc(Oc2ccc(F)cc2F)cn1]

### 3-[5-(2,4-difluorophenoxy)-2-pyridyl]-1-methyl-1-[(2R)-3,3,3-trifluoro-2-hydroxy-2-methyl-propyl]urea

Synthesized using a similar method to that used in Compound E139. 1HNMR(500 MHz, Chloroform-d) δ 8.02-7.98 (m, 2H), 7.43 (s, 1H), 7.30 (dd,J=9.1, 3.0 Hz, 1H), 7.06 (td,J=9.0, 5.4 Hz, 1H), 7.01-6.95 (m, 1H), 6.91-6.85 (m, 1H), 5.79 (s, 1H), 3.74 (d,J=15.3 Hz, 1H), 3.64 (d,J=15.3 Hz, 1H), 3.19 (s, 3H), 1.40 (s, 3H). LCMS: m/z 404.2 [M-H]-, (ESI-), RT=3.30 (Method A)

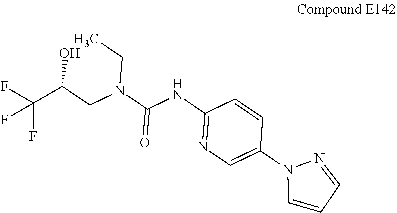 [SMILES: CCN(C[C@@H](O)C(F)(F)F)C(=O)Nc1ccc(-n2cccn2)cn1]

### 1-ethyl-3-(5-pyrazol-1-yl-2-pyridyl)-1-[(2R)-3,3,3-trifluoro-2-hydroxy-propyl]urea

Step 1

5-fluoro-2-nitropyridine (250 mg, 1.76 mmol) and 1H-pyrazole (126 mg, 1.85 mmol) were mixed in DMF-Anhydrous (3.5 mL), The reaction mixture was cooled to 0° C. before sodium hydride (60%, 106 mg, 2.64 mmol) was added portion-wise. The reaction was then stirred at room temperature for 2 hours. Upon completion, the reaction mixture was diluted with water (10 mL) resulting in formation of a pale yellow precipitate. This was filtered, washed with water (50m1) and dried to afford 2-nitro-5-pyrazol-1-yl-pyridine (235 mg, 1.17 mmol, 67% Yield) as a pale yellow solid. 1H NMR(400 MHz, DMSO-d6) δ 9.23-9.16 (m, 1H), 8.81 (d,J=2.4 Hz, 1H), 8.63 (dd,J=8.9, 2.6 Hz, 1H), 8.51-8.45 (m, 1H), 7.97 (d,J=1.6 Hz, 1H), 6.73 (dd,J=2.6, 1.7 Hz, 1H).

Step 2

To a stirred solution of 2-nitro-5-pyrazol-1-yl-pyridine (235 mg, 1.24 mmol) in 1,4-Dioxane (10 mL) and Methanol (5 mL) was added 10% Pd/C (53 mg, 0.25 mmol) and the reaction mixture was put under a balloon of hydrogen and was stirred at RT for 4 hours. The reaction mixture was filtered through Celite which was washed with further dioxane (50 ml). The filtrate was concentrated under reduced pressure to afford 5-pyrazol-1-ylpyridin-2-amine (188 mg, 90% Yield) as a sandy brown solid. 1H NMR(400 MHz, DMSO-d6) δ 8.32 (d,J=2.5 Hz, 1H), 8.25 (d,J=2.1 Hz, 1H), 7.78 (dd,J=8.8, 2.8 Hz, 1H), 7.66 (d,J=1.5 Hz, 1H), 6.54 (d,J=8.9 Hz, 1H), 6.49-6.43 (m, 1H), 6.10 (s, 2H).

Step 3

To a solution of (4-nitrophenyl) carbonochloridate (66 mg, 0.326 mmol) in anhydrous THF (2 mL) was added a solution of 5-pyrazol-1-ylpyridin-2-amine (50 mg, 0.297 mmol) and pyridine (0.026 mL, 0.297 mmol) in anhydrous THF (2 mL) and the reaction was stirred at RT for 1 hour. (2R)-3-(ethylamino)-1,1,1-trifluoro-propan-2-ol (Intermediate I02, 71 mg, 0.386 mmol) and N-ethyl-N-isopropyl-propan-2-amine (78 uL, 0.445 mmol) in anhydrous THF (2 mL) were then added and the reaction was stirred at RT for 1 hour. It was then concentrated under reduced pressure and purified by prep HPLC (Method G) to afford the title compound (47 mg, 0.133 mmol, 45% Yield) as a pale yellow solid. 1HNMR(400 MHz, Chloroform-d) δ 8.58 (s, 1H), 8.11 (d,J=9.0 Hz, 1H), 7.99-7.91 (m, 1H), 7.86 (d,J=2.2 Hz, 1H), 7.76-7.71 (m, 1H), 6.50 (t,J=1.9 Hz, 1H), 5.74 (s, 1H), 4.26-4.14 (m, 1H), 3.80 (dd,J=15.2, 8.7 Hz, 1H), 3.59-3.47 (m, 2H), 3.41 (dq,J=14.8, 7.3 Hz, 1H), 1.31 (t,J=7.1 Hz, 3H). LCMS: m/z 344.3 [M+H]+, (ESI+), RT=2.48 (Method A).

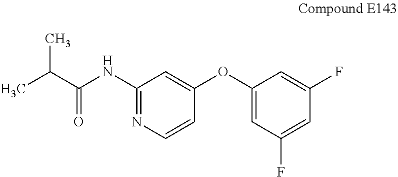 [SMILES: CC(C)C(=O)Nc1cc(Oc2cc(F)cc(F)c2)ccn1]

### N-[4-(3,5-difluorophenoxy)-2-pyridyl]-2-methyl-propanamide

Step 1

To a solution of 4-chloropyridin-2-amine (300 mg, 2.33 mmol) and 3,5-difluorophenol (395 mg, 3.03 mmol) in NMP (5 mL) at RT was added N-ethyl-N-isopropyl-propan-2-amine (0.82 mL, 4.67 mmol). The reaction mixture was stirred at 160° C. for 18 hours. It was then cooled to RT and diluted with EtOAc (30 mL) and water/brine (1:1, 30 mL). The layers were separated and the aqueous layer was extracted with EtOAc (2×20 mL). The combined organic extracts were dried over MgSO4, filtered, concentrated under reduced pressure and purified by prep HPLC (Method F) to afford 4-(3,5-difluorophenoxy)pyridin-2-amine as a light brown solid (85 mg). 1H NMR (400 MHz, DMSO-d6) δ 7.86 (d, J=5.8 Hz, 1H), 7.13 (tt, J=9.4, 2.3 Hz, 1H), 7.03-6.85 (m, 2H), 6.20 (dd, J=5.8, 2.3 Hz, 1H), 6.02 (s, 2H), 5.95 (d, J=2.2 Hz, 1H).

Step 2

To a solution of 4-(3,5-difluorophenoxy)pyridin-2-amine (40 mg, 0.180 mmol) in DCM (1 mL) was added N-ethyl-N-isopropyl-propan-2-amine (63 uL, 0.360 mmol) followed by 2-methylpropanoyl chloride (32 uL, 0.306 mmol) and the reaction was stirred at RT for 0.5 h. Solvent was then removed under a steady stream of nitrogen and the residue was dissolved in MeOH (1 mL), 1M NaOH (1 mL) was added and the reaction mixture was stirred at RT for 0.5 h. MeOH was removed under a steady stream of nitrogen and the aqueous layer was extracted twice with EtOAc. The combined organic extracts were dried over MgSO4, filtered, concentrated under reduced pressure and purified by flash column chromatography (10 g SiO2 column, 0-60% EtOAc in heptane) to afford the title compound as a white solid (39 mg). 1H NMR (500 MHz, DMSO-d6) δ 10.56 (s, 1H), 8.24 (d, J=5.7 Hz, 1H), 7.75 (d, J=2.3 Hz, 1H), 7.21 (tt, J=9.4, 2.3 Hz, 1H), 7.12-7.00 (m, 2H), 6.77 (dd, J=5.7, 2.4 Hz, 1H), 2.72 (hept, J=6.8 Hz, 1H), 1.05 (d, J=6.8 Hz, 6H). LCMS: m/z 292.8 [M+H]+, (ESI+), RT=3.02 (Method A).

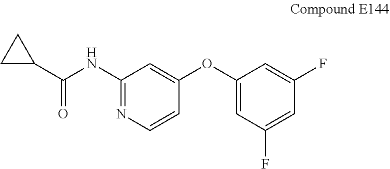 [SMILES: O=C(Nc1cc(Oc2cc(F)cc(F)c2)ccn1)C1CC1]

### N-[4-(3,5-difluorophenoxy)-2-pyridyl]cyclopropanecarboxamide

Synthesized using a similar method to that used in Compound E143. 1H NMR (500 MHz, DMSO-d6) δ 10.92 (s, 1H), 8.25 (d, J=5.7 Hz, 1H), 7.71 (d, J=2.3 Hz, 1H), 7.20 (tt, J=9.4, 2.3 Hz, 1H), 7.12-6.97 (m, 2H), 6.77 (dd, J=5.7, 2.4 Hz, 1H), 1.98 (tt, J=6.2, 6.2 Hz, 1H), 0.78 (d, J=6.2 Hz, 4H). LCMS: m/z 291.1 [M+H]+, (ESI+), RT=2.83 (Method A).

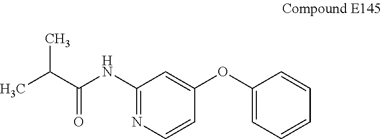 [SMILES: CC(C)C(=O)Nc1cc(Oc2ccccc2)ccn1]

### 2-methyl-N-(4-phenoxy-2-pyridyl)propanamide

Step 1

To a solution of 4-chloropyridin-2-amine (500 mg, 3.89 mmol) in THF (5 mL) at RT was added N-ethyl-N-isopropyl-propan-2-amine (1.4 mL, 7.78 mmol) followed by isobutyric anhydride (0.97 mL, 5.83 mmol) and N,N-dimethylpyridin-4-amine (48 mg, 0.389 mmol). The reaction vial was sealed and heated to 70° C. for 4 hours before cooling to RT. The reaction mixture was evaporated to dryness. Purification by flash chromatography (50 g KP-Sil Biotage SNAP cartridge, 5-40% EtOAc in heptane) gave a white solid. This was dissolved in DCM (30 mL) and washed with 1 N NaOH solution (50 mL). The organic layer was dried over sodium sulfate, filtered and evaporated to dryness to give N-(4-chloro-2-pyridyl)-2-methyl-propanamide (546 mg, 69% Yield) as a white solid. 1H NMR (250 MHz, DMSO-d6) δ 10.68 (s, 1H), 8.29 (d, J=5.4 Hz, 1H), 8.19 (d, J=1.6 Hz, 1H), 7.22 (dd, J=5.4, 2.0 Hz, 1H), 2.75 (hept, J=6.8 Hz, 1H), 1.08 (d, J=6.8 Hz, 6H).

Step 2

To a solution of N-(4-chloro-2-pyridyl)-2-methyl-propanamide (100 mg, 0.498 mmol) and phenol (47 mg, 0.498 mmol) in DMSO (1 mL) at RT was added potassium tert-butoxide (67 mg, 0.598 mmol). The reaction mixture was stirred at 160° C. for 3 hours and allowed to cool to RT. The mixture was diluted with EtOAc (20 mL) and washed with water (30 mL). The organic layer was dried over sodium sulfate, filtered and evaporated to dryness. Purification by prep HPLC (Method F) followed by concentration and lyophilisation gave the title compound as an off-white solid (56 mg, 0.218 mmol, 44% Yield). 1H NMR (250 MHz, DMSO-d6) δ 10.44 (s, 1H), 8.18 (d, J=5.7 Hz, 1H), 7.68 (d, J=2.3 Hz, 1H), 7.57-7.41 (m, 2H), 7.37-7.25 (m, 1H), 7.23-7.10 (m, 2H), 6.67 (dd, J=5.7, 2.4 Hz, 1H), 2.77-2.60 (m, 1H), 1.03 (d, J=6.8 Hz, 6H). LCMS: m/z 257.1 [M+H]+, (ESI+), RT=2.33 (Method A)

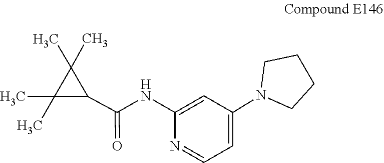

### 2,2,3,3-tetramethyl-N-(4-pyrrolidin-1-yl-2-pyridyl)cyclopropanecarboxamide

Step 1

To a stirred solution of 4-chloropyridin-2-amine (200 mg, 1.56 mmol)and N-ethyl-N-isopropyl-propan-2-amine (0.54 mL, 3.09 mmol)in DCM (3 mL) at RT was added a solution of 2,2,3,3-tetramethylcyclopropanecarbonyl chloride (497 mg, 3.10 mmol)in DCM (1 mL) and stirred for 2 h. The reaction mixture was washed with sat. NaHCO3 (2 mL), dried over sodium sulfate, filtered and evaporated to dryness. The residue was dissolved in MeOH (5 mL) and 1 N NaOH solution (3 mL) and stirred at RT for 1 h before evaporating the solvent in vacuo, and the residue washed with brine (15 mL), extracted with EtOAc (3×15 mL) and concentrated under reduced pressure. Purification by flash column chromatography (Biotage SNAP KP-Sil 50 g, heptane: ethyl acetate, 0-20%) gave N-(4-chloro-2-pyridyl)-2,2,3,3-tetramethyl-cyclopropanecarboxamide (150 mg, 35% Yield) as a white solid. 1H NMR (400 MHz, Chloroform-d) δ 8.29 (d, J=1.8 Hz, 1H), 8.15-8.09 (m, 2H), 6.99 (dd, J=5.4, 1.9 Hz, 1H), 1.32 (s, 6H), 1.22 (s, 6H), 1.02 (s, 1H).

Step 2

A mixture of N-(4-chloro-2-pyridyl)-2,2,3,3-tetramethyl-cyclopropanecarboxamide (70 mg, 0.260 mmol) and pyrrolidine (0.11 mL, 1.30 mmol) in NMP (2 mL) was heated under microwave irradiation at 225° C. for 30 mins. The mixture was washed with ethyl acetate (20 mL) and extracted with water (2×30 mL), and the combined organics were dried using hydrophobic filter and evaporated under reduced pressure. The crude reaction mixture was purified by prep HPLC (Method E) to afford the title compound (28 mg, 37% Yield) as a white solid. 1H NMR (500 MHz, Chloroform-d) δ 8.68 (s, 1H), 7.63 (d, J=6.8 Hz, 1H), 7.54 (d, J=2.4 Hz, 1H), 6.18 (dd, J=6.8, 2.5 Hz, 1H), 3.43-3.45 (m, 4H), 2.08-2.01 (m, 4H), 1.40 (s, 1H), 1.30 (s, 6H), 1.24 (s, 6H). LCMS: m/z 288.2 [M+H]+, (ESI+), RT=2.10 (Method A).

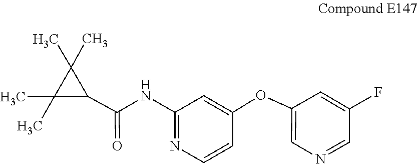

### N-[4-[(5-fluoro-3-pyridyl)oxy]-2-pyridyl]-2,2,3,3-tetramethyl-cyclopropanecarboxamide

Step 1

To a solution of 4-fluoropyridin-2-amine (300 mg, 2.62 mmol)and N-ethyl-N-isopropyl-propan-2-amine (916 uL, 5.25 mmol) in THF-Anhydrous (15 mL) was added 2,2,3,3-tetramethylcyclopropanecarbonyl chloride (506 mg, 3.15 mmol) with stirring at RT for 16 hour after which time, MeOH (5 mL), 1M NaOH (5 mL) were added and the reaction mixture stirred at RT for 0.5 h. The reaction mixture was diluted with brine (25 mL) and extracted with EtOAc (3×25 mL) and the combined organic extracts washed concentrated in vacuo. The crude product was purified by flash column chromatography (50 g silica cartridge, 0-20% EtOAc in heptane) to afford N-(4-fluoro-2-pyridyl)-2,2,3,3-tetramethyl-cyclopropanecarboxamide (302 mg, 46% Yield) as a colourless solid. 1H NMR (400 MHz, Chloroform-d) δ 8.21 (dd,J=8.6, 5.7 Hz, 1H), 8.10 (s, 1H), 8.01 (dd,J=11.4, 2.4 Hz, 1H), 6.75 (ddd,J=7.9, 5.7, 2.4 Hz, 1H), 1.34 (s, 6H), 1.24 (s, 6H), 1.03 (s, 1H).

Step 2

To a stirred solution of 5-fluoropyridin-3-ol (48 mg, 0.422 mmol) in DMF-Anhydrous (3 mL) was added sodium hydride (60% dispersion in oil) (24 mg, 0.603 mmol). After gas evolution ceased, N-(4-fluoro-2-pyridyl)-2,2,3,3-tetramethyl-cyclopropanecarboxamide (100 mg, 0.402 mmol) was added and the reaction mixture heated to 100° C. The reaction mixture was then heated to 120° C. for 16 h. The reaction mixture was washed with water (30 mL) and extracted with diethyl ether (2×20 mL). The combined organic layers were dried (hydrophobic filter) and concentrated to dryness under reduced pressure. Purification (10 g Biotage SNAP KP-Sil cartridge, 0-35% EtOAc in heptane) followed by freeze-drying gave the title product as a colourless solid (37 mg, 27% yield). 1H NMR (500 MHz, Chloroform-d) δ 8.36 (d, J=2.4 Hz, 1H), 8.30 (d, J=2.2 Hz, 1H), 8.17 (d, J=5.7 Hz, 1H), 8.07 (s, 1H), 7.92 (d, J=2.3 Hz, 1H), 7.16 (dt, J=9.1, 2.4 Hz, 1H), 6.58 (dd, J=5.7, 2.3 Hz, 1H), 1.29 (s, 6H), 1.21 (s, 6H), 1.01 (s, 1H). LCMS: m/z 330.3 [M+H]+, (ESI+), RT=3.16 (Method A)

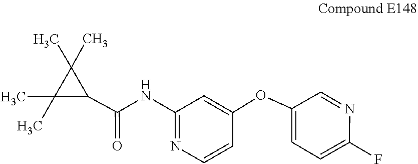

### N-[4-[(6-fluoro-3-pyridyl)oxy]-2-pyridyl]-2,2,3,3-tetramethyl-cyclopropanecarboxamide

Step 1

6-fluoropyridin-3-ol (270 mg, 2.39 mmol) and N,N-dimethylglycine hydrochloride (1:1) (178 mg, 1.27 mmol) were suspended in 1,4-Dioxane-Anhydrous (8 mL) and the reaction mixture was degassed with N2 for 5 minutes. 4-iodopyridin-2-amine (350 mg, 1.59 mmol) was added and the reaction was stirred for 10 min before copper(I) iodide (121 mg, 0.636 mmol) and cesium carbonate (1296 mg, 3.98 mmol) were added and the mixture was heated to 90° C. for lh in a microwave reactor. The reaction mixture was allowed to cool down to RT, diluted with EtOAc (15 mL), filtered through a pad of Celite and further washed with EtOAc (30 mL). It was then washed with water (15 mL) and extracted with EtOAc (2×20 mL), dried, filtered and concentrated under reduced pressure. The crude was purified by column chromatography (Biotage Sfar Duo 50 g cartridge, 0-10% MeOH in EtOAc) to afford 4-[(6-fluoro-3-pyridyl)oxy]pyridin-2-amine (33 mg, 87% purity, 8.7% Yield) as an off-white solid. 1H NMR, (500 MHz, Chloroform-d) δ 8.05 (dd, J=2.8, 1.4 Hz, 1H), 7.98 (d, J=5.9 Hz, 1H), 7.53 (ddd, J =9.3, 6.4, 3.0 Hz, 1H), 6.99 (dd, J=8.7, 3.4 Hz, 1H), 6.26 (dd, J=5.9, 2.2 Hz, 1H), 5.95 (d, J=2.1 Hz, 1H), 4.49 (s, 2H).

Step 2

To a solution of tetramethylcyclopropane-l-carbonyl chloride (22 mg, 0.138 mmol) and N-ethyl-N-isopropyl-propan-2-amine (0.024 mL, 0.138 mmol) in THF-Anhydrous (1.5 mL) was added 4-[(6-fluoro-3-pyridyl)oxy]pyridin-2-amine (87%, 33 mg, 0.138 mmol) and the mixture stirred for 2 hrs at RT. Further tetramethylcyclopropane-l-carbonyl chloride (10 mg, 0.2 eq) and DIPEA (12 uL, 0.2 eq.) were added and left to stir at RT for 45 min. Then the reaction mixture concentrated to dryness and the residue was washed with water (30 mL) and extracted with EtOAc (2×25 mL). The combined organics were dried over MgSO4, and concentrated under reduced pressure. The crude was purified using column chromatography (Biotage Sfar Duo 25 g cartridge, eluents: 0-100% EtOAc in Heptane) to afford the title compound (27 mg, 56% Yield) as a white solid. 1H NMR (500 MHz, Chloroform-d) δ 8.12 (d, J=5.7 Hz, 1H), 8.04 (dd, J=2.8, 1.4 Hz, 1H), 7.97 (s, 1H), 7.85 (d, J=2.3 Hz, 1H), 7.54 (ddd, J=9.1, 6.4, 3.0 Hz, 1H), 6.99 (dd, J=8.8, 3.5 Hz, 1H), 6.51 (dd, J=5.7, 2.4 Hz, 1H), 1.29 (s, 6H), 1.21 (s, 6H), 1.00 (s, 1H). LCMS: m/z 330.2 [M+H]+, (ESI+), RT=3.29 (Method A).

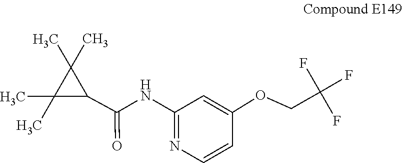

### 2,2,3,3-tetramethyl-N-[4-(2,2,2-trifluoroethoxy)-2-pyridyl]cyclopropanecarboxamide

Step 1

Boc anhydride (1090 mg, 5.00 mmol) N,N-dimethylpyridin-4-amine (12 mg, 0.102 mmol) and triethylamine (1.3 mL, 9.08 mmol) were added to a stirred suspension of 2-aminopyridin-4-ol (500 mg, 4.54 mmol) in MeCN (10 mL). The reaction was stirred at 40° C. overnight. Water (5 mL) was added and the mixture was concentrated to remove MeCN. Trituration with water (5 mL) afforded a gum. The crude material was suspended in DMF (3m1) and dipotassium carbonate (500 mg, 3.62 mmol) was added, followed by 2,2,2-trifluoroethyl trifluoromethanesulfonate (390 uL, 2.71 mmol) and the reaction stirred at 80° C. for 2 hours.

Further dipotassium carbonate (100 mg, 0.724 mmol) and 2,2,2-trifluoroethyl trifluoromethanesulfonate (140 uL, 0.972 mmol) were added and the mixture was stirred at 80° C. for 1 hour. The reaction was allowed to cool and added dropwise to stirred water (30 mL). The mixture was extracted into EtOAc (3×10 mL), dried over MgSO4, and concentrated onto silica. The crude product was purified by FCC (Biotage SNAP KP-Sil 25 g, 0-100% EtOAc in Heptane) to afford tert-butyl N-[4-(2,2,2-trifluoroethoxy)-2-pyridyl]carbamate (70.0%) (160 mg, 70% purity, 8% Yield). 1H NMR (400 MHz, Chloroform-d) δ 8.27 (s, 1H), 8.13 (d, J=5.8 Hz, 1H), 7.62 (d, J=2.3 Hz, 1H), 6.58 (dd, J=5.8, 2.4 Hz, 1H), 4.43 (q, J=8.0 Hz, 2H), 1.54 (s, 9H).

Step 2

tert-butyl N-[4-(2,2,2-trifluoroethoxy)-2-pyridyl]carbamate (70%, 160 mg, 0.383 mmol) was stirred in 4 M hydrogen chloride in Dioxane (1.0 mL, 4.00 mmol) for 2hrs then allowed to stand overnight. The reaction was concentrated under vacuum and purified prep HPLC (Method F) to afford 4-(2,2,2-trifluoroethoxy)pyridin-2-amine (38 mg, 50% Yield). 1H NMR (500 MHz, Chloroform-d) δ 7.95 (d, J=5.9 Hz, 1H), 6.28 (dd, J=5.9, 2.3 Hz, 1H), 6.00 (d, J=2.2 Hz, 1H), 4.53 (s, 2H), 4.33 (q, J=8.0 Hz, 2H).

Step 3

2,2,3,3-tetramethylcyclopropanecarbonyl chloride (62 mg, 0.384 mmol) in THF (1 mL) was added dropwise to a stirred solution of 4-(2,2,2-trifluoroethoxy)pyridin-2-amine (38 mg, 0.192 mmol) and N-ethyl-N-isopropyl-propan-2-amine (0.074 mL, 0.422 mmol) in THF (1 mL) and the reaction stirred at room temperature for 3.5 hours. The reaction was concentrated under vacuum and then diluted with MeOH (2 mL) and 1 M sodium hydroxide (1.0 mL, 1.00 mmol) added. Reaction stirred at room temp for a total of 72 h, then 2 M sodium hydroxide (1.0 mL, 2.00 mmol) added and reaction stirred at 60° C. for 3h. Methanol was removed under vacuum and the mixture was extracted with EtOAc (4×5 mL). The organics were dried over MgSO4, concentrated and purified by prep HPLC (Method G) to afford the title compound (47 mg, 77% Yield) as an off-white solid. 1H NMR (400 MHz, Chloroform-d) δ 8.09 (d, J=5.8 Hz, 1H), 8.02 (s, 1H), 7.85 (d, J=2.4 Hz, 1H), 6.62 (dd, J=5.8, 2.5 Hz, 1H), 4.42 (q, J=8.0 Hz, 2H), 1.32 (s, 6H), 1.22 (s, 6H), 1.02 (s, 1H). LCMS: m/z 317.5 [M+H]+, (ESI+), RT=2.97 (Method A).

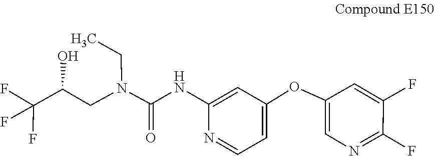

### 3-[4-(3,4-difluorophenoxy)-2-pyridyl]-1-ethyl-1-[(2R)-3,3,3-trifluoro-2-hydroxy-propyl]urea

Step 1

To a solution of 4-fluoropyridin-2-amine (250 mg, 2.19 mmol) and 3,4-difluorophenol (370 mg, 2.84 mmol) in NMP (3 mL) at RT was added N-ethyl-N-isopropyl-propan-2-amine (0.76 mL, 4.37 mmol). The reaction mixture was stirred at 180° C. for 8 h. It was then cooled to RT and diluted with EtOAc (30 mL) and water/brine (1:1, 30 mL). The layers were separated and the aqueous layer was extracted with EtOAc (2×20 mL). The combined organic extracts were dried over MgSO4, filtered, concentrated under reduced pressure and purified by flash column chromatography (25 g SiO2 column, 0-100% EtOAc in heptane) to afford 4-(3,4-difluorophenoxy)pyridin-2-amine (260 mg, 86% purity, 46% yield) as a beige solid. 1H NMR (500 MHz, DMSO-d6) δ 7.82 (d, J=5.8 Hz, 1H), 7.53 (ddd, J=10.5, 9.2, 9.2 Hz, 1H), 7.39 (ddd, J=11.5, 6.9, 2.9 Hz, 1H), 7.02 (dddd, J=8.8, 3.6, 3.5, 1.8 Hz, 1H), 6.14 (dd, J=5.8, 2.3 Hz, 1H), 5.97 (s, 2H), 5.85 (d, J=2.3 Hz, 1H).

Step 2

4-(3,4-difluorophenoxy)pyridin-2-amine (86%, 40 mg, 0.155 mmol) was dissolved in DCM-Anhydrous (1 mL) and CDI (33 mg, 0.201 mmol) was added. The reaction mixture was stirred for 22 hours at RT. A solution of (2R)-3-(ethylamino)-1,1,1-trifluoro-propan-2-ol (Intermediate I02, 32 mg, 0.201 mmol) in DCM-Anhydrous (1 mL) was added and the mixture was stirred for another 1 hour at RT. The reaction mixture was diluted with water, passed through a hydrophobic frit and concentrated. The product was purified with prep HPLC (Method E) followed by SCX cartridge (2 g) eluting first with MeOH (3 CV) then with 2M Ammonia in MeOH (3 CV). The ammonia fractions were combined and concentrated to afford the title compound (8.6 mg, 0.0212 mmol, 14% Yield) as an off white solid. 1H NMR (500 MHz, DMSO-d6) δ 9.03 (s, 1H), 8.13 (d, J=5.7 Hz, 1H), 7.59-7.51 (m, 1H), 7.48-7.39 (m, 2H), 7.10-6.96 (m, 2H), 6.60 (dd, J=5.7, 2.4 Hz, 1H), 4.29-4.20 (m, 1H), 3.57-3.50 (m, 1H), 3.45-3.38 (m, 3H), 1.07 (t, J=7.0 Hz, 3H). LCMS: m/z 406.3 [M+H]+, (ESI+), RT=3.57 (Method B).

TABLE 5
The following compounds were Synthesized using a similar method to that used in
Compound E150 using or amino-alcohols Synthesized as per Intermediate I02 or Compound
E139, Step 1

| Compound | | | LCMS |
|---|---|---|---|
| number | Structure | Analytical data | Method |
| E151 | 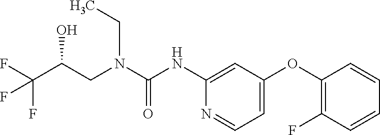 [SMILES: CCN(C[C@@H](O)C(F)(F)F)C(=O)Nc1cc(Oc2ccccc2F)ccn1] | 1H NMR (400 MHz, Methanol-d4) δ 8.10 (d, J = 5.9 Hz, 1H), 7.44 (d, J = 2.3 Hz, 1H), 7.36-7.29 (m, 4H), 6.61 (dd, J = 5.9, 2.3 Hz, 1H), 4.33- 4.24 (m, 1H), 3.67-3.41 (m, 4H), 1.24 (t, J = 7.1 Hz, 3H). LCMS: m/z 388.2 [M + H]+, (ESI+), RT = 2.44 | A |
| E152 | 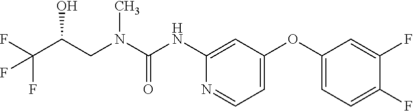 [SMILES: CN(C[C@@H](O)C(F)(F)F)C(=O)Nc1cc(Oc2ccc(F)c(F)c2)ccn1] | 1H NMR (500 MHz, DMSO- d6) δ 9.01 (s, 1H), 8.13 (d, J = 5.7 Hz, 1H), 7.55 (ddd, J = 10.4, 9.2, 9.2 Hz, 1H), 7.44 (ddd, J = 11.4, 6.8, 2.9 Hz, 1H), 7.40 (d, J = 2.3 Hz, 1H), 7.11- 7.02 (m, 1H), 6.79 (s, 1H), 6.61 | A |
| | | (dd, J = 5.7, 2.4 Hz, 1H), 4.39- | |
| | | 4.12 (m, 1H), 3.56 (dd, J = | |
| | | 14.7, 3.1 Hz, 1H), 3.47-3.38 | |
| | | (m, 1H), 2.99 (s, 3H). | |
| | | LCMS: m/z 392.1 [M + H]+, | |
| | | (ESI+), RT = 2.25 | |
| E153 | 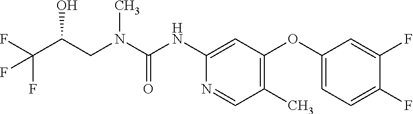 [SMILES: Cc1cnc(NC(=O)N(C)C[C@@H](O)C(F)(F)F)cc1Oc1ccc(F)c(F)c1] | 1H NMR (500 MHz, DMSO- d6) δ 8.86 (s, 1H), 8.08 (s, 1H), 7.61-7.48 (m, 1H), 7.38 (ddd, J = 11.4, 6.8, 2.9 Hz, 1H), 7.23 (s, 1H), 7.08-6.93 (m, 1H), 6.75 (s, 1H), 4.29-4.13 (m, 1H), 3.51 (dd, J = 14.7, 3.2 Hz, | A |
| | | 1H), 3.38 (dd, J = 14.3, 9.3 Hz, | |
| | | 1H), 2.96 (s, 3H), 2.17 (s, 3H). | |
| | | LCMS: m/z 406.2 [M + H]+, | |
| | | (ESI+), RT = 2.28 | |
| E154 | 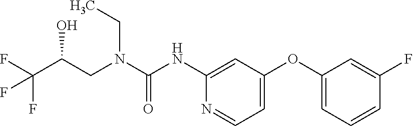 [SMILES: CCN(C[C@@H](O)C(F)(F)F)C(=O)Nc1cc(Oc2cccc(F)c2)ccn1] | 1H NMR (400 MHz, Methanol- d4) δ 8.13 (d, J = 5.8 Hz, 1H), 7.54-7.44 (m, 2H), 7.10- 6.92 (m, 3H), 6.66 (dd, J = 5.8, 2.3 Hz, 1H), 4.29 (dd, J = 11.8, 7.0 Hz, 1H), 3.63-3.42 (m, 4H), 1.24 (t, J = 7.1 Hz, 3H). LCMS: m/z 388.1 [M + H]+, | A |
| | | (ESI+), RT = 2.46 | |
| E155 | 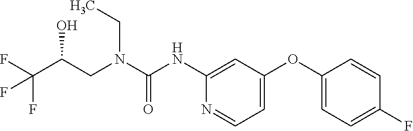 [SMILES: CCN(C[C@@H](O)C(F)(F)F)C(=O)Nc1cc(Oc2ccc(F)cc2)ccn1] | 1H NMR (400 MHz, Methanol- d4) δ 8.07 (d, J = 5.9 Hz, 1H), 7.40 (d, J = 2.3 Hz, 1H), 7.26- 7.10 (m, 4H), 6.59 (dd, J = 5.9, 2.3 Hz, 1H), 4.26 (d, J = 11.8 Hz, 1H), 3.63-3.39 (m, 4H), 1.22 (t, J = 7.1 Hz, 3H). LCMS: m/z 388.1 [M + H]+, | A |
| | | (ESI+), RT = 2.31 | |
| E156 | 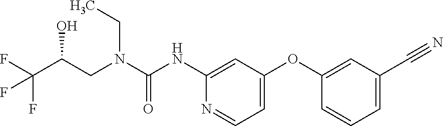 [SMILES: CCN(C[C@@H](O)C(F)(F)F)C(=O)Nc1cc(Oc2cccc(C#N)c2)ccn1] | 1H NMR (400 MHz, DMSO- d6) δ 9.06 (s, 1H), 8.16 (d, J = 5.7 Hz, 1H), 7.78-7.72 (m, 2H), 7.71-7.64 (m, 1H), 7.56- 7.51 (m, 1H), 7.43 (d, J = 2.3 Hz, 1H), 7.01 (s, 1H), 6.63 (dd, J = 5.7, 2.4 Hz, 1H), 4.31- 4.19 (m, 1H), 3.56-3.50 (m, | B |
| | | 1H), 3.44-3.37 (m, 3H), 1.07 | |
| | | (t, J = 7.0 Hz, 3H). | |
| | | LCMS: m/z 395.3 [M + H]+, | |
| | | (ESI+), RT = 3.25 | |
| E157 | 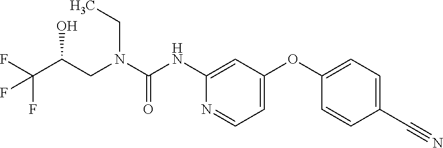 [SMILES: CCN(C[C@@H](O)C(F)(F)F)C(=O)Nc1cc(Oc2ccc(C#N)cc2)ccn1] | 1H NMR (400 MHz, DMSO- d6) δ 9.10 (s, 1H), 8.20 (d, J = 5.7 Hz, 1H), 7.98-7.90 (m, 2H), 7.49 (d, J = 2.2 Hz, 1H), 7.36-7.29 (m, 2H), 7.03 (s, 1H), 6.71 (dd, J = 5.7, 2.3 Hz, 1H), 4.31-4.19 (m, 1H), 3.57- 3.49 (m, 1H), 3.45-3.37 (m, 3H), 1.07 (t, J = 7.0 Hz, 3H). | B |
| | | LCMS: m/z 395.3 [M + H]+, | |
| | | (ESI+), RT = 3.25 | |
| E158 | 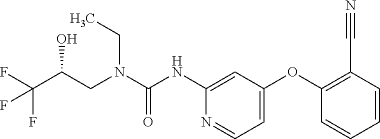 [SMILES: CCN(C[C@@H](O)C(F)(F)F)C(=O)Nc1cc(Oc2ccccc2C#N)ccn1] | 1H NMR (400 MHz, DMSO- d6) δ 9.11 (s, 1H), 8.20 (d, J = 5.7 Hz, 1H), 7.99 (dd, J = 7.8, 1.6 Hz, 1H), 7.81 (ddd, J = 8.4, 7.6, 1.7 Hz, 1H), 7.52-7.43 (m, 2H), 7.36 (d, J = 7.8 Hz, 1H), 7.02 (s, 1H), 6.70 (dd, J = 5.7, 2.4 Hz, 1H), 4.31-4.19 (m, 1H), 3.54 (dd, J = 15.0, 2.7 | A |
| | | Hz, 1H), 3.45-3.37 (m, 3H), | |
| | | 1.07 (t, J = 7.0 Hz, 3H). | |
| | | LCMS: m/z 395.2 [M + H]+, | |
| | | (ESI+), RT = 2.45 | |
| E159 | 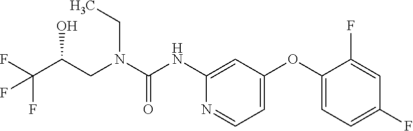 [SMILES: CCN(C[C@@H](O)C(F)(F)F)C(=O)Nc1cc(Oc2ccc(F)cc2F)ccn1] | (400 MHz, Chloroform-d) δ 8.02 (s, 1H), 7.67-7.48 (m, 1H), 7.17 (td, J = 8.9, 5.5 Hz, 1H), 7.02-6.89 (m, 2H), 6.51 (dd, J = 5.8, 2.1 Hz, 1H), 4.19- 4.09 (m, 1H), 3.80 (dd, J = 15.2, 8.6 Hz, 1H), 3.51-3.32 (m, 3H), 1.28 (t, J = 7.2 Hz, | A |
| | | 3H). | |
| | | LCMS: m/z 406.5 [M + H]+, | |
| | | (ESI+), RT = 2.59 | |
| E160 | 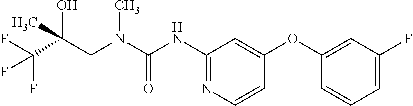 [SMILES: CN(C[C@@](C)(O)C(F)(F)F)C(=O)Nc1cc(Oc2cccc(F)c2)ccn1] | 1H NMR (500 MHz, DMSO- d6) δ 9.26 (s, 1H), 8.14 (d, J = 5.7 Hz, 1H), 7.51 (td, J = 8.2, 6.9 Hz, 1H), 7.41 (d, J = 2.2 Hz, 1H), 7.16-7.09 (m, 2H), 7.04-6.99 (m, 1H), 6.63 (dd, J = 5.7, 2.4 Hz, 1H), 3.70-3.59 | B |
| | | (m, 1H), 3.47-3.40 (m, 1H), | |
| | | 3.00 (s, 3H), 1.27 (s, 3H). | |
| | | LCMS: m/z 388.2 [M + H]+, | |
| | | (ESI+), RT = 3.49 | |
| E161 | 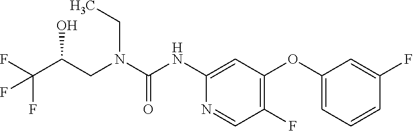 [SMILES: CCN(C[C@@H](O)C(F)(F)F)C(=O)Nc1cc(Oc2cccc(F)c2)c(F)cn1] | (500 MHz, Chloroform-d) δ 8.08 (d, J = 2.4 Hz, 1H), 7.64 (d, J = 6.3 Hz, 1H), 7.56 (s, 1H), 7.37 (td, J = 8.3, 6.5 Hz, 1H), 6.97-6.92 (m, 1H), 6.90 (dd, J = 8.2, 2.2 Hz, 1H), 6.83 (dt, J = 9.5, 2.3 Hz, 1H), 5.24 (s, 1H), 4.12 (p, J = 6.7 Hz, | A |
| | | 1H), 3.75 (dd, J = 15.2, 8.7 Hz, | |
| | | 1H), 3.49-3.40 (m, 2H), 3.35 | |
| | | (dq, J = 14.6, 7.2 Hz, 1H), 1.27 | |
| | | (t, J = 7.2 Hz, 3H). | |
| | | LCMS: m/z 406.2 [M + H]+, | |
| | | (ESI+), RT = 3.51 | |

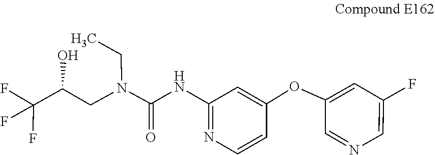

### 1-ethyl-3-[4-[(5-fluoro-3-pyridyl)oxy]-2-pyridyl]-1-[(2R)-3,3,3-trifluoro-2-hydroxy-propyl]urea

Step 1

To a solution of 4-fluoropyridin-2-amine (100 mg, 0.874 mmol) and 5-fluoropyridin-3-ol (129 mg, 1.14 mmol) in NMP (2 mL) at RT was added N-ethyl-N-isopropyl-propan-2-amine (0.31 mL, 1.75 mmol). The reaction mixture was stirred at 180° C. for 5 h. It was then cooled to RT and diluted with EtOAc (30 mL) and water/brine (1:1, 30 mL). The layers were separated and the aqueous layer was extracted with EtOAc (2×20 mL). The combined organic extracts were dried (hydrophobic frit), concentrated under reduced pressure and purified by SCX-2 cartridge 5 g, washing with MeOH (5 CV), eluting with 7N NH3/MeOH (5CV) to afford 4-[(5-fluoro-3-pyridyl)oxy]pyridin-2-amine (180 mg, 85% purity, 85% Yield) as a brown oil. 1H NMR (500 MHz, Chloroform-d) δ 8.39 (d,J=2.4 Hz, 1H), 8.33 (d,J=2.1 Hz, 1H), 8.05-8.00 (m, 1H), 7.20 (dt,J=9.1, 2.4 Hz, 1H), 6.34 (dd,J=5.9, 2.2 Hz, 1H), 6.06 (d,J=2.1 Hz, 1H), 4.57 (s, 2H).

Step 2

To a solution of bis(trichloromethyl) carbonate (30 mg, 0.0994 mmol) in anhydrous DCM (2 mL) cooled in a dry ice-acetone bath was added a solution of 4-[(5-fluoro-3-pyridyl)oxy]pyridin-2-amine (85%, 60 mg, 0.249 mmol) and pyridine (20 uL, 0.249 mmol) in anhydrous DCM (2 mL) dropwise over 10 mins. The reaction was stirred at -78° C. for 5 mins. (2R)-3-(ethylamino)-1,1,1-trifluoro-propan-2-ol (Intermediate I02, 51 mg, 0.323 mmol) and N-ethyl-N-isopropyl-propan-2-amine (65 uL, 0.373 mmol) in anhydrous DCM (2 mL) were then added and the reaction was stirred for 5 mins at -78° C. then in an ice bath for 1 hour. The reaction mixture was then concentrated under reduced pressure and purified by prep HPLC (Method G) to afford the title compound (7.9 mg, 7.9% Yield) as a yellow solid. 1HNMR(500 MHz, Chloroform-d) δ 8.43 (d,J=2.4 Hz, 1H), 8.35 (d,J=2.1 Hz, 1H), 8.15 (s, 1H), 8.09 (d,J=5.9 Hz, 1H), 7.74 (d,J=2.0 Hz, 1H), 7.24 (dt,J=8.9, 2.3 Hz, 1H), 6.63 (dd,J=5.9, 2.3 Hz, 1H), 4.23-4.12 (m, 1H), 3.88-3.77 (m, 1H), 3.60-3.38 (m, 3H), 1.30 (t,J=7.2 Hz, 3H). LCMS: m/z 389.1 [M+H]+, (ESI+), RT=2.08 (Method A).

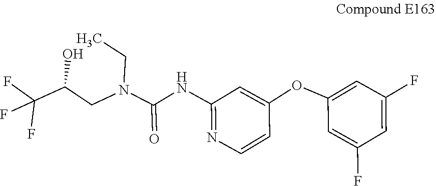

### 3-[4-(3,5-difluorophenoxy)-2-pyridyl]-1-ethyl-1-[(2R)-3,3,3-trifluoro-2-hydroxy-propyl]urea

Synthesized using a similar method to that used to synthesise Compound E162. 1HNMR(500 MHz, Chloroform-d) δ 8.08 (d,J=5.5 Hz, 1H), 7.67 (s, 1H), 6.70 (tt,J=8.8, 2.3 Hz, 1H), 6.67-6.62 (m, 2H), 6.60 (dd,J=5.8, 2.2 Hz, 1H), 4.23-4.13 (m, 1H), 3.82 (dd, J=15.3, 8.8 Hz, 1H), 3.57-3.44 (m, 2H), 3.39 (dq,J=14.6, 7.1 Hz, 1H), 1.30 (t,J=7.2 Hz, 3H). LCMS: m/z 406.1 [M+H]+, (ESI+), RT=2.77 (Method A).

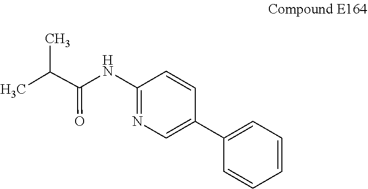

### 2-methyl-N-(5-phenyl-2-pyridyl)propanamide

Step 1

To a stirred solution of isobutyric anhydride (719 uL, 4.34 mmol)and 5-bromopyridin-2-amine (500 mg, 2.89 mmol) in THF (15.5 mL) was added N-ethyl-N-isopropyl-propan-2-amine (1009 uL, 5.78 mmol) followed by N,N-dimethylpyridin-4-amine (35 mg, 0.289 mmol) and stirred at 80° C. in a sealable pressure tube for 2 h. The reaction mixture was then evaporated to dryness. Purification by flash chromatography (Biotage 50 g KP-Sil SNAP cartridge, 10-100% EtOAc in heptane) gave N-(5-bromo-2-pyridyl)-2-methyl-propanamide (502 mg, 71% Yield) as a white solid. 1H NMR (500 MHz, DMSO-d6) δ 10.57 (s, 1H), 8.42 (d, J=2.5 Hz, 1H), 8.08 (d, J=8.9 Hz, 1H), 7.98 (dd, J=8.9, 2.5 Hz, 1H), 2.74 (hept, J=6.8 Hz, 1H), 1.08 (d, J=6.8 Hz, 6H).

Step 2

N-(5-bromo-2-pyridyl)-2-methyl-propanamide (70 mg, 0.288 mmol) and phenylboronic acid (39 mg, 0.317 mmol) were dissolved in 1,4-Dioxane-Anhydrous (2 mL) and 2 M Na2CO3 (0.29 mL, 0.576 mmol) and the reaction mixture was degassed with N2 for 5 minutes. Pd(dppf)C12 (11 mg, 0.0144 mmol) was then added and the reaction was heated to 110° C. for 2 h. It was then washed with water and extracted with ethyl acetate, cooled to RT, concentrated under reduced pressure and purified by prep HPLC (Method F) to afford the title compound as a light brown solid (19.6 mg). 1H NMR (250 MHz, DMSO-d6) δ 10.50 (s, 1H), 8.63 (dd, J=2.5, 0.8 Hz, 1H), 8.19 (dd, J=8.7, 0.8 Hz, 1H), 8.08 (dd, J=8.7, 2.5 Hz, 1H), 7.75-7.66 (m, 2H), 7.54-7.43 (m, 2H), 7.42-7.34 (m, 1H), 2.76 (h, J=6.8 Hz, 1H), 1.10 (d, J=6.8 Hz, 6H). LCMS: m/z 241.2 [M+H]+, (ESI+), RT=2.93 (Method A).

TABLE 6
The following compounds were Synthesized using a similar method to that used in
Compound E164

| Compound | | | LCMS |
|---|---|---|---|
| number | Structure | Analytical data | Method |
| E165 | 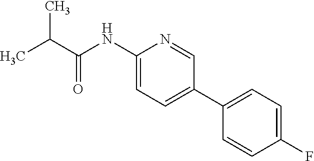 [SMILES: CC(C)C(=O)Nc1ccc(-c2ccc(F)cc2)cn1] | 1H NMR (500 MHz, DMSO-d6) δ 10.50 (s, 1H), 8.61 (dd, J = 2.5, 0.6 Hz, 1H), 8.17 (d, J = 8.7 Hz, 1H), 8.07 (dd, J = 8.7, 2.6 Hz, 1H), 7.78-7.72 (m, 2H), 7.33-7.28 (m, 2H), 2.77 (hept, J = 6.8 Hz, 1H), 1.10 (d, J = 6.8 Hz, 6H). LCMS: m/z 259.2 [M + H]+, (ESI+), RT = 3.05 | A |
| E166 | 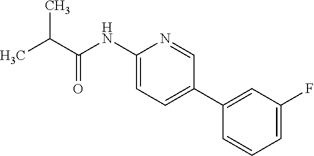 [SMILES: CC(C)C(=O)Nc1ccc(-c2cccc(F)c2)cn1] | 1H NMR (500 MHz, DMSO-d6) δ 10.54 (s, 1H), 8.68 (d, J = 2.5 Hz, 1H), 8.19 (dd, J = 8.0 Hz, 1H), 8.13 (dd, J = 8.7, 2.5 Hz, 1H), 7.62-7.55 (m, 2H), 7.54-7.48 (m, 1H), 7.23-7.18 (m, 1H), 2.81- 2.74 (m, 1H), 1.10 (d, J = 6.8 Hz, 6H). LCMS: m/z 259.2 [M + H]+, (ESI+), RT = 3.15 | A |
| E167 | 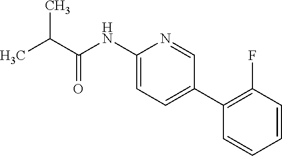 [SMILES: CC(C)C(=O)Nc1ccc(-c2ccccc2F)cn1] | 1H NMR (500 MHz, DMSO-d6) δ 10.54 (s, 1H), 8.51-8.47 (m, 1H), 8.23-8.17 (m, 1H), 8.00- 7.94 (m, 1H), 7.61-7.56 (m, 1H), 7.47-7.41 (m, 1H), 7.36-7.30 (m, 2H), 2.78 (hept, J = 6.8 Hz, 1H), 1.11 (d, J = 6.8 Hz, 6H). LCMS: m/z 259.2 [M + H]+, (ESI+), RT = 3.11 | A |
| E168 | 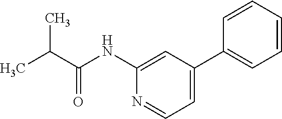 [SMILES: CC(C)C(=O)Nc1cc(-c2ccccc2)ccn1] | 1H NMR (500 MHz, DMSO-d6) δ 10.49 (s, 1H), 8.43 (s, 1H), 8.36 (d, J = 5.2 Hz, 1H), 7.75-7.69 (m, 2H), 7.58-7.44 (m, 3H), 7.40 (dd, J = 5.2, 1.7 Hz, 1H), 2.78 (hept, J = 6.8 Hz, 1H), 1.10 (d, J = 6.8 Hz, 6H). LCMS: m/z 241.1 [M + H]+, (ESI+), RT = 2.49 | A |
| E169 | 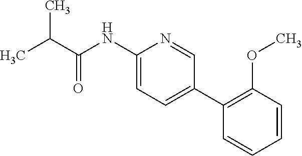 [SMILES: COc1ccccc1-c1ccc(NC(=O)C(C)C)nc1] | 1H NMR (500 MHz, Chloroform-d) δ 8.42 (d, J = 2.1 Hz, 1H), 8.27 (d, J = 8.6 Hz, 1H), 8.02 (s, 1H), 7.89 (dd, J = 8.6, 2.3 Hz, 1H), 7.37-7.32 (m, 1H), 7.30 (dd, J = 7.5, 1.7 Hz, 1H), 7.07-7.02 (m, 1H), 6.99 (d, J = 8.2 Hz, 1H), 3.82 (s, 3H), 2.57 (hept, J = 6.9 Hz, 1H), 1.28 (d, J = 6.9 Hz, 6H). LCMS: m/z 271.2 [M + H]+, (ESI+), RT = 2.83 | A |
| E170 | 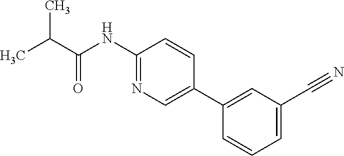 [SMILES: CC(C)C(=O)Nc1ccc(-c2cccc(C#N)c2)cn1] | 1H NMR (500 MHz, Chloroform-d) δ 8.48 (d, J = 2.4 Hz, 1H), 8.36 (d, J = 8.7 Hz, 1H), 7.96 (s, 1H), 7.90 (dd, J = 8.7, 2.5 Hz, 1H), 7.85-7.83 (m, 1H), 7.80-7.77 (m, 1H), 7.68-7.65 (m, 1H), 7.60- 7.55 (m, 1H), 2.59 (hept, J = 6.9 Hz, 1H), 1.30 (d, J = 6.9 Hz, 6H). LCMS: m/z 266.2 [M + H]+, (ESI+), RT = 2.89 | A |
| E171 | 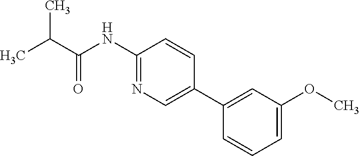 [SMILES: COc1cccc(-c2ccc(NC(=O)C(C)C)nc2)c1] | 1H NMR (500 MHz, Chloroform-d) δ 8.49 (d, J = 2.4 Hz, 1H), 8.30 (d, J = 8.6 Hz, 1H), 7.95 (s, 1H), 7.91 (dd, J = 8.6, 2.4 Hz, 1H), 7.40-7.35 (m, 1H), 7.16-7.12 (m, 1H), 7.09-7.06 (m, 1H), 6.92 (dd, J = 8.2, 2.5 Hz, 1H), 3.87 (s, 3H), 2.58 (hept, J = 6.8 Hz, 1H), 1.29 (d, J = 6.9 Hz, 6H). LCMS: m/z 271.2 [M + H]+, (ESI+), RT = 3.01 | A |
| E172 | 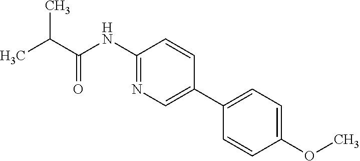 [SMILES: COc1ccc(-c2ccc(NC(=O)C(C)C)nc2)cc1] | 1H NMR (500 MHz, Chloroform-d) δ 8.45 (s, 1H), 8.28 (d, J = 8.6 Hz, 1H), 7.92 (s, 1H), 7.87 (dd, J = 8.6, 2.3 Hz, 1H), 7.51-7.45 (m, 2H), 7.02-6.96 (m, 2H), 3.86 (s, 3H), 2.57 (hept, J = 7.0 Hz, 1H), 1.29 (d, J = 6.9 Hz, 6H). LCMS: m/z 271.2 [M + H]+, (ESI+), RT = 2.82 | A |
| E173 | 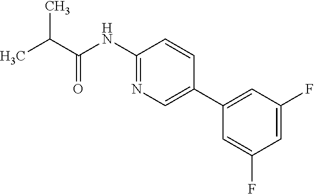 [SMILES: CC(C)C(=O)Nc1ccc(-c2cc(F)cc(F)c2)cn1] | 1H NMR (500 MHz, Chloroform-d) δ 8.46 (dd, J = 2.5, 0.7 Hz, 1H), 8.33 (dd, J = 8.7, 0.6 Hz, 1H), 7.95 (s, 1H), 7.87 (dd, J = 8.7, 2.5 Hz, 1H), 7.10-7.05 (m, 2H), 6.84-6.79 (m, 1H), 2.58 (hept, J = 6.9 Hz, 1H), 1.29 (d, J = 6.9 Hz, 6H). LCMS: m/z 277.2 [M + H]+, (ESI+), RT = 3.37 | A |
| E174 | 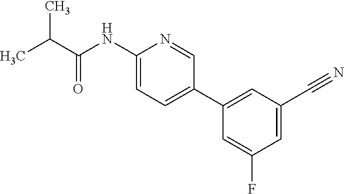 [SMILES: CC(C)C(=O)Nc1ccc(-c2cc(F)cc(C#N)c2)cn1] | 1H NMR (500 MHz, DMSO-d6) δ 10.63 (s, 1H), 8.79 (d, J = 1.9 Hz, 1H), 8.24 (dd, J = 8.8, 2.4 Hz, 1H), 8.22-8.18 (m, 1H), 8.16 (dd, J = 1.3 Hz, 1H), 8.10-7.99 (m, 1H), 7.88-7.79 (m, 1H), 2.78 (hept, J = 7.3 Hz, 1H), 1.10 (d, J = 6.8 Hz, 6H). LCMS: m/z 284.2 [M + H]+, (ESI+), RT = 3.14 | A |
| E175 | 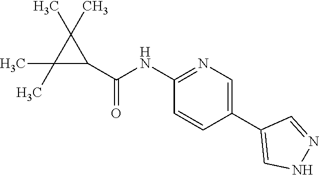 [SMILES: CC1(C)C(C(=O)Nc2ccc(-c3cn[nH]c3)cn2)C1(C)C] | (400 MHz, Chloroform-d) δ 8.41 (d, J = 1.9 Hz, 1H), 8.20 (d, J = 8.6 Hz, 1H), 7.94 (s, 1H), 7.85 (s, 2H), 7.76 (dd, J = 8.6, 2.4 Hz, 1H), 1.33 (s, 6H), 1.23 (s, 6H), 1.04 (s, 1H). LCMS: m/z 285.3 [M + H]+, (ESI+), RT = 2.37 | A |
| E176 | 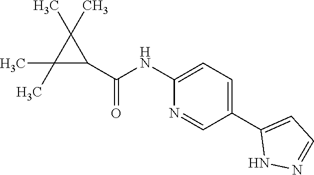 [SMILES: CC1(C)C(C(=O)Nc2ccc(-c3ccn[nH]3)cn2)C1(C)C] | (500 MHz, Chloroform-d) δ 10.43 (s, 1H), 8.71- 8.65 (m, 1H), 8.24 (d, J = 8.7 Hz, 1H), 8.13 (s, 1H), 8.05 (d, J = 7.0 Hz, 1H), 7.64 (d, J = 2.4 Hz, 1H), 6.62 (d, J = 2.3 Hz, 1H), 1.33 (s, 6H), 1.22 (s, 6H), 1.06 (s, 1H). LCMS: m/z 285.5 [M + H]+, (ESI+), RT = 2.67 | A |

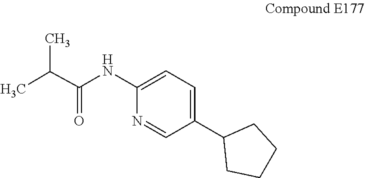

### N-(5-cyclopentyl-2-pyridyl)-2-methyl-propanamide

To a solution of N-[5-(cyclopenten-l-yl)-2-pyridyl]-2-methyl-propanamide (Synthesized using a similar method to that used in Compound E164, 43 mg, 0.188 mmol) in Ethanol (5 mL) was added 10% Pd/C (3.9 mg, 0.0363 mmol) and the reaction mixture was put under a balloon of hydrogen and stirred at RT for 2 hr. It was then filtered through Celite, washed with EtOAc and concentrated under reduced pressure and purified by flash chromatography (Biotage Isolera, C18 12 g Ultra SNAP cartridge) eluting with a solution of water (+0.1% CH202) in MeCN (+0.1% CH202) (10 to 100%) to afford the title compound as a light brown solid (10.7 mg, 24.3%). 1H NMR (500 MHz, Chloroform-d) δ 8.24 (s, 1H), 8.17 (d, J=8.6 Hz, 1H), 8.09 (d, J=2.3 Hz, 1H), 7.59 (dd, J=8.6, 2.4 Hz, 1H), 3.00-2.92 (m, 1H), 2.56 (hept, J=6.9 Hz, 1H), 2.11-2.04 (m, 2H), 1.86-1.65 (m, 4H), 1.61-1.50 (m, 2H), 1.26 (d, J=6.9 Hz, 6H). LCMS: m/z 233.2 [M+H]+, (ESI+), RT=2.43 (Method A).

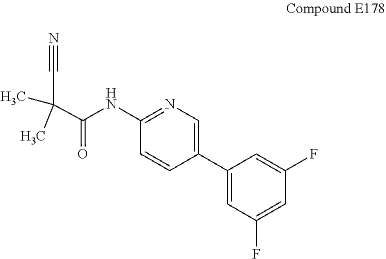 [SMILES: Cc1cc(-c2cc(F)cc(F)c2)cnc1NC(=O)C(C)(C)C#N]

### 2-cyano-N-[5-(3,5-difluorophenyl)-2-pyridyl]-2-methyl-propanamide

Step 1

5-bromopyridin-2-amine (1.00 g, 5.78 mmol) and (3,5-difluorophenyl)boronic acid (913 mg, 5.78 mmol) were dissolved in 1,4-Dioxane-Anhydrous (55 mL) and 2 M Na2CO3 (6.0 mL, 12.0 mmol) and the reaction mixture was degassed with N2 for 5 minutes. To the reaction was added Pd(dppf)C12 (212 mg, 0.289 mmol) and the reaction was heated to 110° C. for 2.5 hrs. It was then cooled to RT, concentrated under reduced pressure to 20 mL and the reaction was washed with water and extracted with ethyl acetate. The organic layer was concentrated under reduced pressure and purified by SCX-2 cartridge (washing with MeOH, eluting with 7N NH3/MeOH). This was concentrated under reduced pressure to afford 5-(3,5-difluorophenyl)pyridin-2-amine (Intermediate I03, 947 mg, 74%) as a brown solid. 1H NMR (500 MHz, DMSO-d6) δ 8.37-8.28 (m, 1H), 7.77 (dd, J=8.7, 2.6 Hz, 1H), 7.38-7.29 (m, 2H), 7.12-7.02 (m, 1H), 6.51 (dd, J=8.7, 0.7 Hz, 1H), 6.23 (s, 2H).

Step 2

A solution of 2-cyano-2-methylpropanoic acid (42 mg, 0.373 mmol), HATU (142 mg, 0.373 mmol) and DIPEA (0.18 mL, 1.02 mmol) in Acetonitrile-Anhydrous (3 mL) was stirred at RT for 1 hr. To the solution was added 5-(3,5-difluorophenyl)pyridin-2-amine (70 mg, 0.339 mmol) and the reaction was stirred at 70° C. for 4 hr, it was then stirred at 80° C. for 2 hr. The reaction was retreated with HATU (142 mg, 0.373 mmol) and 2-cyano-2-methylpropanoic acid (42 mg, 0.373 mmol) and stirred at 80° C. overnight. It was then washed with water, extracted with EtOAc (30 mL), dried over Na2SO4, filtered, concentrated under reduced pressure and purified by prep HPLC (Method E) to afford the title compound (12.6 mg, 12%) as an off- white solid. 1H NMR (500 MHz, DMSO-d6) δ 10.86 (s, 1H), 8.82 (dd, J=2.6, 0.7 Hz, 1H), 8.25 (dd, J=8.7, 2.6 Hz, 1H), 8.09 (dd, J=8.7, 0.7 Hz, 1H), 7.61-7.54 (m, 2H), 7.30-7.23 (m, 1H), 1.70 (s, 6H). LCMS: m/z 302.2 [M+H]+, (ESI+), RT=3.45 (Method A).

TABLE 7
The following compounds were Synthesized using a similar method to that used in Compound E178

| Compound | | | LCMS |
|---|---|---|---|
| number | Structure | Analytical data | Method |
| E179 | 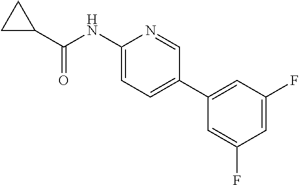 [SMILES: O=C(Nc1ccc(-c2cc(F)cc(F)c2)cn1)C1CC1] | 1H NMR (500 MHz, DMSO-d6) δ 10.95 (s, 1H), 8.74 (dd, J = 2.0, 1.4 Hz, 1H), 8.21-8.13 (m, 2H), 7.57-7.49 (m, 2H), 7.23 (tt, J = 9.3, 2.3 Hz, 1H), 2.03 (tt, J = 7.5, 5.1 Hz, 1H), 0.89-0.77 (m, 4H). LCMS: m/z 275.1 [M + H]+, (ESI+), RT = 3.23 | A |
| E180 | 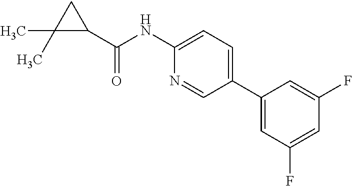 [SMILES: CC1(C)CC1C(=O)Nc1ccc(-c2cc(F)cc(F)c2)cn1] | δ 8.46 (d, J = 2.3 Hz, 1H), 8.29 (d, J = 8.7 Hz, 1H), 8.10 (s, 1H), 7.85 (dd, J = 8.7, 2.5 Hz, 1H), 7.12- 7.02 (m, 2H), 6.81 (tt, J = 8.8, 2.2 Hz, 1H), 1.47 (dd, J = 7.9, 5.3 Hz, 1H), 1.25 (s, 4H), 1.22 (s, 3H), 0.91 (dd, J = 7.9, 4.4 Hz, 1H). LCMS: m/z 303.3 [M + H]+, (ESI+), RT = 3.83 | A |
| E181 | 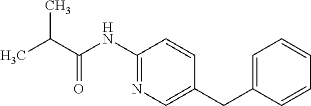 [SMILES: CC(C)C(=O)Nc1ccc(Cc2ccccc2)cn1] | 1H NMR (500 MHz, DMSO-d6) δ 10.32 (s, 1H), 8.20 (d, J = 2.1 Hz, 1H), 8.00 (d, J = 8.5 Hz, 1H), 7.60 (dd, J = 8.5, 2.3 Hz, 1H), 7.35-7.26 (m, 2H), 7.26-7.15 (m, 3H), 3.91 (s, 2H), 2.72 (hept, J = 6.8 Hz, 1H), 1.06 (d, J = 6.8 Hz, 6H). LCMS: m/z 255.2 [M + H]+, (ESI+), RT = 2.69 | A |
| E182 | 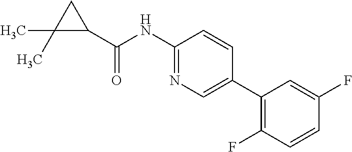 [SMILES: CC1(C)CC1C(=O)Nc1ccc(-c2cc(F)ccc2F)cn1] | (500 MHz, Chloroform-d) δ 8.44 (s, 1H), 8.29 (d, J = 8.7 Hz, 1H), 8.18 (s, 1H), 7.86 (dt, J = 8.7, 1.7 Hz, 1H), 7.17-7.09 (m, 2H), 7.06-6.99 (m, 1H), 1.47 (dd, J = 7.9, 5.3 Hz, 1H), 1.26 (t, J = 4.9 Hz, 1H), 1.24 (s, 3H), 1.21 (s, 3H), 0.91 (dd, J = 7.9, 4.4 Hz, 1H). LCMS: m/z 303.1 [M + H]+, (ESI+), RT = 3.71 | A |
| E183 | 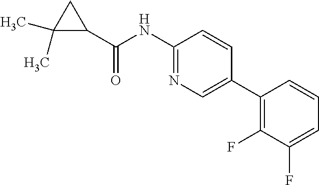 [SMILES: CC1(C)CC1C(=O)Nc1ccc(-c2cccc(F)c2F)cn1] | (500 MHz, Chloroform-d) δ 8.46 (d, J = 2.0 Hz, 1H), 8.29 (d, J = 8.7 Hz, 1H), 8.16 (s, 1H), 7.85 (dd, J = 8.7, 2.5 Hz, 1H), 7.10-7.03 (m, 2H), 6.81 (tt, J = 8.8, 2.3 Hz, 1H), 1.47 (dd, J = 7.9, 5.3 Hz, 1H), 1.28-1.25 (m, 1H), 1.24 (s, 3H), 1.22 (s, 3H), 0.91 (dd, J = 7.9, 4.4 Hz, 1H). LCMS: m/z 303.1 [M + H]+, (ESI+), RT = 3.80 | A |
| E184 | 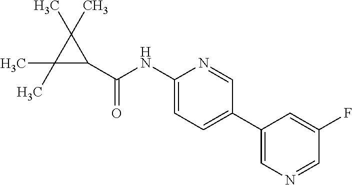 [SMILES: CC1(C)C(C(=O)Nc2ccc(-c3cncc(F)c3)cn2)C1(C)C] | (500 MHz, Chloroform-d) δ 8.64 (t, J = 1.6 Hz, 1H), 8.48 (d, J = 2.6 Hz, 2H), 8.31 (dd, J = 8.7, 0.7 Hz, 1H), 8.02 (s, 1H), 7.87 (dd, J = 8.7, 2.5 Hz, 1H), 7.57 (ddd, J = 9.3, 2.7, 1.9 Hz, 1H), 1.34 (s, 6H), 1.24 (s, 6H), 1.06 (s, 1H). LCMS: m/z 314.1 [M + H]+, (ESI+), RT = 3.52 | A |
| E185 | 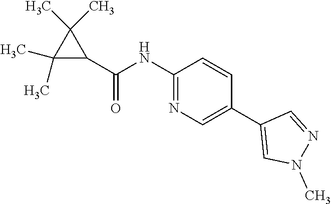 [SMILES: Cn1cc(-c2ccc(NC(=O)C3C(C)(C)C3(C)C)nc2)cn1] | (500 MHz, Chloroform-d) δ 8.37 (d, J = 1.9 Hz, 1H), 8.17 (d, J = 8.6 Hz, 1H), 7.98 (s, 1H), 7.74- 7.70 (m, 2H), 7.60 (s, 1H), 3.95 (s, 3H), 1.33 (s, 6H), 1.22 (s, 6H), 1.03 (s, 1H). LCMS: m/z 299.2 [M + H]+, (ESI+), RT = 2.65 | A |
| E186* | 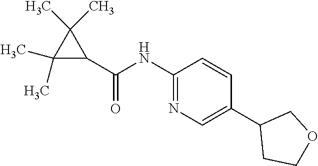 [SMILES: CC1(C)C(C(=O)Nc2ccc(C3CCOC3)cn2)C1(C)C] | 1H NMR (400 MHz, Chloroform-d) δ 8.16-8.07 (m, 2H), 7.91 (s, 1H), 7.55 (dd, J = 8.5, 2.5 Hz, 1H), 4.15-4.02 (m, 2H), 3.92 (q, J = 7.7 Hz, 1H), 3.69 (dd, J = 8.5, 7.1 Hz, 1H), 3.36 (p, J = 7.6 Hz, 1H), 2.37 (dtd, J = 12.4, 7.8, 4.7 Hz, 1H), 1.95 (dq, J = 12.4, 7.9 Hz, 1H), 1.31 (s, 6H), 1.21 (s, 6H), 1.02 (s, 1H). LCMS: m/z 289.2 [M + H]+, (ESI+), RT = 3.46 | B |
| E187 | 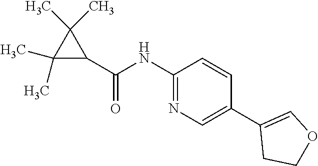 [SMILES: CC1(C)C(C(=O)Nc2ccc(C3=COCC3)cn2)C1(C)C] | 1H NMR (400 MHz, Chloroform-d) δ 8.12 (d, J = 1.9 Hz, 1H), 8.10 (d, J = 8.7 Hz, 1H), 7.97 (s, 1H), 7.52 (dd, J = 8.7, 2.4 Hz, 1H), 6.85 (t, J = 1.9 Hz, 1H), 4.51 (t, J = 9.5 Hz, 2H), 2.95 (td, J = 9.6, 2.0 Hz, 2H), 1.32 (s, 6H), 1.21 (s, 6H), 1.02 (s, 1H). LCMS: m/z 287.1 [M + H]+, (ESI+), RT = 3.80 | B |
| E188 | 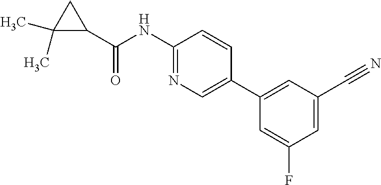 [SMILES: CC1(C)CC1C(=O)Nc1ccc(-c2cc(F)cc(C#N)c2)cn1] | 1H NMR (400 MHz, Chloroform-d) δ 8.49 (d, J = 2.4 Hz, 1H), 8.36 (d, J = 8.7 Hz, 1H), 8.17 (s, 1H), 7.89 (dd, J = 8.7, 2.5 Hz, 1H), 7.67 (t, J = 1.4 Hz, 1H), 7.52 (dt, J = 9.4, 2.0 Hz, 1H), 7.38 (ddd, J = 7.8, 2.4, 1.3 Hz, 1H), 1.50 (dd, J = 7.9, 5.3 Hz, 1H), 1.30 (t, J = 4.9 Hz, 1H), 1.27 (s, 3H), 1.25 (s, 3H), 0.95 (dd, J = 7.9, 4.4 Hz, 1H). LCMS: m/z 310.1 [M + H]+, (ESI+), RT = 3.57 | A |

*Compound E186 was isolated as a byproduct from the synthesis of E187

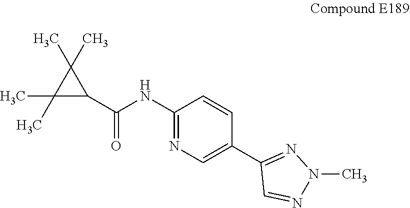

### 2,2,3,3-tetramethyl-N-[5-(2-methyltriazol-4-yl)-2-pyridyl]cyclopropanecarboxamide

Step 1

5-(4,4,5,5-tetramethyl-1,3,2-dioxaborolan-2-yl)pyridin-2-amine (100 mg, 0.454 mmol) and 4-bromo-2-methyl-triazole (81 mg, 0.500 mmol) were dissolved in 1,4-Dioxane-Anhydrous (2.15 mL) and 2 M Na2CO3 (0.47 mL, 0.949 mmol) was added to the reaction mixture and degassed with Nitrogen for 5 mins. Palladium-triphenylphosphane (1:4) (26 mg, 0.0227 mmol) was then added, degassed for another 5 mins and stirred at 110° C. for 1.5 hours. The reaction mixture was concentrated and EtOAc was added. The resultant precipitate was filtered off and purified by SCX cartridge (2 g) eluting first with MeOH (3 CV) then with 2M Ammonia in MeOH (3 CV). The ammonia fractions were concentrated to give 5-(2-methyltriazol-4-yl)pyridin-2-amine (60.0%) (EV-PGN001-228-002) (45 mg, 60% purity, 34% Yield) as a green solid. LCMS: m/z 175.8 [M+H]+, (ESI+), RT=0.25 (Method D).

Step 2

To a solution of 5-(2-methyltriazol-4-yl)pyridin-2-amine (60%, 45 mg, 0.154 mmol) in THF-Anhydrous (1.2889 mL) was added tetramethylcyclopropane-l-carbonyl chloride (27 mg, 0.170 mmol) and N-ethyl-N-(propan-2-yl)propan-2-amine (0.054 mL, 0.308 mmol). The reaction mixture was stirred at RT for 4.5 hours. Water (2 mL) was added to the reaction mixture and extracted with EtOAc (3×2 mL). The combined organic layers were dried over MgSO4, filtered and concentrated. The crude product was purified by silica flash column chromatography (0-40% EtOAc in heptane) followed by purification by SCX cartridge (2 g) eluting first with MeOH (3 CV) then with 2M Ammonia in MeOH (3 CV). The crude product was then further purified by silica flash column chromatography (0-100% EtOAc in heptane) to afford the title compound (4.5 mg, 8.8% Yield) as a white solid. 1H NMR (400 MHz, DMSO-d6) δ 10.50 (s, 1H), 8.74 (t, J=1.6 Hz, 1H), 8.53 (s, 1H), 8.14 (d, J=1.6 Hz, 2H), 4.10 (s, 3H), 1.57 (s, 1H), 1.26 (s, 6H), 1.18 (s, 6H). LCMS: m/z 300.2 [M+H]+, (ESI+), RT=3.10 (Method B).

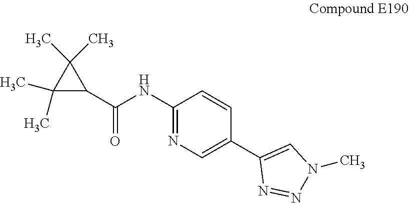

### 2,2,3,3-tetramethyl-N-[5-(1-methyltriazol-4-yl)-2-pyridyl]cyclopropanecarboxamide

Synthesized using a similar method to that used in Compound E189. 1H NMR (500 MHz, Chloroform-d) δ 8.71 (dd, J=2.3, 0.7 Hz, 1H), 8.28-8.22 (m, 1H), 8.08 (dd, J=8.6, 2.3 Hz, 1H), 7.97 (s, 1H), 7.75 (s, 1H), 4.16 (s, 3H), 1.33 (s, 6H), 1.23 (s, 6H), 1.04 (s, 1H). LCMS: m/z 300.2 [M+H]+, (ESI+), RT=3.10 (Method B).

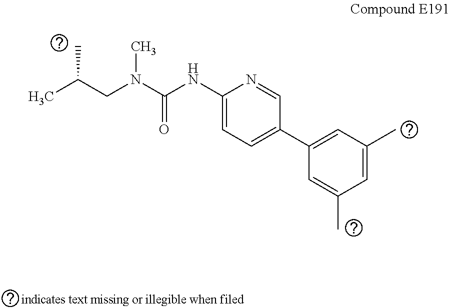

### 3-[5-(3,5-difluorophenyl)-2-pyridyl]-1-[(2S)-2-hydroxypropyl]-1-methyl-urea

A solution of 5-(3,5-difluorophenyl)pyridin-2-amine (Intermediate I03, 50 mg, 0.242 mmol) and pyridine (25 uL, 0.310 mmol) in THF (2 mL) was added to a solution of (4-nitrophenyl) carbonochloridate (54 mg, 0.268 mmol) in THF (1 mL) and the mixture was stirred at RT for 1 hour. (2S)-1-(methylamino)propan-2-ol;hydrochloride (46 mg, 0.366 mmol) and N-ethyl-N-isopropyl-propan-2-amine (128 uL, 0.733 mmol) were added and the mixture was stirred at RT for a further 1 hour. The mixture was concentrated under vacuum and the residue purified by prep HPLC (Method F) followed by FCC (10 g Biotage SNAP cartridge, 0-100% gradient of EtOAc in heptane) to afford the title compound (17 mg, 22% Yield) as a white solid.

TABLE 8
The following compounds were Synthesized using a similar method to that used in Compound E191, using aminopyridines
Synthesized according to the method for Intermediate 103 or from commercial sources.

| Compound | | | LCMS |
|---|---|---|---|
| number | Structure | Analytical data | Method |
| E192 | 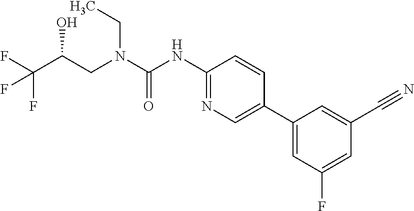 [SMILES: CCN(C[C@@H](O)C(F)(F)F)C(=O)Nc1ccc(-c2cc(F)cc(C#N)c2)cn1] | (500 MHz, Chloroform-d) δ 8.41 (s, 1H), 8.14 (d, J = 8.7 Hz, 1H), 7.85 (dd, J = 8.8, 2.4 Hz, 1H), 7.67 (s, 1H), 7.63 (s, 1H), 7.52-7.46 (m, 1H), 7.38-7.33 (m, 1H), 5.18 (s, 1H), 4.26-4.16 (m, 1H), 3.89-3.78 (m, 1H), 3.61-3.48 (m, 2H), 3.48-3.36 (m, 1H), 1.34 (t, J = 7.2 Hz, 3H). LCMS: m/z 397.2 [M + H]+, (ESI+), RT = 3.18 | A |
| E193 | 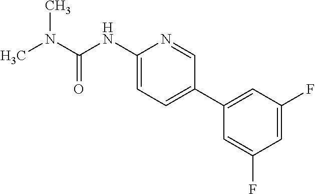 [SMILES: CN(C)C(=O)Nc1ccc(-c2cc(F)cc(F)c2)cn1] | 1H NMR (500 MHz, DMSO-d6) δ 9.04 (s, 1H), 8.65 (dd, J = 2.6, 0.6 Hz, 1H), 8.09 (dd, J = 8.8, 2.6 Hz, 1H), 7.90 (dd, J = 8.8, 0.5 Hz, 1H), 7.55-7.47 (m, 2H), 7.20 (tt, J = 9.3, 2.3 Hz, 1H), 2.96 (s, 6H). LCMS: m/z 278.2 [M + H]+, (ESI+), RT = 2.32 | A |
| E194 | 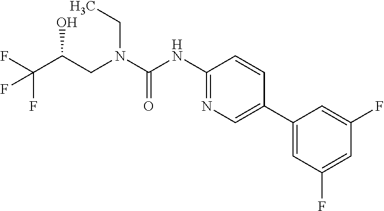 [SMILES: CCN(C[C@@H](O)C(F)(F)F)C(=O)Nc1ccc(-c2cc(F)cc(F)c2)cn1] | (500 MHz, Chloroform-d) δ 8.40 (s, 1H), 8.10 (d, J = 8.6 Hz, 1H), 7.84 (dd, J = 8.7, 2.5 Hz, 1H), 7.65 (s, 1H), 7.10- 7.01 (m, 2H), 6.81 (tt, J = 8.8, 2.3 Hz, 1H), 5.35 (s, 1H), 4.25-4.13 (m, 1H), 3.89-3.77 (m, 1H), 3.57-3.47 (m, 2H), 3.47-3.36 (m, 1H), 1.34 (t, J = 7.2 Hz, 3H).LCMS: m/z 390.2 [M + H]+, (ESI+), RT = 3.36 | A |
| E195 | 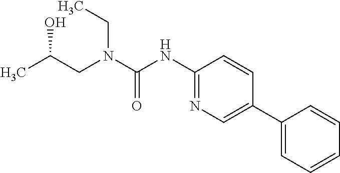 [SMILES: CCN(C[C@H](C)O)C(=O)Nc1ccc(-c2ccccc2)cn1] | 1H NMR (500 MHz, DMSO-d6) δ 9.52 (s, 1H), 8.50 (dd, J = 2.5, 0.7 Hz, 1H), 7.99 (dd, J = 8.7, 2.5 Hz, 1H), 7.87 (dd, J = 8.7, 0.7 Hz, 1H), 7.72-7.59 (m, 2H), 7.51-7.40 (m, 2H), 7.40-7.31 (m, 1H), 5.76 (s, 1H), 4.02-3.81 (m, 1H), 3.36 (dd, J = 7.0, 2.1 Hz, 2H), 3.30- 3.18 (m, 2H), 1.12 (d, J = 6.3 Hz, 3H), 1.10 (t, J = 7.0 Hz, 3H). LCMS: m/z 300.2 [M + H]+, (ESI+), RT = 2.12 | A |
| E196 | 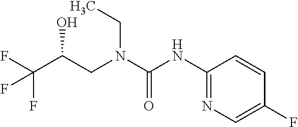 [SMILES: CCN(C[C@@H](O)C(F)(F)F)C(=O)Nc1ccc(F)cn1] | 1H NMR (500 MHz, DMSO-d6) δ 9.03 (s, 1H), 8.22 (d, J = 3.1 Hz, 1H), 7.81 (dd, J = 9.2, 4.1 Hz, 1H), 7.65 (td, J = 8.7, 3.1 Hz, 1H), 7.00 (s, 1H), 4.33- 4.21 (m, 1H), 3.58 (dd, J = 14.9, 2.3 Hz, 1H), 3.47-3.38 (m, 3H), 1.10 (t, J = 7.0 Hz, 3H). LCMS: m/z 296.1 [M + H]+, (ESI+), RT = | B |
| | | 2.62 | |

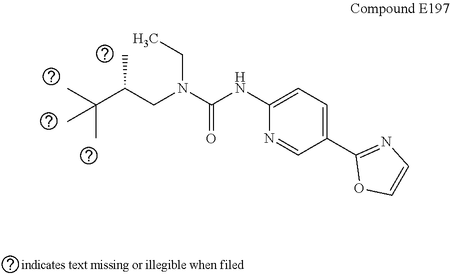 [SMILES: CCN(C[C@@H](C)C(C)(C)C)C(=O)Nc1ccc(-c2ncco2)cn1]

### 1-ethyl-3-(5-oxazol-2-yl-2-pyridyl)-1-[(2R)-3,3,3-trifluoro-2-hydroxy-propyl]urea

Step 1

5-bromo-2-nitropyridine (300 mg, 1.48 mmol) and palladium-triphenylphosphane (1:4) (200 mg, 0.173 mmol) were stirred in anhydrous toluene (4 mL) and degassed with nitrogen for 5 min, before 2-(tributylstannanyl)-1,3-oxazole (0.79 mL, 2.58 mmol) was added and reaction degassed before sealing and stirring at 90° C. for 18h. The reaction was allowed to cool to room temp and treated with KF aqueous solution (˜8M) (5 mL) and MeOH (5 mL) and stirred vigorously for 30 min. The mixture was then filtered through a pad of Celite and washed with EtOAc (20 mL). The filtrated was washed with water (15 mL) and the layers were separated, before the aqueous was extracted again with EtOAc (20 mL). The organics were combined, washed with brine (15 mL), dried over MgSO4, filtrated and concentrated under reduce pressure. Crude was purified using normal phase Biotage (Sfar Duo, 100 g, eluents: EtOAc in Heptane, 0-100%) to afford 2-(6-nitro-3-pyridyl)oxazole (139 mg, 92% purity, 45% Yield) as a pale yellow solid. 1H NMR, (400 MHz, DMSO-d6) δ 9.22 (dd, J=2.2, 0.6 Hz, 1H), 8.73 (dd, J=8.5, 2.2 Hz, 1H), 8.47 (dd, J=7.7, 0.7 Hz, 2H), 7.59 (d, J=0.7 Hz, 1H).

Step 2

A stirred solution of 2-(6-nitro-3-pyridyl)oxazole (92%, 139 mg, 0.667 mmol) in EtOAc (10 mL) and 1,4-Dioxane (3 mL) was evacuated and charged with nitrogen three times, 10% palladium on carbon (14 mg, 0.133 mmol) was added and the reaction mixture was put under a balloon of hydrogen and stirred at RT. Upon completion, the reaction mixture was filtered through a Celite pad and the filter cake washed with ethyl acetate (25 mL). The filtrate was concentrated to dryness under reduced pressure to afford 5-oxazol-2-ylpyridin-2-amine (Intermediate I04, 110 mg, 95% purity, 97% Yield) as a pale yellow solid. 1H NMR, (400 MHz, DMSO-d6) δ 8.51 (dd, J=2.4, 0.6 Hz, 1H), 8.07 (d, J=0.8 Hz, 1H), 7.88 (dd, J=8.7, 2.4 Hz, 1H), 7.25 (d, J=0.8 Hz, 1H), 6.55-6.53 (m, 2H), 6.52 (d, J=0.7 Hz, 1H).

Step 3

To a solution of 4-nitrophenyl carbonochloridate (72 mg, 0.357 mmol) in anhydrous THF (1.5 mL) was added a solution of 5-oxazol-2-ylpyridin-2-amine (95%, 55 mg, 0.324 mmol) and pyridine (29 uL, 0.357 mmol) in anhydrous THF (1 mL) and the reaction was stirred at RT for 3 hours. Next, (2R)-3-(ethylamino)-1,1,1-trifluoro-propan-2-ol (Intermediate I02, 51 mg, 0.324 mmol) and N-ethyl-N-isopropyl-propan-2-amine (85 uL, 0.486 mmol) in anhydrous THF (1 mL) were added and the reaction was stirred at RT for 45 min. It was then concentrated under reduced pressure and purified using prep HPLC (Method H) to afford the title compound (32 mg, 28% Yield) as a white solid. 1H NMR, (500 MHz, Chloroform-d) δ 8.89 (d, J=1.8 Hz, 1H), 8.29 (dd, J=8.9, 2.3 Hz, 1H), 8.15 (d, J=8.8 Hz, 1H), 7.73 (d, J=0.7 Hz, 1H), 7.24 (d, J=0.7 Hz, 1H), 4.27-4.18 (m, 1H), 3.77 (dd, J=15.2, 8.8 Hz, 1H), 3.63-3.53 (m, 2H), 3.48 (dq, J=14.6, 7.0 Hz, 1H), 1.32 (t, J=7.2 Hz, 3H). OH and NH not observed. LCMS: m/z 345.1 [M+H]+, (ESI+), RT=2.47 (Method A)

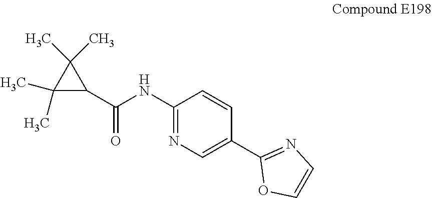 [SMILES: CC1(C)C(C(=O)Nc2ccc(-c3ncco3)cn2)C1(C)C]

### 2,2,3,3-tetramethyl-N-(5-oxazol-2-yl-2-pyridyl)cyclopropanecarboxamide

Synthesized from Intermediate I04 using a similar method to that used in Compound E093, Step 2. 1H NMR (400 MHz, Chloroform-d) δ 8.92 (dd, J=2.0, 1.0 Hz, 1H), 8.34-8.23 (m, 2H), 8.06 (s, 1H), 7.72 (d, J=0.6 Hz, 1H), 7.25-7.23 (m, 1H), 1.33 (s, 6H), 1.24 (s, 6H), 1.04 (s, 1H). LCMS: m/z 286.2 [M+H]+, (ESI+), RT=3.64 (Method B)

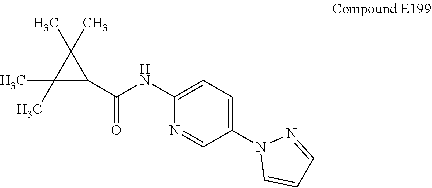 [SMILES: CC1(C)C(C(=O)Nc2ccc(-n3cccn3)cn2)C1(C)C]

### 2,2,3,3-tetramethyl-N-(5-pyrazol-1-yl-2-pyridyl)cyclopropanecarboxamide

Step 1

5-iodopyridin-2-amine (250 mg, 1.14 mmol), copper(I) iodide (22 mg, 0.114 mmol), tripotassium phosphate (734 mg, 3.41 mmol) and 1H-pyrazole (85 mg, 1.25 mmol) were added to a sealed tube. After purging it with nitrogen, anhydrous 2-propanol (5 mL) was added to it followed by ethane-1,2-diol (6.3 uL, 0.114 mmol). The reaction mixture was again purged with nitrogen and then the tube was sealed and heated to 110° C. for 15 hours. The reaction mixture was then cooled to RT, filtered through a pad of celite, washing with MeOH (20 mL). The filtrate was concentrated under reduced pressure and purified by flash column chromatography (25 g SiO2 column, 0-15% MeOH in DCM) to afford the title compound as a beige solid (145 mg, 50% purity).

Step 2

To a solution of 5-pyrazol-1-ylpyridin-2-amine (140 mg, 0.874 mmol) and N-ethyl-N-isopropyl-propan-2-amine (305 uL, 1.75 mmol) in anhydrous THF (2 mL) was added 2,2,3,3-tetramethylcyclopropanecarbonyl chloride (211 mg, 1.31 mmol) with stirring at RT for 30 mins. Solvent was then removed under a steady stream of nitrogen and MeOH (2 mL) and 1M NaOH (2 mL) were added and the reaction mixture was stirred at RT for 1 h. MeOH was then removed under a steady stream of nitrogen resulting in formation of a beige precipitate. This was filtered, washed with water and the solid purified by prep HPLC (Method F) to afford the title compound as a white solid (62 mg). 1H NMR (400 MHz, DMSO-d6) δ 10.54 (s, 1H), 8.81-8.73 (m, 1H), 8.48 (dd, J=2.5, 0.5 Hz, 1H), 8.22-8.07 (m, 2H), 7.76 (dd, J=1.8, 0.5 Hz, 1H), 6.56 (dd, J=2.5, 1.8 Hz, 1H), 1.55 (s, 1H), 1.25 (s, 6H), 1.17 (s, 6H). LCMS: m/z 285.2 [M+H]+, (ESI+), RT=3.58 (Method B).

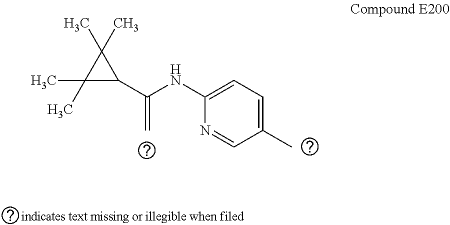

### N-(5-iodo-2-pyridyl)-2,2,3,3-tetramethyl-cyclopropanecarboxamide

Title compound was isolated as a byproduct from the synthesis of Compound E199. 1H NMR (500 MHz, DMSO-d6) δ 10.49 (s, 1H), 8.47 (dd, J=2.3, 0.6 Hz, 1H), 8.03 (dd, J=8.8, 2.3 Hz, 1H), 7.98-7.87 (m, 1H), 1.53 (s, 1H), 1.22 (s, 6H), 1.15 (s, 6H). LCMS: m/z 345.2 [M+H]+, (ESI+), RT=4.27 (Method B).

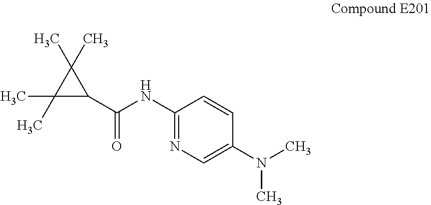

### N-[5-(dimethylamino)-2-pyridyl]-2,2,3,3-tetramethyl-cyclopropanecarboxamide

To a solution of N5,N5-dimethylpyridine-2,5-diamine (100 mg, 0.729 mmol) and N-ethyl-N-isopropyl-propan-2-amine (255 uL, 1.46 mmol) in THF-Anhydrous (5 mL) was added 2,2,3,3-tetramethylcyclopropanecarbonyl chloride (117 mg, 0.729 mmol) and the reaction mixture was stirred at RT for 1 h. Further 2,2,3,3-tetramethylcyclopropanecarbonyl chloride (39 mg, 0.243 mmol) was added and the mixture was stirred for another 0.5 h. Solvent was then removed under a steady stream of nitrogen and MeOH (4 mL) and 1M NaOH (4 mL) were added and the reaction mixture was stirred at 45° C. for 4 h and at RT for 16 h. Methanol was removed under reduced pressure and the precipitate was filtered, washed with water (10 mL) and purified by flash column chromatography (10 g SiO2 column, 0-60% EtOAc in heptane) to afford the title compound as an off-white solid (125 mg). 1H NMR (500 MHz, DMSO-d6) δ 9.99 (s, 1H), 7.85 (d, J=9.0 Hz, 1H), 7.79 (d, J=3.0 Hz, 1H), 7.14 (dd, J=9.1, 3.2 Hz, 1H), 2.86 (s, 6H), 1.46 (s, 1H), 1.22 (s, 6H), 1.15 (s, 6H). LCMS: m/z 262.3 [M+H]+, (ESI+), RT=2.10 (Method A)

TABLE 9
The following compounds were Synthesized using a similar method to that used in Compound E201

| Compound | | | LCMS |
|---|---|---|---|
| number | Structure | Analytical data | Method |
| E202 | 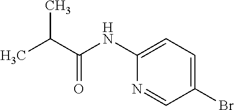 | 1H NMR (500 MHz, DMSO-d6) δ 10.57 (s, 1H), 8.42 (d, J = 2.5 Hz, 1H), 8.08 (d, J = 8.9 Hz, 1H), 7.98 (dd, J = 8.9, 2.5 Hz, 1H), 2.74 (hept, J = 6.8 Hz, 1H), 1.08 (d, J = 6.8 Hz, 6H). LCMS: m/z 243/245 [M + H]+, (ESI+), RT = 2.70 | A |
| E203 | 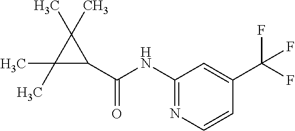 [SMILES: CC1(C)C(C(=O)Nc2cc(C(F)(F)F)ccn2)C1(C)C] | 1H NMR (500 MHz, Chloroform-d) δ 8.48 (s, 1H), 8.39 (d, J = 5.2 Hz, 1H), 8.11 (s, 1H), 7.22-7.17 (m, 1H), 1.33 (s, 6H), 1.23 (s, 6H), 1.03 (s, 1H). LCMS: m/z 287.1 [M + H]+, (ESI+), RT = 4.14 | A |
| E204 | 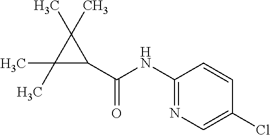 [SMILES: CC1(C)C(C(=O)Nc2ccc(Cl)cn2)C1(C)C] | 1H NMR (400 MHz, Chloroform-d) δ 8.19 (d, J = 2.5 Hz, 1H), 8.16 (d, J = 9.0 Hz, 2H), 8.00 (s, 1H), 7.61 (dd, J = 8.9, 2.6 Hz, 1H), 1.31 (s, 6H), 1.22 (s, 6H), 1.01 (s, 1H). LCMS: m/z 253.2, 255.2 [M + H]+, (ESI+), RT = 3.93 | A |
| E205 | 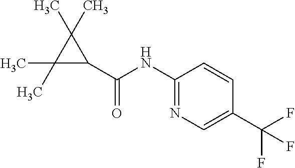 [SMILES: CC1(C)C(C(=O)Nc2ccc(C(F)(F)F)cn2)C1(C)C] | 1H NMR (500 MHz, Chloroform-d) δ 8.52-8.49 (m, 1H), 8.31 (d, J = 8.8 Hz, 1H), 8.09 (s, 1H), 7.87 (dd, J = 8.8, 2.4 Hz, 1H), 1.32 (s, 6H), 1.24 (s, 6H), 1.05 (s, 1H). LCMS: m/z 287.1 [M + H]+, (ESI+), RT = 4.13 | A |
| E206 | 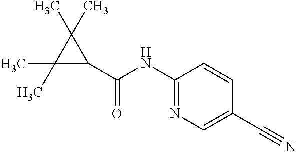 [SMILES: CC1(C)C(C(=O)Nc2ccc(C#N)cn2)C1(C)C] | 1H NMR (500 MHz, Chloroform-d) δ 8.56-8.53 (m, 1H), 8.34 (dd, J = 8.8, 0.7 Hz, 1H), 8.12 (s, 1H), 7.91 (dd, J = 8.8, 2.2 Hz, 1H), 1.34 (s, 6H), 1.26 (s, 6H), 1.07 (s, 1H). LCMS: m/z 244.2 [M + H]+, (ESI+), RT = 3.54 | A |
| E207 | 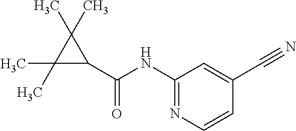 [SMILES: CC1(C)C(C(=O)Nc2cc(C#N)ccn2)C1(C)C] | 1H NMR (500 MHz, Chloroform-d) δ 8.55-8.51 (m, 1H), 8.40 (dd, J = 5.1, 0.8 Hz, 1H), 8.07 (s, 1H), 7.21 (dd, J = 5.1, 1.4 Hz, 1H), 1.35 (s, 6H), 1.26 (s, 6H), 1.05 (s, 1H). LCMS: m/z 244.2 [M + H]+, (ESI+), RT = 3.58 | A |
| E208 | 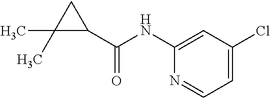 | 1H NMR (400 MHz, Chloroform-d) δ 8.30 (d, J = 1.8 Hz, 1H), 8.14 (d, J = 5.4 Hz, 1H), 8.07 (s, 1H), 7.01 (dd, J = 5.4, 1.9 Hz, 1H), 1.43 (dd, J = 7.9, 5.3 Hz, 1H), 1.27-1.24 (m, 1H), 1.23 (s, 3H), 1.21 (s, 3H), 0.90 (dd, J = 7.9, 4.4 Hz, 1H). | A |
| | | LCMS: m/z 22 [M + H]+, (ESI+), RT = | |
| | | 3.17 | |
| E209 | 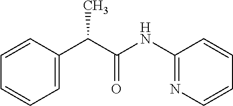 [SMILES: C[C@H](C(=O)Nc1ccccn1)c1ccccc1] | 1H NMR (400 MHz, DMSO-d6) δ 10.58 (s, 1H), 8.31-8.25 (m, 1H), 8.06 (d, J = 8.4 Hz, 1H), 7.74 (td, J = 8.6, 8.1, 1.9 Hz, 1H), 7.45-7.37 (m, 2H), 7.36-7.27 (m, 2H), 7.27-7.18 (m, 1H), 7.07 (ddd, J = 7.3, 4.9, 1.0 Hz, 1H), 4.02 (q, J = 7.0 Hz, 1H), 1.40 (d, J = | A |
| | | 7.0 Hz, 3H). | |
| | | LCMS: m/z 227.2 [M + H]+, (ESI+), RT = | |
| | | 2.24 | |
| E210 | 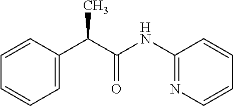 [SMILES: C[C@@H](C(=O)Nc1ccccn1)c1ccccc1] | 1H NMR (400 MHz, DMSO-d6) δ 10.58 (s, 1H), 8.31-8.25 (m, 1H), 8.06 (d, J = 8.4 Hz, 1H), 7.79-7.69 (m, 1H), 7.44-7.37 (m, 2H), 7.36-7.26 (m, 2H), 7.27-7.18 (m, 1H), 7.07 (ddd, J = 7.3, 4.9, 1.0 Hz, 1H), 4.02 (q, J = 7.0 Hz, 1H), 1.40 (d, J = 7.0 Hz, 3H). | A |
| | | LCMS: m/z 227.2 [M + H]+, (ESI+), RT = | |
| | | 2.23 | |
| E211 | 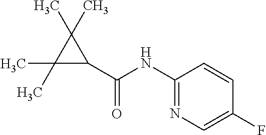 [SMILES: CC1(C)C(C(=O)Nc2ccc(F)cn2)C1(C)C] | 1H NMR (400 MHz, Chloroform-d) δ 8.19 (dd, J = 9.2, 4.1 Hz, 1H), 8.09 (d, J = 3.0 Hz, 1H), 7.94 (s, 1H), 7.39 (ddd, J = 9.2, 7.8, 2.9 Hz, 1H), 1.32 (s, 6H), 1.22 (s, 6H), 1.01 (s, 1H). LCMS: m/z 237.2 [M + H]+, (ESI+), RT = 3.52 | A |
| E212 | 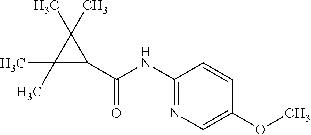 | 1H NMR (500 MHz, Chloroform-d) δ 8.10 (d, J = 9.0 Hz, 1H), 7.98-7.90 (m, 2H), 7.22 (dd, J = 9.1, 3.0 Hz, 1H), 3.83 (s, 3H), 1.31 (s, 6H), 1.20 (s, 6H), 1.00 (s, 1H). LCMS: m/z 249.2 [M + H]+, (ESI+), RT = 2.86 | A |
| E213 | 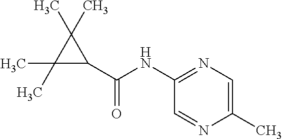 | 1H NMR (400 MHz, Chloroform-d) δ 9.36 (d, J = 1.3 Hz, 1H), 8.07 (s, 1H), 7.72 (s, 1H), 2.51 (s, 3H), 1.33 (s, 6H), 1.23 (s, 7H), 1.04 (s, 1H). LCMS: m/z 234 [M + H]+, (ESI+), RT = 3.01 | A |
| E214 | 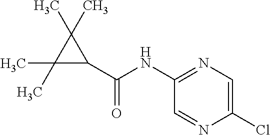 [SMILES: CC1(C)C(C(=O)Nc2cnc(Cl)cn2)C1(C)C] | 1H NMR (400 MHz, Chloroform-d) δ 9.29 (d, J = 1.4 Hz, 1H), 8.21 (d, J = 1.4 Hz, 1H), 7.82 (s, 1H), 1.33 (s, 6H), 1.24 (s, 6H), 1.05 (s, 1H). LCMS: m/z 254 [M + H]+, (ESI+), RT = 3.69 | A |
| E215 | 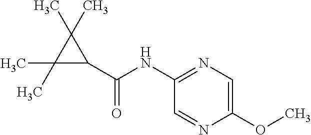 | 1H NMR (400 MHz, Chloroform-d) δ 9.03-8.90 (m, 1H), 7.87 (d, J = 1.4 Hz, 1H), 7.64 (s, 1H), 3.94 (s, 3H), 1.32 (s, 6H), 1.22 (s, 6H), 1.03 (s, 1H). LCMS: m/z 250.0 [M + H]+, (ESI+), RT = 3.42 | A |
| E216 | 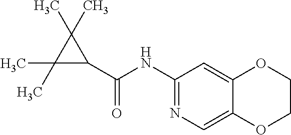 [SMILES: CC1(C)C(C(=O)Nc2cc3c(cn2)OCCO3)C1(C)C] | 1H NMR (500 MHz, Chloroform-d) δ 7.84 (s, 1H), 7.82 (s, 1H), 7.76 (s, 1H), 4.35-4.30 (m, 2H), 4.28-4.23 (m, 2H), 1.32 (s, 6H), 1.22 (s, 6H), 1.00 (s, 1H). LCMS: m/z 277.1 [M + H]+, (ESI+), RT = 2.22 | A |

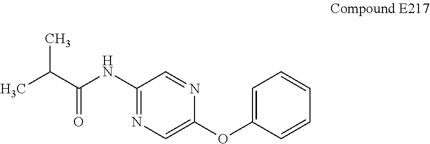 [SMILES: CC(C)C(=O)Nc1cnc(Oc2ccccc2)cn1]

### 2-methyl-N-(5-phenoxypyrazin-2-yl)propanamide

Step 1

5-bromopyrazin-2-amine (300 mg, 1.72 mmol), phenol (178 mg, 1.90 mmol) and cesium carbonate (1.12 g, 3.45 mmol) were suspended in anhydrous 1,4-Dioxane (4 mL) and the reaction mixture was degassed with N2 for 5 minutes. N,N-dimethylglycine hydrochloride (1:1) (24 mg, 0.172 mmol) was added followed by copper (I) iodide (33 mg, 0.172 mmol) and the reaction was heated to 115° C. in a sealed tube for 2.5 h. The reaction mixture was then cooled to RT, diluted with EtOAc and water and filtered. The organic phase was then separated and the aqueous layer was extracted twice with EtOAc. The combined organic extracts were dried over MgSO4, filtered, concentrated under reduced pressure and purified by flash column chromatography (25 g SiO2 column, 0-70% EtOAc in heptane) to afford 5-phenoxypyrazin-2-amine (Intermediate I05) as a pale yellow oil that solidified upon standing (205 mg). 1H NMR (500 MHz, DMSO-d6) δ 7.82 (d, J=1.4 Hz, 1H), 7.56 (d, J=1.4 Hz, 1H), 7.41-7.28 (m, 2H), 7.14-7.05 (m, 1H), 7.00-6.93 (m, 2H), 6.22 (s, 2H).

Step 2

5-phenoxypyrazin-2-amine (50 mg, 0.267 mmol) was dissolved in DCM (2 mL) and N-ethyl-N-isopropyl-propan-2-amine (70 uL, 0.401 mmol) was added followed by 2-methylpropanoyl chloride (31 uL, 0.294 mmol). The reaction was stirred at RT for 15 minutes. Further N-ethyl-N-isopropyl-propan-2-amine (70 uL, 0.401 mmol) and 2-methylpropanoyl chloride (31 uL, 0.294 mmol) were added and the reaction was stirred at RT for 15 minutes. The reaction mixture was washed with sat. aq. NaHCO3, passed through a TELOS phase separator and concentrated under reduced pressure. The residue was dissolved in MeOH (1 mL) and 1M aq. NaOH (1 mL) and stirred for 5 minutes at RT. The solvents were removed under reduced pressure and the crude mixture was purified by prep HPLC (Method E) to afford the title compound as a white solid (57 mg). 1H NMR (250 MHz, DMSO-d6) δ 10.66 (s, 1H), 8.89 (d, J=1.4 Hz, 1H), 8.28 (d, J=1.4 Hz, 1H), 7.50-7.34 (m, 2H), 7.29-7.08 (m, 3H), 2.74 (h, J=6.9 Hz, 1H), 1.10 (d, J=6.8 Hz, 6H). LCMS: m/z 258.3 [M+H]+, (ESI+), RT=2.99 (Method A).

TABLE 10
The following compounds were Synthesized using a similar method to that used in Compound E217

| Compound | | | LCMS |
|---|---|---|---|
| number | Structure | Analytical data | Method |
| E218 | 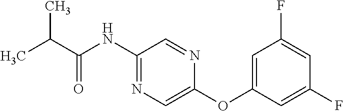 [SMILES: CC(C)C(=O)Nc1cnc(Oc2cc(F)cc(F)c2)cn1] | 1HNMR(400 MHz, DMSO-d6) δ 10.75 (s, 1H), 8.96 (d, J = 1.4 Hz, 1H), 8.40- 8.32 (m, 1H), 7.10 (tt, J = 9.4, 2.3 Hz, 1H), 7.06-6.96 (m, 2H), 2.78 (hept, J = 6.8 Hz, 1H), 1.11 (d, J = 6.8 Hz, 6H). LCMS: m/z 294.1 [M + H]+, (ESI+), RT = 3.30 | A |
| E219 | 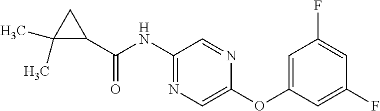 [SMILES: CC1(C)CC1C(=O)Nc1cnc(Oc2cc(F)cc(F)c2)cn1] | 1H NMR (500 MHz, DMSO-d6) δ 10.94 (s, 1H), 8.93 (d, J = 1.2 Hz, 1H), 8.33 (d, J = 1.4 Hz, 1H), 7.09 (tt, J = 9.4, 2.3 Hz, 1H), 7.05-6.95 (m, 2H), 1.88 (dd, J = 7.9, 5.5 Hz, 1H), 1.15 (d, J = 8.3 Hz, 6H), 1.01 (dd, J = 5.3, 4.1 Hz, 1H), 0.85 (dd, J = 7.9, 3.9 Hz, 1H). LCMS: m/z 320.2 [M + H]+, (ESI+), RT = | A |
| | | 3.79 | |
| E220 | 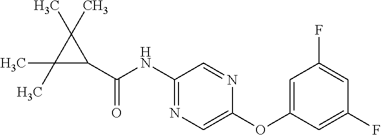 [SMILES: CC1(C)C(C(=O)Nc2cnc(Oc3cc(F)cc(F)c3)cn2)C1(C)C] | 1H NMR (500 MHz, DMSO-d6) δ 10.73 (s, 1H), 8.91 (s, 1H), 8.33 (s, 1H), 7.43-6.51 (m, 3H), 1.57 (s, 1H), 1.25 (s, 6H), 1.19 (s, 6H). LCMS: m/z 348.3 [M + H]+, (ESI+), RT = 4.34 | A |
| E221 | 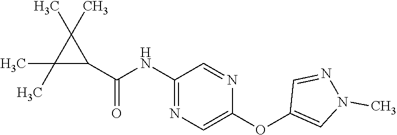 | (500 MHz, Chloroform-d) δ 9.02 (d, J = 1.2 Hz, 1H), 8.04 (d, J = 1.4 Hz, 1H), 7.75 (s, 1H), 7.60 (s, 1H), 7.47 (s, 1H), 3.89 (s, 3H), 1.32 (s, 6H), 1.23 (s, 6H), 1.04 (s, 1H). LCMS: m/z 316.2 [M + H]+, (ESI+), RT = 3.25 | A |
| E222 | 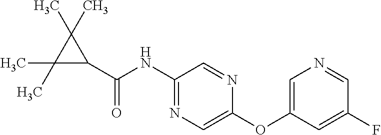 [SMILES: CC1(C)C(C(=O)Nc2cnc(Oc3cncc(F)c3)cn2)C1(C)C] | 1HNMR(400 MHz, Chloroform-d) δ 9.07 (d, J = 1.4 Hz, 1H), 8.37 (dd, J = 4.3, 2.3 Hz, 2H), 8.15 (d, J = 1.4 Hz, 1H), 7.86 (s, 1H), 7.35 (dt, J = 9.4, 2.4 Hz, 1H), 1.35 (s, 6H), 1.26 (s, 6H), 1.08 (s, 1H). LCMS: m/z 331.2 [M + H]+, (ESI+), RT = 3.63 | A |
| E223 | 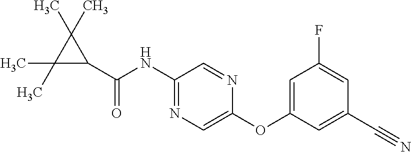 [SMILES: CC1(C)C(C(=O)Nc2cnc(Oc3cc(F)cc(C#N)c3)cn2)C1(C)C] | HNMR(400 MHz, Chloroform-d) δ 9.10 (d, J = 1.4 Hz, 1H), 8.14 (d, J = 1.4 Hz, 1H), 7.86 (s, 1H), 7.28-7.25 (m, 1H), 7.23-7.15 (m, 2H), 1.35 (s, 6H), 1.27 (s, 6H), 1.09 (s, 1H). LCMS: m/z 355.2 [M + H]+, (ESI+), RT = 4.10 | A |
| E224 | 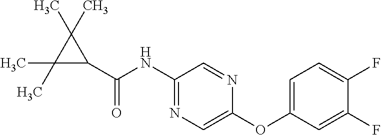 [SMILES: CC1(C)C(C(=O)Nc2cnc(Oc3ccc(F)c(F)c3)cn2)C1(C)C] | 1HNMR(500 MHz, Chloroform-d) δ 9.04 (d, J = 1.3 Hz, 1H), 8.07 (d, J = 1.4 Hz, 1H), 7.80 (s, 1H), 7.23-7.14 (m, 1H), 7.02 (ddd, J = 10.9, 6.7, 2.9 Hz, 1H), 6.89 (dddd, J = 9.0, 3.5, 2.9, 1.9 Hz, 1H), 1.34 (s, 6H), 1.26 (s, 6H), 1.07 (s, 1H). LCMS: m/z 348.3 [M + H]+, (ESI+), RT = 4.27 | A |
| E225 | 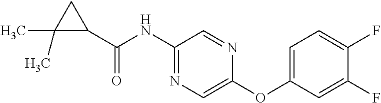 [SMILES: CC1(C)CC1C(=O)Nc1cnc(Oc2ccc(F)c(F)c2)cn1] | δ 9.07-9.03 (m, 1H), 8.07 (d, J = 1.4 Hz, 1H), 7.90 (s, 1H), 7.22-7.12 (m, 1H), 7.05-6.97 (m, 1H), 6.91-6.84 (m, 1H), 1.46 (dd, J = 7.9, 5.3 Hz, 1H), 1.26 (t, J = 4.9 Hz, 1H), 1.23 (s, 3H), 1.22 (s, 3H), 0.92 (dd, J = 7.9, 4.5 Hz, 1H). | A |
| | | LCMS: m/z 320.3 [M + H]+, (ESI+), RT = | |
| | | 3.70 | |

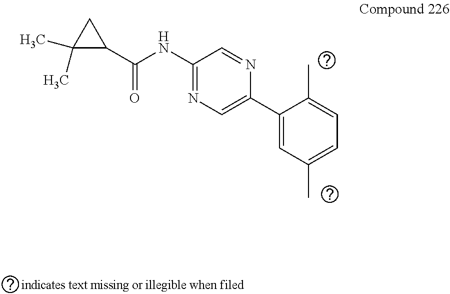 [SMILES: Cc1ccc(C)c(-c2cnc(NC(=O)C3CC3(C)C)cn2)c1]

### N-[5-(2,5-difluorophenyl)pyrazin-2-yl]-2,2-dimethyl-cyclopropanecarboxamide

Step 1

5-bromopyrazin-2-amine (300 mg, 1.72 mmol) and (2,5-difluorophenyl)boronic acid (275 mg, 1.74 mmol) were dissolved in 1,4-Dioxane-Anhydrous (10 mL) and 2 M Na2CO3 (1.8 mL, 3.60 mmol) and the reaction mixture was degassed with N2 for 5 minutes. To the reaction was added Pd(dppf)Cl2 (63 mg, 0.0862 mmol) and the reaction was heated to 110° C. for 2.5 h. It was then cooled to RT, diluted with water (20 mL) and EtOAc (20 mL) and filtered through celite. The layers in the filtrate were separated and the aqueous was extracted with EtOAc (2×20 mL). The combined organic extracts were dried over MgSO4, filtered, concentrated under reduced pressure and purified by flash column chromatography (25 g SiO2 column, 0-100% EtOAc in heptane) to afford 5-(2,5-difluorophenyl)pyrazin-2-amine (Intermediate I06) as a yellow solid (295 mg). 1H NMR (400 MHz, DMSO-d6) δ 8.37 (dd, J=2.3, 1.6 Hz, 1H), 8.01 (d, J=1.5 Hz, 1H), 7.62 (ddd, J=9.6, 6.1, 3.3 Hz, 1H), 7.33 (ddd, J=10.8, 9.1, 4.6 Hz, 1H), 7.26-7.08 (m, 1H), 6.78 (s, 2H).

Step 2

To a stirred solution of 2,2-dimethylcyclopropanecarboxylic acid (43 mg, 0.380 mmol) in Ethyl acetate (2.5 mL) was added N-ethyl-N-isopropyl-propan-2-amine (0.19 mL, 1.09 mmol) and T3P (50% in EtOAc) (0.32 mL, 0.543 mmol) and the reaction was stirred for 10 min. To this was added 5-(2,5-difluorophenyl)pyrazin-2-amine (75 mg, 0.362 mmol) and the reaction was stirred at 80° C. for 20 h. It was then cooled to RT, washed with sat. aq. NaHCO3, dried over MgSO4, filtered, concentrated under reduced pressure and purified by flash column chromatography (10 g SiO2 column, 0-50% EtOAc in heptane) to afford the title compound as an off-white solid (39 mg). 1H NMR (500 MHz, DMSO-d6) δ 11.13 (s, 1H), 9.45 (d, J=1.5 Hz, 1H), 8.92-8.67 (m, 1H), 7.73 (ddd, J=9.3, 6.0, 3.3 Hz, 1H), 7.44 (ddd, J=10.5, 9.2, 4.5 Hz, 1H), 7.40-7.30 (m, 1H), 1.94 (dd, J=7.8, 5.5 Hz, 1H), 1.17 (s, 3H), 1.16 (s, 3H), 1.05 (dd, J=5.3, 4.0 Hz, 1H), 0.89 (dd, J=7.8, 3.9 Hz, 1H). LCMS: m/z 304.2 [M+H]+, (ESI+), RT=3.81 (Method A)

TABLE 11
The following compounds were Synthesized using a similar method to that used in Compound E226

| Compound | | | LCMS |
|---|---|---|---|
| number | Structure | Analytical data | Method |
| E227 | 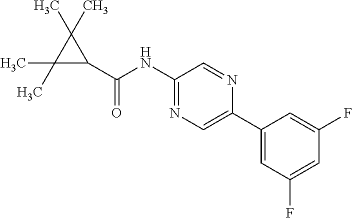 [SMILES: CC1(C)C(C(=O)Nc2cnc(-c3cc(F)cc(F)c3)cn2)C1(C)C] | 1H NMR (400 MHz, Chloroform-d) δ 9.54 (d, J = 1.5 Hz, 1H), 8.60 (d, J = 1.5 Hz, 1H), 7.89 (s, 1H), 7.56-7.47 (m, 2H), 6.85 (tt, J = 8.7, 2.3 Hz, 1H), 1.35 (s, 6H), 1.26 (s, 6H), 1.09 (s, 1H). LCMS: m/z 332.2 [M + H]+, (ESI+), RT = 4.61 | A |
| E228 | 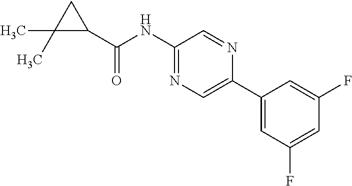 [SMILES: CC1(C)CC1C(=O)Nc1cnc(-c2cc(F)cc(F)c2)cn1] | 1H NMR (500 MHz, Chloroform-d) δ 9.60 (d, J = 1.4 Hz, 1H), 8.64 (d, J = 1.5 Hz, 1H), 8.03 (s, 1H), 7.59-7.51 (m, 2H), 6.89 (tt, J = 8.7, 2.3 Hz, 1H), 1.53 (dd, J = 7.9, 5.3 Hz, 1H), 1.33 (t, J = 4.9 Hz, 1H), 1.28 (s, 3H), 1.26 (s, 3H), 0.99 (dd, J = 7.8, 4.5 Hz, 1H). LCMS: m/z 304.2 [M + H]+, (ESI+), RT = 4.05 | A |
| E229 | 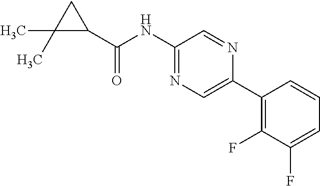 [SMILES: CC1(C)CC1C(=O)Nc1cnc(-c2cccc(F)c2F)cn1] | 1H NMR (500 MHz, DMSO-d6) δ 11.13 (s, 1H), 9.46 (d, J = 1.5 Hz, 1H), 8.81-8.74 (m, 1H), 7.77-7.71 (m, 1H), 7.58-7.50 (m, 1H), 7.41-7.32 (m, 1H), 1.94 (dd, J = 7.8, 5.5 Hz, 1H), 1.18 (s, 3H), 1.17 (s, 3H), 1.08-1.03 (m, 1H), 0.92-0.86 (m, 1H). LCMS: m/z 304.1 [M + H]+, (ESI+), RT = 3.86 | B |
| E230 | 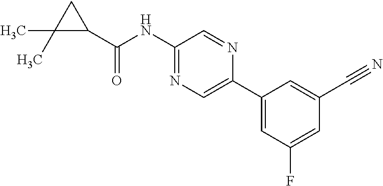 [SMILES: CC1(C)CC1C(=O)Nc1cnc(-c2cc(F)cc(C#N)c2)cn1] | (400 MHz, Chloroform-d) δ 9.61 (d, J = 1.3 Hz, 1H), 8.66 (d, J = 1.5 Hz, 1H), 8.15-8.03 (m, 2H), 8.02-7.94 (m, 1H), 7.40 (ddd, J = 7.6, 2.4, 1.3 Hz, 1H), 1.52 (dd, J = 7.8, 5.3 Hz, 1H), 1.32 (t, J = 4.9 Hz, 1H), 1.27 (s, 3H), 1.25 (s, 3H), 0.98 (dd, J = 7.8, 4.5 Hz, 1H). LCMS: m/z 311.2 [M + H]+, (ESI+), RT = 3.71 | A |

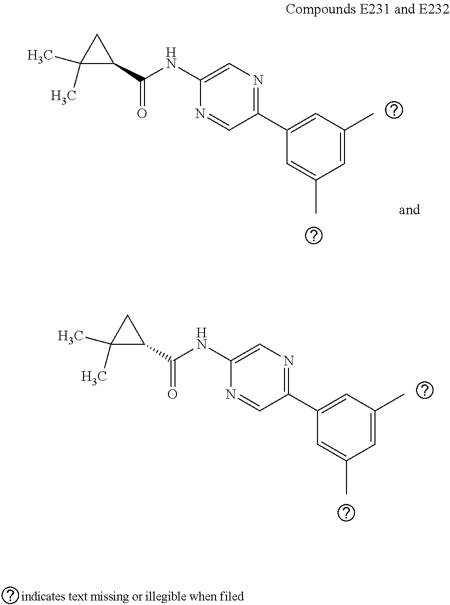

### Unknown Single Enantiomers of N-[5-(3,5-difluorophenyl)pyrazin-2-yl]-2,2-dimethyl-cyclopropanecarboxamide

Chiral separation of N-[5-(3,5-difluorophenyl)pyrazin-2-yl]-2,2-dimethyl-cyclopropanecarboxamide (Compound E228) to afford unknown single enantiomers. Method: Cellulose-4 column, 21.2×250 mm, 5 μm, 90:10 Heptane: Ethanol, 18 mL/min flow rate.

Compound E231 (first eluting):

1H NMR (500 MHz, DMSO-d6) δ 11.12 (s, 1H), 9.40 (d, J=1.4 Hz, 1H), 9.08 (d, J=1.4 Hz, 1H), 7.90-7.76 (m, 2H), 7.32 (tt, J=9.1, 2.2 Hz, 1H), 1.94 (dd, J=7.8, 5.5 Hz, 1H), 1.17 (s, 3H), 1.17 (s, 3H), 1.05 (dd, J=5.3, 4.1 Hz, 1H), 0.89 (dd, J=7.8, 3.9 Hz, 1H). LCMS: m/z 304.2 [M+H]+, (ESI+), RT=3.93 (Method A).

Compound E232 (second eluting):

1H NMR (500 MHz, DMSO-d6) δ 11.12 (s, 1H), 9.40 (d, J=1.5 Hz, 1H), 9.08 (d, J=1.5 Hz, 1H), 7.90-7.76 (m, 2H), 7.32 (tt, J=9.2, 2.3 Hz, 1H), 1.94 (dd, J=7.8, 5.5 Hz, 1H), 1.17 (s, 3H), 1.17 (s, 3H), 1.08-1.03 (m, 1H), 0.89 (dd, J=7.8, 3.9 Hz, 1H). LCMS: m/z 304.2 [M+H]+, (ESI+), RT=3.93 (Method A).

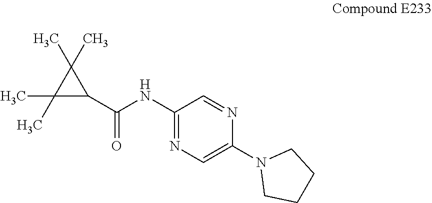 [SMILES: CC1(C)C(C(=O)Nc2cnc(N3CCCC3)cn2)C1(C)C]

### 2,2,3,3-tetramethyl-N-(5-pyrrolidin-1-ylpyrazin-2-yl)cyclopropanecarboxamide

Step 1

5-bromopyrazin-2-amine (200 mg, 1.15 mmol) was suspended in pyrrolidine (0.30 mL, 3.59 mmol) and the mixture was stirred at 180° C. for a total of 4 hours in Biotage initiator microwave. The reaction was concentrated under vacuum and purified by FCC (Biotage SNAP KP-Sil 10 g, 50-100% EtOAc in heptane) to afford 5-pyrrolidin-l-ylpyrazin-2-amine (100 mg, 50% Yield). 1H NMR (400 MHz, Chloroform-d) δ 7.66 (d, J=1.6 Hz, 1H), 7.43 (d, J=1.6 Hz, 1H), 3.86 (s, 2H), 3.45-3.35 (m, 4H), 2.07-1.94 (m, 4H).

Step 2

2,2,3,3-tetramethylcyclopropanecarbonyl chloride (54 mg, 0.335 mmol) in THF (1 mL) was added dropwise to a stirred solution of 5-pyrrolidin-l-ylpyrazin-2-amine (50 mg, 0.304 mmol) and N-ethyl-N-isopropyl-propan-2-amine (120 μL, 0.670 mmol) in THF (1 mL). The reaction was stirred for approximately 20 hours at RT. Further N-ethyl-N-isopropyl-propan-2-amine (58 uL, 0.335 mmol) and 2,2,3,3-tetramethylcyclopropanecarbonyl chloride (20 mg, 0.125 mmol) in THF (0.5 mL) were added and reaction stirred for 1 hour. The reaction was diluted with water (5 mL) and extracted into EtOAc (3×5 mL), organics dried over MgSO4, filtered and concentrated under vacuum. The crude product was purified by prep HPLC (Method G) to afford the title compound (22 mg, 25% Yield) as a tan solid. 1H NMR (500 MHz, Chloroform-d) δ 8.91 (s, 1H), 7.53 (s, 1H), 7.48 (s, 1H), 3.51-3.45 (m, 4H), 2.09-2.00 (m, 4H), 1.33 (s, 6H), 1.23 (s, 6H), 1.04 (s, 1H). LCMS: m/z 289.2 [M+H]+, (ESI+), RT=3.45 (Method A).

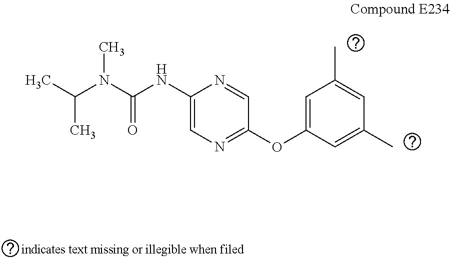 [SMILES: Cc1cc(C)cc(Oc2cnc(NC(=O)N(C)C(C)C)cn2)c1]

### 3-[5-(3,5-difluorophenoxy)pyrazin-2-yl]-1-isopropyl-1-methyl-urea

To a solution of (4-nitrophenyl) carbonochloridate (50 mg, 0.246 mmol) in anhydrous THF (2 mL) was added a solution of 5-(3,5-difluorophenoxy)pyrazin-2-amine (prepared using a similar method to Intermediate I05, 50 mg, 0.224 mmol) and pyridine (20 uL, 0.246 mmol) in anhydrous THF (2 mL) and the reaction was stirred at RT for 1.5 hour. Next, N-methylpropan-2-amine (30 uL, 0.291 mmol) and N-ethyl-N-isopropyl-propan-2-amine (59 uL, 0.336 mmol) in anhydrous THF (2 mL) were added and the reaction was stirred at RT for 2.5 hours. It was then concentrated under reduced pressure and purified by prep HPLC (Method E) followed by flash column chromatography (SNAP KP-Sil, 10 g, 0-55% EtOAc in Heptane), to afford the title compound (29 mg, 40% Yield) as a white solid. 1H NMR, (400 MHz, Chloroform-d) δ 9.01 (d, J =1.4 Hz, 1H), 8.03 (d, J=1.4 Hz, 1H), 7.03 (s, 1H), 6.70-6.60 (m, 3H), 4.64-4.50 (m, 1H), 2.89 (s, 3H), 1.19 (d, J=6.8 Hz, 6H). LCMS: m/z 323.2 [M+H]+, (ESI+), RT=3.27 (Method A)

TABLE 12
The following compounds were Synthesized using a similar method to that used in Compound E234, with precursors
Synthesized using similar methods to Intermediates I02, I05, I06 and Compound E139, Step 1.

| Compound | | | LCMS |
|---|---|---|---|
| number | Structure | Analytical data | Method |
| E235 | 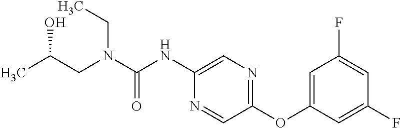 [SMILES: CCN(C[C@H](C)O)C(=O)Nc1cnc(Oc2cc(F)cc(F)c2)cn1] | 1H NMR (500 MHz, Chloroform-d) δ 8.88 (d, J = 1.2 Hz, 1H), 8.81 (s, 1H), 8.00 (d, J = 1.3 Hz, 1H), 6.67-6.57 (m, 3H), 4.17-4.09 (m, 1H), 3.49-3.34 (m, 3H), 3.22 (dd, J = 15.5, 1.9 Hz, 1H), 1.27 (d, J = 6.3 Hz, 3H), 1.22 (t, J = 7.1 Hz, 3H). LCMS: m/z 353.2 [M + H]+, (ESI+), RT = 3.15 | A |
| E236 | 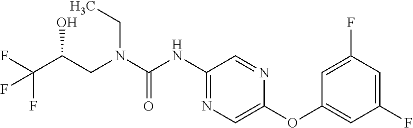 [SMILES: CCN(C[C@@H](O)C(F)(F)F)C(=O)Nc1cnc(Oc2cc(F)cc(F)c2)cn1] | 1H NMR (500 MHz, Chloroform-d) δ 8.90 (d, J = 1.3 Hz, 1H), 8.06 (d, J = 1.4 Hz, 1H), 7.47 (s, 1H), 6.71-6.62 (m, 3H), 4.79 (s, 1H), 4.25-4.15 (m, 1H), 3.82 (dd, J = 15.3, 8.6 Hz, 1H), 3.57- 3.46 (m, 2H), 3.46-3.35 (m, 1H), 1.33 (t, J = 7.2 Hz, 3H). LCMS: m/z 407.2 [M + H]+, (ESI+), RT = 3.43 | A |
| E237 | 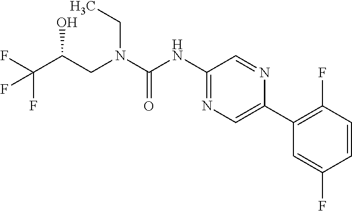 [SMILES: CCN(C[C@@H](O)C(F)(F)F)C(=O)Nc1cnc(-c2cc(F)ccc2F)cn1] | 1H NMR (400 MHz, DMSO-d6) δ 9.52 (s, 1H), 9.18 (d, J = 1.4 Hz, 1H), 8.75- 8.69 (m, 1H), 7.78-7.68 (m, 1H), 7.49- 7.38 (m, 1H), 7.39-7.28 (m, 1H), 6.98 (s, 1H), 4.37-4.27 (m, 1H), 3.68- 3.60 (m, 1H), 3.56-3.41 (m, 3H), 1.13 (t, J = 7.0 Hz, 3H). LCMS: m/z 391.2 [M + H]+, (ESI+), RT = 3.51 | B |
| E238 | 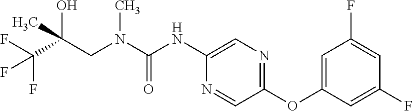 [SMILES: CN(C[C@@](C)(O)C(F)(F)F)C(=O)Nc1cnc(Oc2cc(F)cc(F)c2)cn1] | 1H NMR (400 MHz, DMSO-d6) δ 9.45 (s, 1H), 8.66 (d, J = 1.4 Hz, 1H), 8.28 (d, J = 1.4 Hz, 1H), 7.08 (tt, J = 9.4, 2.3 Hz, 1H), 7.02-6.92 (m, 2H), 6.66 (s, 1H), 3.77 (d, J = 14.3 Hz, 1H), 3.56- 3.39 (m, 1H), 3.08 (s, 3H), 1.28 (s, 3H). LCMS: m/z 407.2 [M + H]+, (ESI+), RT = | A |
| | | 3.36 | |
| E239 | 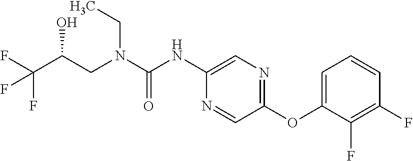 [SMILES: CCN(C[C@@H](O)C(F)(F)F)C(=O)Nc1cnc(Oc2cccc(F)c2F)cn1] | 1H NMR (500 MHz, DMSO-d6) δ 9.25 (s, 1H), 8.55 (d, J = 1.4 Hz, 1H), 8.37 (d, J = 1.4 Hz, 1H), 7.42-7.31 (m, 1H), 7.27 (dddd, J = 8.3, 8.3 5.8, 1.9 Hz, 1H), 7.23-7.14 (m, 1H), 6.91 (s, 1H), 4.36-4.19 (m, 1H), 3.61 (dd, J = 14.8, 2.6 Hz, 1H), 3.53-3.37 (m, 3H), 1.11 (t, J = 7.0 Hz, 3H). LCMS: m/z 407.2 [M + H]+, (ESI+), RT = 3.36 | A |
| E240 | 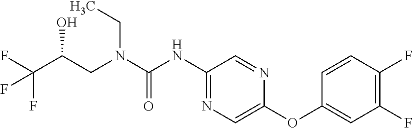 [SMILES: CCN(C[C@@H](O)C(F)(F)F)C(=O)Nc1cnc(Oc2ccc(F)c(F)c2)cn1] | 1HNMR(400 MHz, Chloroform-d) δ 8.83 (d, J = 1.2 Hz, 1H), 8.03 (d, J = 1.2 Hz, 1H), 7.69 (s, 1H), 7.20 (q, J = 9.1 Hz, 1H), 7.02 (ddd, J = 10.8, 6.7, 2.8 Hz, 1H), 6.92-6.84 (m, 1H), 5.35 (s, 1H), 4.26-4.15 (m, 1H), 3.82 (dd, J = 15.3, 8.7 Hz, 1H), 3.59-3.47 (m, 2H), 3.39 (dq, J = 14.5, 7.1 Hz, 1H), 1.32 (t, J = 7.2 | A |
| | | Hz, 3H). | |
| | | LCMS: m/z 407.2 [M + H]+, (ESI+), RT = | |
| | | 3.37 | |
| E241 | 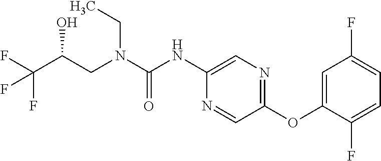 [SMILES: CCN(C[C@@H](O)C(F)(F)F)C(=O)Nc1cnc(Oc2cc(F)ccc2F)cn1] | 1H NMR (400 MHz, DMSO-d6) δ 9.23 (s, 1H), 8.54 (d, J = 1.4 Hz, 1H), 8.34 (d, J = 1.4 Hz, 1H), 7.49-7.40 (m, 1H), 7.38-7.31 (m, 1H), 7.19-7.10 (m, 1H), 6.92 (s, 1H), 4.31-4.21 (m, 1H), 3.61 (dd, J = 14.8, 2.8 Hz, 1H), 3.49- 3.42 (m, 3H), 1.11 (t, J = 7.0 Hz, 3H). LCMS: m/z 407.2 [M + H]+, (ESI+), RT = 3.45 | B |
| E242 | 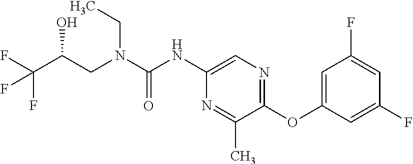 [SMILES: CCN(C[C@@H](O)C(F)(F)F)C(=O)Nc1cnc(Oc2cc(F)cc(F)c2)c(C)n1] | 1HNMR(500 MHz, DMSO-d6) δ 9.21 (s, 1H), 8.52-8.47 (m, 1H), 7.07 (tt, J = 9.4, 2.3 Hz, 1H), 6.97-6.91 (m, 2H), 4.28 (s, 1H), 3.62 (dd, J = 14.8, 2.7 Hz, 1H), 3.54-3.36 (m, 3H), 2.46-2.42 (m, 3H), 1.11 (t, J = 7.0 Hz, 3H). LCMS: m/z 421.2 [M + H]+, (ESI+), RT = 3.62 | A |
| E243 | 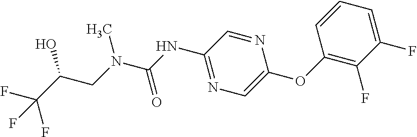 [SMILES: CN(C[C@@H](O)C(F)(F)F)C(=O)Nc1cnc(Oc2cccc(F)c2F)cn1] | 1H NMR (500 MHz, Chloroform-d) δ 8.80 (d, J = 1.4 Hz, 1H), 8.12 (d, J = 1.4 Hz, 1H), 7.32 (s, 1H), 7.14-7.03 (m, 2H), 7.01 (ddt, J = 8.3, 6.5, 1.9 Hz, 1H), 4.75 (s, 1H), 4.20 (d, J = 6.8 Hz, 1H), 3.79 (dd, J = 15.3, 7.9 Hz, 1H), 3.63 (dd, J = 15.3, 2.3 Hz, 1H), 3.16 (s, 3H). LCMS: m/z 393.1 [M + H]+, (ESI+), RT = 3.09 | A |
| E244 | 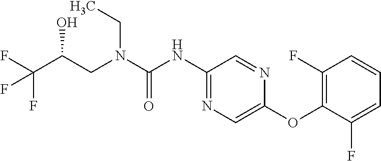 [SMILES: CCN(C[C@@H](O)C(F)(F)F)C(=O)Nc1cnc(Oc2c(F)cccc2F)cn1] | 1H NMR (500 MHz, Chloroform-d) δ 8.79 (d, J = 1.4 Hz, 1H), 8.20 (d, J = 1.4 Hz, 1H), 7.35 (s, 1H), 7.25-7.17 (m, 1H), 7.08-6.99 (m, 2H), 4.95 (s, 1H), 4.24-4.15 (m, 1H), 3.84 (dd, J = 15.3, 8.5 Hz, 1H), 3.55-3.48 (m, 2H), 3.48- 3.38 (m, 1H), 1.34 (t, J = 7.2 Hz, 3H). LCMS: m/z 407.2 [M + H]+, (ESI+), RT = 3.32 | A |
| E245 | 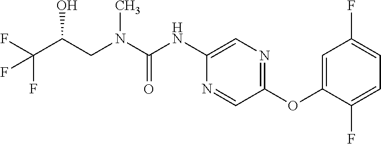 [SMILES: CN(C[C@@H](O)C(F)(F)F)C(=O)Nc1cnc(Oc2cc(F)ccc2F)cn1] | 1H NMR (400 MHz, DMSO-d6) δ 9.24 (s, 1H), 8.54 (d, J = 1.4 Hz, 1H), 8.35 (d, J = 1.4 Hz, 1H), 7.50-7.31 (m, 2H), 7.22-7.08 (m, 1H), 6.71 (s, 1H), 4.33- 4.22 (m, 1H), 3.64 (dd, J = 14.6, 3.4 Hz, 1H), 3.49-3.39 (m, 1H), 3.05 (s, 3H). LCMS: m/z 393.1 [M + H]+, (ESI+), RT = 3.16 | B |
| E246 | 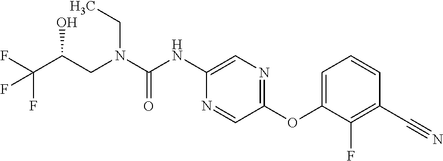 [SMILES: CCN(C[C@@H](O)C(F)(F)F)C(=O)Nc1cnc(Oc2cccc(C#N)c2F)cn1] | 1H NMR (400 MHz, Methanol-d4) δ 8.57 (d, J = 1.4 Hz, 1H), 8.28 (d, J = 1.4 Hz, 1H), 7.70-7.62 (m, 2H), 7.47- 7.40 (m, 1H), 4.36-4.26 (m, 1H), 3.72- 3.46 (m, 4H), 1.26 (t, J = 7.1 Hz, 3H). LCMS: m/z 414.2 [M + H]+, (ESI+), RT = 3.14 | A |
| E247 | 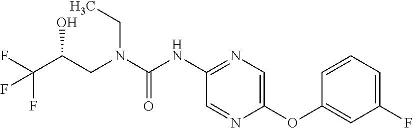 [SMILES: CCN(C[C@@H](O)C(F)(F)F)C(=O)Nc1cnc(Oc2cccc(F)c2)cn1] | 1H NMR (400 MHz, DMSO-d6) δ 9.08 (s, 1H), 8.64 (d, J = 1.4 Hz, 1H), 8.25 (d, J = 1.4 Hz, 1H), 7.44 (td, J = 8.2, 7.0 Hz, 1H), 7.12-7.00 (m, 2H), 6.98 (dd, J = 8.1, 1.7 Hz, 1H), 4.36-4.12 (m, 1H), 3.62 (dd, J = 14.8, 2.8 Hz, 1H), 3.54-3.36 (m, 3H), 1.11 (t, J = 7.0 Hz, 3H). | B |
| | | LCMS: m/z 389.1 [M + H]+, (ESI+), RT = | |
| | | 3.38 | |
| E248 | 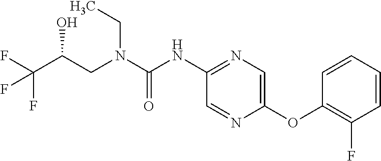 [SMILES: CCN(C[C@@H](O)C(F)(F)F)C(=O)Nc1cnc(Oc2ccccc2F)cn1] | 1H NMR (400 MHz, DMSO-d6) δ 9.19 (s, 1H), 8.52 (d, J = 1.4 Hz, 1H), 8.32 (d, J = 1.4 Hz, 1H), 7.46-7.10 (m, 4H), 6.90 (s, 1H), 4.43-4.13 (m, 1H), 3.61 (dd, J = 14.8, 2.9 Hz, 1H), 3.51-3.36 (m, 3H), 1.11 (t, J = 7.0 Hz, 3H). LCMS: m/z 389.2 [M + H]+, (ESI+), RT = 3.34 | B |
| E249 | 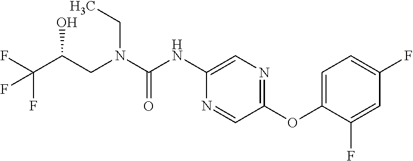 [SMILES: CCN(C[C@@H](O)C(F)(F)F)C(=O)Nc1cnc(Oc2ccc(F)cc2F)cn1] | 1H NMR (500 MHz, DMSO-d6) δ 9.20 (s, 1H), 8.51 (d, J = 1.4 Hz, 1H), 8.33 (d, J = 1.4 Hz, 1H), 7.57-7.37 (m, 2H), 7.23-7.08 (m, 1H), 6.92 (s, 1H), 4.40- 4.18 (m, 1H), 3.61 (dd, J = 14.8, 2.7 Hz, 1H), 3.53-3.39 (m, 3H), 1.10 (t, J = 7.0 Hz, 3H). LCMS: m/z 407.2 [M + H]+, (ESI+), RT = 3.32 | A |
| E250 | 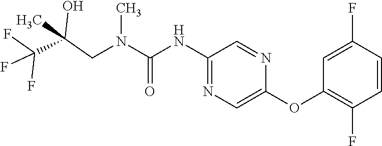 [SMILES: CN(C[C@@](C)(O)C(F)(F)F)C(=O)Nc1cnc(Oc2cc(F)ccc2F)cn1] | 1HNMR(500 MHz, Chloroform-d) δ 8.85 (d, J = 1.3 Hz, 1H), 8.13 (d, J = 1.4 Hz, 1H), 7.43 (s, 1H), 7.16 (td, J = 9.4, 5.0 Hz, 1H), 7.01 (ddd, J = 8.9, 6.1, 3.1 Hz, 1H), 6.93 (ddt, J = 9.1, 7.5, 3.3 Hz, 1H), 5.47 (s, 1H), 3.74 (d, J = 15.3 Hz, 1H), 3.65 (d, J = 15.4 Hz, 1H), 3.20 (s, 3H), 1.42 (s, 3H). LCMS: m/z 407.2 [M + H]+, (ESI+), RT = 3.27 | A |
| E251 | 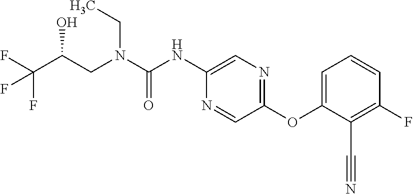 [SMILES: CCN(C[C@@H](O)C(F)(F)F)C(=O)Nc1cnc(Oc2cccc(F)c2C#N)cn1] | 1H NMR (500 MHz, DMSO-d6) δ 9.40 (s, 1H), 8.69 (d, J = 1.4 Hz, 1H), 8.44 (d, J = 1.4 Hz, 1H), 7.79 (td, J = 8.5, 6.8 Hz, 1H), 7.38 (t, J = 8.8 Hz, 1H), 7.19 (d, J = 8.5 Hz, 1H), 6.92 (s, 1H), 4.33- 4.24 (m, 1H), 3.63 (dd, J = 14.8, 2.5 Hz, 1H), 3.53-3.39 (m, 3H), 1.12 (t, J = 7.0 Hz, 3H). LCMS: m/z 414.2 [M + H]+, (ESI+), RT = 3.21 | A |
| E252 | 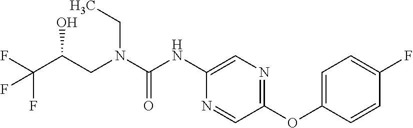 [SMILES: CCN(C[C@@H](O)C(F)(F)F)C(=O)Nc1cnc(Oc2ccc(F)cc2)cn1] | (500 MHz, Chloroform-d) δ 8.81 (d, J = 1.3 Hz, 1H), 8.01 (d, J = 1.4 Hz, 1H), 7.41 (s, 1H), 7.13-7.02 (m, 4H), 5.03 (s, 1H), 4.23-4.13 (m, 1H), 3.81 (dd, J = 5.3, 8.6 Hz, 1H), 3.55-3.45 (m, 2H), 3.39 (dq, J = 14.6, 7.2 Hz, 1H), 1.31 (t, J = 7.2 Hz, 3H). LCMS: m/z 389.2 [M + H]+, (ESI+), RT = | A |
| | | 3.41 | |
| E253 | 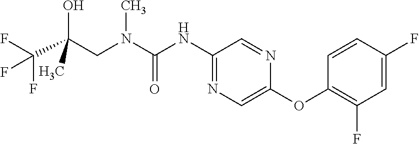 [SMILES: CN(C[C@@](C)(O)C(F)(F)F)C(=O)Nc1cnc(Oc2ccc(F)cc2F)cn1] | 1HNMR(400 MHz, Chloroform-d) δ 8.82 (d, J = 1.4 Hz, 1H), 8.12 (d, J = 1.4 Hz, 1H), 7.37 (s, 1H), 7.22 (td, J = 8.8, 5.5 Hz, 1H), 7.01-6.89 (m, 2H), 5.42 (s, 1H), 3.78-3.60 (m, 2H), 3.20 (s, 3H), 1.41 (s, 3H). LCMS: m/z 407.2 [M + H]+, (ESI+), RT = 3.26 | A |
| E254 | 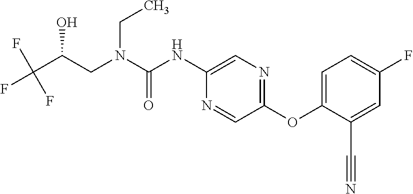 [SMILES: CCN(C[C@@H](O)C(F)(F)F)C(=O)Nc1cnc(Oc2ccc(F)cc2C#N)cn1] | 1HNMR(400 MHz, Chloroform-d) δ 8.84 (d, J = 1.4 Hz, 1H), 8.21 (d, J = 1.4 Hz, 1H), 7.47 (s, 1H), 7.43-7.38 (m, 1H), 7.38-7.33 (m, 1H), 7.33-7.29 (m, 1H), 4.80 (s, 1H), 4.21 (s, 1H), 3.84 (dd, J = 15.3, 8.6 Hz, 1H), 3.60-3.36 (m, 3H), 1.35 (t, J = 7.2 Hz, 3H). LCMS: m/z 414.1 [M + H]+, (ESI+), RT = 3.17 | A |
| E255 | 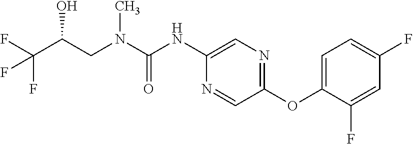 [SMILES: CN(C[C@@H](O)C(F)(F)F)C(=O)Nc1cnc(Oc2ccc(F)cc2F)cn1] | 1H NMR (500 MHz, Methanol-d4) δ 8.51 (d, J = 1.4 Hz, 1H), 8.17 (d, J = 1.4 Hz, 1H), 7.30 (td, J = 8.9, 5.5 Hz, 1H), 7.12 (ddd, J = 10.9, 8.7, 3.0 Hz, 1H), 7.03-6.98 (m, 1H), 4.35-4.23 (m, 1H), 3.72 (d, J = 14.2 Hz, 1H), 3.59- 3.47 (m, 1H), 3.13 (s, 3H). LCMS: m/z 393.1 [M + H]+, (ESI+), RT = 3.07 | A |
| E256 | 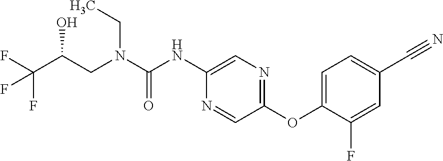 [SMILES: CCN(C[C@@H](O)C(F)(F)F)C(=O)Nc1cnc(Oc2ccc(C#N)cc2F)cn1] | 1H NMR (500 MHz, DMSO-d6) δ 9.30 (s, 1H), 8.58 (d, J = 1.4 Hz, 1H), 8.40 (d, J = 1.4 Hz, 1H), 8.08 (dd, J = 10.7, 1.9 Hz, 1H), 7.85-7.71 (m, 1H), 7.56 (dd, J = 8.2, 8.2 Hz, 1H), 6.91 (s, 1H), 4.38-4.18 (m, 1H), 3.62 (dd, J = 14.8, 2.6 Hz, 1H), 3.53-3.36 (m, 3H), 1.11 (t, J = 7.0 Hz, 3H). LCMS: m/z 414.1 [M + H]+, (ESI+), RT = 3.17 | A |
| E257 | 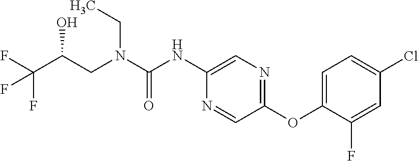 [SMILES: CCN(C[C@@H](O)C(F)(F)F)C(=O)Nc1cnc(Oc2ccc(Cl)cc2F)cn1] | 1H NMR (500 MHz, DMSO-d6) δ 9.23 (s, 1H), 8.52 (d, J = 1.4 Hz, 1H), 8.35 (d, J = 1.4 Hz, 1H), 7.64 (dd, J = 10.5, 2.4 Hz, 1H), 7.41 (t, 1H), 7.35 (ddd, J = 8.7, 2.4, 1.2 Hz, 1H), 6.91 (s, 1H), 4.27 (s, 1H), 3.61 (dd, J = 14.8, 2.7 Hz, 1H), 3.51-3.38 (m, 3H), 1.10 (t, J = 7.0 Hz, 3H). LCMS: m/z 423.2 [M + H]+, (ESI+), RT = 3.76 | B |
| E258 | 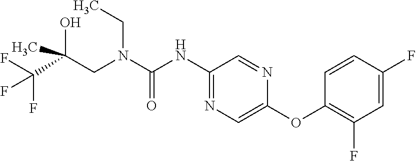 [SMILES: CCN(C[C@@](C)(O)C(F)(F)F)C(=O)Nc1cnc(Oc2ccc(F)cc2F)cn1] | 1HNMR(500 MHz, Chloroform-d) δ 8.82 (d, J = 1.4 Hz, 1H), 8.12 (d, J = 1.4 Hz, 1H), 7.41 (s, 1H), 7.22 (td, J = 8.8, 5.5 Hz, 1H), 7.01-6.90 (m, 2H), 5.57 (s, 1H), 3.74 (d, J = 15.3 Hz, 1H), 3.61- 3.49 (m, 2H), 3.38 (dq, J = 14.7, 7.2 Hz, 1H), 1.41 (s, 3H), 1.33 (t, J = 7.2 Hz, 3H). LCMS: m/z 421.2 [M + H]+, (ESI+), RT = 3.52 | A |
| E259 | 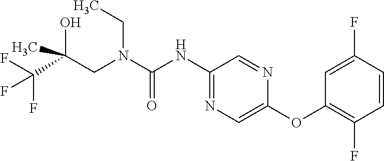 [SMILES: CCN(C[C@@](C)(O)C(F)(F)F)C(=O)Nc1cnc(Oc2cc(F)ccc2F)cn1] | 1HNMR(500 MHz, Chloroform-d) δ 8.86 (d, J = 1.4 Hz, 1H), 8.13 (d, J = 1.4 Hz, 1H), 7.44 (s, 1H), 7.16 (td, J = 9.4, 5.0 Hz, 1H), 7.04-6.98 (m, 1H), 6.96- 6.89 (m, 1H), 5.54 (s, 1H), 3.74 (d, J = 15.3 Hz, 1H), 3.65-3.47 (m, 2H), 3.47- 3.29 (m, 1H), 1.42 (s, 3H), 1.33 (t, J = 7.2 Hz, 3H). LCMS: m/z 421.2 [M + H]+, (ESI+), RT = 3.53 | A |
| E260 | 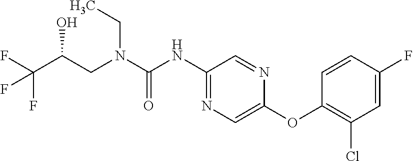 [SMILES: CCN(C[C@@H](O)C(F)(F)F)C(=O)Nc1cnc(Oc2ccc(F)cc2Cl)cn1] | 1HNMR(500 MHz, Chloroform-d) δ 8.80 (d, J = 1.4 Hz, 1H), 8.12 (d, J = 1.4 Hz, 1H), 7.40 (s, 1H), 7.25 (dd, J = 8.0, 3.0 Hz, 1H), 7.21 (dd, J = 9.0, 5.1 Hz, 1H), 7.09-7.03 (m, 1H), 4.99 (s, 1H), 4.20 (p, J = 6.7 Hz, 1H), 3.83 (dd, J = 15.3, 8.6 Hz, 1H), 3.56-3.46 (m, 2H), 3.46-3.35 (m, 1H), 1.34 (t, J = 7.2 Hz, 3H). LCMS: m/z 423.2 [M + H]+, (ESI+), RT = 3.51 | A |
| E261 | 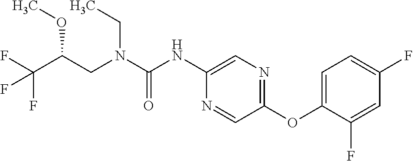 [SMILES: CCN(C[C@@H](OC)C(F)(F)F)C(=O)Nc1cnc(Oc2ccc(F)cc2F)cn1] | 1H NMR (500 MHz, DMSO-d6) δ 9.18 (s, 1H), 8.52 (d, J = 1.4 Hz, 1H), 8.35 (d, J = 1.4 Hz, 1H), 7.51-7.39 (m, 2H), 7.19-7.12 (m, 1H), 4.22-4.12 (m, 1H), 3.66 (dd, J = 14.9, 3.4 Hz, 1H), 3.54-3.42 (m, 6H), 1.11 (t, J = 7.0 Hz, 3H).LCMS: m/z 421.1 [M + H]+, (ESI+), RT = 3.79 | A |
| E262 | 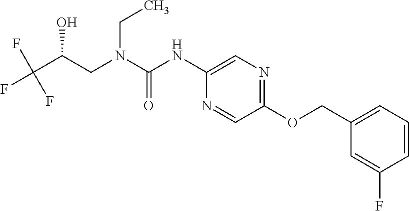 [SMILES: CCN(C[C@@H](O)C(F)(F)F)C(=O)Nc1cnc(OCc2cccc(F)c2)cn1] | 1H NMR (500 MHz, Chloroform-d) δ 8.79 (d, J = 1.4 Hz, 1H), 7.92 (d, J = 1.4 Hz, 1H), 7.33 (td, J = 7.9, 5.9 Hz, 1H), 7.21 (d, J = 7.7 Hz, 1H), 7.16 (d, J = 9.5 Hz, 1H), 7.01 (td, J = 8.4, 2.4 Hz, 1H), 5.36 (s, 2H), 5.20 (d, J = 14.4 Hz, 1H), 4.18 (p, J = 6.6 Hz, 1H), 3.82 (dd, J = 15.2, 8.6 Hz, 1H), 3.53-3.44 (m, 2H), 3.43-3.35 (m, 1H), 1.31 (t, J = 7.2 Hz, 3H). LCMS: m/z 403.1 [M + H]+, (ESI+), RT = 3.46 | A |
| E263 | 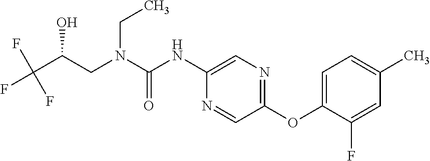 [SMILES: CCN(C[C@@H](O)C(F)(F)F)C(=O)Nc1cnc(Oc2ccc(C)cc2F)cn1] | 1H NMR (500 MHz, DMSO-d6) δ 9.19 (s, 1H), 8.50 (d, J = 1.4 Hz, 1H), 8.29 (d, J = 1.4 Hz, 1H), 7.24-7.17 (m, 2H), 7.08-7.02 (m, 1H), 6.90 (s, 1H), 4.33- 4.22 (m, 1H), 3.60 (dd, J = 14.8, 2.6 Hz, 1H), 3.52-3.40 (m, 3H), 2.33 (s, 3H), 1.10 (t, J = 7.0 Hz, 3H). LCMS: m/z 403.3 [M + H]+, (ESI+), RT = 3.64 | B |
| E264 | 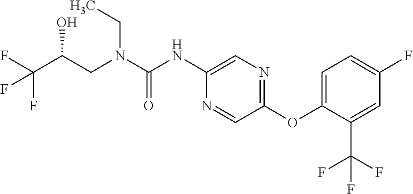 [SMILES: CCN(C[C@@H](O)C(F)(F)F)C(=O)Nc1cnc(Oc2ccc(F)cc2C(F)(F)F)cn1] | 1H NMR (400 MHz, Chloroform-d) δ 8.79 (d, J= 1.4 Hz, 1H), 8.11 (d, J = 1.4 Hz, 1H), 7.41 (dd, J = 8.2, 2.9 Hz, 2H), 7.32-7.27 (m, 1H), 7.23 (dd, J = 9.1, 4.7 Hz, 1H), 4.85 (s, 1H), 4.18 (d, J = 4.5 Hz, 1H), 3.81 (dd, J = 15.3, 8.6 Hz, 1H), 3.54-3.48 (m, 2H), 3.42 (td, J = 15.3, 7.5 Hz, 1H), 1.32 (t, J = 7.2 Hz, 3H). LCMS: m/z 457.2 [M + H]+, (ESI+), RT = 3.64 | A |

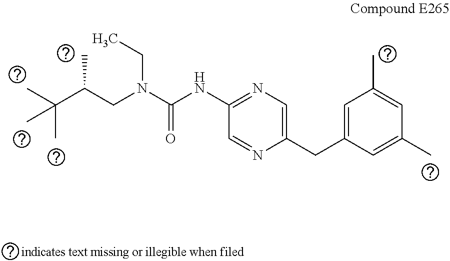 [SMILES: CCN(C[C@@H](C)C(C)(C)C)C(=O)Nc1cnc(Cc2cc(C)cc(C)c2)cn1]

### 3-[5-[(3,5-difluorophenyl)methyl]pyrazin-2-yl]-1-ethyl-1-[(2R)-3,3,3-trifluoro-2-hydroxy-propyl]urea

Step 1

tert-butyl N-(5-bromopyrazin-2-yl)carbamate (500 mg, 1.70 mmol) and dichloro(1,3-bis(diphenylphosphino)propane)nickel (92 mg, 0.170 mmol) were suspended in 1,4-Dioxane-Anhydrous (4.7 mL) and the reaction mixture was degassed with N2 at rt for 5 minutes. 0.5 M bromo-[(3,5-difluorophenyl)methyl]zinc (14 mL, 6.79 mmol) was then slowly added and the reaction stirred at 60° C. for 2 hours. The reaction mixture was then cooled to RT, diluted with EtOAc (25 mL), aq. NaHCO3 (25 mL) and brine (15 mL). The aqueous layer was separated and extracted with EtOAc (25 mL). The combined organics were washed with brine (15 mL), dried with MgSO4, filtered and concentrated. The crude material was then purified by normal phase flash column chromatography (Sfar Duo, 100 g, eluents: EtOAc in Heptane, 0-60%) to afford tert-butyl N-[5-[(3,5-difluorophenyl)methyl]pyrazin-2-yl]carbamate (424 mg, 75% Yield) as a white solid. 1H NMR, (400 MHz, DMSO-d6) δ 10.09 (s, 1H), 8.94 (d, J=1.5 Hz, 1H), 8.30 (d, J=1.5 Hz, 1H), 7.11-6.94 (m, 3H), 4.09 (s, 2H), 1.47 (s, 9H).

Step 2

To tert-butyl N-[5-[(3,5-difluorophenyl)methyl]pyrazin-2-yl]carbamate (424 mg, 1.28 mmol) was added 4 M hydrogen chloride in dioxane (3.5 mL, 14.1 mmol), and stirred at RT for 3h. Further 4 M hydrogen chloride in dioxane (3.5 mL, 14.1 mmol) was added and the mixture was stirred at RT for 7h. This was then concentrated in vacuo, passed through an SCX-2 column (5 g) (flushed with MeOH (2CV) then 7N NH3 in MeOH (2.5CV)) to afford 5-[(3,5-difluorophenyl)methyl]pyrazin-2-amine (264 mg, 88% Yield) as a brown solid. 1H NMR, (500 MHz, DMSO-d6) δ 7.84 (dd, J=33.4, 1.4 Hz, 2H), 7.03 (tt, J=9.4, 2.4 Hz, 1H), 6.98-6.90 (m, 2H), 6.27 (s, 2H), 3.90 (s, 2H).

Step 3

Urea formation using a similar method to that used in Compound E191, to afford the title compound (36 mg, 34% Yield) as a white solid. 1H NMR, (500 MHz, Chloroform-d) δ 9.26 (d, J=1.4 Hz, 1H), 8.04 (d, J=1.4 Hz, 1H), 7.47 (s, 1H), 6.81-6.74 (m, 2H), 6.67 (tt, J=9.0, 2.3 Hz, 1H), 4.82 (s, 1H), 4.20 (s, 1H), 4.08 (s, 2H), 3.81 (dd, J=15.3, 8.7 Hz, 1H), 3.56-3.46 (m, 2H), 3.40 (dq, J=14.7, 7.2 Hz, 1H), 1.32 (t, J=7.2 Hz, 3H). LCMS: m/z 405.2 [M+H]+, (ESI+), RT=3.42 (Method A).

TABLE 13
The following compounds were Synthesized using a similar method to that used in Compound E265.

| Compound | | | LCMS |
|---|---|---|---|
| number | Structure | Analytical data | Method |
| E266 | 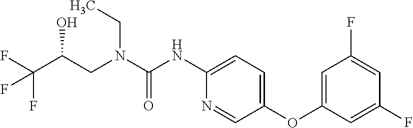 [SMILES: CCN(C[C@@H](O)C(F)(F)F)C(=O)Nc1ccc(Oc2cc(F)cc(F)c2)cn1] | 1H NMR (500 MHz, DMSO-d6) δ 8.90 (s, 1H), 8.18 (d, J = 2.0 Hz, 1H), 7.72 (d, J = 8.6 Hz, 1H), 7.59 (dd, J = 8.6, 2.4 Hz, 1H), 7.11-6.91 (m, 4H), 4.26 (s, 1H), 3.92 (s, 2H), 3.58 (dd, J = 14.9, 2.4 Hz, 1H), 3.48-3.39 (m, 3H), 1.10 (t, J = 7.0 Hz, 3H). LCMS: m/z 404.2 [M + H]+, (ESI+), RT = | A |
| | | 2.82 | |
| E267 | 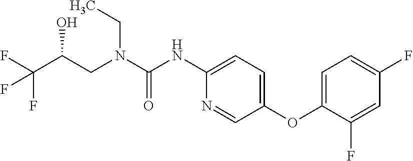 [SMILES: CCN(C[C@@H](O)C(F)(F)F)C(=O)Nc1ccc(Oc2ccc(F)cc2F)cn1] | (500 MHz, Chloroform-d) δ 8.05 (s, 1H), 7.89 (s, 1H), 7.49 (dd, J = 8.6, 2.3 Hz, 1H), 7.12-7.04 (m, 1H), 6.83- 6.77 (m, 2H), 4.19-4.11 (m, 1H), 3.89 (s, 2H), 3.82 (dd, J = 15.2, 8.5 Hz, 1H), 3.52-3.34 (m, 3H), 1.30 (t, J = 7.2 Hz, 3H). LCMS: m/z 405.1 [M + H]+, (ESI+), RT = 2.81 | A |
| E268 | 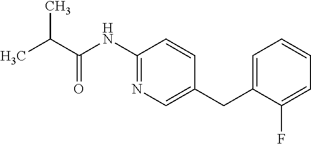 [SMILES: CC(C)C(=O)Nc1ccc(Cc2ccccc2F)cn1] | 1H NMR (250 MHz, DMSO-d6) δ 10.34 (s, 1H), 8.18 (d, J = 2.3 Hz, 1H), 8.01 (d, J = 8.5 Hz, 1H), 7.64-7.52 (m, 1H), 7.38-7.20 (m, 2H), 7.20-7.07 (m, 2H), 3.94 (s, 2H), 2.77-2.64 (m, 1H), 1.06 (d, J = 6.8 Hz, 6H). LCMS: m/z 273.1 [M + H]+, (ESI+), RT = 2.83 | A |

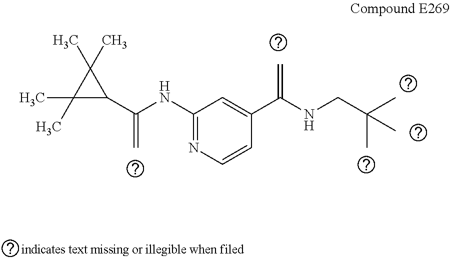 [SMILES: C=C(NCC(C)(C)C)c1ccnc(NC(=C)C2C(C)(C)C2(C)C)c1]

### 2-[(2,2,3,3-tetramethylcyclopropanecarbonyl)amino]-N-(2,2,2-trifluoroethyl)pyridine-4-carboxamide

Step 1

Starting from methyl 2-aminopyridine-4-carboxylate (200 mg, 1.31 mmol), a similar method to Compound E199 (Step 2) was used to afford 2-[(2,2,3,3-tetramethylcyclopropanecarbonyl)amino]pyridine-4-carboxylic acid (260 mg, 90% purity, 68% yield). 1H NMR (400 MHz, DMSO-d6) δ 10.61 (s, 1H), 8.61-8.51 (m, 1H), 8.43 (d, J=5.0 Hz, 1H), 7.45 (dd, J=5.1, 1.3 Hz, 1H), 1.57 (s, 1H), 1.24 (s, 6H), 1.17 (s, 6H), OH not observed.

Step 2

2-[(2,2,3,3-tetramethylcyclopropanecarbonyl)amino]pyridine-4-carboxylic acid (50 mg, 0.172 mmol) was dissolved in DMF-Anhydrous (1.5 mL) and N-ethyl-N-isopropyl-propan-2-amine (90 uL, 0.515 mmol) was added followed by HATU (98 mg, 0.257 mmol). After stirring for 10 minutes, 2,2,2-trifluoroethanamine (20 uL, 0.257 mmol) was added and the reaction was stirred at RT overnight. It was then diluted with EtOAc (10 mL) and washed with sat. aq. NaHCO3 (10 mL). The aqueous layer was extracted with EtOAc (2×10 mL) and the combined organic extracts were dried over MgSO4, filtered, concentrated under reduced pressure and purified by prep HPLC (Method F) to afford the title compound as a white solid (48 mg). 1H NMR (500 MHz, DMSO-d6) δ 10.59 (s, 1H), 9.42-9.22 (m, 1H), 8.49-8.37 (m, 2H), 7.40 (dd, J=5.1, 1.6 Hz, 1H), 4.08 (td, J=9.6, 5.6 Hz, 2H), 1.57 (s, 1H), 1.25 (s, 6H), 1.17 (s, 6H). LCMS: m/z 344.3 [M+H]+, (ESI+), RT=3.30 (Method A).

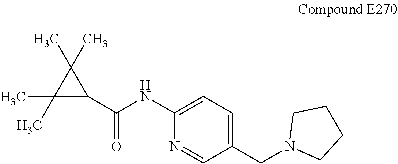 [SMILES: CC1(C)C(C(=O)Nc2ccc(CN3CCCC3)cn2)C1(C)C]

### 2,2,3,3-tetramethyl-N-[5-(pyrrolidin-1-ylmethyl)-2-pyridyl]cyclopropanecarboxamide

To a stirred solution of N-(5-formyl-2-pyridyl)-2,2,3,3-tetramethyl-cyclopropanecarboxamide (Synthesized using a similar method to E199 (step 2), 100 mg, 0.406 mmol) in DCE (2 mL) was added pyrrolidine (41 uL, 0.487 mmol) followed by acetic acid (2.3 uL, 0.0406 mmol). After 4 h, sodium triacetoxyborohydride (172 mg, 0.812 mmol) was added. The reaction mixture was washed with saturated aqueous sodium bicarbonate solution (25 mL) and extracted with ethyl acetate (2×20 mL). The combined organic layers were washed with brine (20 mL), dried (hydrophobic filter) and concentrated to dryness under reduced pressure. The crude material was purified by flash chromatography (Biotage 11 g SNAP-KPNH cartridge, 0-25% EtOAc in heptane) followed by further chromatography (C18 silica gel, 12 g SNAP Ultra cartridge, eluent: 0.1% formic acid in acetonitrile-0.1% formic acid in water, 15-30%). The crude product was then washed with saturated aqueous sodium bicarbonate solution (20 mL) and extracted with ethyl acetate (2×20 mL). Final purification by flash chromatography (Biotage 11 g SNAP-KPNH cartridge, 0-20% EtOAc in heptane) gave the title product as a colourless solid (15 mg, 12% yield). 1H NMR (500 MHz, Chloroform-d) δ 8.17 (d, J=1.9 Hz, 1H), 8.11 (d, J=8.5 Hz, 1H), 7.96 (s, 1H), 7.64 (dd, J=8.5, 2.2 Hz, 1H), 3.56 (s, 2H), 2.53-2.43 (m, 4H), 1.82 1.74 (m, 4H), 1.32 (s, 6H), 1.21 (s, 6H), 1.02 (s, 1H). LCMS: m/z 302.4 [M+H]+, (ESI+), RT=1.79 (Method A).

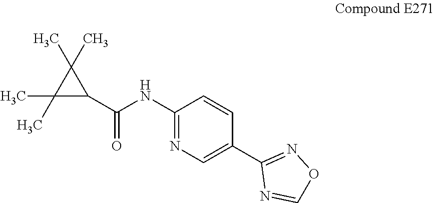 [SMILES: CC1(C)C(C(=O)Nc2ccc(-c3ncon3)cn2)C1(C)C]

### 2,2,3,3-tetramethyl-N-[5-(1,2,4-oxadiazol-3-yl)-2-pyridyl]cyclopropanecarboxamide

Step 1

To a solution of N-(5-cyano-2-pyridyl)-2,2,3,3-tetramethyl-cyclopropanecarboxamide (Synthesized using a similar method to E199 (step 2), 247 mg, 0.934 mmol) in Ethanol (5.8974 mL) was added hydroxylamine (50%, 0.50 mL, 0.934 mmol). The reaction mixture was stirred at room temperature for 5 mins then at 80° C. for 3 hours. The reaction mixture was concentrated under reduced pressure. To the mixture was added DCM (10 ml) and Water (10 ml). A colourless precipitate was isolated by filtration to afford N-[5-(N-hydroxycarbamimidoyl)-2-pyridyl]-2,2,3,3-tetramethyl-cyclopropanecarboxamide (176 mg, 67% Yield) as a colourless solid. 1H NMR (400 MHz, DMSO-d6) δ 10.48 (s, 1H), 9.64 (s, 1H), 8.55 (dd,J=2.3, 0.7 Hz, 1H), 8.08-8.01 (m, 1H), 7.96 (dd,J=8.8, 2.4 Hz, 1H), 5.87 (s, 2H), 1.56 (s, 1H), 1.24 (s, 6H), 1.17 (s, 6H).

Step 2

To a stirred solution of N-[5-(N-hydroxycarbamimidoyl)-2-pyridyl]-2,2,3,3-tetramethyl-cyclopropanecarboxamide (100%, 50 mg, 0.181 mmol) in trimethoxymethane (3.0 mL, 0.181 mmol) was added a catalytic amount of 2,2,2-trifluoroacetic acid (0.0013 mL). The reaction was stirred at room temperature for 5 mins then at 60° C. for 30 mins. The reaction mixture was then concentrated under reduced pressure. The crude product was purified by prep HPLC (Method G) to afford the title compound (39 mg, 75% Yield) as a colourless solid. 1H NMR (400 MHz, Chloroform-d) δ 9.02-8.99 (m, 1H), 8.79 (s, 1H), 8.38-8.35 (m, 2H), 8.17 (s, 1H), 1.36 (s, 6H), 1.26 (s, 6H), 1.08 (s, 1H). LCMS: m/z 287.2 [M+H]+, (ESI+), RT=3.50 (Method A).

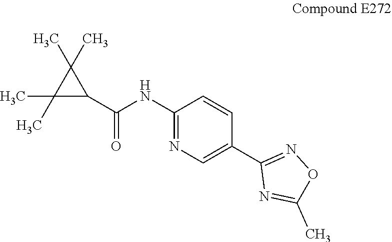 [SMILES: Cc1nc(-c2ccc(NC(=O)C3C(C)(C)C3(C)C)nc2)no1]

### 2,2,3,3-tetramethyl-N-[5-(5-methyl-1,2,4-oxadiazol-3-yl)-2-pyridyl]cyclopropanecarboxamide

Synthesized using a similar method to that used in Compound E271. 1H NMR (400 MHz, Chloroform-d) δ 8.97-8.92 (m, 1H), 8.36-8.29 (m, 2H), 8.15 (s, 1H), 2.68 (s, 3H), 1.36 (s, 6H), 1.26 (s, 6H), 1.07 (s, 1H). LCMS: m/z 301.2 [M+H]+, (ESI+), RT=3.78 (Method B).

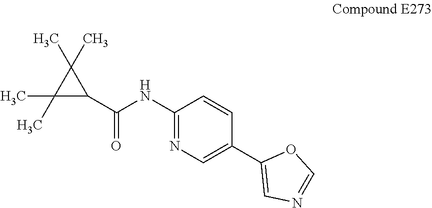 [SMILES: CC1(C)C(C(=O)Nc2ccc(-c3cnco3)cn2)C1(C)C]

### 2,2,3,3-tetramethyl-N-(5-oxazol-5-yl-2-pyridyl)cyclopropanecarboxamide

Step 1

To a mixture of 1-(isocyanomethylsulfonyl)-4-methyl-benzene (320 mg, 1.64 mmol) and dipotassium carbonate (226 mg, 1.64 mmol) in Methanol (4.5 mL) was added 6-aminopyridine-3-carbaldehyde (200 mg, 1.64 mmol). The reaction mixture was held at reflux for 2.5 h and then cooled, concentrated under reduced pressure and partitioned between MTBE (35m1) and water (25 mL). After separation of the layers, the aqueous layer was extracted twice with EtOAc (2×30 mL). The combined organics were dried (MgSO4), filtered and concentrated to provide 5-oxazol-5-ylpyridin-2-amine (199 mg, 35% purity) as a yellow oil that was used directly to the next step without further purification. 1H NMR, (500 MHz, DMSO-d6) δ 8.31 (s, 1H), 8.30-8.27 (m, 1H), 7.46-7.44 (m, 1H), 7.40 (s, 1H), 6.52 (dd, J=8.7, 0.7 Hz, 1H), 6.31 (s, 2H).

Step 2

Starting from 5-oxazol-5-ylpyridin-2-amine (65 mg, 35% pure), a similar method to Compound E199 (Step 2) was used to afford the title compound (6.1 mg, 15% Yield) as a white solid. 1H NMR (500 MHz, Chloroform-d) δ 8.56 (dd, J=2.3, 0.6 Hz, 1H), 8.29-8.24 (m, 1H), 8.03 (s, 1H), 7.93 (s, 1H), 7.90 (dd, J=8.7, 2.3 Hz, 1H), 7.34 (s, 1H), 1.33 (s, 6H), 1.23 (s, 6H), 1.04 (s, 1H). LCMS: m/z 286.2 [M+H]+, (ESI+), RT=3.25 (Method A).

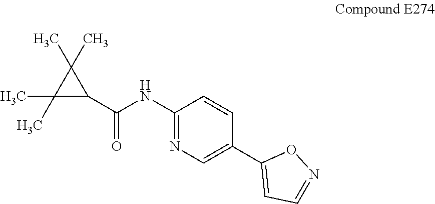 [SMILES: CC1(C)C(C(=O)Nc2ccc(-c3ccno3)cn2)C1(C)C]

### N-(5-isoxazol-5-yl-2-pyridyl)-2,2,3,3-tetramethyl-cyclopropanecarboxamide

Step 1

N-(5-acetyl-2-pyridyl)-2,2,3,3-tetramethyl-cyclopropanecarboxamide (Synthesized using a similar method to E199 (step 2), 90 mg, 0.346 mmol) was dissolved in 1,1-dimethoxy-N,N-dimethylmethanamine (1.0 mL, 7.53 mmol) . The reaction mixture was allowed to stir at 110° C. for 16 h. After completion of the reaction, excess volatiles were removed under reduced pressure to afford N-[5-[(E)-3-(dimethylamino)prop-2-enoyl]-2-pyridyl]-2,2,3,3-tetramethyl-cyclopropanecarboxamide (109 mg, 85% pure) as a yellow oil. 1H NMR (400 MHz, Chloroform-d) δ 8.72 (d,J=1.4 Hz, 1H), 8.17-8.12 (m, 2H), 8.10 (dd,J=8.7, 2.2 Hz, 1H), 7.76 (d,J=12.2 Hz, 1H), 5.58 (d,J=12.3 Hz, 1H), 3.27 (d,J=6.9 Hz, 3H), 2.21 (d,J=9.2 Hz, 3H), 1.26 (s, 6H), 1.15 (s, 6H), 0.98 (s, 1H).

Step 2

N-[5-[(E)-3-(dimethylamino)prop-2-enoyl]-2-pyridyl]-2,2,3,3-tetramethyl-cyclopropanecarboxamide (85%, 109 mg, 0.294 mmol) and hydroxylamine hydrochloride (1:1) (24 mg, 0.352 mmol) were dissolved in Ethanol (2 mL) and the reaction heated to 80° C. with stirring for 1 hour. After this time the reaction mixture was cooled to room temperature and the solvent removed under reduced pressure. The crude product was purified prep HPLC (Method G) to afford the title compound (14 mg, 16% Yield) as a pale yellow solid. 1H NMR (500 MHz, Chloroform-d) δ 8.72 (d,J=1.8 Hz, 1H), 8.35-8.31 (m, 2H), 8.10 (s, 1H), 8.06 (dd,J=8.8, 2.3 Hz, 1H), 6.54 (d,J=1.9 Hz, 1H), 1.36 (s, 6H), 1.26 (s, 6H), 1.08 (s, 1H). LCMS: m/z 286.2 [M+H]+, (ESI+), RT=3.55 (Method A).

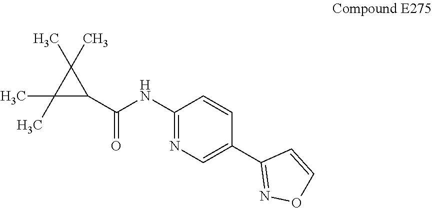 [SMILES: CC1(C)C(C(=O)Nc2ccc(-c3ccon3)cn2)C1(C)C]

### N-(5-isoxazol-3-yl-2-pyridyl)-2,2,3,3-tetramethyl-cyclopropanecarboxamide

Step 1

N-(5-formyl-2-pyridyl)-2,2,3,3-tetramethyl-cyclopropanecarboxamide (Synthesized using a similar method to E199 (step 2), 90 mg, 0.345 mmol) and hydroxylamine hydrochloride (1:1) (36 mg, 0.518 mmol) were dissolved in Water (9 mL) and Methanol (4 mL). Disodium carbonate (66 mg, 0.621 mmol) was added slowly to the reaction mixture. The reaction mixture was stirred at room temperature for 5 hours. A further portion of hydroxylamine hydrochloride (1:1) (36 mg, 0.518 mmol) and disodium carbonate (66 mg, 0.621 mmol) was added along with THF (5 mL). The reaction mixture was stirred at room temperature overnight. The reaction mixture was concentrated under reduced pressure. To the residue was added water (20 ml) and EtOAc (20 ml). The organic was separated and the aqueous extracted with further EtOAc (2×20 ml). The combined organics were dried (hydrophobic frit) and concentrated under reduced pressure to afford N-[5-[hydroxyiminomethyl]-2-pyridyl]-2,2,3,3-tetramethyl-cyclopropanecarboxamide (92 mg, 0.316 mmol, 92% Yield) as a colourless solid.1H NMR (500 MHz, Chloroform-d) δ 8.31 (d,J=1.9 Hz, 1H), 8.16 (d,J=8.7 Hz, 1H), 8.11 (s, 1H), 8.03 (s, 1H), 7.85 (dd,J=8.7, 2.2 Hz, 1H), 7.75 (s, 1H), 1.25 (s, 6H), 1.14 (s, 6H), 0.96 (s, 1H).

Step 2

N-[5-[hydroxyiminomethyl]-2-pyridyl]-2,2,3,3-tetramethyl-cyclopropanecarboxamide (92 mg, 0.351 mmol), calcium ethynediide (70%, 225 mg, 2.46 mmol) and 1-chloropyrrolidine-2,5-dione (59 mg, 0.439 mmol) were dissolved in benzene (1 mL) and DCM (1 mL). The resulting solution was stirred until the oxime dissolved. Water (1.1475 mL) was then added and the reaction stirred at room temperature overnight. The reaction mixture was filtered and the solid washed with chloroform (2×10 ml). The aqueous was then separated and the organics dried (hydrophobic frit). The organics were concentrated under reduced pressure and purified by prep HPLC (Method H) to afford the title compound (8.4 mg, 7.5% Yield) as an off white solid. 1H NMR (400 MHz, Chloroform-d) δ 8.75-8.70 (m, 1H), 8.51 (d,J=1.7 Hz, 1H), 8.32 (d,J=8.7 Hz, 1H), 8.12 (dd,J=8.7, 2.3 Hz, 1H), 8.07 (s, 1H), 6.68 (d,J=1.7 Hz, 1H), 1.36 (s, 6H), 1.26 (s, 6H), 1.07 (s, 1H). LCMS: m/z 286.2 [M+H]+, (ESI+), RT=3.50 (Method A).

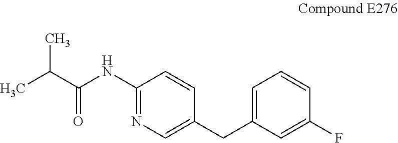 [SMILES: CC(C)C(=O)Nc1ccc(Cc2cccc(F)c2)cn1]

### N-[5-[(3-fluorophenyl)methyl]-2-pyridyl]-2-methyl-propanamide

Step 1

To a solution of 5-bromopyridin-2-amine (1.00 g, 5.78 mmol), N,N-dimethylpyridin-4-amine (71 mg, 0.578 mmol) and triethylamine (1.6 mL, 11.6 mmol) in DCM (20 mL) at 0° C. was added portionwise Boc anhydride (2.78 g, 12.7 mmol) and stirred overnight at RT. The reaction mixture was evaporated to dryness. Purification by flash chromatography (Biotage Isolera, 100 g KP-Sil SNAP cartridge, 2-20% EtOAc in heptane) gave tert-butyl N-(5-bromo-2-pyridyl)-N-tert-butoxycarbonyl-carbamate (1.98 g, 90% pure, 83% Yield) as a white solid. To a solution of tert-butyl N-(5-bromo-2-pyridyl)-N-tert-butoxycarbonyl-carbamate (90%, 1.00 g, 2.41 mmol) in Methanol (10 mL) was added 1 M Sodium hydroxide (2.5 mL, 2.53 mmol), stirred at 50° C. for 2 hours and allowed to cool to RT. This was then concentrated in vacuo, neutralised with 1N HC1 solution, extracted with DCM (2×50 mL), dried over sodium sulfate, filtered and evaporated to dryness to give tert-butyl N-(5-bromo-2-pyridyl)carbamate (99.0%) (EV-PIJ001-088-001) (630 mg, 95% Yield) as a white solid. 1H NMR (250 MHz, Chloroform-d) δ 8.61 (s, 1H), 8.36 (d, J=2.4 Hz, 1H), 7.93 (d, J=8.9 Hz, 1H), 7.75 (dd, J=9.0, 2.5 Hz, 1H), 1.55 (s, 9H).

Step 2

To a stirred solution of tert-butyl N-(5-bromo-2-pyridyl)carbamate (630 mg, 2.28 mmol) in THF-Anhydrous (10 mL) under nitrogen at 0° C. was added a suspension of potassium hydride (30%, 0.42 mL, 4.57 mmol) in THF (5 mL). After 10 minutes the reaction mixture was cooled to −78° C. 3-fluorobenzaldehyde (0.29 mL, 2.74 mmol) was added and stirred for 10 minutes before the addition of 1.6 M butyllithium (2.9 mL, 4.57 mmol) in one portion. The reaction mixture was allowed to stir for 1 h and then allowed to warm to RT and quenched slowly with sat. NH4C1 solution (3 mL). The reaction mixture was diluted with EtOAc (30 mL), washed with water (30 mL) then brine (30 mL), dried over sodium sulfate, filtered and evaporated to dryness. The solid was then adsorbed onto silica gel and purified by flash chromatography (Biotage 100 g KP-Sil SNAP cartridge, 10-80% EtOAc in heptane) to afford tert-butyl N-[5-[(3-fluorophenyl)-hydroxy-methyl]-2-pyridyl]carbamate (396 mg, 52% Yield) as an off-white solid. 1H NMR (250 MHz, Chloroform-d) δ 8.29-8.20 (m, 1H), 7.98-7.78 (m, 2H), 7.68-7.56 (m, 1H), 7.39-7.27 (m, 1H), 7.17-7.04 (m, 2H), 7.04-6.89 (m, 1H), 5.81 (s, 1H), 2.41 (d, J=3.2 Hz, 1H), 1.51 (s, 9H).

Step 3

To a solution of tert-butyl N-[5-[(3-fluorophenyl)-hydroxy-methyl]-2-pyridyl]carbamate (396 mg, 1.19 mmol) in DCE (10 mL) was added 2,2,2-trifluoroacetic acid (3.0 mL, 40.4 mmol) followed by triethylsilane (3.0 mL, 18.8 mmol) and stirred at 50° C. overnight. The reaction mixture was then cooled to RT and evaporated to dryness. Purification by flash chromatography (Biotage C18 30 g KP-Ultra SNAP cartridge, eluting with 10-100% water (+0.1% NH4OH) in MeCN (+0.1% NH4OH)) afforded 5-[(3-fluorophenyl)methyl]pyridin-2-amine (196 mg, 80% Yield) as a beige solid. 1H NMR (250 MHz, Chloroform-d) δ 7.94 (d, J=1.9 Hz, 1H), 7.28-7.15 (m, 2H), 6.99-6.79 (m, 3H), 6.45 (d, J=8.4 Hz, 1H), 4.35 (s, 2H), 3.82 (s, 2H).

Step 4

Starting from 5-[(3-fluorophenyl)methyl]pyridin-2-amine (50 mg, 0.242 mmol), a similar method to Compound E164 (Step 1) was used to afford the title compound (49 mg, 74% Yield) as a white solid. 1H NMR (500 MHz, DMSO-d6) δ 10.34 (s, 1H), 8.22 (d, J=2.0 Hz, 1H), 8.01 (d, J=8.5 Hz, 1H), 7.62 (dd, J=8.5, 2.4 Hz, 1H), 7.37-7.29 (m, 1H), 7.11-7.05 (m, 2H), 7.05-6.98 (m, 1H), 3.93 (s, 2H), 2.78-2.66 (m, 1H), 1.06 (d, J=6.8 Hz, 6H). LCMS: m/z 273.1 [M+H]+, (ESI+), RT=2.85 (Method A).

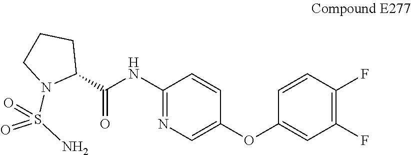 [SMILES: NS(=O)(=O)N1CCC[C@@H]1C(=O)Nc1ccc(Oc2ccc(F)c(F)c2)cn1]

### (2R)-N-[5-(3,4-difluorophenoxy)-2-pyridyl]-1-sulfamoyl-pyrrolidine-2-carboxamide

Step 1

To tert-butyl (2R)-2-[[5-(3,4-difluorophenoxy)-2-pyridyl]carbamoyl]pyrrolidine-1-carboxylate (Synthesized using a similar method to E001 (step 3), 90% pure, 446 mg, 0.957 mmol) was added 4 M hydrogen chloride in dioxane (2.9 mL, 11.5 mmol) and stirred at RT for 1 hr. This was then concentrated in vacuo and purified by SCX-2 cartridge, washing with MeOH and eluting with 7N NH3/MeOH to afford (2R)-N-[5-(3,4-difluorophenoxy)-2-pyridyl]pyrrolidine-2-carboxamide (285 mg, 90% purity, 84% Yield) as an orange oil. 1HNMR(400 MHz, DMSO-d6) δ 10.35 (s, 1H), 8.16 (dd,J=6.0, 2.6 Hz, 2H), 7.59 (dd,J=9.1, 2.9 Hz, 1H), 7.45 (dt,J=10.4, 9.2 Hz, 1H), 7.24 (ddd,J=11.8, 6.8, 3.0 Hz, 1H), 6.88 (dtt,J=8.5, 3.2, 1.8 Hz, 1H), 3.77 (dd,J=9.1, 5.4 Hz, 1H), 3.18 (s, 1H), 2.95 (dt,J=10.2, 6.7 Hz, 1H), 2.84 (dt,J=10.2, 6.4 Hz, 1H), 2.07 (ddt,J=12.5, 8.9, 7.3 Hz, 1H), 1.86-1.74 (m, 1H), 1.65 (p,J=6.5 Hz, 2H).

Step 2

(2R)-N-[5-(3,4-difluorophenoxy)-2-pyridyl]pyrrolidine-2-carboxamide (90%, 47 mg, 0.132 mmol) and sulfuric diamide (22 mg, 0.225 mmol) were stirred in 1,4-Dioxane-Anhydrous (0.9 mL) at 95° C. for 16 hrs. The reaction mixture was concentrated under reduced pressure to dryness and purified by prep HPLC (Method F) to afford the title compound (18 mg, 0.0457 mmol, 35% Yield) as an off white solid. 1HNMR(400 MHz, DMSO-d6) δ 9.81 (s, 1H), 8.20-8.16 (m, 1H), 8.13 (d,J=9.3 Hz, 1H), 7.60 (dd,J=9.0, 3.0 Hz, 1H), 7.46 (dt,J=10.4, 9.2 Hz, 1H), 7.26 (ddd,J=11.8, 6.8, 3.0 Hz, 1H), 7.05 (s, 2H), 6.93-6.85 (m, 1H), 4.28 (dd,J=8.7, 4.4 Hz, 1H), 3.45-3.36 (m, 2H), 2.21-2.09 (m, 1H), 2.07-2.01 (m, 1H), 1.93-1.75 (m, 2H). LCMS: m/z 399.2 [M+H]+, (ESI+), RT=3.03 (Method B).

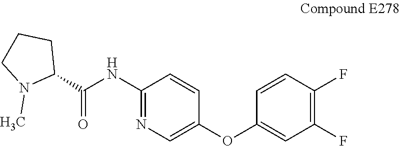 [SMILES: CN1CCC[C@@H]1C(=O)Nc1ccc(Oc2ccc(F)c(F)c2)cn1]

### (2R)-N-[5-(3,4-difluorophenoxy)-2-pyridyl]-1-methyl-pyrrolidine-2-carboxamide

Synthesized from Intermediate I01 using a similar method to that used in Compound E001 (step 3) to afford the title compound as an off-white solid. 1H NMR (400 MHz, DMSO-d6) δ 9.94 (s, 1H), 8.17 (s, 1H), 8.17-8.14 (m, 1H), 7.60 (dd, J=8.9, 3.0 Hz, 1H), 7.46 (dt, J=10.5, 9.2 Hz, 1H), 7.25 (ddd, J=11.8, 6.8, 3.0 Hz, 1H), 6.88 (ddq, J=8.4, 3.4, 1.8 Hz, 1H), 3.18 -3.10 (m, 1H), 3.03 (dd, J=9.8, 5.1 Hz, 1H), 2.42-2.35 (m, 4H), 2.20 (ddd, J=17.0, 11.6, 7.8 Hz, 1H), 1.85-1.70 (m, 3H). LCMS: m/z 334.2 [M+H]+, (ESI+), RT=3.89 (Method B)/

TABLE 14
The following compounds were Synthesized using a similar method to that used in Compound E277.

| Compound | | | LCMS |
|---|---|---|---|
| number | Structure | Analytical data | Method |
| E279 | 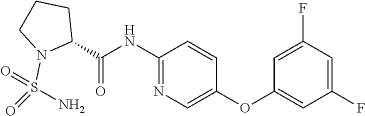 [SMILES: NS(=O)(=O)N1CCC[C@@H]1C(=O)Nc1ccc(Oc2cc(F)cc(F)c2)cn1] | 1H NMR (500 MHz, DMSO-d6) δ 9.83 (s, 1H), 8.26-8.22 (m, 1H), 8.16 (d, J = 9.0 Hz, 1H), 7.68 (dd, J = 9.0, 3.0 Hz, 1H), 7.05 (s, 2H), 7.00 (tt, J = 9.3, 2.3 Hz, 1H), 6.84-6.74 (m, 2H), 4.28 (dd, J = 8.8, 4.4 Hz, 1H), 3.40 (ddd, J = 9.9, 6.8, 4.8 Hz, 1H), 2.20-2.10 (m, 1H), 2.09-2.00 (m, 1H), 1.92-1.77 (m, | A |
| | | 2H), 1H under water peak. | |
| | | LCMS: m/z 399.1 [M + H]+, (ESI+), RT = | |
| | | 2.96 | |
| E280 | 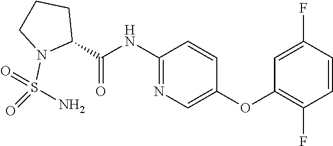 [SMILES: NS(=O)(=O)N1CCC[C@@H]1C(=O)Nc1ccc(Oc2cc(F)ccc2F)cn1] | 1H NMR (500 MHz, DMSO-d6) δ 9.80 (s, 1H), 8.21 (d, J = 3.0 Hz, 1H), 8.12 (d, J = 9.1 Hz, 1H), 7.60 (dd, J = 9.1, 3.0 Hz, 1H), 7.46 (ddd, J = 10.5, 9.2, 5.2 Hz, 1H), 7.16-7.05 (m, 2H), 7.04 (s, 2H), 4.27 (dd, J = 8.8, 4.4 Hz, 1H), 3.43-3.37 (m, 1H), 2.19-2.09 (m, 1H), 2.07-2.00 (m, 1H), 1.93-1.76 (m, 2H), 1H under water peak. | A |
| | | LCMS: m/z 399.1 [M + H]+, (ESI+), RT = | |
| | | 2.80 | |
| E281 | 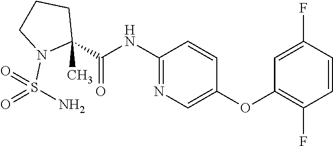 [SMILES: C[C@]1(C(=O)Nc2ccc(Oc3cc(F)ccc3F)cn2)CCCN1S(N)(=O)=O] | 1HNMR(400 MHz, DMSO-d6) δ 8.22 (d, J = 3.0 Hz, 1H), 8.11-8.07 (m, 1H), 7.61 (dd, J = 9.1, 3.0 Hz, 1H), 7.47 (ddd, J = 10.6, 9.1, 5.2 Hz, 1H), 7.15- 7.04 (m, 2H), 3.59-3.50 (m, 1H), 3.44- 3.36 (m, 1H), 2.27 (dd, J = 11.2, 4.0 Hz, 1H), 1.97-1.80 (m, 3H), 1.59 (s, 3H). LCMS: m/z 413.3 [M + H]+, (ESI+), RT = 3.08 | B |
| E282 | 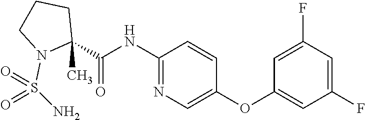 [SMILES: C[C@]1(C(=O)Nc2ccc(Oc3cc(F)cc(F)c3)cn2)CCCN1S(N)(=O)=O] | 1HNMR(400 MHz, DMSO-d6) δ 8.26- 8.22 (m, 1H), 8.14 (dd, J = 9.0, 0.5 Hz, 1H), 7.68 (dd, J = 9.0, 3.0 Hz, 1H), 7.01 (tt, J = 9.3, 2.3 Hz, 1H), 6.83-6.75 (m, 2H), 3.59-3.51 (m, 1H), 3.44-3.36 (m, 1H), 2.28 (dd, J = 10.9, 3.8 Hz, 1H), 1.97-1.82 (m, 3H), 1.60 (s, 3H). LCMS: m/z 413.3 [M + H]+, (ESI+), RT = | B |
| | | 3.24 | |
| E283 | 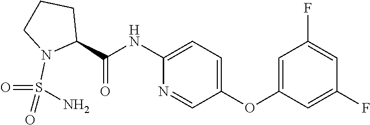 [SMILES: NS(=O)(=O)N1CCC[C@H]1C(=O)Nc1ccc(Oc2cc(F)cc(F)c2)cn1] | 1H NMR (400 MHz, DMSO-d6) δ 8.21- 8.19 (m, 1H), 8.14 (dd, J = 9.0, 0.5 Hz, 1H), 7.64 (dd, J = 9.0, 3.0 Hz, 1H), 6.92 (tt, J = 9.3, 2.3 Hz, 1H), 6.79- 6.69 (m, 2H), 4.32 (dd, J = 8.6, 4.4 Hz, 1H), 3.44-3.34 (m, 2H), 2.24-2.06 (m, 2H), 1.97-1.79 (m, 2H). LCMS: m/z 399.2 [M + H]+, (ESI+), RT = | A |
| | | 2.97 | |

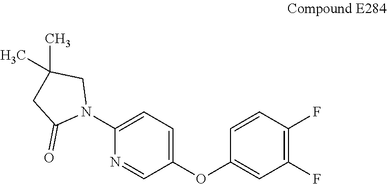 [SMILES: CC1(C)CC(=O)N(c2ccc(Oc3ccc(F)c(F)c3)cn2)C1]

### 1-[5-(3,4-difluorophenoxy)-2-pyridyl]-4,4-dimethyl-pyrrolidin-2-one

Step 1

A mixture of 6-chloropyridin-3-ol (500 mg, 3.86 mmol), (3,4-difluorophenyl)boronic acid (0.91 g, 5.79 mmol), copper(II) diacetate (729 mg, 4.01 mmol), triethylamine (2.7 mL, 19.3 mmol) and powdered activated 4Å molecular sieves in DCM-Anhydrous (38 mL) was stirred under air for 23h. Further copper(II) diacetate (145mg, 0.2 eq) and DCM-Anhydrous (10 mL) were added and the mixture was stirred for 17h. The suspension was diluted with dichloromethane, filtered twice through Celite, washing with water (40 mL) and sat.aq. Rochelle salt (50 mL). The organics were washed with brine (30 mL), dried over MgSO4 and the solvent removed under reduced pressure. The crude product was purified FCC (Biotage KP-Sil 100 g, eluents: 0-100% DCM in heptane) to afford 2-chloro-5-(3,4-difluorophenoxy)pyridine (426 mg, 45% Yield) as a pale brown oil. 1H NMR, (500 MHz, Chloroform-d) δ 8.15 (dd, J=2.9, 0.6 Hz, 1H), 7.33-7.26 (m, 2H), 7.22-7.12 (m, 1H), 6.88 (ddd, J=10.8, 6.5, 2.9 Hz, 1H), 6.75 (dtd, J =9.0, 3.2, 1.9 Hz, 1H).

Step 2

A solution of dipotassium carbonate (90 mg, 0.649 mmol), 2-chloro-5-(3,4-difluorophenoxy)pyridine (80 mg, 0.324 mmol) in Toluene Anhydrous (2.5 mL) was degassed under nitrogen for 15 min at RT and then 4,4-dimethylpyrrolidin-2-one (37 mg, 0.324 mmol) and XPhos Pd G₃ (14 mg, 0.0162 mmol) were added. The reaction vessel was sealed and heated to 90° C. for 16 hours with stirring. It was then allowed to cool to RT, and the reaction mixture was diluted with water (15 mL) and extracted with EtOAc (20 mL), the aqueous layer was then extracted with EtOAc (3×20 mL), the organic extracts combined, dried over hydrophobic filter and evaporated to dryness. The crude compound was purified using flash column chromatography (Biotage Sfar Duo 10 g, 0-80% DCM in heptane, followed by 0-40% MeOH in EtOAc) to afford the title compound (20 mg, 19% Yield) as an orange solid. 1H NMR, (400 MHz, Chloroform-d) δ 8.43 (d, J=9.1 Hz, 1H), 8.11 (d, J=2.9 Hz, 1H), 7.37 (dd, J=9.1, 2.9 Hz, 1H), 7.12 (q, J=9.1 Hz, 1H), 6.82 (ddd, J=11.1, 6.6, 3.0 Hz, 1H), 6.73-6.66 (m, 1H), 3.82 (s, 2H), 2.49 (s, 2H), 1.24 (s, 6H). LCMS: m/z 319.2 [M+H]+, (ESI+), RT=3.89 (Method A)

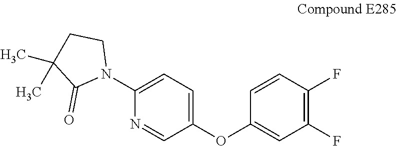 [SMILES: CC1(C)CCN(c2ccc(Oc3ccc(F)c(F)c3)cn2)C1=O]

### 1-[5-(3,4-difluorophenoxy)-2-pyridyl]-3,3-dimethyl-pyrrolidin-2-one

Synthesized using a similar method to that used in Compound E284. 1H NMR (400 MHz, Chloroform-d) δ 8.46 (d, J=9.1 Hz, 1H), 8.13 (d, J=2.9 Hz, 1H), 7.37 (dd, J=9.1, 3.0 Hz, 1H), 7.12 (q, J=9.0 Hz, 1H), 6.82 (ddd, J=11.1, 6.6, 2.9 Hz, 1H), 6.74-6.66 (m, 1H), 4.03 -3.97 (m, 2H), 2.03-1.96 (m, 2H), 1.27 (s, 6H). LCMS: m/z 319.2 [M+H]+, (ESI+), RT=3.93 (Method A).

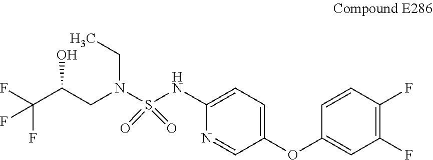 [SMILES: CCN(C[C@@H](O)C(F)(F)F)S(=O)(=O)Nc1ccc(Oc2ccc(F)c(F)c2)cn1]

### 5-(3,4-difluorophenoxy)-2-[[ethyl-[(2R)-3,3,3-trifluoro-2-hydroxy-propyl]sulfamoyl]amino]pyridine

Step 1

Sulfurisocyanatidoyl chloride (58 uL, 0.662 mmol) was added to an ice-cooled solution of DCM (3 mL). 2-chloroethanol (44 uL, 0.662 mmol) was then added via syringe over one minute to maintain an internal temperature of less than 2° C. After the reaction mixture was stirred for lh, N-ethyl-N-isopropyl-propan-2-amine (347 uL, 1.98 mmol) was added. A solution of 5-(3,4-difluorophenoxy)pyridin-2-amine (Intermediate I01, 150 mg, 0.662 mmol) in DCM (3 mL) was added dropwise over 5 min, then stirred for an additional 16h at 0° C. to RT overnight. The reaction was quenched by adding 0.2M HC1(10 mL) and DCM (15 mL). The organic layer was separated and concentrated in vacuo. The residue was triturated with water (2 mL) and then diluted with 3 mL of DCM to form a white solid that was filtered to afford N-[5-(3,4-difluorophenoxy)-2-pyridyl]-2-oxo-oxazolidine-3-sulfonamide (83 mg, 32% Yield) as a white solid. 1H NMR, (500 MHz, Chloroform-d) δ 7.84 (d, J=2.6 Hz, 1H), 7.51 (dd, J=9.4, 2.9 Hz, 1H), 7.27 (s, 1H), 7.25 (s, 1H), 7.17 (q, J=9.0 Hz, 1H), 6.88 (ddd, J=10.7, 6.5, 3.0 Hz, 1H), 6.77-6.70 (m, 1H), 4.40 (dd, J=8.7, 7.0 Hz, 2H), 4.16 (dd, J=8.7, 7.1 Hz, 2H).

Step 2

N-[5-(3,4-difluorophenoxy)-2-pyridyl]-2-oxo-oxazolidine-3-sulfonamide (40 mg, 0.102 mmol), (2R)-3-(ethylamino)-1,1,1-trifluoro-propan-2-ol (80%, 26 mg, 0.133 mmol) and N-ethyl-N-isopropyl-propan-2-amine (0.18 mL, 1.02 mmol) were dissolved in acetonitrile (2 mL). The reaction mixture was warmed to 130° C. using microwave heating for lh. After cooling to RT, it was diluted with EtOAc (10 mL) and washed with aq. sat. ammonium chloride (10 mL) followed by aq. sat. sodium bicarbonate (2×10 mL). The organic layer was separated and concentrated in vacuo. Crude was purified using FCC (Biotage SNAP KP-Sil 10 g, 0-100% EtOAc in heptane) to afford the title compound (4.0 mg, 8.4% Yield) as a pale brown glass. 1H NMR, (500 MHz, Chloroform-d) δ 8.07 (d, J=2.8 Hz, 1H), 7.40 (dd, J=8.8, 2.9 Hz, 1H), 7.16 (q, J=9.0 Hz, 1H), 7.12 (d, J=8.8 Hz, 1H), 6.85 (ddd, J=10.8, 6.5, 2.9 Hz, 1H), 6.75-6.71 (m, 1H), 4.31 (dqd, J=9.6, 6.8, 2.9 Hz, 1H), 3.67 (dd, J=15.2, 9.9 Hz, 1H), 3.58 (dd, J=15.2, 2.8 Hz, 1H), 3.40 (dq, J=14.4, 7.2 Hz, 1H), 3.28 (dq, J=14.3, 7.1 Hz, 1H), 1.18 (t, J=7.1 Hz, 3H), NH and OH not observed. LCMS: m/z 442.2 [M+H]+, (ESI+), RT=3.48 (Method A)

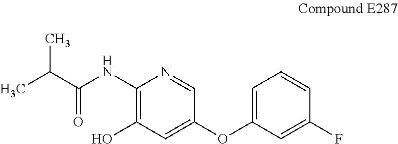 [SMILES: CC(C)C(=O)Nc1ncc(Oc2cccc(F)c2)cc1O]

### N-[5-(3-fluorophenoxy)-3-hydroxy-2-pyridyl]-2-methyl-propanamide

Step 1

5-chloro-2-nitropyridin-3-ol (300.0 mg, 1.72 mmol), cesium carbonate (616.02 mg, 1.89 mmol) and chloromethylbenzene (239.33 mg, 1.89 mmol) were mixed in DMF (4.5 mL), purged with nitrogen and stirred in a sealed vial at RT ° C. for 18 hours. An additional cesium carbonate (616 mg, 1.89 mmol) was added to the reaction mixture, and it was stirred for additional 24 hours at room temperature. The reaction mixture was filtered, and the crude product purified using prep HPLC (Method F) to afford 3-benzyloxy-5-chloro-2-nitro-pyridine (330 mg, 73% Yield) as an off-white solid.

Step 2

3-benzyloxy-5-chloro-2-nitro-pyridine (330 mg, 1.25 mmol), cesium carbonate (406.26 mg, 1.25 mmol), and 3-fluorophenol (139.78 mg, 1.25 mmol) were mixed in DMSO (5 mL), purged with nitrogen and stirred in a sealed vial at 50° C. for 18 hours. Upon completion, the reaction mixture was filtered, and the crude product purified using prep HPLC (Method F) to afford 3-benzyloxy-5-(3-fluorophenoxy)-2-nitro-pyridine (225 mg, 53% Yield) as a yellow solid.

Step 3

3-benzyloxy-5-(3-fluorophenoxy)-2-nitro-pyridine (112.0 mg, 0.33 mmol) was dissolved in Methanol (20 mL) and hydrogenated using H-Cube (RT, 3 hours, 2 mL/min; recirculation mode, 10% Pd/C cartridge). Upon completion, the material was dried in vacuo, mixed with N-ethyl-N-isopropyl-propan-2-amine (0.11 mL, 0.658 mmol) and isoutyric anhydride (52 mg, 0.329 mmol) in THF (4 mL) and stirred in a sealed vial at 80° C. for 18 hours. Upon completion, solvents were removed in vacuo, and the crude material purified using prep HPLC (Method E) to afford the title compound (44 mg, 46% yield) as an off-white solid. 1H NMR (500 MHz, DMSO-d6) δ 10.76 (s, 1H), 10.53 (s, 1H), 7.74 (d, J=2.5 Hz, 1H), 7.44 (td, J =8.3, 6.9 Hz, 1H), 7.06-6.94 (m, 3H), 6.89 (dd, J=8.2, 2.2 Hz, 1H), 2.86 (sept, J=6.8 Hz, 1H), 1.13 (d, J=6.8 Hz, 6H). LCMS: m/z 291.2 [M+H]+, (ESI+), RT=3.33 (Method A).

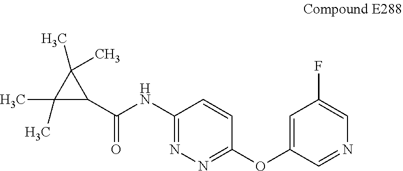 [SMILES: CC1(C)C(C(=O)Nc2ccc(Oc3cncc(F)c3)nn2)C1(C)C]

### N-[6-[(5-fluoro-3-pyridyl)oxy]pyridazin-3-yl]-2,2,3,3-tetramethyl-cyclopropanecarboxamide

Synthesized using a similar method to that used in Compound E217, starting from 6-bromopyridazin-3-amine. 1H NMR (500 MHz, Chloroform-d) δ 8.67 (s, 1H), 8.60 (d,J=9.5 Hz, 1H), 8.40 (d,J=14.0 Hz, 2H), 7.42 (dt,J=9.2, 2.3 Hz, 1H), 7.28 (d,J=9.5 Hz, 1H), 1.35 (s, 6H), 1.22 (s, 6H), 1.18 (s, 1H). LCMS: m/z 331.5 [M+H]+, (ESI+), RT=3.44 (Method A).

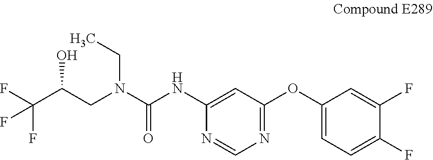 [SMILES: CCN(C[C@@H](O)C(F)(F)F)C(=O)Nc1cc(Oc2ccc(F)c(F)c2)ncn1]

### 3-[6-(3,4-difluorophenoxy)pyrimidin-4-yl]-1-ethyl-1-[(2R)-3,3,3-trifluoro-2-hydroxy-propyl]urea

Step 1

6-chloropyrimidin-4-amine (250 mg, 1.93 mmol), cesium carbonate (1.26 g, 3.86 mmol) and 3,4-difluorophenol (251 mg, 1.93 mmol) were mixed in DMSO (5 mL), purged with nitrogen and stirred in a sealed tube at 50° C. for 3 h followed by 80° C. for 14 h and at 100° C. for 2.5 h. The reaction mixture was then cooled to RT, diluted with water (30 mL) and extracted with EtOAc (2×30 mL). The combined organic extracts were dried over MgSO4, filtered, concentrated under reduced pressure and purified by flash column chromatography (25 g SiO2 column, 25-100% EtOAc in heptane) to afford 6-(3,4-difluorophenoxy)pyrimidin-4-amine (180mg, 70% pure) as an off-white solid. 1H NMR (400 MHz, DMSO-d6) δ 8.07 (d, J=0.8 Hz, 1H), 7.48 (ddd, J=10.6, 9.2, 9.2 Hz, 1H), 7.39 (ddd, J=11.6, 6.9, 2.8 Hz, 1H), 7.03 (dddd, J=9.0, 3.7, 2.9, 1.8 Hz, 1H), 6.90 (s, 2H), 5.80 (d, J=0.9 Hz, 1H).

Step 2

Starting from 6-(3,4-difluorophenoxy)pyrimidin-4-amine (70%, 85 mg, 0.267 mmol), a similar method to Compound E191 was used to afford the title compound (48 mg, 44% Yield) as a white solid. 1H NMR (500 MHz, DMSO-d6) δ 9.57 (s, 1H), 8.43 (d, J=0.8 Hz, 1H), 7.59-7.44 (m, 2H), 7.35 (s, 1H), 7.26-6.73 (m, 2H), 4.40-4.21 (m, 1H), 3.68-3.54 (m, 1H), 3.54-3.38 (m, 3H), 1.10 (t, J=7.0 Hz, 3H). LCMS: m/z 407.2 [M+H]+, (ESI+), RT=3.31 (Method A).

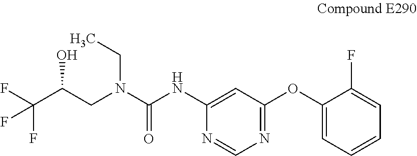 [SMILES: CCN(C[C@@H](O)C(F)(F)F)C(=O)Nc1cc(Oc2ccccc2F)ncn1]

### 1-ethyl-3-[6-(2-fluorophenoxy)pyrimidin-4-yl]-1-[(2R)-3,3,3-trifluoro-2-hydroxy-propyl]urea

Synthesized using a similar method to that used in Compound E289. 1H NMR (500 MHz, DMSO-d6) δ 9.58 (s, 1H), 8.39 (d, J=0.9 Hz, 1H), 7.46-7.30 (m, 4H), 7.30-7.23 (m, 1H), 7.13 (s, 1H), 4.39-4.20 (m, 1H), 3.65-3.54 (m, 1H), 3.54-3.37 (m, 3H), 1.10 (t, J=7.0 Hz, 3H). LCMS: m/z 389.1 [M+H]+, (ESI+), RT=3.19 (Method A).

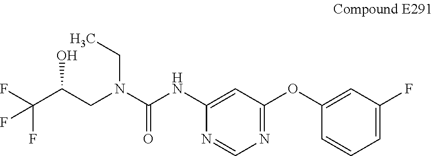 [SMILES: CCN(C[C@@H](O)C(F)(F)F)C(=O)Nc1cc(Oc2cccc(F)c2)ncn1]

### 1-ethyl-3-[6-(3-fluorophenoxy)pyrimidin-4-yl]-1-[(2R)-3,3,3-trifluoro-2-hydroxy-propyl]urea

Synthesized using a similar method to that used in Compound E289. 1H NMR (400 MHz, DMSO-d6) δ 9.55 (s, 1H), 8.42 (d, J=0.9 Hz, 1H), 7.53-7.44 (m, 1H), 7.33 (s, 1H), 7.20 -7.09 (m, 2H), 7.06 (dd, J=8.1, 1.6 Hz, 1H), 4.34-4.20 (m, 1H), 3.62-3.41 (m, 5H), 1.09 (t, J=7.0 Hz, 3H). LCMS: m/z 389.2 [M+H]+, (ESI+), RT=3.33 (Method A).

### HPLC Methods

### Analytical LCMS

Method A

Analytical uHPLC-MS were performed on a Waters Acquity uPLC system using a Phenomenex Kinetex-XB C18 column (2.1 mm×100 mm, 1.7 μM; temperature: 40° C.) and a gradient of 5-100% B (A =0.1% formic acid in H2O; B =0.1% formic acid in ACN) over 5.3 min then 100% B for 0.5 min. A second gradient of 100-5% B was then applied over 0.02 min and held for 1.18 min with an injection volume of 1 μL at flow rate of 0.6 mL/min. UV spectra were recorded at 215 nm using a Waters Acquity PDA detector spectrum range: 200-400 nm, ELS data was collected using a Water Acquity ELS detector (where fitted) were reported. Mass spectra were obtained using a Waters SQD (MSQ1) or Waters Acquity QDA (MSQ2). Data were integrated and reported using Waters MassLynx and OpenLynx software.

Method B

Analytical uPLC-MS were performed on a Waters Acquity uPLC system using a Waters UPLC® BEHTM C18 column (2.1 mm×100 mm, 1.7 μm column; temperature: 40° C.) and a gradient of 5-100% (A=2 mM ammonium bicarbonate, buffered to pH 10; B =ACN) over 5.3 min then 100% B for 0.5 min. A second gradient of 100-5% B was then applied over 0.02 min and held for 1.18 min with an injection volume of 1 μL and at flow rate of 0.6 mL/min. UV spectra were recorded at 215 nm using a Waters Acquity photo diode array detector Spectrum range: 200-400 nm. Mass spectra were obtained using a Waters Quattro Premier XE mass detector. Data were integrated and reported using Waters MassLynx and OpenLynx software.

Method C

Analytical HPLC-MS were performed on a Shimadzu LCMS systems using a Kinetex Core shell C18 column (2.1 mm×50 mm, 5μm; temperature: 40° C.) and a gradient of 5-100% B (A=0.1% formic acid in H2O; B=0.1% formic acid in ACN) over 1.2 min then 100% B for 0.1 min. A second gradient of 100-5% B was then applied over 0.01 min with an injection volume of 3 μL at a flow rate of 1.2 mL/min. UV spectra were recorded at 215 nm using a SPD-M20A photo diode array detector spectrum range: 200-400 nm. Mass spectra were obtained using a 2010EV detector. Data were integrated and reported using Shimadzu LCMS-Solutions and PsiPort software.

Method D

Analytical uHPLC-MS were performed on a Waters Acquity uPLC system using Waters UPLC® BEHTM C18 column (2.1 mm×30 mm, 1.7 μm; temperature 40° C.) and a gradient of 5-100% B (A: 2mM ammonium bicarbonate, buffered to pH 10, B: ACN over 0.75 min, then 100% B for 0.1 min. A second gradient of 100-5% B was then applied over 0.05 min and held for 0.1 min with an injection volume of 1 μL at a flow rate of 1 mL/min. UV spectra were recorded 215 nm Waters Acquity PDA spectrum range of 200-400 nm. Mass spectra were obtained using a Waters Quattro Premier XE. Data were integrated and reported using Waters MassLynx and OpenLynx software.

### Preparative HPLC Methods

Purification methods are as follows:

Method E: ACIDIC EARLY METHOD

Purifications were performed on a Gilson LC system using a Waters Sunfire C18 column (30 mm×100 mm, 10 μM; temperature: r.t.) and a gradient of 10-95% B (A=0.1% formic acid in H2O; B=0.1% formic acid in ACN) over 14.44 min then 95% B for 2.11 min. A second gradient of 95-10% B was then applied over 0.2 min with an injection volume of 1500 μL at flow rate of 40 mL/min. UV spectra were recorded at 215 nm using a Gilson detector.

Method F: BASIC EARLY METHOD

Purifications were performed on a Gilson LC system using a Waters X-Bridge C18 column (30 mm×100 mm, 10 μM; temperature: r.t.) and a gradient of 10-95% B (A=0.2% ammonium hydroxide in H2O; B=0.2% ammonium hydroxide in ACN) over 14.44 min then 95% B for 2.11 min. A second gradient of 95-10% B was then applied over 0.2 min with an injection volume of 1500 μL at flow rate of 40 mL/min. UV spectra were recorded at 215 nm using a Gilson detector.

Method G: ACIDIC STANDARD METHOD

Purifications were performed on a Gilson LC system using a Waters Sunfire C18 column (30 mm×10 mm, 10 μM; temperature: r.t.) and a gradient of 30-95% B (A=0.1% formic acid in water; B=0.1% formic acid in ACN) over 11.00 min then 95% B for 2.10 min. A second gradient of 95-30% B was then applied over 0.2 min with an injection volume of 1500 μL at flow rate of 40 mL/min. UV spectra were recorded at 215 nm using a Gilson detector.

Method H: BASIC STANDARD METHOD

Purifications were performed on a Gilson LC system using a Waters X-Bridge C18 column (30 mm×10 mm, 10 μM; temperature: r.t.) and a gradient of 30-95% B (A=0.2% ammonium hydroxide in water; B=0.2% ammonium hydroxide in ACN) over 11.00 min then 95% B for 2.10 min. A second gradient of 95-30% B was then applied over 0.21 min with an injection volume of 1500 μL at flow rate of 40 mL/min. UV spectra were recorded at 215 nm using a Gilson detector.

### Example 2

### Screening of Compounds

Potent and selective hMrgpMRGPRX2 compounds have been generated from compounds identified during a high throughput screening (HTS) campaign and followed up with cycles of structure activity based medicinal chemistry efforts. These compounds were characterized in recombinant hMrgpMRGPRX2 expressing cells for their antagonist activity and the potency was confirmed in the human mast cell line LAD-2, where the target is endogenously expressed. The assays used to determine potencies are functional read-out looking at intracellular calcium mobilization using the FLIPR™ technology. In these FLIPR assays, we test the identified compounds using recombinant cellular systems expressing mouse MrgprB2, mouse MrgprA1, gerbil MrgpMRGPRX2 orthologue, Chinese hamster MrgpMRGPRX2 orthologue and cynomolgus monkey MrgpMRGPRX2 orthologue, respectively for orthologue activity.

Results are summarized below in Table 15.

TABLE 15

| | | Human |
|---|---|---|
| | | MrgprX2 |
| | | Antagonist |
| Compound | Structure | pIC50 |
| E001 | 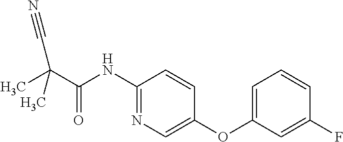 [SMILES: CC(C)(C#N)C(=O)Nc1ccc(Oc2cccc(F)c2)cn1] | 6.4 |
| E002 | 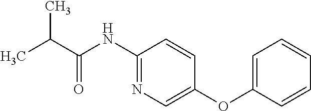 [SMILES: CC(C)C(=O)Nc1ccc(Oc2ccccc2)cn1] | 6.3 |
| E003 | 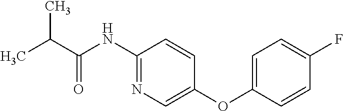 [SMILES: CC(C)C(=O)Nc1ccc(Oc2ccc(F)cc2)cn1] | 6.5 |
| E004 | 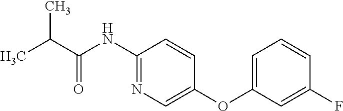 [SMILES: CC(C)C(=O)Nc1ccc(Oc2cccc(F)c2)cn1] | 7.2 |
| E005 | 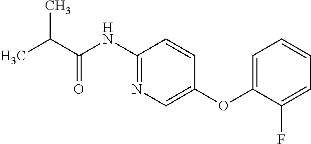 [SMILES: CC(C)C(=O)Nc1ccc(Oc2ccccc2F)cn1] | 7.1 |
| E006 | 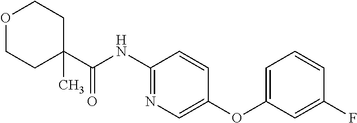 [SMILES: CC1(C(=O)Nc2ccc(Oc3cccc(F)c3)cn2)CCOCC1] | 5.5 |
| E007 | 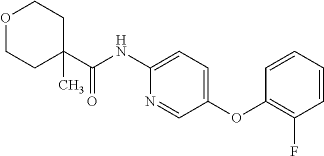 [SMILES: CC1(C(=O)Nc2ccc(Oc3ccccc3F)cn2)CCOCC1] | 5.6 |
| E008 | 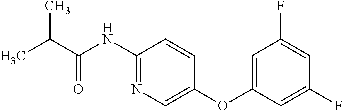 [SMILES: CC(C)C(=O)Nc1ccc(Oc2cc(F)cc(F)c2)cn1] | 8.2 |
| E009 | 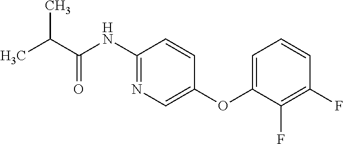 [SMILES: CC(C)C(=O)Nc1ccc(Oc2cccc(F)c2F)cn1] | 7.1 |
| E010 | 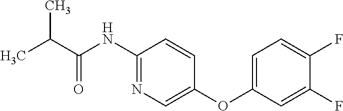 [SMILES: CC(C)C(=O)Nc1ccc(Oc2ccc(F)c(F)c2)cn1] | 7.4 |
| E011 | 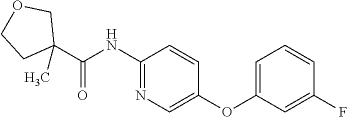 [SMILES: CC1(C(=O)Nc2ccc(Oc3cccc(F)c3)cn2)CCOC1] | 5.2 |
| E012 | 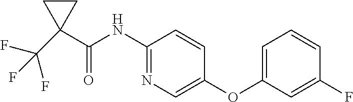 [SMILES: O=C(Nc1ccc(Oc2cccc(F)c2)cn1)C1(C(F)(F)F)CC1] | 6.4 |
| E013 | 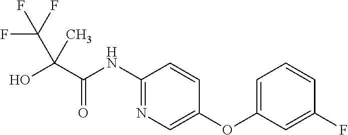 [SMILES: CC(O)(C(=O)Nc1ccc(Oc2cccc(F)c2)cn1)C(F)(F)F] | 6.1 |
| E014 | 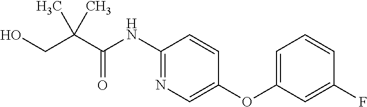 [SMILES: CC(C)(CO)C(=O)Nc1ccc(Oc2cccc(F)c2)cn1] | 5.4 |
| E015 | 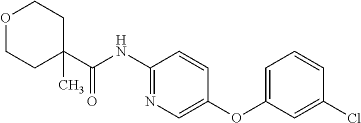 [SMILES: CC1(C(=O)Nc2ccc(Oc3cccc(Cl)c3)cn2)CCOCC1] | 7.6 |
| E016 | 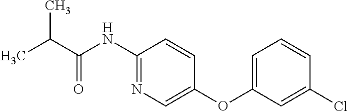 [SMILES: CC(C)C(=O)Nc1ccc(Oc2cccc(Cl)c2)cn1] | 7.6 |
| E017 | 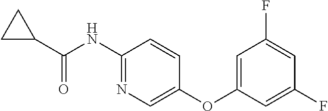 [SMILES: O=C(Nc1ccc(Oc2cc(F)cc(F)c2)cn1)C1CC1] | 7.1 |
| E018 | 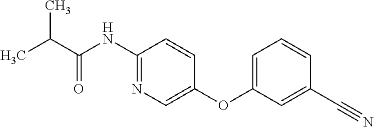 [SMILES: CC(C)C(=O)Nc1ccc(Oc2cccc(C#N)c2)cn1] | 5.2 |
| E019 | 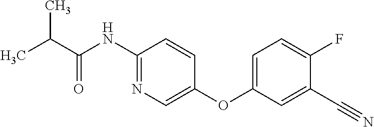 [SMILES: CC(C)C(=O)Nc1ccc(Oc2ccc(F)c(C#N)c2)cn1] | 5.1 |
| E020 | 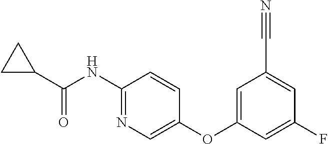 [SMILES: N#Cc1cc(F)cc(Oc2ccc(NC(=O)C3CC3)nc2)c1] | 5.0 |
| E021 | 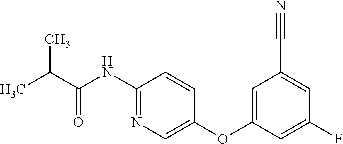 [SMILES: CC(C)C(=O)Nc1ccc(Oc2cc(F)cc(C#N)c2)cn1] | 5.8 |
| E022 | 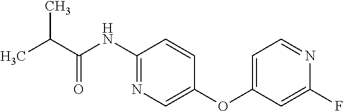 [SMILES: CC(C)C(=O)Nc1ccc(Oc2ccnc(F)c2)cn1] | 5.0 |
| E023 | 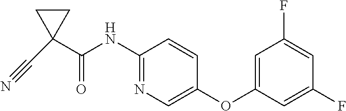 [SMILES: N#CC1(C(=O)Nc2ccc(Oc3cc(F)cc(F)c3)cn2)CC1] | 5.5 |
| E024 | 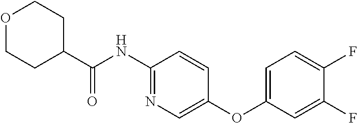 [SMILES: O=C(Nc1ccc(Oc2ccc(F)c(F)c2)cn1)C1CCOCC1] | 5.7 |
| E025 | 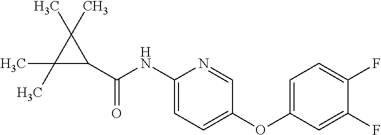 [SMILES: CC1(C)C(C(=O)Nc2ccc(Oc3ccc(F)c(F)c3)cn2)C1(C)C] | 8.6 |
| E026 | 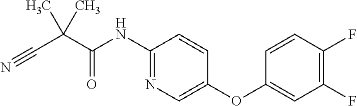 [SMILES: CC(C)(C#N)C(=O)Nc1ccc(Oc2ccc(F)c(F)c2)cn1] | 5.7 |
| E027 | 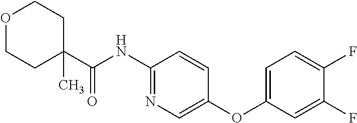 [SMILES: CC1(C(=O)Nc2ccc(Oc3ccc(F)c(F)c3)cn2)CCOCC1] | 5.5 |
| E028 | 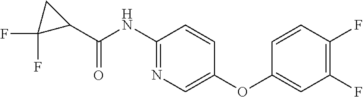 [SMILES: O=C(Nc1ccc(Oc2ccc(F)c(F)c2)cn1)C1CC1(F)F] | 6.7 |
| E029 | 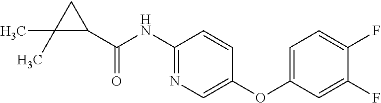 [SMILES: CC1(C)CC1C(=O)Nc1ccc(Oc2ccc(F)c(F)c2)cn1] | 8.4 |
| E030 | 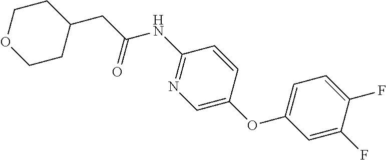 [SMILES: O=C(CC1CCOCC1)Nc1ccc(Oc2ccc(F)c(F)c2)cn1] | 5.1 |
| E031 | 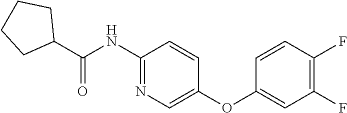 [SMILES: O=C(Nc1ccc(Oc2ccc(F)c(F)c2)cn1)C1CCCC1] | 7.2 |
| E032 | 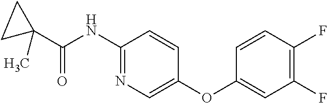 [SMILES: CC1(C(=O)Nc2ccc(Oc3ccc(F)c(F)c3)cn2)CC1] | 6.7 |
| E033 | 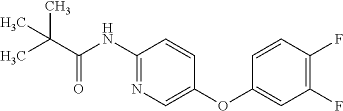 [SMILES: CC(C)(C)C(=O)Nc1ccc(Oc2ccc(F)c(F)c2)cn1] | 7.1 |
| E034 | 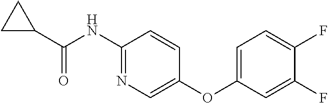 [SMILES: O=C(Nc1ccc(Oc2ccc(F)c(F)c2)cn1)C1CC1] | 6.7 |
| E035 | 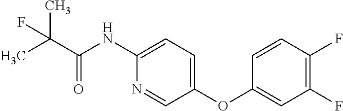 [SMILES: CC(C)(F)C(=O)Nc1ccc(Oc2ccc(F)c(F)c2)cn1] | 6.4 |
| E036 | 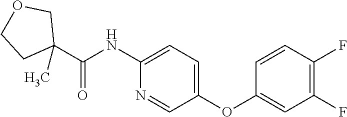 [SMILES: CC1(C(=O)Nc2ccc(Oc3ccc(F)c(F)c3)cn2)CCOC1] | 5.3 |
| E037 | 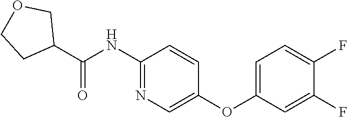 [SMILES: O=C(Nc1ccc(Oc2ccc(F)c(F)c2)cn1)C1CCOC1] | 5.2 |
| E038 | 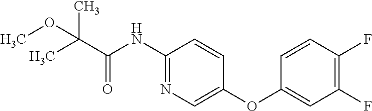 [SMILES: COC(C)(C)C(=O)Nc1ccc(Oc2ccc(F)c(F)c2)cn1] | 6.0 |
| E039 | 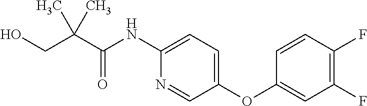 [SMILES: CC(C)(CO)C(=O)Nc1ccc(Oc2ccc(F)c(F)c2)cn1] | 5.4 |
| E040 | 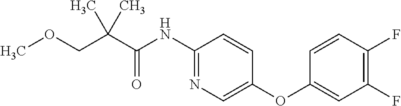 [SMILES: COCC(C)(C)C(=O)Nc1ccc(Oc2ccc(F)c(F)c2)cn1] | 6.3 |
| E041 | 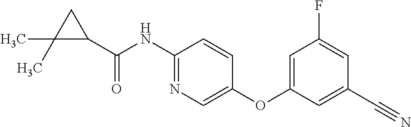 [SMILES: CC1(C)CC1C(=O)Nc1ccc(Oc2cc(F)cc(C#N)c2)cn1] | 6.8 |
| E042 | 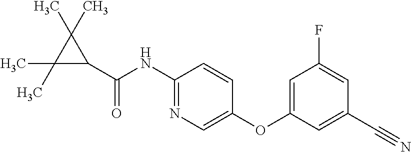 [SMILES: CC1(C)C(C(=O)Nc2ccc(Oc3cc(F)cc(C#N)c3)cn2)C1(C)C] | 8.5 |
| E043 | 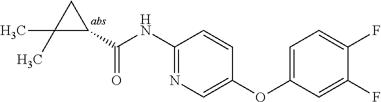 [SMILES: CC1(C)C[C@@H]1C(=O)Nc1ccc(Oc2ccc(F)c(F)c2)cn1] | 8.4 |
| E044 | 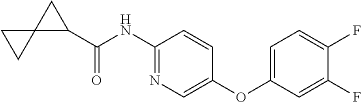 [SMILES: O=C(Nc1ccc(Oc2ccc(F)c(F)c2)cn1)C1CC12CC2] | 7.9 |
| E045 | 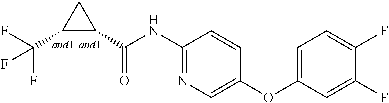 [SMILES: O=C(Nc1ccc(Oc2ccc(F)c(F)c2)cn1)[C@H]1C[C@H]1C(F)(F)F] | 7.7 |
| E046 | 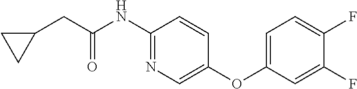 [SMILES: O=C(CC1CC1)Nc1ccc(Oc2ccc(F)c(F)c2)cn1] | 6.4 |
| E047 | 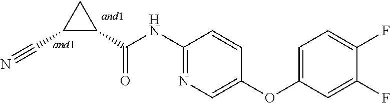 [SMILES: N#C[C@@H]1C[C@@H]1C(=O)Nc1ccc(Oc2ccc(F)c(F)c2)cn1] | 5.5 |
| E048 | 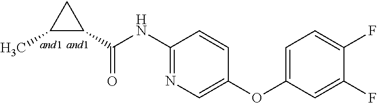 [SMILES: C[C@@H]1C[C@@H]1C(=O)Nc1ccc(Oc2ccc(F)c(F)c2)cn1] | 7.8 |
| E048.1 | 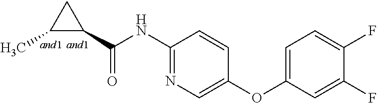 [SMILES: C[C@@H]1C[C@H]1C(=O)Nc1ccc(Oc2ccc(F)c(F)c2)cn1] | 7.1 |
| E049 | 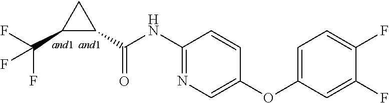 [SMILES: O=C(Nc1ccc(Oc2ccc(F)c(F)c2)cn1)[C@H]1C[C@@H]1C(F)(F)F] | 7.6 |
| E050 | 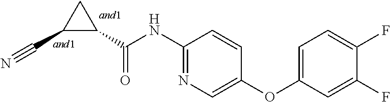 [SMILES: N#C[C@H]1C[C@@H]1C(=O)Nc1ccc(Oc2ccc(F)c(F)c2)cn1] | 6.4 |
| E051 | 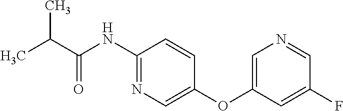 [SMILES: CC(C)C(=O)Nc1ccc(Oc2cncc(F)c2)cn1] | 5.4 |
| E052 | 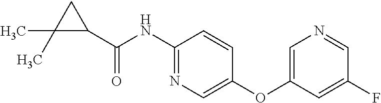 [SMILES: CC1(C)CC1C(=O)Nc1ccc(Oc2cncc(F)c2)cn1] | 6.3 |
| E0530 | 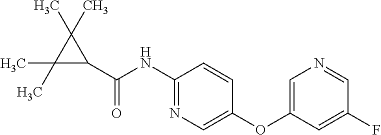 [SMILES: CC1(C)C(C(=O)Nc2ccc(Oc3cncc(F)c3)cn2)C1(C)C] | 8.7 |
| E054 | 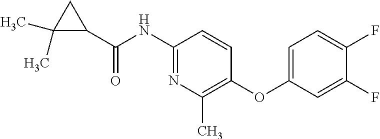 [SMILES: Cc1nc(NC(=O)C2CC2(C)C)ccc1Oc1ccc(F)c(F)c1] | 6.8 |
| E055 | 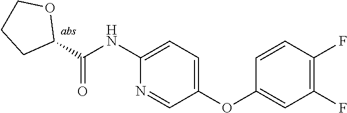 [SMILES: O=C(Nc1ccc(Oc2ccc(F)c(F)c2)cn1)[C@@H]1CCCO1] | 5.2 |
| E056 | 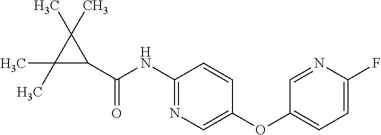 [SMILES: CC1(C)C(C(=O)Nc2ccc(Oc3ccc(F)nc3)cn2)C1(C)C] | 8.3 |
| E057 | 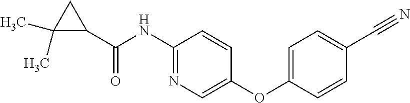 [SMILES: CC1(C)CC1C(=O)Nc1ccc(Oc2ccc(C#N)cc2)cn1] | 5.9 |
| E058 | 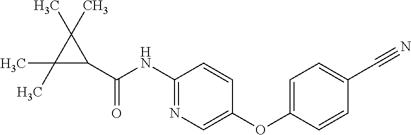 [SMILES: CC1(C)C(C(=O)Nc2ccc(Oc3ccc(C#N)cc3)cn2)C1(C)C] | 7.5 |
| E059 | 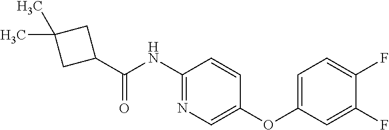 [SMILES: CC1(C)CC(C(=O)Nc2ccc(Oc3ccc(F)c(F)c3)cn2)C1] | 6.7 |
| E060 | 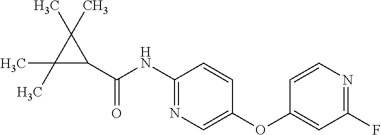 [SMILES: CC1(C)C(C(=O)Nc2ccc(Oc3ccnc(F)c3)cn2)C1(C)C] | 8.5 |
| E061 | 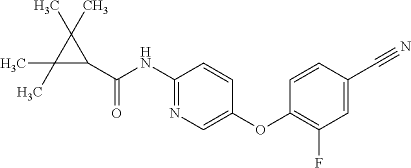 [SMILES: CC1(C)C(C(=O)Nc2ccc(Oc3ccc(C#N)cc3F)cn2)C1(C)C] | 7.7 |
| E062 | 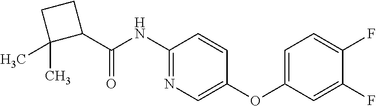 [SMILES: CC1(C)CCC1C(=O)Nc1ccc(Oc2ccc(F)c(F)c2)cn1] | 8.0 |
| E063 | 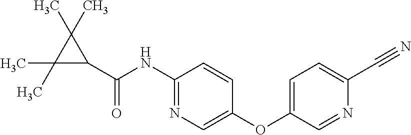 [SMILES: CC1(C)C(C(=O)Nc2ccc(Oc3ccc(C#N)nc3)cn2)C1(C)C] | 5.9 |
| E064 | 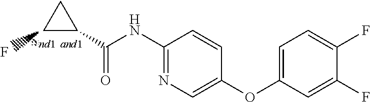 | 6.64 |
| E065 | 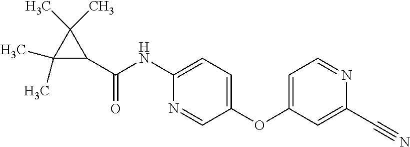 [SMILES: CC1(C)C(C(=O)Nc2ccc(Oc3ccnc(C#N)c3)cn2)C1(C)C] | 6.45 |
| E066 | 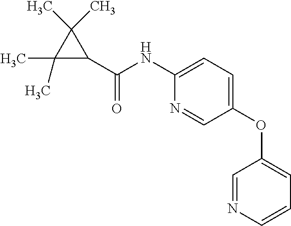 [SMILES: CC1(C)C(C(=O)Nc2ccc(Oc3cccnc3)cn2)C1(C)C] | 7.51 |
| E067 | 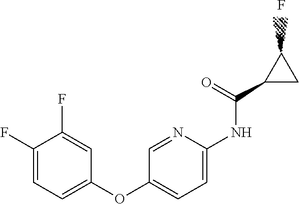 | 6.1 |
| E068 | 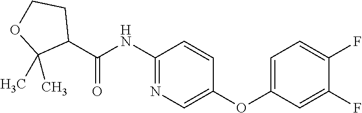 [SMILES: CC1(C)OCCC1C(=O)Nc1ccc(Oc2ccc(F)c(F)c2)cn1] | 6.58 |
| E069 | 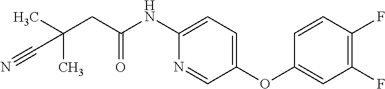 [SMILES: CC(C)(C#N)CC(=O)Nc1ccc(Oc2ccc(F)c(F)c2)cn1] | 5.72 |
| E070 | 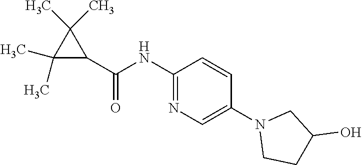 [SMILES: CC1(C)C(C(=O)Nc2ccc(N3CCC(O)C3)cn2)C1(C)C] | 5.98 |
| E071 | 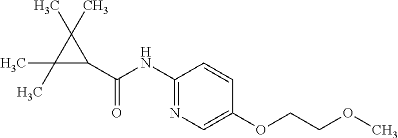 | 5.96 |
| E072 | 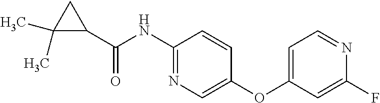 [SMILES: CC1(C)CC1C(=O)Nc1ccc(Oc2ccnc(F)c2)cn1] | 5.72 |
| E073 | 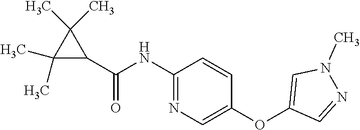 [SMILES: Cn1cc(Oc2ccc(NC(=O)C3C(C)(C)C3(C)C)nc2)cn1] | 8.44 |
| E074 | 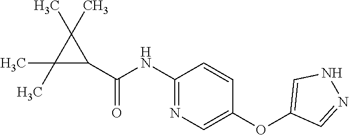 [SMILES: CC1(C)C(C(=O)Nc2ccc(Oc3cn[nH]c3)cn2)C1(C)C] | 6.59 |
| E075 | 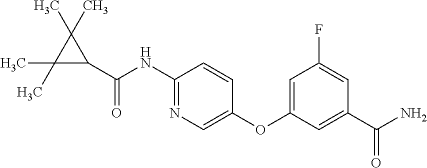 [SMILES: CC1(C)C(C(=O)Nc2ccc(Oc3cc(F)cc(C(N)=O)c3)cn2)C1(C)C] | 6.3 |
| E076 | 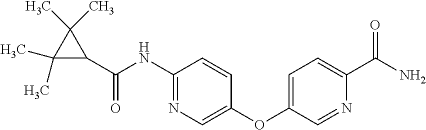 [SMILES: CC1(C)C(C(=O)Nc2ccc(Oc3ccc(C(N)=O)nc3)cn2)C1(C)C] | 5.9 |
| E077 | 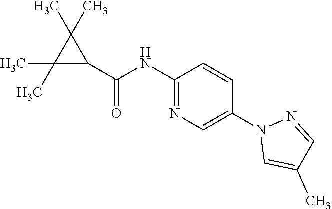 [SMILES: Cc1cnn(-c2ccc(NC(=O)C3C(C)(C)C3(C)C)nc2)c1] | 6.04 |
| E078 | 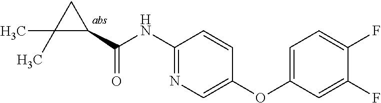 [SMILES: CC1(C)C[C@H]1C(=O)Nc1ccc(Oc2ccc(F)c(F)c2)cn1] | 8.1 |
| E079 | 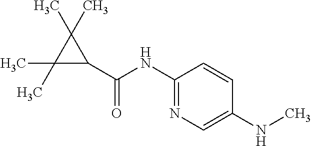 | 5.78 |
| E080 | 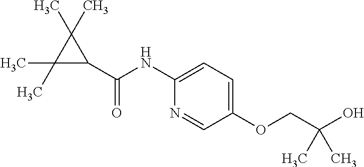 [SMILES: CC(C)(O)COc1ccc(NC(=O)C2C(C)(C)C2(C)C)nc1] | 5.36 |
| E081 | 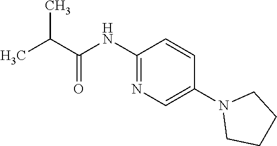 [SMILES: CC(C)C(=O)Nc1ccc(N2CCCC2)cn1] | 5.4 |
| E082 | 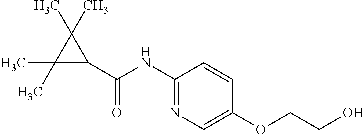 [SMILES: CC1(C)C(C(=O)Nc2ccc(OCCO)cn2)C1(C)C] | 5.36 |
| E083 | 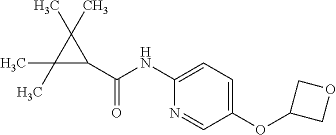 [SMILES: CC1(C)C(C(=O)Nc2ccc(OC3COC3)cn2)C1(C)C] | 5.97 |
| E084 | 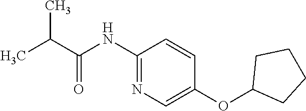 [SMILES: CC(C)C(=O)Nc1ccc(OC2CCCC2)cn1] | 5.8 |
| E085 | 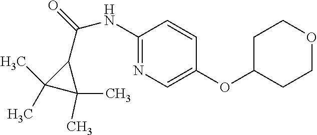 [SMILES: CC1(C)C(C(=O)Nc2ccc(OC3CCOCC3)cn2)C1(C)C] | 6.0 |
| E086 | 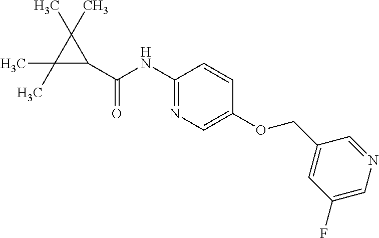 [SMILES: CC1(C)C(C(=O)Nc2ccc(OCc3cncc(F)c3)cn2)C1(C)C] | 6.69 |
| E087 | 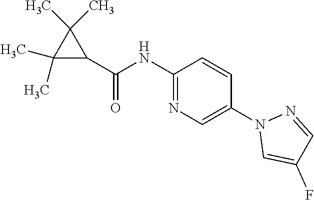 [SMILES: CC1(C)C(C(=O)Nc2ccc(-n3cc(F)cn3)cn2)C1(C)C] | 7.36 |
| E088 | 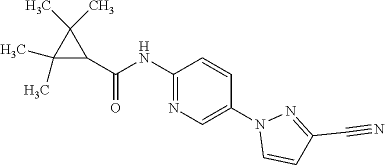 [SMILES: CC1(C)C(C(=O)Nc2ccc(-n3ccc(C#N)n3)cn2)C1(C)C] | 7.55 |
| E089 | 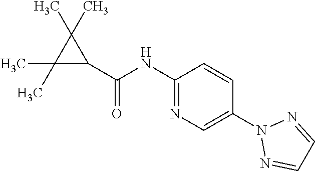 [SMILES: CC1(C)C(C(=O)Nc2ccc(-n3nccn3)cn2)C1(C)C] | 7.82 |
| E090 | 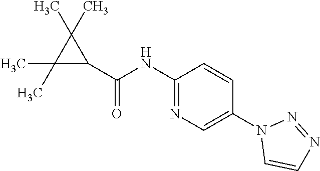 [SMILES: CC1(C)C(C(=O)Nc2ccc(-n3ccnn3)cn2)C1(C)C] | 5.47 |
| E091 | 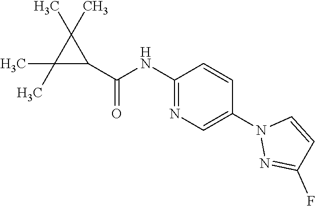 [SMILES: CC1(C)C(C(=O)Nc2ccc(-n3ccc(F)n3)cn2)C1(C)C] | 8.09 |
| E092 | 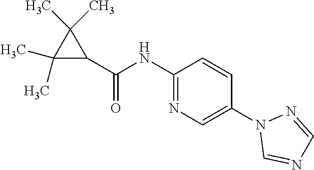 [SMILES: CC1(C)C(C(=O)Nc2ccc(-n3cncn3)cn2)C1(C)C] | 5.09 |
| E093 | 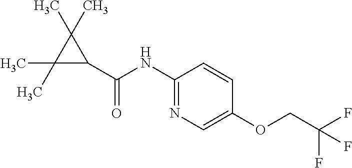 [SMILES: CC1(C)C(C(=O)Nc2ccc(OCC(F)(F)F)cn2)C1(C)C] | 7.5 |
| E094 | 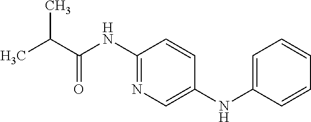 [SMILES: CC(C)C(=O)Nc1ccc(Nc2ccccc2)cn1] | 5.4 |
| E095 | 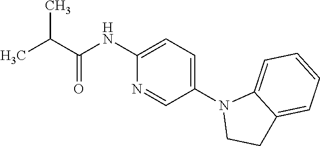 [SMILES: CC(C)C(=O)Nc1ccc(N2CCc3ccccc32)cn1] | 5.4 |
| E096 | 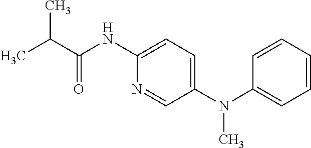 [SMILES: CC(C)C(=O)Nc1ccc(N(C)c2ccccc2)cn1] | 5.6 |
| E097 | 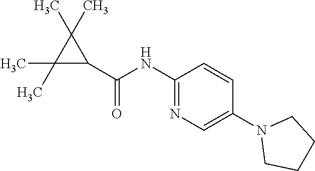 [SMILES: CC1(C)C(C(=O)Nc2ccc(N3CCCC3)cn2)C1(C)C] | 8.6 |
| E098 | 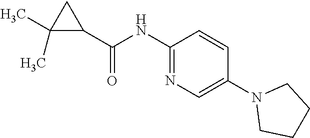 [SMILES: CC1(C)CC1C(=O)Nc1ccc(N2CCCC2)cn1] | 7.06 |
| E099 | 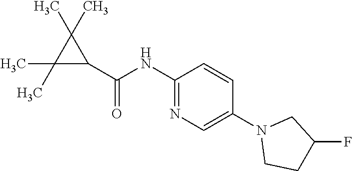 [SMILES: CC1(C)C(C(=O)Nc2ccc(N3CCC(F)C3)cn2)C1(C)C] | 7.6 |
| E100 | 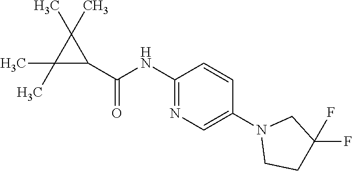 [SMILES: CC1(C)C(C(=O)Nc2ccc(N3CCC(F)(F)C3)cn2)C1(C)C] | 7.46 |
| E101 | 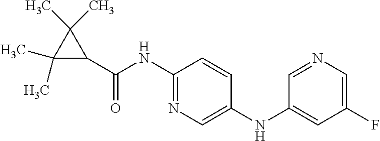 [SMILES: CC1(C)C(C(=O)Nc2ccc(Nc3cncc(F)c3)cn2)C1(C)C] | 6.04 |
| E102 | 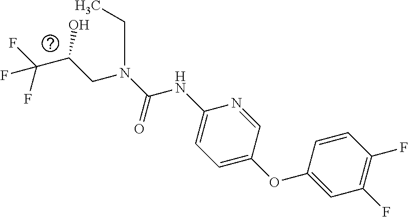 [SMILES: CCN(C[C@@H](O)C(F)(F)F)C(=O)Nc1ccc(Oc2ccc(F)c(F)c2)cn1] | 7.97 |
| E103 | 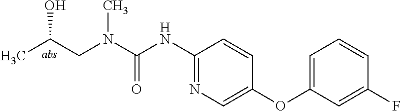 [SMILES: C[C@H](O)CN(C)C(=O)Nc1ccc(Oc2cccc(F)c2)cn1] | 5.8 |
| E104 | 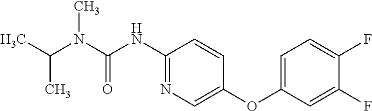 [SMILES: CC(C)N(C)C(=O)Nc1ccc(Oc2ccc(F)c(F)c2)cn1] | 6.9 |
| E105 | 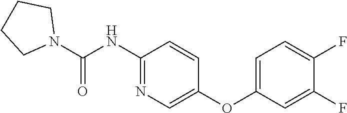 [SMILES: O=C(Nc1ccc(Oc2ccc(F)c(F)c2)cn1)N1CCCC1] | 5.1 |
| E106 | 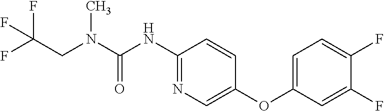 [SMILES: CN(CC(F)(F)F)C(=O)Nc1ccc(Oc2ccc(F)c(F)c2)cn1] | 6.5 |
| E107 | 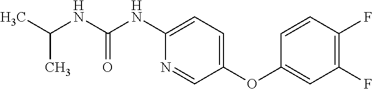 [SMILES: CC(C)NC(=O)Nc1ccc(Oc2ccc(F)c(F)c2)cn1] | 5.2 |
| E108 | 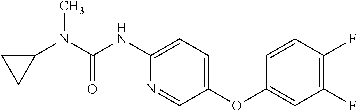 [SMILES: CN(C(=O)Nc1ccc(Oc2ccc(F)c(F)c2)cn1)C1CC1] | 6.2 |
| E109 | 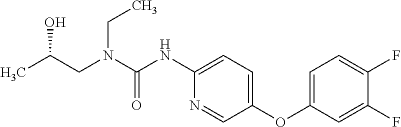 [SMILES: CCN(C[C@H](C)O)C(=O)Nc1ccc(Oc2ccc(F)c(F)c2)cn1] | 6.44 |
| E110 | 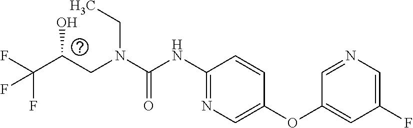 [SMILES: CCN(C[C@@H](O)C(F)(F)F)C(=O)Nc1ccc(Oc2cncc(F)c2)cn1] | 5.29 |
| E111 | 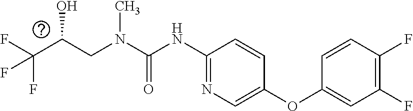 [SMILES: CN(C[C@@H](O)C(F)(F)F)C(=O)Nc1ccc(Oc2ccc(F)c(F)c2)cn1] | 7.24 |
| E112 | 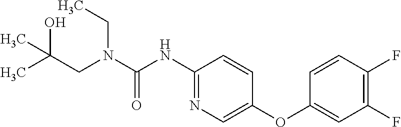 [SMILES: CCN(CC(C)(C)O)C(=O)Nc1ccc(Oc2ccc(F)c(F)c2)cn1] | 6.81 |
| E113 | 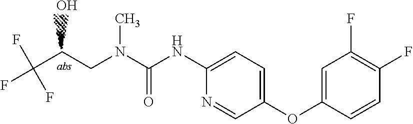 | 6.24 |
| E114 | 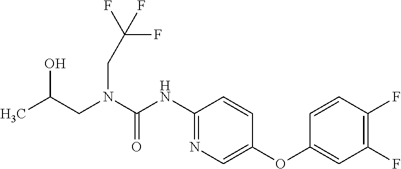 [SMILES: CC(O)CN(CC(F)(F)F)C(=O)Nc1ccc(Oc2ccc(F)c(F)c2)cn1] | 5.92 |
| E115 | 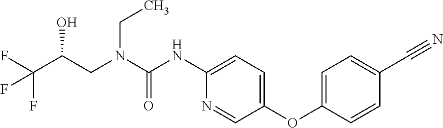 [SMILES: CCN(C[C@@H](O)C(F)(F)F)C(=O)Nc1ccc(Oc2ccc(C#N)cc2)cn1] | 5.05 |
| E116 | 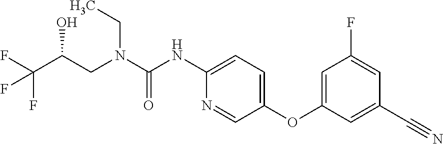 [SMILES: CCN(C[C@@H](O)C(F)(F)F)C(=O)Nc1ccc(Oc2cc(F)cc(C#N)c2)cn1] | 5.87 |
| E117 | 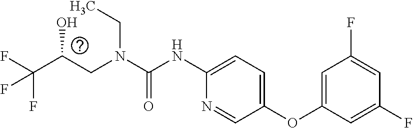 [SMILES: CCN(C[C@@H](O)C(F)(F)F)C(=O)Nc1ccc(Oc2cc(F)cc(F)c2)cn1] | 8.69 |
| E118 | 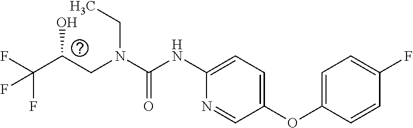 [SMILES: CCN(C[C@@H](O)C(F)(F)F)C(=O)Nc1ccc(Oc2ccc(F)cc2)cn1] | 7.3 |
| E119 | 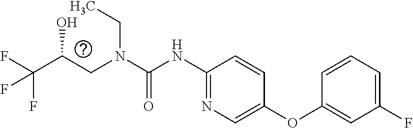 [SMILES: CCN(C[C@@H](O)C(F)(F)F)C(=O)Nc1ccc(Oc2cccc(F)c2)cn1] | 7.91 |
| E120 | 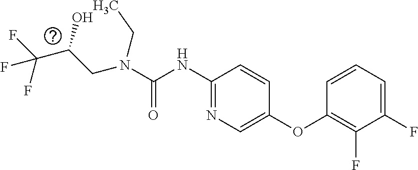 [SMILES: CCN(C[C@@H](O)C(F)(F)F)C(=O)Nc1ccc(Oc2cccc(F)c2F)cn1] | 8.43 |
| E121 | 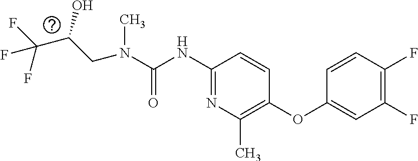 [SMILES: Cc1nc(NC(=O)N(C)C[C@@H](O)C(F)(F)F)ccc1Oc1ccc(F)c(F)c1] | 6.15 |
| E122 | 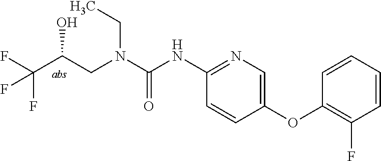 [SMILES: CCN(C[C@@H](O)C(F)(F)F)C(=O)Nc1ccc(Oc2ccccc2F)cn1] | 8.42 |
| E123 | 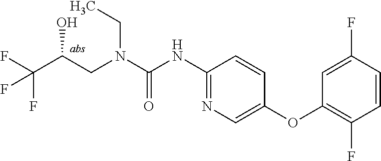 [SMILES: CCN(C[C@@H](O)C(F)(F)F)C(=O)Nc1ccc(Oc2cc(F)ccc2F)cn1] | 8.47 |
| E124 | 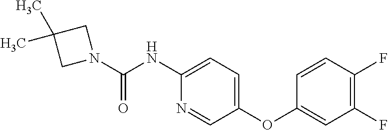 [SMILES: CC1(C)CN(C(=O)Nc2ccc(Oc3ccc(F)c(F)c3)cn2)C1] | 5.49 |
| E125 | 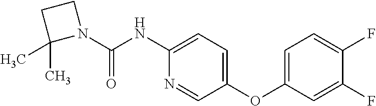 [SMILES: CC1(C)CCN1C(=O)Nc1ccc(Oc2ccc(F)c(F)c2)cn1] | 6.33 |
| E126 | 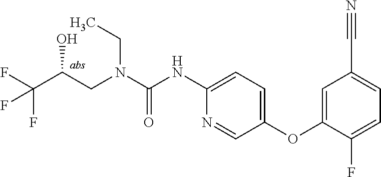 [SMILES: CCN(C[C@@H](O)C(F)(F)F)C(=O)Nc1ccc(Oc2cc(C#N)ccc2F)cn1] | 5.11 |
| E127 | 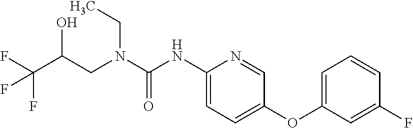 [SMILES: CCN(CC(O)C(F)(F)F)C(=O)Nc1ccc(Oc2cccc(F)c2)cn1] | 7.8 |
| E128 | 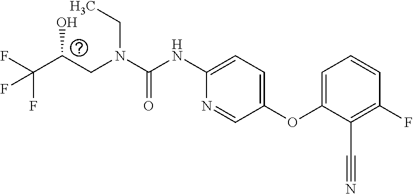 [SMILES: CCN(C[C@@H](O)C(F)(F)F)C(=O)Nc1ccc(Oc2cccc(F)c2C#N)cn1] | 6.92 |
| E129 | 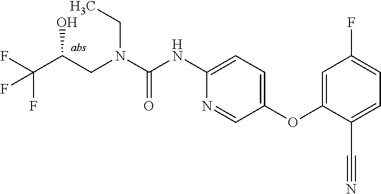 [SMILES: CCN(C[C@@H](O)C(F)(F)F)C(=O)Nc1ccc(Oc2cc(F)ccc2C#N)cn1] | 5.71 |
| E130 | 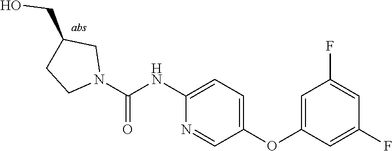 [SMILES: O=C(Nc1ccc(Oc2cc(F)cc(F)c2)cn1)N1CC[C@@H](CO)C1] | 5.15 |
| E131 | 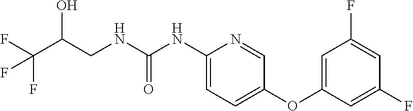 [SMILES: O=C(NCC(O)C(F)(F)F)Nc1ccc(Oc2cc(F)cc(F)c2)cn1] | 6 |
| E132 | 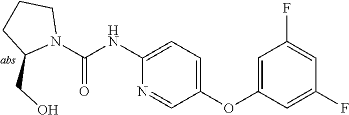 [SMILES: O=C(Nc1ccc(Oc2cc(F)cc(F)c2)cn1)N1CCC[C@@H]1CO] | 6.24 |
| E133 | 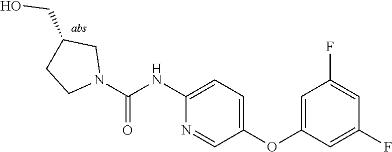 [SMILES: O=C(Nc1ccc(Oc2cc(F)cc(F)c2)cn1)N1CC[C@H](CO)C1] | 5.21 |
| E134 | 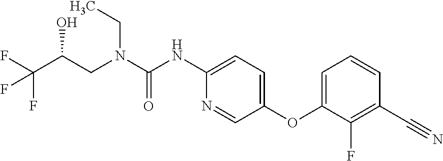 [SMILES: CCN(C[C@@H](O)C(F)(F)F)C(=O)Nc1ccc(Oc2cccc(C#N)c2F)cn1] | 6.33 |
| E135 | 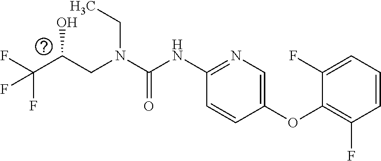 [SMILES: CCN(C[C@@H](O)C(F)(F)F)C(=O)Nc1ccc(Oc2c(F)cccc2F)cn1] | 8.09 |
| E136 | 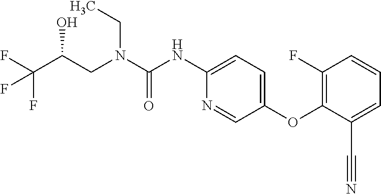 [SMILES: CCN(C[C@@H](O)C(F)(F)F)C(=O)Nc1ccc(Oc2c(F)cccc2C#N)cn1] | 6.8 |
| E137 | 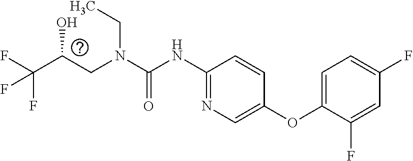 [SMILES: CCN(C[C@@H](O)C(F)(F)F)C(=O)Nc1ccc(Oc2ccc(F)cc2F)cn1] | 8.68 |
| E138 | 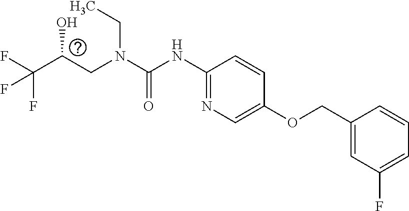 [SMILES: CCN(C[C@@H](O)C(F)(F)F)C(=O)Nc1ccc(OCc2cccc(F)c2)cn1] | 6.06 |
| E139 | 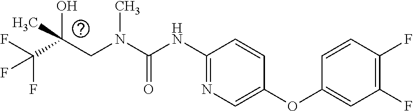 [SMILES: CN(C[C@@](C)(O)C(F)(F)F)C(=O)Nc1ccc(Oc2ccc(F)c(F)c2)cn1] | 7.82 |
| E140 | 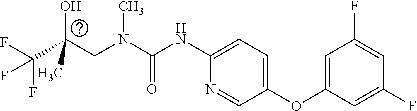 [SMILES: CN(C[C@@](C)(O)C(F)(F)F)C(=O)Nc1ccc(Oc2cc(F)cc(F)c2)cn1] | 8.8 |
| E141 | 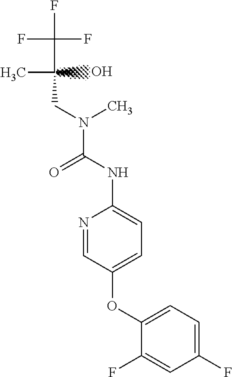 | 8.69 |
| E142 | 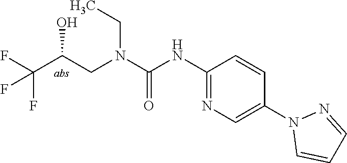 [SMILES: CCN(C[C@@H](O)C(F)(F)F)C(=O)Nc1ccc(-n2cccn2)cn1] | 5.06 |
| E143 | 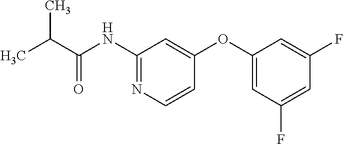 [SMILES: CC(C)C(=O)Nc1cc(Oc2cc(F)cc(F)c2)ccn1] | 6.7 |
| E144 | 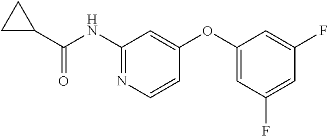 [SMILES: O=C(Nc1cc(Oc2cc(F)cc(F)c2)ccn1)C1CC1] | 6.0 |
| E145 | 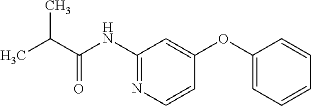 [SMILES: CC(C)C(=O)Nc1cc(Oc2ccccc2)ccn1] | 6.1 |
| E146 | 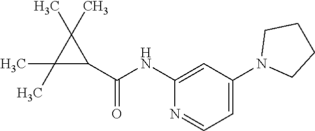 [SMILES: CC1(C)C(C(=O)Nc2cc(N3CCCC3)ccn2)C1(C)C] | 5.1 |
| E147 | 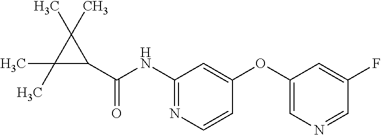 [SMILES: CC1(C)C(C(=O)Nc2cc(Oc3cncc(F)c3)ccn2)C1(C)C] | 8.1 |
| E148 | 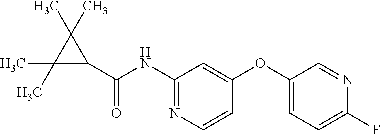 [SMILES: CC1(C)C(C(=O)Nc2cc(Oc3ccc(F)nc3)ccn2)C1(C)C] | 7.52 |
| E149 | 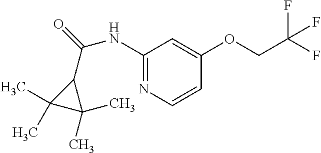 [SMILES: CC1(C)C(C(=O)Nc2cc(OCC(F)(F)F)ccn2)C1(C)C] | 5.7 |
| E150 | 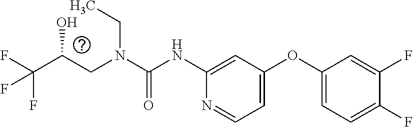 [SMILES: CCN(C[C@@H](O)C(F)(F)F)C(=O)Nc1cc(Oc2ccc(F)c(F)c2)ccn1] | 8.46 |
| E151 | 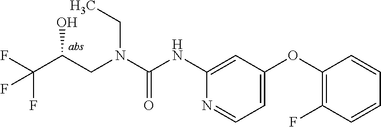 [SMILES: CCN(C[C@@H](O)C(F)(F)F)C(=O)Nc1cc(Oc2ccccc2F)ccn1] | 8.44 |
| E152 | 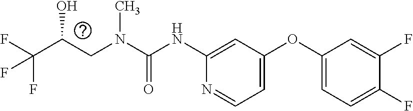 [SMILES: CN(C[C@@H](O)C(F)(F)F)C(=O)Nc1cc(Oc2ccc(F)c(F)c2)ccn1] | 6.58 |
| E153 | 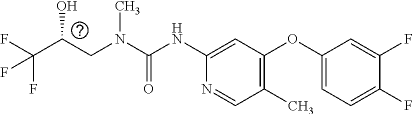 [SMILES: Cc1cnc(NC(=O)N(C)C[C@@H](O)C(F)(F)F)cc1Oc1ccc(F)c(F)c1] | 6.61 |
| E154 | 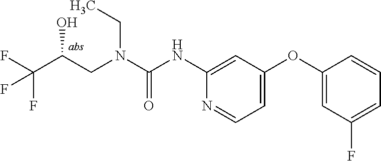 [SMILES: CCN(C[C@@H](O)C(F)(F)F)C(=O)Nc1cc(Oc2cccc(F)c2)ccn1] | 8.89 |
| E155 | 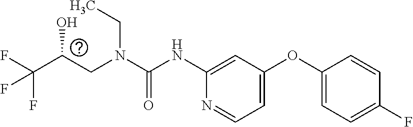 [SMILES: CCN(C[C@@H](O)C(F)(F)F)C(=O)Nc1cc(Oc2ccc(F)cc2)ccn1] | 7.6 |
| E156 | 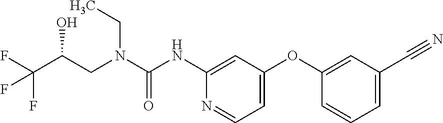 [SMILES: CCN(C[C@@H](O)C(F)(F)F)C(=O)Nc1cc(Oc2cccc(C#N)c2)ccn1] | 5.81 |
| E157 | 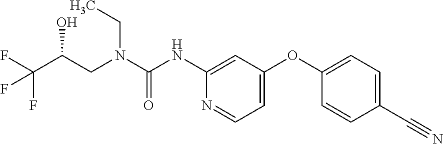 [SMILES: CCN(C[C@@H](O)C(F)(F)F)C(=O)Nc1cc(Oc2ccc(C#N)cc2)ccn1] | 5.8 |
| E158 | 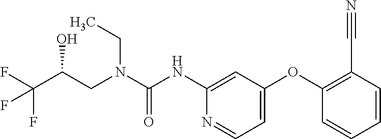 [SMILES: CCN(C[C@@H](O)C(F)(F)F)C(=O)Nc1cc(Oc2ccccc2C#N)ccn1] | 7.75 |
| E159 | 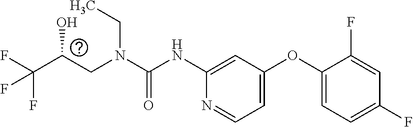 [SMILES: CCN(C[C@@H](O)C(F)(F)F)C(=O)Nc1cc(Oc2ccc(F)cc2F)ccn1] | 7.85 |
| E160 | 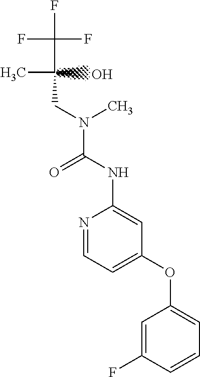 | 7.37 |
| E161 | 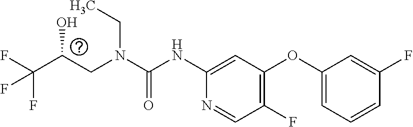 [SMILES: CCN(C[C@@H](O)C(F)(F)F)C(=O)Nc1cc(Oc2cccc(F)c2)c(F)cn1] | 9.05 |
| E162 | 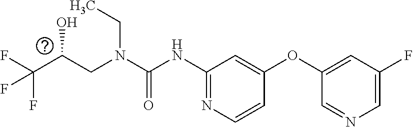 [SMILES: CCN(C[C@@H](O)C(F)(F)F)C(=O)Nc1cc(Oc2cncc(F)c2)ccn1] | 7.72 |
| E163 | 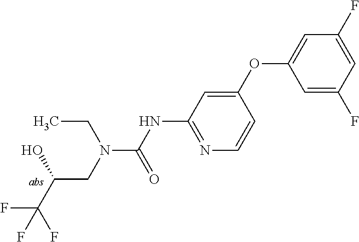 [SMILES: CCN(C[C@@H](O)C(F)(F)F)C(=O)Nc1cc(Oc2cc(F)cc(F)c2)ccn1] | 9.24 |
| E164 | 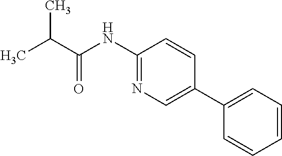 [SMILES: CC(C)C(=O)Nc1ccc(-c2ccccc2)cn1] | 6.6 |
| E165 | 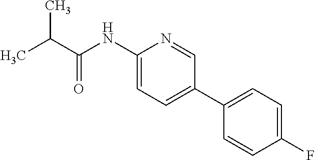 [SMILES: CC(C)C(=O)Nc1ccc(-c2ccc(F)cc2)cn1] | 5.9 |
| E166 | 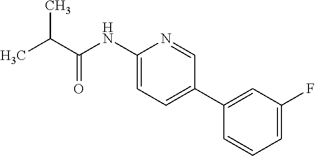 [SMILES: CC(C)C(=O)Nc1ccc(-c2cccc(F)c2)cn1] | 7.2 |
| E167 | 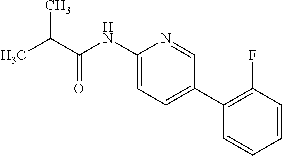 [SMILES: CC(C)C(=O)Nc1ccc(-c2ccccc2F)cn1] | 6.5 |
| E168 | 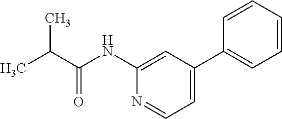 [SMILES: CC(C)C(=O)Nc1cc(-c2ccccc2)ccn1] | 5.1 |
| E169 | 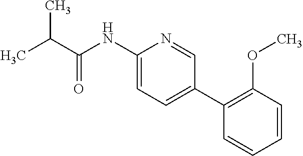 [SMILES: COc1ccccc1-c1ccc(NC(=O)C(C)C)nc1] | 5.7 |
| E170 | 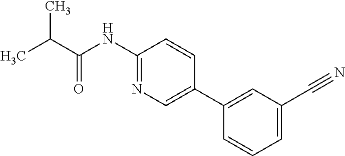 [SMILES: CC(C)C(=O)Nc1ccc(-c2cccc(C#N)c2)cn1] | 6.7 |
| E171 | 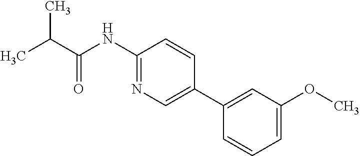 [SMILES: COc1cccc(-c2ccc(NC(=O)C(C)C)nc2)c1] | 6.5 |
| E172 | 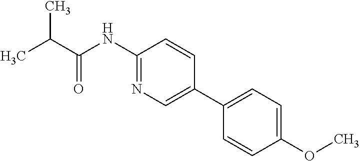 [SMILES: COc1ccc(-c2ccc(NC(=O)C(C)C)nc2)cc1] | 6.5 |
| E173 | 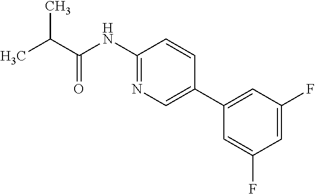 [SMILES: CC(C)C(=O)Nc1ccc(-c2cc(F)cc(F)c2)cn1] | 7.5 |
| E174 | 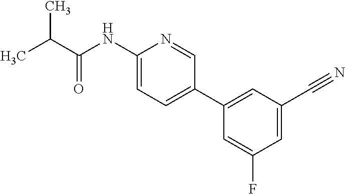 [SMILES: CC(C)C(=O)Nc1ccc(-c2cc(F)cc(C#N)c2)cn1] | 7.0 |
| E175 | 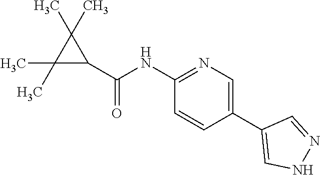 [SMILES: CC1(C)C(C(=O)Nc2ccc(-c3cn[nH]c3)cn2)C1(C)C] | 5.6 |
| E176 | 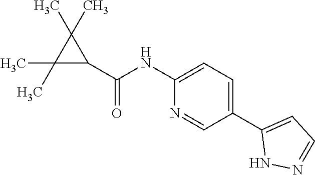 [SMILES: CC1(C)C(C(=O)Nc2ccc(-c3ccn[nH]3)cn2)C1(C)C] | 6.92 |
| E177 | 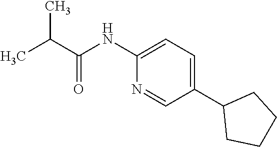 [SMILES: CC(C)C(=O)Nc1ccc(C2CCCC2)cn1] | 5.2 |
| E178 | 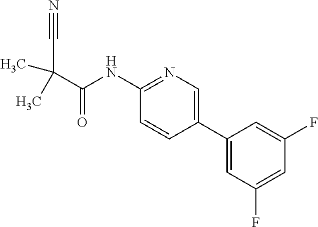 [SMILES: CC(C)(C#N)C(=O)Nc1ccc(-c2cc(F)cc(F)c2)cn1] | 5.9 |
| E179 | 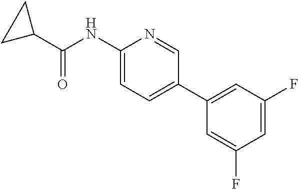 [SMILES: O=C(Nc1ccc(-c2cc(F)cc(F)c2)cn1)C1CC1] | 6.7 |
| E180 | 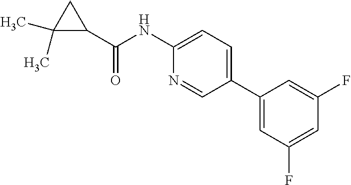 [SMILES: CC1(C)CC1C(=O)Nc1ccc(-c2cc(F)cc(F)c2)cn1] | 7.9 |
| E181 | 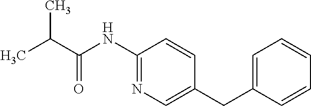 [SMILES: CC(C)C(=O)Nc1ccc(Cc2ccccc2)cn1] | 5.2 |
| E182 | 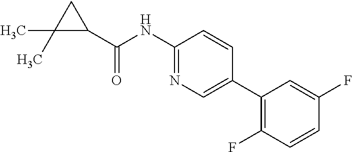 [SMILES: CC1(C)CC1C(=O)Nc1ccc(-c2cc(F)ccc2F)cn1] | 7.6 |
| E183 | 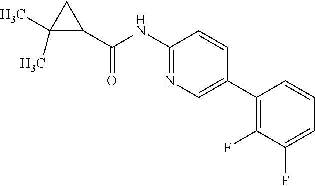 [SMILES: CC1(C)CC1C(=O)Nc1ccc(-c2cccc(F)c2F)cn1] | 7.71 |
| E184 | 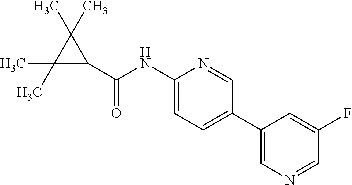 [SMILES: CC1(C)C(C(=O)Nc2ccc(-c3cncc(F)c3)cn2)C1(C)C] | 7.15 |
| E185 | 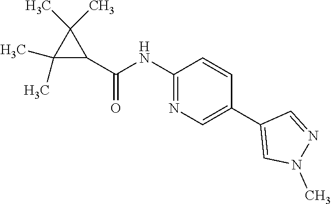 [SMILES: Cn1cc(-c2ccc(NC(=O)C3C(C)(C)C3(C)C)nc2)cn1] | 7.03 |
| E186 | 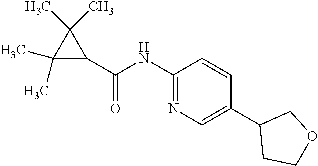 [SMILES: CC1(C)C(C(=O)Nc2ccc(C3CCOC3)cn2)C1(C)C] | 6.73 |
| E187 | 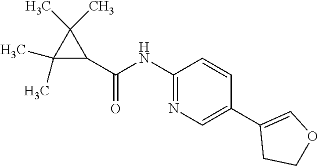 [SMILES: CC1(C)C(C(=O)Nc2ccc(C3=COCC3)cn2)C1(C)C] | 7.84 |
| E188 | 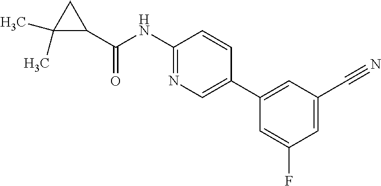 [SMILES: CC1(C)CC1C(=O)Nc1ccc(-c2cc(F)cc(C#N)c2)cn1] | 7.3 |
| E189 | 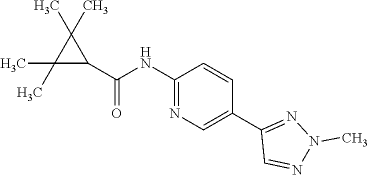 | 5.93 |
| E190 | 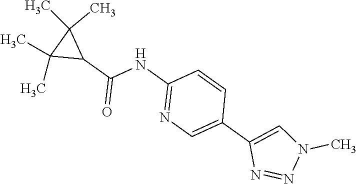 | 6.2 |
| E191 | 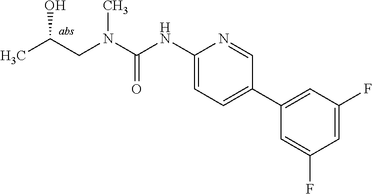 [SMILES: C[C@H](O)CN(C)C(=O)Nc1ccc(-c2cc(F)cc(F)c2)cn1] | 5.6 |
| E192 | 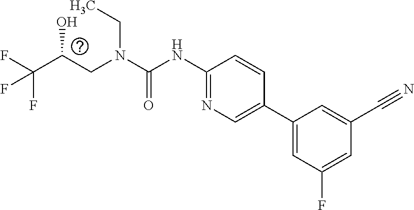 [SMILES: CCN(C[C@@H](O)C(F)(F)F)C(=O)Nc1ccc(-c2cc(F)cc(C#N)c2)cn1] | 5.92 |
| E193 | 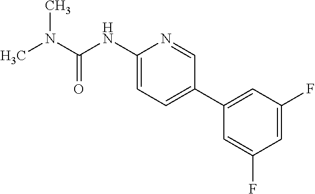 [SMILES: CN(C)C(=O)Nc1ccc(-c2cc(F)cc(F)c2)cn1] | 5.2 |
| E194 | 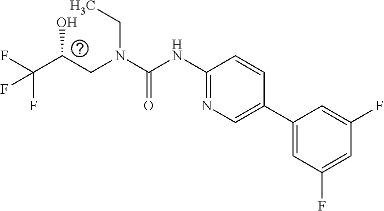 [SMILES: CCN(C[C@@H](O)C(F)(F)F)C(=O)Nc1ccc(-c2cc(F)cc(F)c2)cn1] | 6.67 |
| E195 | 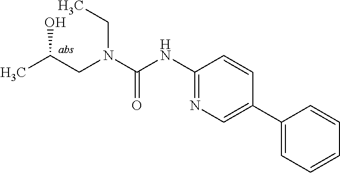 [SMILES: CCN(C[C@H](C)O)C(=O)Nc1ccc(-c2ccccc2)cn1] | 5.3 |
| E196 | 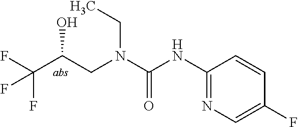 [SMILES: CCN(C[C@@H](O)C(F)(F)F)C(=O)Nc1ccc(F)cn1] | 6.07 |
| E197 | 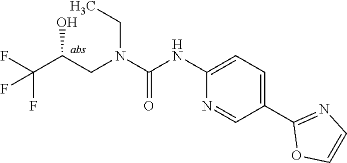 [SMILES: CCN(C[C@@H](O)C(F)(F)F)C(=O)Nc1ccc(-c2ncco2)cn1] | 5.51 |
| E198 | 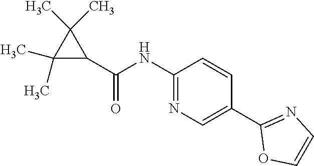 [SMILES: CC1(C)C(C(=O)Nc2ccc(-c3ncco3)cn2)C1(C)C] | 7.47 |
| E199 | 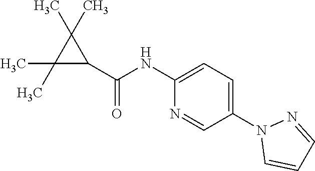 [SMILES: CC1(C)C(C(=O)Nc2ccc(-n3cccn3)cn2)C1(C)C] | 7.47 |
| E200 | 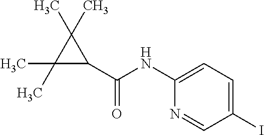 [SMILES: CC1(C)C(C(=O)Nc2ccc(I)cn2)C1(C)C] | 7.83 |
| E201 | 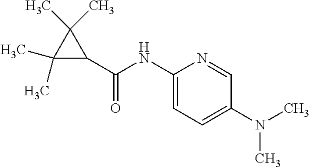 | 6.79 |
| E202 | 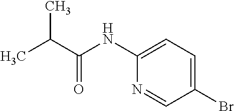 | 5.2 |
| E203 | 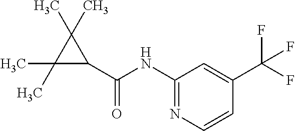 [SMILES: CC1(C)C(C(=O)Nc2cc(C(F)(F)F)ccn2)C1(C)C] | 5.8 |
| E204 | 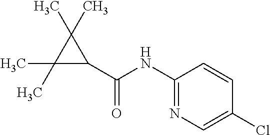 [SMILES: CC1(C)C(C(=O)Nc2ccc(Cl)cn2)C1(C)C] | 8.8 |
| E205 | 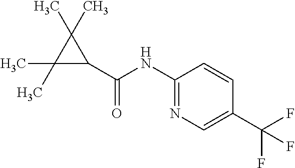 [SMILES: CC1(C)C(C(=O)Nc2ccc(C(F)(F)F)cn2)C1(C)C] | 6.3 |
| E206 | 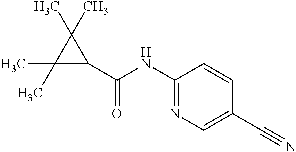 [SMILES: CC1(C)C(C(=O)Nc2ccc(C#N)cn2)C1(C)C] | 6.6 |
| E207 | 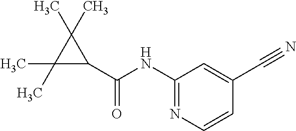 [SMILES: CC1(C)C(C(=O)Nc2cc(C#N)ccn2)C1(C)C] | 5.5 |
| E208 | 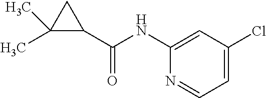 | 5.5 |
| E209 | 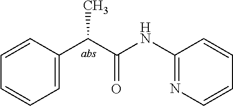 [SMILES: C[C@H](C(=O)Nc1ccccn1)c1ccccc1] | 5.6 |
| E210 | 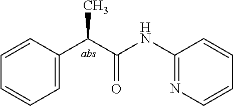 [SMILES: C[C@@H](C(=O)Nc1ccccn1)c1ccccc1] | 5.7 |
| E211 | 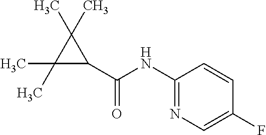 [SMILES: CC1(C)C(C(=O)Nc2ccc(F)cn2)C1(C)C] | 8.9 |
| E212 | 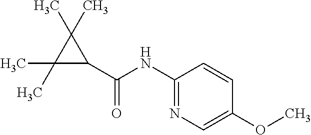 | 7.7 |
| E213 | 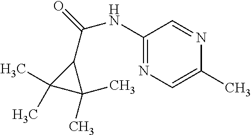 | 5.8 |
| E214 | 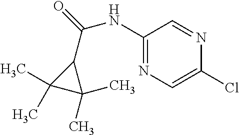 [SMILES: CC1(C)C(C(=O)Nc2cnc(Cl)cn2)C1(C)C] | 7.0 |
| E215 | 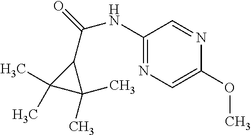 | 6.7 |
| E216 | 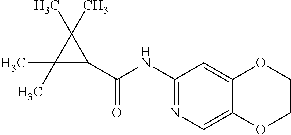 [SMILES: CC1(C)C(C(=O)Nc2cc3c(cn2)OCCO3)C1(C)C] | 6.48 |
| E217 | 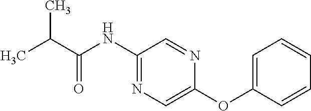 [SMILES: CC(C)C(=O)Nc1cnc(Oc2ccccc2)cn1] | 5.1 |
| E218 | 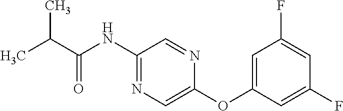 [SMILES: CC(C)C(=O)Nc1cnc(Oc2cc(F)cc(F)c2)cn1] | 6.5 |
| E219 | 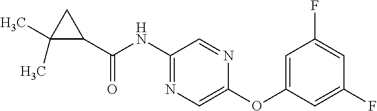 [SMILES: CC1(C)CC1C(=O)Nc1cnc(Oc2cc(F)cc(F)c2)cn1] | 7.6 |
| E220 | 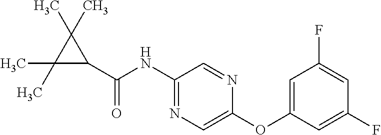 [SMILES: CC1(C)C(C(=O)Nc2cnc(Oc3cc(F)cc(F)c3)cn2)C1(C)C] | 8.8 |
| E221 | 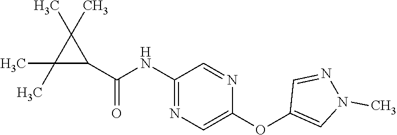 | 7.54 |
| E222 | 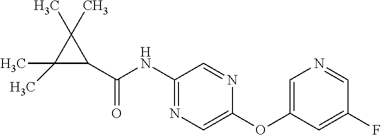 [SMILES: CC1(C)C(C(=O)Nc2cnc(Oc3cncc(F)c3)cn2)C1(C)C] | 7.9 |
| E223 | 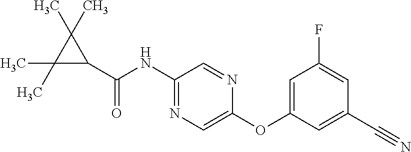 [SMILES: CC1(C)C(C(=O)Nc2cnc(Oc3cc(F)cc(C#N)c3)cn2)C1(C)C] | 7.5 |
| E224 | 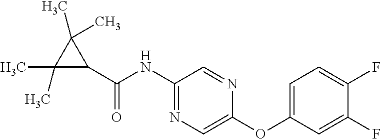 [SMILES: CC1(C)C(C(=O)Nc2cnc(Oc3ccc(F)c(F)c3)cn2)C1(C)C] | 8.4 |
| E225 | 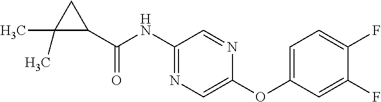 [SMILES: CC1(C)CC1C(=O)Nc1cnc(Oc2ccc(F)c(F)c2)cn1] | 7.2 |
| E226 | 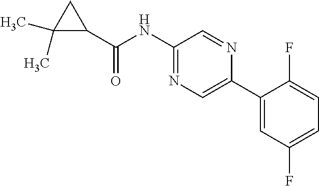 [SMILES: CC1(C)CC1C(=O)Nc1cnc(-c2cc(F)ccc2F)cn1] | 7.46 |
| E227 | 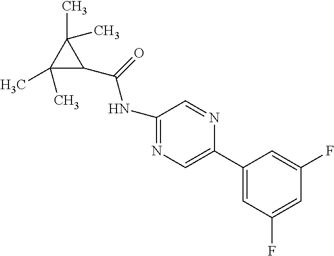 [SMILES: CC1(C)C(C(=O)Nc2cnc(-c3cc(F)cc(F)c3)cn2)C1(C)C] | 7.47 |
| E228 | 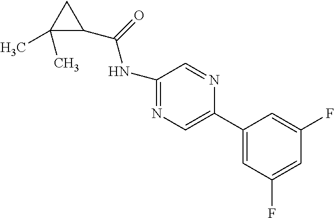 [SMILES: CC1(C)CC1C(=O)Nc1cnc(-c2cc(F)cc(F)c2)cn1] | 7.02 |
| E229 | 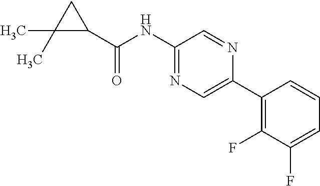 [SMILES: CC1(C)CC1C(=O)Nc1cnc(-c2cccc(F)c2F)cn1] | 5.18 |
| E230 | 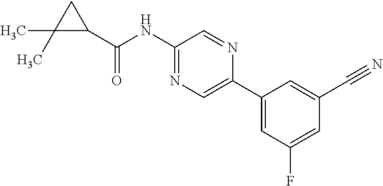 [SMILES: CC1(C)CC1C(=O)Nc1cnc(-c2cc(F)cc(C#N)c2)cn1] | 6.54 |
| E231 | 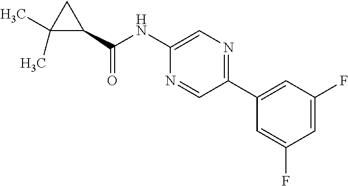 [SMILES: CC1(C)C[C@H]1C(=O)Nc1cnc(-c2cc(F)cc(F)c2)cn1] | 6.99 |
| E233 | 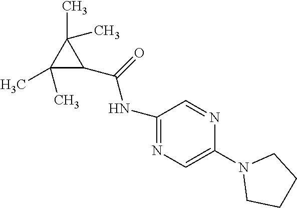 [SMILES: CC1(C)C(C(=O)Nc2cnc(N3CCCC3)cn2)C1(C)C] | 6.48 |
| E234 | 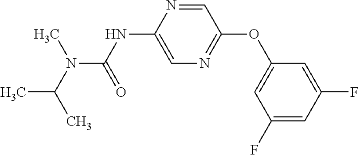 [SMILES: CC(C)N(C)C(=O)Nc1cnc(Oc2cc(F)cc(F)c2)cn1] | 6.2 |
| E235 | 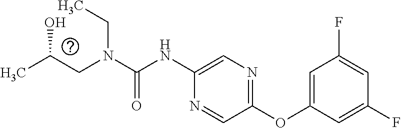 [SMILES: CCN(C[C@H](C)O)C(=O)Nc1cnc(Oc2cc(F)cc(F)c2)cn1] | 5.85 |
| E236 | 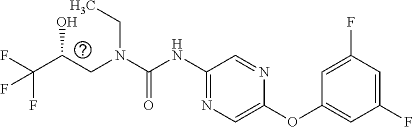 [SMILES: CCN(C[C@@H](O)C(F)(F)F)C(=O)Nc1cnc(Oc2cc(F)cc(F)c2)cn1] | 7.5 |
| E237 | 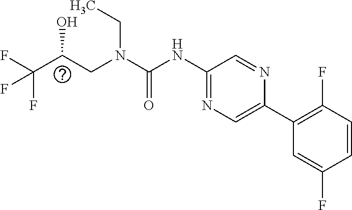 [SMILES: CCN(C[C@@H](O)C(F)(F)F)C(=O)Nc1cnc(-c2cc(F)ccc2F)cn1] | 6.22 |
| E238 | 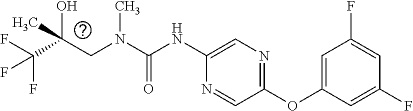 [SMILES: CN(C[C@@](C)(O)C(F)(F)F)C(=O)Nc1cnc(Oc2cc(F)cc(F)c2)cn1] | 7.34 |
| E239 | 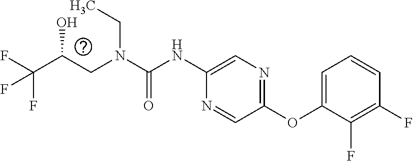 [SMILES: CCN(C[C@@H](O)C(F)(F)F)C(=O)Nc1cnc(Oc2cccc(F)c2F)cn1] | 8.18 |
| E240 | 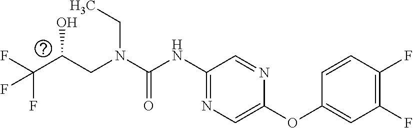 [SMILES: CCN(C[C@@H](O)C(F)(F)F)C(=O)Nc1cnc(Oc2ccc(F)c(F)c2)cn1] | 6.96 |
| E241 | 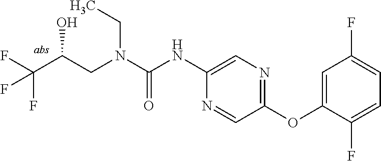 [SMILES: CCN(C[C@@H](O)C(F)(F)F)C(=O)Nc1cnc(Oc2cc(F)ccc2F)cn1] | 8.77 |
| E242 | 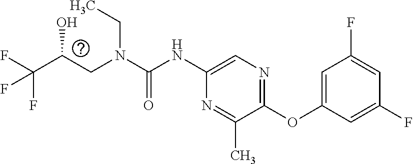 [SMILES: CCN(C[C@@H](O)C(F)(F)F)C(=O)Nc1cnc(Oc2cc(F)cc(F)c2)c(C)n1] | 5.13 |
| E243 | 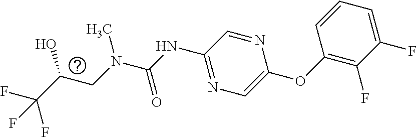 [SMILES: CN(C[C@@H](O)C(F)(F)F)C(=O)Nc1cnc(Oc2cccc(F)c2F)cn1] | 7.23 |
| E244 | 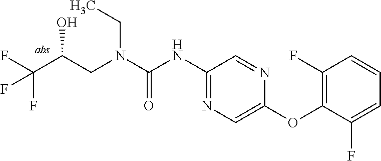 [SMILES: CCN(C[C@@H](O)C(F)(F)F)C(=O)Nc1cnc(Oc2c(F)cccc2F)cn1] | 7.57 |
| E245 | 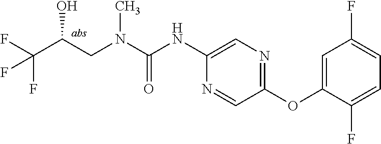 [SMILES: CN(C[C@@H](O)C(F)(F)F)C(=O)Nc1cnc(Oc2cc(F)ccc2F)cn1] | 8.07 |
| E246 | 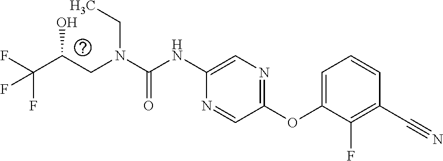 [SMILES: CCN(C[C@@H](O)C(F)(F)F)C(=O)Nc1cnc(Oc2cccc(C#N)c2F)cn1] | 5.67 |
| E247 | 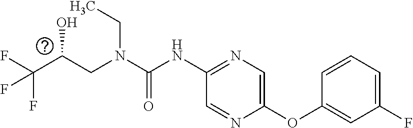 [SMILES: CCN(C[C@@H](O)C(F)(F)F)C(=O)Nc1cnc(Oc2cccc(F)c2)cn1] | 6.52 |
| E248 | 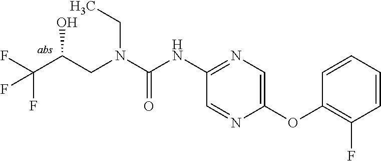 [SMILES: CCN(C[C@@H](O)C(F)(F)F)C(=O)Nc1cnc(Oc2ccccc2F)cn1] | 8.07 |
| E249 | 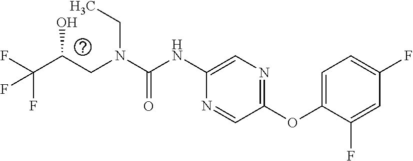 [SMILES: CCN(C[C@@H](O)C(F)(F)F)C(=O)Nc1cnc(Oc2ccc(F)cc2F)cn1] | 8.79 |
| E250 | 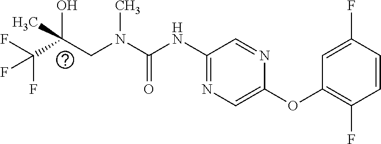 [SMILES: CN(C[C@@](C)(O)C(F)(F)F)C(=O)Nc1cnc(Oc2cc(F)ccc2F)cn1] | 8.59 |
| E251 | 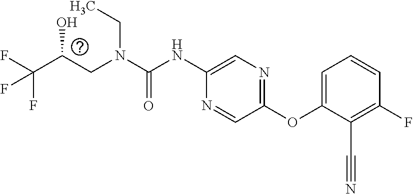 [SMILES: CCN(C[C@@H](O)C(F)(F)F)C(=O)Nc1cnc(Oc2cccc(F)c2C#N)cn1] | 7.57 |
| E252 | 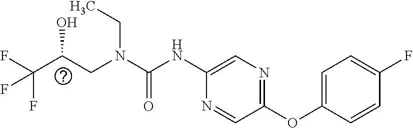 [SMILES: CCN(C[C@@H](O)C(F)(F)F)C(=O)Nc1cnc(Oc2ccc(F)cc2)cn1] | 6.23 |
| E253 | 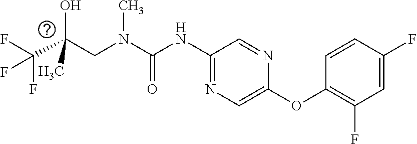 [SMILES: CN(C[C@@](C)(O)C(F)(F)F)C(=O)Nc1cnc(Oc2ccc(F)cc2F)cn1] | 8.41 |
| E254 | 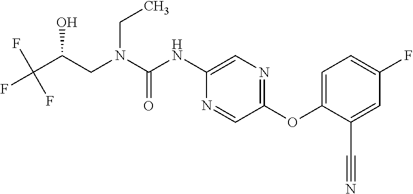 [SMILES: CCN(C[C@@H](O)C(F)(F)F)C(=O)Nc1cnc(Oc2ccc(F)cc2C#N)cn1] | 7.51 |
| E255 | 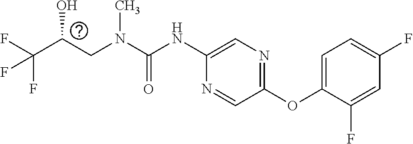 [SMILES: CN(C[C@@H](O)C(F)(F)F)C(=O)Nc1cnc(Oc2ccc(F)cc2F)cn1] | 7.88 |
| E256 | 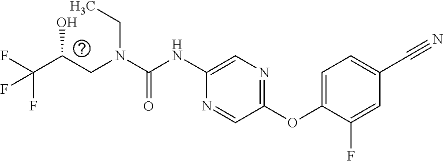 [SMILES: CCN(C[C@@H](O)C(F)(F)F)C(=O)Nc1cnc(Oc2ccc(C#N)cc2F)cn1] | 6.46 |
| E257 | 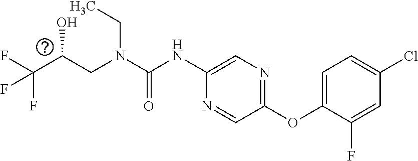 [SMILES: CCN(C[C@@H](O)C(F)(F)F)C(=O)Nc1cnc(Oc2ccc(Cl)cc2F)cn1] | 7.83 |
| E258 | 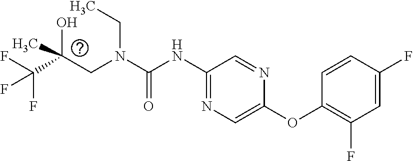 [SMILES: CCN(C[C@@](C)(O)C(F)(F)F)C(=O)Nc1cnc(Oc2ccc(F)cc2F)cn1] | 8.81 |
| E259 | 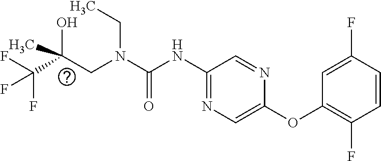 [SMILES: CCN(C[C@@](C)(O)C(F)(F)F)C(=O)Nc1cnc(Oc2cc(F)ccc2F)cn1] | 8.77 |
| E260 | 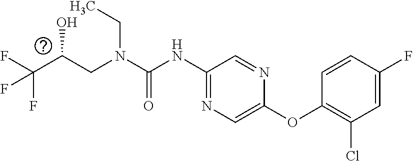 [SMILES: CCN(C[C@@H](O)C(F)(F)F)C(=O)Nc1cnc(Oc2ccc(F)cc2Cl)cn1] | 8.39 |
| E261 | 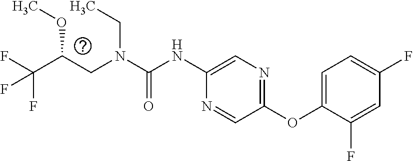 [SMILES: CCN(C[C@@H](OC)C(F)(F)F)C(=O)Nc1cnc(Oc2ccc(F)cc2F)cn1] | 6.42 |
| E262 | 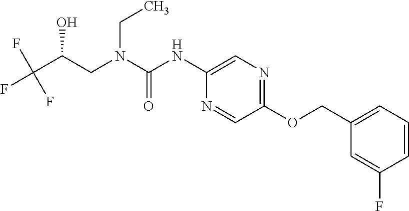 [SMILES: CCN(C[C@@H](O)C(F)(F)F)C(=O)Nc1cnc(OCc2cccc(F)c2)cn1] | 6.17 |
| E263 | 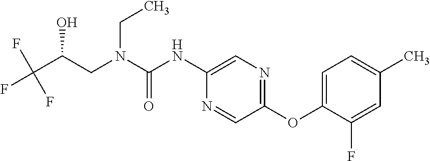 [SMILES: CCN(C[C@@H](O)C(F)(F)F)C(=O)Nc1cnc(Oc2ccc(C)cc2F)cn1] | 7.61 |
| E264 | 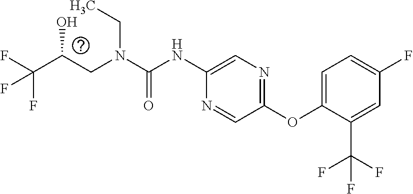 [SMILES: CCN(C[C@@H](O)C(F)(F)F)C(=O)Nc1cnc(Oc2ccc(F)cc2C(F)(F)F)cn1] | 6.53 |
| E265 | 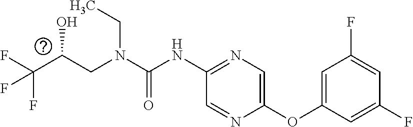 [SMILES: CCN(C[C@@H](O)C(F)(F)F)C(=O)Nc1cnc(Oc2cc(F)cc(F)c2)cn1] | 6.17 |
| E266 | 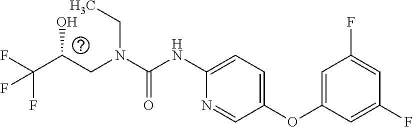 [SMILES: CCN(C[C@@H](O)C(F)(F)F)C(=O)Nc1ccc(Oc2cc(F)cc(F)c2)cn1] | 8.62 |
| E267 | 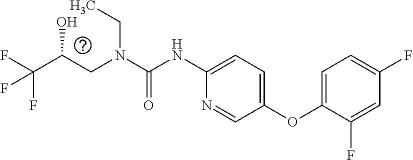 [SMILES: CCN(C[C@@H](O)C(F)(F)F)C(=O)Nc1ccc(Oc2ccc(F)cc2F)cn1] | 7.55 |
| E268 | 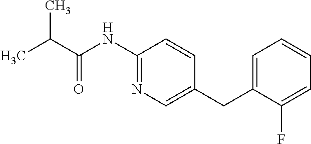 [SMILES: CC(C)C(=O)Nc1ccc(Cc2ccccc2F)cn1] | 6.3 |
| E269 | 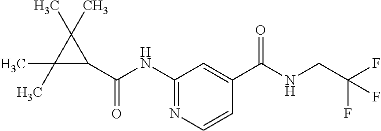 [SMILES: CC1(C)C(C(=O)Nc2cc(C(=O)NCC(F)(F)F)ccn2)C1(C)C] | 5.5 |
| E270 | 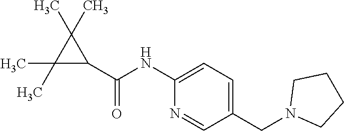 [SMILES: CC1(C)C(C(=O)Nc2ccc(CN3CCCC3)cn2)C1(C)C] | 5.28 |
| E271 | 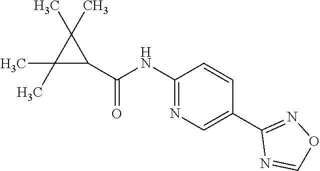 [SMILES: CC1(C)C(C(=O)Nc2ccc(-c3ncon3)cn2)C1(C)C] | 7.5 |
| E272 | 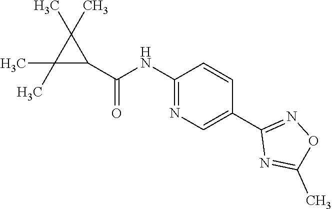 [SMILES: Cc1nc(-c2ccc(NC(=O)C3C(C)(C)C3(C)C)nc2)no1] | 8.07 |
| E273 | 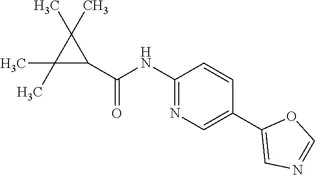 [SMILES: CC1(C)C(C(=O)Nc2ccc(-c3cnco3)cn2)C1(C)C] | 6.18 |
| E274 | 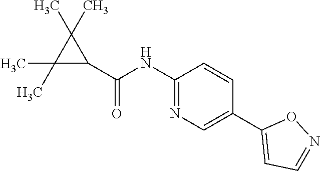 [SMILES: CC1(C)C(C(=O)Nc2ccc(-c3ccno3)cn2)C1(C)C] | 7.69 |
| E275 | 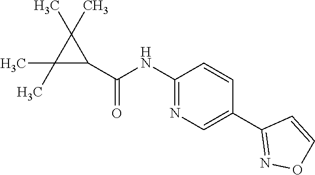 [SMILES: CC1(C)C(C(=O)Nc2ccc(-c3ccon3)cn2)C1(C)C] | 7.91 |
| E276 | 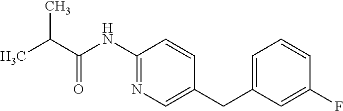 [SMILES: CC(C)C(=O)Nc1ccc(Cc2cccc(F)c2)cn1] | 6.3 |
| E277 | 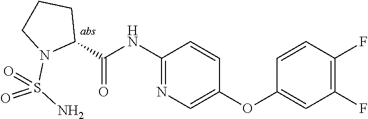 [SMILES: NS(=O)(=O)N1CCC[C@@H]1C(=O)Nc1ccc(Oc2ccc(F)c(F)c2)cn1] | 5.72 |
| E278 | 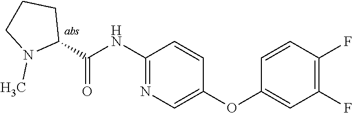 [SMILES: CN1CCC[C@@H]1C(=O)Nc1ccc(Oc2ccc(F)c(F)c2)cn1] | 5.58 |
| E279 | 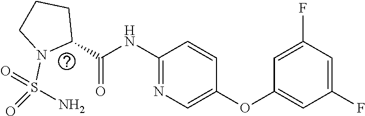 [SMILES: NS(=O)(=O)N1CCC[C@@H]1C(=O)Nc1ccc(Oc2cc(F)cc(F)c2)cn1] | 6.62 |
| E280 | 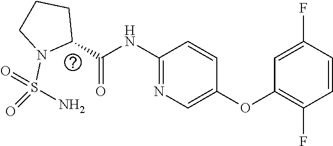 [SMILES: NS(=O)(=O)N1CCC[C@@H]1C(=O)Nc1ccc(Oc2cc(F)ccc2F)cn1] | 6.23 |
| E281 | 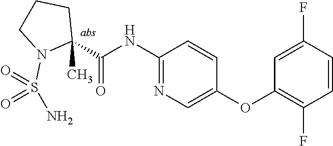 [SMILES: C[C@]1(C(=O)Nc2ccc(Oc3cc(F)ccc3F)cn2)CCCN1S(N)(=O)=O] | 7.09 |
| E282 | 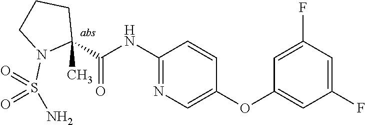 [SMILES: C[C@]1(C(=O)Nc2ccc(Oc3cc(F)cc(F)c3)cn2)CCCN1S(N)(=O)=O] | 7.56 |
| E283 | 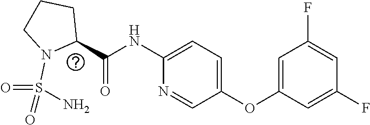 [SMILES: NS(=O)(=O)N1CCC[C@H]1C(=O)Nc1ccc(Oc2cc(F)cc(F)c2)cn1] | 6.25 |
| E284 | 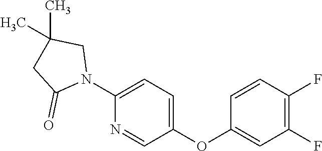 [SMILES: CC1(C)CC(=O)N(c2ccc(Oc3ccc(F)c(F)c3)cn2)C1] | 5.75 |
| E285 | 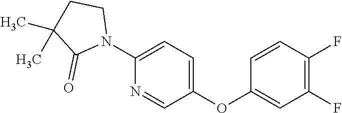 [SMILES: CC1(C)CCN(c2ccc(Oc3ccc(F)c(F)c3)cn2)C1=O] | 6.59 |
| E286 | 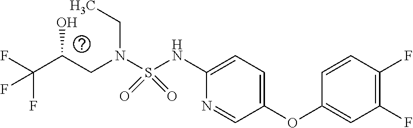 [SMILES: CCN(C[C@@H](O)C(F)(F)F)S(=O)(=O)Nc1ccc(Oc2ccc(F)c(F)c2)cn1] | 5.64 |
| E287 | 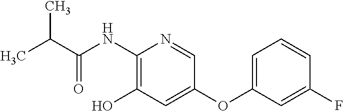 [SMILES: CC(C)C(=O)Nc1ncc(Oc2cccc(F)c2)cc1O] | 6.1 |
| E288 | 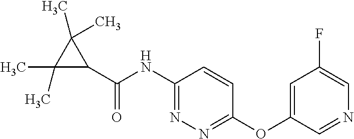 [SMILES: CC1(C)C(C(=O)Nc2ccc(Oc3cncc(F)c3)nn2)C1(C)C] | 5.04 |
| E289 | | 6.14 |
| E290 | 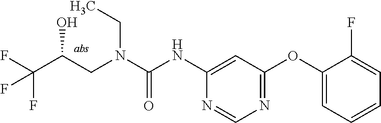 [SMILES: CCN(C[C@@H](O)C(F)(F)F)C(=O)Nc1cc(Oc2ccccc2F)ncn1] | 6.16 |
| E291 | 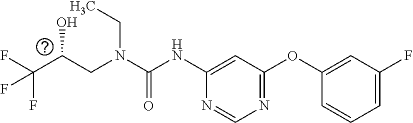 [SMILES: CCN(C[C@@H](O)C(F)(F)F)C(=O)Nc1cc(Oc2cccc(F)c2)ncn1] | 6.05 |

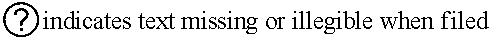

What is claimed is:

1. A compound having the following Formula I: 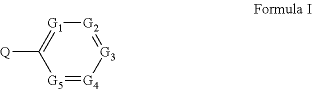
wherein:
Q is 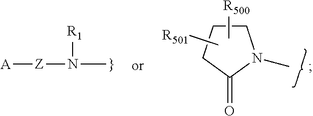
Z is —C(═O)—(CR₂₀R₂₁)ₙ or —S(═O)₂—;
R₁ is H or C₁₋₃ alkyl;
n is 0 or 1;
each R₂₀ and R₂₁ is independently H or C₁₋₃ alkyl;
G₁, G₂, G₃, G₄ and G₅ are each independently N or —C—L₁—M₁, provided that at least one of G₁, G₂, G₃, G₄ and G₅ is N;
each L₁ is independently a bond, O, —C(═O), —C(═O)—NH—, —CH₂—, —O—(CH₂)_{w}— where w is 1, 2 or 3, or —N(R₉₀)—; or any two L₁—M groups on adjacent carbon atoms can together form a group of formula —O—(CH₂)ᵥ—O— where v is 1 or 2;
each R₉₀ is independently H or C₁₋₃ alkyl;
each M₁is independently H, —OH, halogen, cyano, C₆₋₁₀ aryl; 5-10 member heteroaryl having 1-3 ring heteroatoms independently selected from N, O and S; C₁₋₆ alkyl; C₃₋₆ cycloalkyl; —NR₅₀R₅₁; 4-10 member heterocycloalkyl having 1-3 ring heteroatoms independently selected from N, O and S; wherein each of the C₆₋₁₀ aryl; 5-10 member heteroaryl; C₁₋₆ alkyl; C₃₋₆ cycloalkyl and 4-10 member heterocycloalkyl is each optionally substituted with 1, 2 or 3 substituents independently selected from the group consisting of halogen, cyano, —OH, C₁₋₃ alkyl, C₁₋₃ haloalkyl, C₁₋₃ alkoxy, and —C(═O)—N(R₉₁)(R₉₂);
each R₉₁ and R₉₂ is independently selected from the group consisting of H and C₁₋₃ alkyl;
each R₅₀ and R₅₁ is independently selected from the group consisting of H, C₁₋₃ alkyl, and C₆₋₁₀ aryl;
A is —L₂—M2;
L₂ is selected from a bond and —(CR₆ₒR₆₁)ₖ—;
R₆₀ and R₆₁are each independently H or C₁₋₃ alkyl optionally substituted with 1, 2 or 3 substituents independently selected from —OH and halogen;
k is 1, 2 or 3;
M₂ is C₁₋₆ alkyl; C₃₋₆ cycloalkyl; C₅₋₁₀ spiroalkyl; 4-10 member heterocycloalkyl having 1-3 ring heteroatoms independently selected from N, O and S; —N(R₈₁)(R₈₂); and C₆₋₁₀ aryl; wherein each C₁₋₆ alkyl, C₃₋₆ cycloalkyl, C₆₋₁₀ spiroalkyl, 5-10 member heteroaryl, 4-10 member heterocycloalkyl, and C₆₋₁₀ aryl is each optionally substituted with 1, 2, 3 or 4 independently selected R₂₀₀ groups;
each R₂₀₀ is independently selected from C₁₋₆ alkyl; C₁₋₆ hydroxyalkyl; C₃₋₆ cycloalkyl; 5-10 member heterocycloalkyl having 1-3 ring heteroatoms independently selected from N, O and S; C₁₋₆ mono-, di- or trihaloalkyl; halogen; cyano; —OH; C₁₋₆ alkoxy; —S(═O)₂NR₅₀₂R₅₀₃ and C₆₋₁₀ aryl;
each R₈₁ and R₈₂ is independently selected from H; C₁₋₆ alkyl and C₃₋₆ cycloalkyl; wherein the C₁₋₆ alkyl and C₃₋₆ cycloalkyl are optionally substituted with 1, 2, 3, or 4 substituents independently selected from —OH and halogen;
R₅₀₀ and R₅₀₁ are independently absent or C₁₋₆ alkyl;
R₅₀₂ and R₅₀₃ are independently H or C₁₋₆ alkyl;
or a stereoisomer, solvates, tautomers, or pharmaceutically acceptable salts thereof.

2. The compound of claim 1, wherein G₁ is N.

3. The compound of claim 1, wherein G₂ is N

4. The compound of claim 1, wherein G₁ and G₄ are N.

5. The compound of claim 1, wherein G₁ and G₂ are N.

6. The compound of claim 1, wherein G₁ and G₅ are N.

7. The compound of claim 1, wherein G₃ is —C—L₁—M₁.

8. The compound of claim 1, wherein G₄ is —C—L₁—M₁.

9. The compound of claim 1, wherein G₂ is —C—L₁—M₁.

10. The compound of any of claims 1-9, wherein L₁ is O.

11. The compound of any of claims 1-9, wherein L₁ is —CH₂—.

12. The compound of any of claims 1-9, wherein L₁ is a bond.

13. The compound of any of claims 1-9, wherein L₁ is —C(═O).

14. The compound of any of claims 1-9, wherein L₁ is —C(═O)—NH—.

15. The compound of any of claims 1-9, wherein L₁ is —N(R₉₀)—.

16. The compound of any preceding claim, wherein R₁ is H.

17. The compound of any preceding claim 1, wherein n is 0.

18. The compound of any of claims 1-15, wherein n is 1.

19. The compound of any preceding claim, wherein M₁is selected from C₆ aryl; C₆ heteroaryl having 1 or 2 ring heteroatoms independently selected from N and O, C₅ or C₆ heterocycloalkyl having 1 or 2 ring heteroatoms independently selected from N and O, and a 5-10 member heterocycloalkyl having 1-3 ring heteroatoms independently selected from N, O and S; each of the foregoing of which is optionally substituted.

20. The compound of any preceding claim, wherein M₁is phenyl, pyridyl, pyrrolidine, pyridazine, tetrahydrofuran, tetrahydropyran or dihydroindiole, each of which is optionally substituted.

21. The compound of any preceding claim, wherein the substituents for M₁are independently selected from halogen; CN; —OH; —C(═O)—NH₂; CF₃; and —OCH₃.

22. The compound any preceding claim, wherein M₁is optionally substituted phenyl.

23. The compound of any of claims 1-21, wherein the phenyl is substituted in the 4-position.

24. The compound of any of claims 1-21, wherein the phenyl is substituted in the 3-position and the 4-position.

25. The compound of any of claims 1-21, wherein the phenyl is substituted in the 3-position and the 5-position.

26. The compound of any preceding claim, wherein M₁is optionally substituted pyridyl.

27. The compound of any preceding claim, wherein M₁is optionally substituted pyridyl-4-yl.

28. The compound of any preceding claim, wherein M₁is optionally substituted pyridyl-3-yl.

29. The compound of any preceding claim, wherein the pyridyl is substituted at a carbon ortho (i.e., adjacent) to the pyridyl nitrogen.

30. The compound of any preceding claim, wherein the pyridyl is substituted at a carbon meta to the pyridyl nitrogen;

31. The compound of any of claims 1-29, wherein the pyridyl is substituted at a carbon meta to the pyridyl nitrogen.

32. The compound of any preceding claim, wherein M₁is optionally substituted heterocycloalkyl.

33. The compound of any preceding claim, wherein M₁is optionally substituted pyrrolidinyl.

34. The compound of any of claims 1-18, wherein M₁is optionally substituted pyrrolidine-1-yl.

35. The compound of any of claims 1-18, wherein M₁is optionally substituted tetrahydropyranyl.

36. The compound of any of claims 1-18, wherein M₁is optionally substituted tetrahydropyran-4-yl.

37. The compound of any of claims 1-18, wherein M₁is optionally substituted cycloalkyl.

38. The compound of any of claims 1-18, wherein M₁is optionally substituted C₁₋₆ alkyl.

39. The compound of any of claims 1-18, wherein M₁is —NR₅₀R5i.

40. The compound of any preceding claim, wherein L₂ is a bond.

41. The compound of any of claims 1-39, wherein L₂ is —(CR₆₀R₆₁)ₖ—.

42. The compound of any preceding claim, wherein M₂ is optionally substituted C₁₋₆ alkyl.

43. The compound of any preceding claim, wherein M₂ is optionally substituted isopropyl.

44. The compound of any of claims 1-41, wherein M₂ is optionally substituted C₃₋₆ cycloalkyl.

45. The compound of any of claims 1-41, wherein M₂ is cyclopropyl or cyclobutyl, each of which is optionally substituted with from 1 to 4 methyl groups.

46. The compound of any of claims 1-41, wherein M₂ is cyclopropyl or cyclobutyl, each of which is optionally substituted with 1 or 2 substituents independently selected from methyl, halogen, mono-, di- or trihalomethyl, cyano, hydroxymethyl and hydroxy.

47. The compound of any of claims 1-41, wherein M₂ is cyclopropyl optionally substituted with 1 to 4 methyl groups, halogen or trihalomethyl.

48. The compound of any of claims 1-41, wherein M₂ is heterocycloalkyl optionally substituted with 1 or 2 groups independently selected from methyl and hydroxy.

49. The compound of any of claims 1-41, wherein M₂ is tetrahydrofuran, pyrrolidine, tetrahydropyran or morpholine, each of which is optionally substituted with 1 or 2 groups independently selected from methyl and hydroxy.

50. The compound of any preceding claim, wherein M₂ is —N(R₈₁)(R₈₂).

51. The compound of any preceding claim, wherein R₈₁ and R₈₂ are independently selected from C₁₋₃ alkyl and C₃₋₄ cycloalkyl; each of which is optionally substituted with 1 or 2 substituents independently selected from —OH and halogen.

52. A compound according to any of the preceding claims, selected from the Compounds in Table 1 herein, or a stereoisomer, solvates, tautomers, or pharmaceutically acceptable salts thereof.

53. A composition comprising a dermatologically or orally acceptable excipient and a compound according to any preceding claim.

54. A method for treating an inflammatory disorder, the method comprising administering to a subject in need thereof a composition comprising a therapeutically effective amount of compound of claim 1, and a dermatologically or orally acceptable excipient.

55. The method of claim 54, wherein the composition is in the form of a cream, a gel, a spray, an ointment, or an oral unit dosage form.

56. The method of claim 54, wherein the MrgprX2 antagonist is present at a concentration of about 0.001 wt. % to about 10 wt. %, based on the total weight of the composition.

57. The method of claim 54, wherein the MrgprX2 antagonist is present at a concentration of about 0.1 wt. % to about 5 wt. %, based on the total weight of the composition.

58. The method of claim 54, wherein the composition further comprises a skin absorption enhancer.

59. The method of claim 54, wherein the composition further comprises a skin absorption enhancer comprising one or more of mannitol, sulphoxides (e.g., dimethylsulphoxide, DMSO), Azones (e.g. laurocapram), pyrrolidones (e.g., 2-pyrrolidone, 2P), alcohols and alkanols (e.g., ethanol, or decanol), glycols (e.g., propylene glycol, hexylene glycol, polyoxyethylene glycol, diethylene glycol), surfactants (also common in dosage forms) and terpenes.

60. The method of any of claims 54-59, wherein the composition is applied to a patient's skin once daily.

61. The method of any of claims 54-59, wherein the composition is applied to a patient's skin twice daily.

62. The method of any of claims 54-59, wherein the composition is applied to a patient's skin three times daily.

63. The method of any of claims 54-62, wherein the composition is administered to a patient suffering from an inflammatory disorder.

64. The method of any of claims 54-63, wherein the inflammatory disorder is a disorder of the skin.

65. The method of any of claims 54-64, wherein the skin is human skin.

66. The method of any of claims 63-65 wherein the inflammatory disorder activates or is consequent to activation, of MrgprX2.

67. The method of any of claims 63-66, wherein the inflammatory disorder is atopic dermatitis (e.g., Asian atopic dermatitis, European atopic dermatitis), chronic urticaria, pseudo-allergic reactions triggered by small molecules for example anaphylactoid drug reactions, anaphylactic shock, rosacea, asthma, systemic itch such as cholestatic or uremic itch, chronic itch triggered by systemic diseases, or drug-adverse reactions.

68. The method of any of claims 63-67, wherein the inflammatory disorder is atopic dermatitis (e.g., Asian atopic dermatitis, European atopic dermatitis).

69. The method of any of claims 54-68, wherein the subject is a human.

70. The method of any of claims 54-68, wherein the mammalian skin is human skin.

71. The method of any preceding claim, wherein the composition is for oral administration.
